(19) United States
(12) Patent Application Publication
(10) Pub. No.: US 2011/0010792 A1
(43) Pub. Date: Jan. 13, 2011

(54) PLANT POLYNUCLEOTIDES FOR IMPROVED YIELD AND QUALITY

(75) Inventors: James Zhang, Palo Alto, CA (US); Frederick D. Hempel, Sunol, CA (US)

Correspondence Address: Mendel Biotechnology, Inc., 3935 Point Eden Way, Hayward, CA, 94545, US

(73) Assignee: Mendel Biotechnology, Inc., Hayward, CA (US)

(21) Appl. No.: 12/885,749

(22) Filed: Sep. 20, 2010

Related U.S. Application Data
(62) Division of application No. 11/632,390, filed on Dec. 17, 2008, now abandoned.
(60) Provisional application No. 60/588,405, filed on Jul. 14, 2004.

(51) Int. Cl. A01H 1/02; A01H 5/00; C12N 15/82

(52) U.S. Cl. 800/265; 800/301; 800/298; 800/317.4; 800/279; 800/290; 800/282; 800/278; 800/287; 800/260

(57) ABSTRACT

The invention relates to plant transcription factor polypeptides, polynucleotides that encode them, homologs from a variety of plant species, and methods of using the polynucleotides and polypeptides to produce transgenic plants having advantageous properties, including increased soluble solids, lycopene, and improved plant volume or yield, as compared to wild-type or control plants. The invention also pertains to expression systems that may be used to regulate these transcription factor polynucleotides, providing constitutive, transient, inducible and tissue-specific regulation.

### RELATIONSHIP TO COPENDING APPLICATIONS

This application is a divisional application of U.S. application Ser. No. 11/632,390 (pending), filed on Dec. 17, 2008, which is the National Stage of the International Application PCT/US05/025010, filed on Jul. 14, 2005, which claims the benefit of U.S. provisional application 60/588,405, filed Jul. 14, 2004 (expired). The entire contents of each of these applications are hereby incorporated by reference.

### FIELD OF THE INVENTION

The present invention relates to compositions and methods for transforming plants for the purpose of improving plant traits, including yield and fruit quality.

### BACKGROUND OF THE INVENTION

### Biotechnological Improvement of Plants

To date, almost all improvements in agricultural crops have been achieved using traditional plant breeding techniques. These techniques involve crossing parental plants with different genetic backgrounds to generate progeny with genetic diversity, which are then selected to obtain those plants that express the desired traits. The desired traits are then fixed and deleterious traits eliminated via multiple backcrossings or selfings to eventually yield progeny with the desired characteristics. Hybrid corn, low erucic acid oilseed rape, high oil corn, and hard white winter wheat are examples of significant agricultural advances achieved with traditional breeding. However, the amount of genetic diversity in the germplasm of a particular crop limits what can be accomplished by breeding. Although traditional breeding has proven to be very powerful, as advances in crop yields over the last century demonstrate, recent data suggest that the rate of yield improvement is tapering off for major food crops (Lee (1998)). The introduction of molecular mapping markers into breeding programs may accelerate the process of crop improvement in the near term, but ultimately the lack of new sources of genetic diversity will become limiting. Additionally, traditional breeding has proved rather ineffective for improving many polygenic traits such as increased disease resistance.

In recent years, biotechnology approaches involving the expression of single transgenes in crops have resulted in the successful commercial introduction of new plant traits, including herbicide resistance (glyphosate (Roundup) resistance), insect resistance (expression of Bacillus thuringiensis toxins) and virus resistance (over expression of viral coat proteins). However, the list of single gene traits of significant value is relatively small. The greatest potential of biotechnology lies in engineering complex polygenic traits to fundamentally change plant physiology and biochemistry. Step change improvements in crop yields, nutritional quality, plant architecture and resistance to environmental stresses are expected using genetic engineering approaches. Engineering polygenic traits has proven extremely challenging. As a result, companies have turned to plant genomics to achieve control over polygenic traits.

In general most agricultural biotechnology research programs being presently conducted involve large-scale expressed sequence tag projects (EST sequencing), gene expression profiling, quantitative trait loci mapping (QTL mapping), and/or positional cloning of quantitative trait loci. Presently, only a few research programs are engaged in functional genomics programs that analyze the effects of gene over-expression and null mutants, particularly the systematical identification and functional characterization of plant transcription factors.

Increased lycopene levels. Lycopene is a pigment responsible for color of fruits (e.g., the red color of tomatoes). For most consumers an attractive, bright color is the most important component to a fruit's visual appeal. The initial decision to purchase a fruit product is most often based on color, with taste influencing follow-on purchase decisions. There are immediate aesthetic benefits to robust color in fruit. Consumers in the U.S. and elsewhere have a clear preference for fruit products with good color, and often specifically buy fruit and fruit products based on lycopene levels.

In addition to being responsible for color, lycopene, and other carotenoids are valuable anti-oxidants in the diet. Lycopene is the subject of an increasing number of medical studies that demonstrate its efficacy in preventing certain cancers—including prostate, lung, stomach and breast cancers. Potential impacts also include ultraviolet protection and coronary heard disease prevention.

Increased soluble solids. Increased soluble solids are highly valuable to fruit processors for the production of various products. Grapes, for example, are harvested when soluble solids have reached an appropriate level, and the quality of wine produced from grapes is to a large extent dependent on soluble solid content.

Increased soluble solids are also of considerable importance in the production of tomato paste, sauces and ketchup. Tomato paste is sold on the basis of soluble solids. Increasing soluble solids in tomatoes increases the value of processed tomato products and decreases processing costs. Savings come from reduced processing time and less energy consumption due to shortened cooking times needed to achieve desired soluble solids levels. A one percent increase in tomato soluble solids may be worth $100 to $200 million to the tomato processing industry.

Disease Resistance. Fungal diseases are a perpetual problem in agriculture. Fungal diseases reduce yields, increase input costs for producers and lead to increased post-harvest spoilage of fruits and vegetables. Significant post-harvest losses occur due to fruit rot caused by the fungal disease, Botrytis. A disease resistant tomato, for example, would reduce these losses, thus lowering consumer prices and increasing overall profitability in the industry. Additionally, reducing post-harvest spoilage could extend the possible shipping range, thereby allowing access to new export markets.

Improvements that May not be Achievable with Traditional Breeding Methods

Most agronomic and quality traits are polygenic, which means many genes control them. Polygenic traits are extremely difficult to manipulate by traditional breeding or current single gene genetic engineering approaches. Difficulties in manipulating polygenic traits include:
obtaining all the genes necessary in a single variety,
linkage between genes for the desired trait and nearby deleterious traits,
lack of sufficient diversity in the germplasm (the collection of plant genetic material that can be selected and combined by traditional breeding techniques) to allow introduction of the desired polygenic trait by traditional breeding techniques.

For example, high solid tomato varieties have been obtained by breeding, but they are commercially unacceptable because the genes that control solids content are tightly linked to genes that also cause reduced yields and poor viscosity, consistency, and firmness.

Traditional biotechnology approaches have failed to improve these traits, since complex polygenic control requires insertion of multiple genes. These techniques also suffered difficulties caused by complex feedback mechanisms and multiple rate-limiting steps in the pathways.

Control of Cellular Processes in Plants with Transcription Factors

Multiple cellular processes in plants are controlled to a significant extent by transcription factors, proteins that influence the expression of a particular gene or sets of genes. Transcription factors can modulate gene expression, either increasing or decreasing (inducing or repressing) the rate of transcription. This modulation results in differential levels of gene expression at various developmental stages, in different tissues and cell types, and in response to different exogenous (e.g., environmental) and endogenous stimuli throughout the life cycle of the organism. Because transcription factors are key controlling elements of biological pathways, altering the levels of at least one selected transcription factor in transformed and transgenic plants can change entire biological pathways in an organism, conferring advantageous or desirable traits. For example, overexpression of a transcription factor gene can be brought about when, for example, the genes encoding one or more transcription factors is placed under the control of a strong expression signal, such as the constitutive cauliflower mosaic virus 35S transcription initiation region (henceforth referred to as the 35S promoter). Conversely, various means exist to reduce the level of expression of a transcription factor, including gene silencing or knocking out a gene with a site-specific insertion.

Strategies for manipulating traits by altering a plant cell's transcription factor content can result in plants and crops with new and/or improved commercially valuable properties. For example, manipulation of the levels of selected transcription factors may result in increased expression of economically useful proteins or biomolecules in plants or improvement in other agriculturally relevant characteristics. Conversely, blocked or reduced expression of a transcription factor may reduce biosynthesis of unwanted compounds or remove an undesirable trait. Therefore, manipulating transcription factor levels in a plant offers tremendous potential in agricultural biotechnology for modifying a plant's traits, including traits that improve a plant's survival, yield and product quality.

Plant transcription factors are regulatory proteins, and therefore critical “switches” that control complex, polygenic pathways. Controlling the expression level of plant transcription factors represents a critical, yet previously difficult, approach to manipulating plant traits. In order to control transcription factor levels in plants, a “Plant Transcription Factor Tool Kit” (PTF Tool Kit) has been developed that makes it possible to investigate readily phenotypic effects due to the expression of specific plant transcription factors at different levels, at different stages of development, under different types of stress, and in different plant tissues. This capability may be made available to plant breeders merely by making specific crosses in a “combinatorial-like” manner between two sets of plants: one set genetically engineered to contain transcription factors and a second set engineered to contain specific promoters. Our “Two-Component Multiplication System” expresses the transcription factor under control of the engineered promoter in the progeny plant, providing the same effect as if each plant had been engineered with the specific gene-promoter combination. A plant “library” comprising tens of thousands of plant transcription factor-promoter combinations can therefore be investigated with minimal time and expense. The PTF Tool Kit technology can be used with a wide range of other commercially important fruit, vegetable and row crops. This innovative technology is expected to increase agricultural productivity, improve the quality of agricultural products, and translate directly into higher profits for farmers and agricultural processors, as well as benefiting consumers.

The sizable fraction of the 1,800 plant transcription factor genes found in Arabidopsis thaliana have been investigated using the PTF Tool Kit, and their utility in an active breeding program is presented herein.

### SUMMARY OF THE INVENTION

The present invention relates to compositions and methods for modifying the genotype of a higher plant for the purpose of impart desirable characteristics. These characteristics are generally yield and/or quality-related, and may specifically pertain to the fruit of the plant. The method steps involve first transforming a host plant cell with a DNA construct (such as an expression vector or a plasmid); the DNA construct comprises a polynucleotide that encodes a transcription factor polypeptide, and the polynucleotide is homologous to any of the polynucleotides of the invention. These include the transcription factor polynucleotides found in the Sequence Listing, and related sequences, such as:

(a) a nucleotide sequence encoding SEQ ID NO: 2N, where N=1 to 201 or 413 to 419, or a complementary nucleotide sequence;

(b) a nucleotide sequence comprising SEQ ID NO: 2N=1, where N=1 to 201 or 413 to 419, or SEQ ID NO: 403-824, or a complementary nucleotide sequence;

(c) a nucleotide sequence that hybridizes under stringent conditions to nucleotide sequence of either (a) or (b),

(d) a nucleotide sequence that comprises a subsequence or fragment of any of the nucleotide sequences of (a), (b) or (c), the subsequence or fragment encoding a polypeptide that imparts the desired characteristic to the fruit of the higher plant; or

(e) a nucleotide sequence encoding a polypeptide having a conserved domain with at least 80% sequence identity to a conserved domain of SEQ ID NO: 2N, where N=1 to 201 or 413 to 419.

Once the host plant cell is transformed with the DNA construct, a plant may be regenerated from the transformed host plant cell. This plant may then be grown to produce a plant having the desired yield or quality characteristic. Examples of yield characteristics that may be improved by these method steps include increased fungal disease tolerance, increased fruit weight, increased fruit number, and increased plant size. Examples of quality characteristics that may be improved by these method steps include increased fungal disease tolerance, increased lycopene levels, reduced fruit softening, and increased soluble solids.

### BRIEF DESCRIPTION OF THE SEQUENCE LISTING AND FIGURES

The Sequence Listing provides exemplary polynucleotide and polypeptide sequences of the invention. The traits associated with the use of the sequences are included in the Examples.

Incorporation of the Sequence Listing. The copy of the Sequence Listing, being submitted electronically with this patent application, provided under 37 CFR §1.821-1.825, is a read-only memory computer-readable file in ASCII text format. The Sequence Listing is named “MBI0060US DIV1_ST25.txt”, the electronic file of the Sequence Listing was created on Sep. 17, 2010, and is 1,253 kilobytes in size as measured in MS-WINDOWS. The Sequence Listing is hereby incorporated by reference in their entirety.

FIG. 1 shows a conservative estimate of phylogenetic relationships among the orders of flowering plants (modified from Angiosperm Phylogeny Group (1998)). Those plants with a single cotyledon (monocots) are a monophyletic clade nested within at least two major lineages of dicots; the eudicots are further divided into rosids and asterids. Arabidopsis is a rosid eudicot classified within the order Brassicales; rice is a member of the monocot order Poales. FIG. 1 was adapted from Daly et al. (2001).

FIG. 2 shows a phylogenic dendogram depicting phylogenetic relationships of higher plant taxa, including clades containing tomato and Arabidopsis; adapted from Ku et al. (2000) and Chase et al. (1993).

FIG. 3 is a schematic diagram of activator and target vectors used for transformation of tomato to achieve regulated expression of 1700 Arabidopsis transcription factors in tomato. The activator vector contained a promoter and a LexA/GAL4 or a-LacI/GAL4 transactivator (the transactivator comprises a LexA or LacI DNA binding domain fused to the GAL4 activation domain, and encodes a LexA or LacI transcriptional activator product), a GUS marker, and a neomycin phosphotransferase II (nptII) selectable marker. The target vector contains a transactivator binding site operably linked to a transgene encoding a polypeptide of interest (for example, a transcription factor of the invention), and a sulfonamide selectable marker (in this case, sulII; which encodes the dihydropteroate synthase enzyme for sulfonamide-resistance) useful in the selection for and identification of transformed plants. Binding of the transcriptional activator product encoded by the activator vector to the transactivator binding sites of the target vector initiates transcription of the transgenes of interest.

### DESCRIPTION OF THE INVENTION

In an important aspect, the present invention relates to combinations of gene promoters and polynucleotides for modifying phenotypes of plants, including those associated with improved plant or fruit yield, or improved fruit quality. Throughout this disclosure, various information sources are referred to and/or are specifically incorporated. The information sources include scientific journal articles, patent documents, textbooks, and World Wide Web browser-active and inactive page addresses, for example. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a,” “an,” and “the” include plural reference unless the context clearly dictates otherwise. Thus, for example, a reference to “a plant” includes a plurality of such plants.

### DEFINITIONS

“Nucleic acid molecule” refers to an oligonucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA).

“Polynucleotide” is a nucleic acid molecule comprising a plurality of polymerized nucleotides, e.g., at least about 15 consecutive polymerized nucleotides, optionally at least about 30 consecutive nucleotides, at least about 50 consecutive nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, e.g., genomic DNA or RNA, a transcript (such as an mRNA), a cDNA, a polymerase chain reaction (PCR) product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single stranded.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as splicing and folding to obtain a functional protein or polypeptide. A gene may be isolated, partially isolated, or be found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and which may be used to determine the limits of the genetically active unit (Rieger et al. (1976)). A gene generally includes regions preceding (“leaders”; upstream) and following (“trailers”; downstream) of the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “polypeptide” is an amino acid sequence comprising a plurality of consecutive polymerized amino acid residues e.g., at least about 15 consecutive polymerized amino acid residues, optionally at least about 30 consecutive polymerized amino acid residues, at least about 50 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules which specifically bind to that portion or for the production of antibodies.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence. Additionally, the terms “homology” and “homologous sequence(s)” may refer to one or more polypeptide sequences that are modified by chemical or enzymatic means. The homologous sequence may be a sequence modified by lipids, sugars, peptides, organic or inorganic compounds, by the use of modified amino acids or the like. Protein modification techniques are illustrated in Ausubel et al. (1998).

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences.

With regard to polypeptides, the terms “substantial identity” or “substantially identical” may refer to sequences of sufficient similarity and structure to the transcription factors in the Sequence Listing to produce similar function when expressed, overexpressed, or knocked-out in a plant; in the present invention, this function is improved yield and/or fruit quality. Polypeptide sequences that are at least about 55% identical to the instant polypeptide sequences are considered to have “substantial identity” with the latter. Sequences having lesser degrees of identity but comparable biological activity are considered to be equivalents. The structure required to maintain proper functionality is related to the tertiary structure of the polypeptide. There are discreet domains and motifs within a transcription factor that must be present within the polypeptide to confer function and specificity. These specific structures are required so that interactive sequences will be properly oriented to retain the desired activity. “Substantial identity” may thus also be used with regard to subsequences, for example, motifs that are of sufficient structure and similarity, being at least about 55% identical to similar motifs in other related sequences. Thus, related polypeptides within the G1950 clade have the physical characteristics of substantial identity along their full length and within their AKR-related domains. These polypeptides also share functional characteristics, as the polypeptides within this clade bind to a transcription-regulating region of DNA and improve yield and/or fruit quality in a plant when the polypeptides are overexpressed.

“Alignment” refers to a number of nucleotide or amino acid residue sequences aligned by lengthwise comparison so that components in common (i.e., nucleotide bases or amino acid residues) may be visually and readily identified. The fraction or percentage of components in common is related to the homology or identity between the sequences. Alignments may be used to identify conserved domains and relatedness within these domains. An alignment may suitably be determined by means of computer programs known in the art, such as MacVector (1999) (Accelrys, Inc., San Diego, Calif.).

A “conserved domain” or “conserved region” as used herein refers to a region in heterologous polynucleotide or polypeptide sequences where there is substantial identity between the distinct sequences. bZIPT2-related domains are examples of conserved domains.

With respect to polynucleotides encoding presently disclosed transcription factors, a conserved domain is encoded by a sequence preferably at least 10 base pairs (bp) in length.

A “conserved domain”, with respect to presently disclosed polypeptides refers to a domain within a transcription factor family that exhibits a higher degree of sequence homology or substantial identity, such as at least about 55% identity, including conservative substitutions, and preferably at least 65% sequence identity, or at least about 70% sequence identity, or at least about 75% sequence identity, or at least about 77% sequence identity, and more preferably at least about 80% sequence identity, or at least 85%, or at least about 86%, or at least about 87%, or at least about 88%, or at least about 90%, or at least about 95%, or at least about 98% amino acid residue sequence identity to a sequence of consecutive amino acid residues.

A fragment or domain can be referred to as outside a conserved domain, outside a consensus sequence, or outside a consensus DNA-binding site that is known to exist or that exists for a particular transcription factor class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence. Thus, by using alignment methods well known in the art, the conserved domains of the plant transcription factors of the invention (e.g., bZIPT2, MYB-related, CCAAT-box binding, AP2, and AT-hook family transcription factors) may be determined. An alignment of any of the polypeptides of the invention with another polypeptide allows one of skill in the art to identify conserved domains for any of the polypeptides listed or referred to in this disclosure.

“Complementary” refers to the natural hydrogen bonding by base pairing between purines and pyrimidines. For example, the sequence A-CG-T (5′->3) forms hydrogen bonds with its complements AC-G-T (5′->3) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of the hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985), Sambrook et al. (1989), and by Hames and Higgins (1985), which references are incorporated herein by reference.

In general, stringency is determined by the temperature, ionic strength, and concentration of denaturing agents (e.g., formamide) used in a hybridization and washing procedure (for a more detailed description of establishing and determining stringency, see below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known transcription factor sequences. Numerous variations are possible in the conditions and means by which nucleic acid hybridization can be performed to isolate transcription factor sequences having similarity to transcription factor sequences known in the art and are not limited to those explicitly disclosed herein. Such an approach may be used to isolate polynucleotide sequences having various degrees of similarity with disclosed transcription factor sequences, such as, for example, transcription factors having 60% identity, or more preferably greater than about 70% identity, most preferably 72% or greater identity with disclosed transcription factors.

The terms “paralog” and “ortholog” are defined below in the section entitled “Orthologs and Paralogs”. In brief, orthologs and paralogs are evolutionarily related genes that have similar sequences and functions. Orthologs are structurally related genes in different species that are derived by a speciation event. Paralogs are structurally related genes within a single species that are derived by a duplication event.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) World Wide Web (www) website, “tigr.org” under the heading “Terms associated with TIGRFAMs”.

The term “variant”, as used herein, may refer to polynucleotides or polypeptides that differ from the presently disclosed polynucleotides or polypeptides, respectively, in sequence from each other, and as set forth below.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a transcription factor nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the terms refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. This, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent transcription factor. Thus, it will be readily appreciated by those of skill in the art, that any of a variety of polynucleotide sequences is capable of encoding the transcription factors and transcription factor homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as the functional or biological activity of the transcription factor is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine (for more detail on conservative substitutions, see Table 3). More rarely, a variant may have “non-conservative” changes, for example, replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (see U.S. Pat. No. 5,840,544).

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A polynucleotide fragment” refers to any subsequence of a polynucleotide, typically, of at least about 9 consecutive nucleotides, preferably at least about 30 nucleotides, more preferably at least about 50 nucleotides, of any of the sequences provided herein. Exemplary polynucleotide fragments are the first sixty consecutive nucleotides of the transcription factor polynucleotides listed in the Sequence Listing. Exemplary fragments also include fragments that comprise a region that encodes an conserved domain of a transcription factor. Exemplary fragments also include fragments that comprise a conserved domain of a transcription factor. Exemplary fragments include fragments that comprise a conserved domain of a transcription factor, for example, amino acids 135-195 of G1543, SEQ ID NO: 84, as noted in Table 1.

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as three amino acid residues to the full length of the intact polypeptide, but are preferably at least about 30 amino acid residues in length and more preferably at least about 60 amino acid residues in length.

The invention also encompasses production of DNA sequences that encode transcription factors and transcription factor derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding transcription factors or any fragment thereof.

“Derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

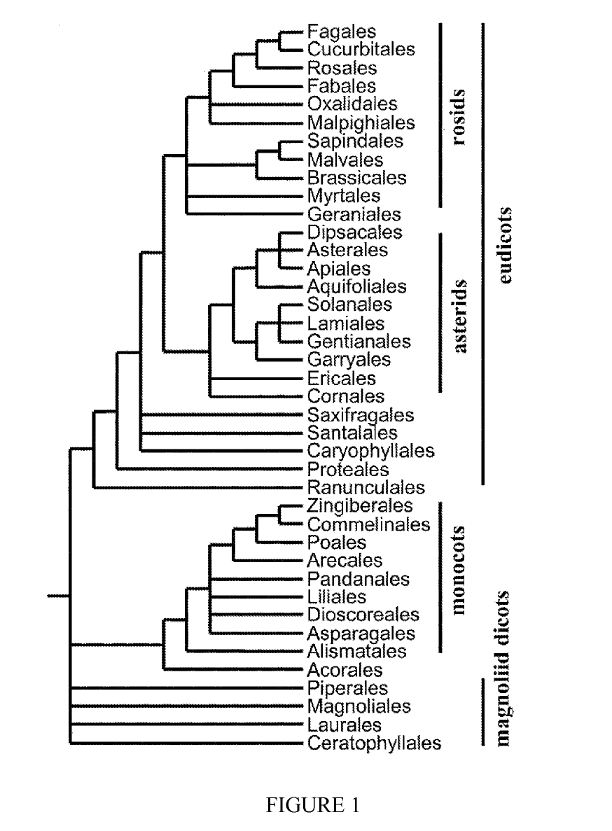

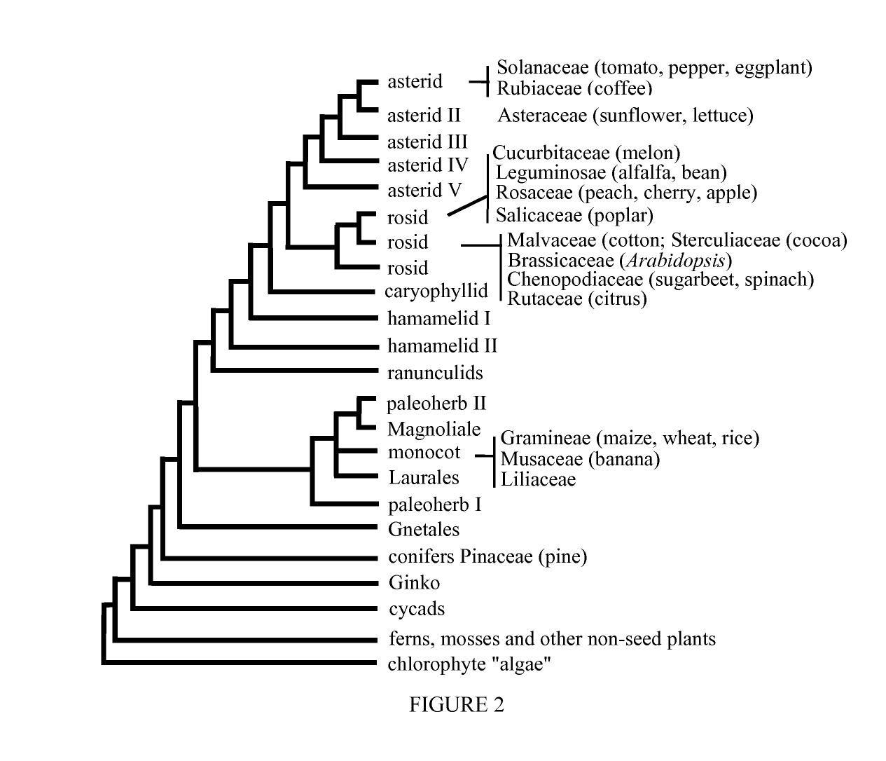

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (see for example, FIG. 1, adapted from Daly et al. (2001) Plant Physiol. 127: 1328-1333; FIG. 2, adapted from Ku et al. (2000) Proc. Natl. Acad. Sci. USA 97: 9121-9126; and see also Tudge in The Variety of Life, Oxford University Press, New York N.Y. (2000) pp. 547-606).

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence operably linked (i.e., under regulatory control of) to appropriate inducible or constitutive regulatory sequences that allow for the controlled expression of polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a transcription factor expression is altered, e.g., in that it has been knocked out, overexpressed, or ectopically expressed.

A “control plant” as used in the present invention refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as osmotic stress tolerance or yield. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease, or an even greater difference, in an observed trait as compared with a control or wild-type plant. It is known that there can be a natural variation in the modified trait. Therefore, the trait modification observed entails a change of the normal distribution and magnitude of the trait in the plants as compared to control or wild-type plants.

When two or more plants have “similar morphologies”, “substantially similar morphologies”, “a morphology that is substantially similar”, or are “morphologically similar”, the plants have comparable forms or appearances, including analogous features such as overall dimensions, height, width, mass, root mass, shape, glossiness, color, stem diameter, leaf size, leaf dimension, leaf density, internode distance, branching, root branching, number and form of inflorescences, and other macroscopic characteristics, and the individual plants are not readily distinguishable based on morphological characteristics alone.

“Modulates” refers to a change in activity (biological, chemical, or immunological) or lifespan resulting from specific binding between a molecule and either a nucleic acid molecule or a protein.

The term “transcript profile” refers to the expression levels of a set of genes in a cell in a particular state, particularly by comparison with the expression levels of that same set of genes in a cell of the same type in a reference state. For example, the transcript profile of a particular transcription factor in a suspension cell is the expression levels of a set of genes in a cell knocking out or overexpressing that transcription factor compared with the expression levels of that same set of genes in a suspension cell that has normal levels of that transcription factor. The transcript profile can be presented as a list of those genes whose expression level is significantly different between the two treatments, and the difference ratios. Differences and similarities between expression levels may also be evaluated and calculated using statistical and clustering methods.

“Ectopic expression or altered expression” in reference to a polynucleotide indicates that the pattern of expression in, e.g., a transgenic plant or plant tissue, is different from the expression pattern in a wild-type or control plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more transcription factors are under the control of a strong promoter (e.g., the cauliflower mosaic virus 35S transcription initiation region). Overexpression may also under the control of an inducible or tissue specific promoter. Thus, overexpression may occur throughout a plant, in specific tissues of the plant, or in the presence or absence of particular environmental signals, depending on the promoter used.

Overexpression may take place in plant cells normally lacking expression of polypeptides functionally equivalent or identical to the present transcription factors. Overexpression may also occur in plant cells where endogenous expression of the present transcription factors or functionally equivalent molecules normally occurs, but such normal expression is at a lower level. Overexpression thus results in a greater than normal production, or “overproduction” of the transcription factor in the plant, cell or tissue.

The term “transcription regulating region” refers to a DNA regulatory sequence that regulates expression of one or more genes in a plant when a transcription factor having one or more specific binding domains binds to the DNA regulatory sequence. Transcription factors of the present invention possess an AT-hook domain and a second conserved domain. Examples of similar AT-hook and second conserved domain of the sequences of the invention may be found in Table 1. The transcription factors of the invention also comprise an amino acid subsequence that forms a transcription activation domain that regulates expression of one or more abiotic stress tolerance genes in a plant when the transcription factor binds to the regulating region.

### DETAILED DESCRIPTION

### Transcription Factors Modify Expression of Endogenous Genes

A transcription factor may include, but is not limited to, any polypeptide that can activate or repress transcription of a single gene or a number of genes. As one of ordinary skill in the art recognizes, transcription factors can be identified by the presence of a region or domain of structural similarity or identity to a specific consensus sequence or the presence of a specific consensus DNA-binding site or DNA-binding site motif (see, for example, Riechmann et al. (2000). The plant transcription factors may belong to, for example, the bZIPT2-related or other transcription factor families.

Generally, the transcription factors encoded by the present sequences are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with improved traits related to improved yield and/or fruit quality. The sequences of the invention may also be used to transform a plant and introduce desirable traits not found in the wild-type cultivar or strain. Plants may then be selected for those that produce the most desirable degree of over- or under-expression of target genes of interest and coincident trait improvement.

The sequences of the present invention may be from any species, particularly plant species, in a naturally occurring form or from any source whether natural, synthetic, semi-synthetic or recombinant. The sequences of the invention may also include fragments of the present amino acid sequences. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

In addition to methods for modifying a plant phenotype by employing one or more polynucleotides and polypeptides of the invention described herein, the polynucleotides and polypeptides of the invention have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, for example, mutation reactions, PCR reactions, or the like; as substrates for cloning for example, including digestion or ligation reactions; and for identifying exogenous or endogenous modulators of the transcription factors. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, for example, genomic DNA or RNA, a transcript (such as an mRNA), a cDNA, a PCR product, a cloned DNA, a synthetic DNA or RNA, or the like. The polynucleotide can comprise a sequence in either sense or antisense orientations.

Expression of genes that encode transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) and Peng et al. (1999). In addition, many others have demonstrated that an Arabidopsis transcription factor expressed in an exogenous plant species elicits the same or very similar phenotypic response (see, for example, Fu et al. (2001); Nandi et al. (2000); Coupland (1995); and Weigel and Nilsson (1995)).

In another example, Mandel et al. (1992b) and Suzuki et al. (2001) teach that a transcription factor expressed in another plant species elicits the same or very similar phenotypic response of the endogenous sequence, as often predicted in earlier studies of Arabidopsis transcription factors in Arabidopsis (see Mandel et al. (1992b); Suzuki et al. (2001)).

Other examples include Müller et al. (2001); Kim et al. (2001); Kyozuka and Shimamoto (2002); Boss and Thomas (2002); He et al. (2000); and Robson et al. (2001).

In yet another example, Gilmour et al. (1998) teach an Arabidopsis AP2 transcription factor, CBF1, which, when overexpressed in transgenic plants, increases plant freezing tolerance. Jaglo et al. (2001) further identified sequences in Brassica napus that encode CBF-like genes and that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues, PKK/RPAGRxKFxETRHP and DSAWR, which bracket the AP2/EREBP DNA binding domains of the proteins and distinguish them from other members of the AP2/EREBP protein family (Jaglo et al. (2001).

Transcription factors mediate cellular responses and control traits through altered expression of genes containing cis-acting nucleotide sequences that are targets of the introduced transcription factor. It is well appreciated in the art that the effect of a transcription factor on cellular responses or a cellular trait is determined by the particular genes whose expression is either directly or indirectly (for example, by a cascade of transcription factor binding events and transcriptional changes) altered by transcription factor binding. In a global analysis of transcription comparing a standard condition with one in which a transcription factor is overexpressed, the resulting transcript profile associated with transcription factor overexpression is related to the trait or cellular process controlled by that transcription factor. For example, the PAP2 gene and other genes in the MYB family have been shown to control anthocyanin biosynthesis through regulation of the expression of genes known to be involved in the anthocyanin biosynthetic pathway (Bruce et al. (2000); Borevitz et al. (2000)). Further, global transcript profiles have been used successfully as diagnostic tools for specific cellular states (for example, cancerous vs. non-cancerous; Bhattacharjee et al. (2001); Xu et al. (2001)). Consequently, it is evident to one skilled in the art that similarity of transcript profile upon overexpression of different transcription factors would indicate similarity of transcription factor function.

### Polypeptides and Polynucleotides of the Invention

The present invention provides, among other things, transcription factors, and transcription factor homolog polypeptides, and isolated or recombinant polynucleotides encoding the polypeptides, or novel sequence variant polypeptides or polynucleotides encoding novel variants of transcription factors derived from the specific sequences provided here.

The polynucleotides of the invention can be or were ectopically expressed in overexpressor plant cells and the changes in the expression levels of a number of genes, polynucleotides, and/or proteins of the plant cells observed. Therefore, the polynucleotides and polypeptides can be employed to change expression levels of a genes, polynucleotides, and/or proteins of plants. These polypeptides and polynucleotides may be employed to modify a plant's characteristics, particularly improvement of yield and/or fruit quality. The polynucleotides of the invention can be or were ectopically expressed in overexpressor or knockout plants and the changes in the characteristic(s) or trait(s) of the plants observed. Therefore, the polynucleotides and polypeptides can be employed to improve the characteristics of plants. The polypeptide sequences of the sequence listing, including Arabidopsis sequences G3, G22, G24, G47, G156, G159, G187, G190, G226, G237, G270, G328, G363, G383, G435, G450, G522, G551, G558, G567, G580, G635, G675, G729, G812, G843, G881, G937, G989, G1007, G1053, G1078, G1226, G1273, G1324, G1328, G1444, G1462, G1463, G1481, G1504, G1543, G1635, G1638, G1640, G1645, G1650, G1659, G1752, G1755, G1784, G1785, G1791, G1808, G1809, G1815, G1865, G1884, G1895, G1897, G1903, G1909, G1935, G1950, G1954, G1958, G2052, G2072, G2108, G2116, G2132, G2137, G2141, G2145, G2150, G2157, G2294, G2296, G2313, G2417, G2425, G2505, conferred improved characteristics when these polypeptides were overexpressed in tomato plants. These polynucleotides have been shown to have a strong association with improved biomass, which is related to yield, and greater lycopene or soluble solids, which impacts fruit quality. Paralogs of these sequences that may be expected to function in a similar manner include G10, G12, G28, G30, G65, G195, G198, G225, G248, G448, G455, G456, G506, G554, G555, G556, G568, G577, G578, G629, G682, G730, G761, G798, G900, G986, G1006, G1040, G1047, G1198, G1264, G1277, G1309, G1354, G1355, G1379, G1453, G1461, G1464, G1465, G1754, G1766, G1792, G1795, G1806, G1816, G1846, G1917, G2058, G2067, G2115, G2133, G2148, G2424, G2436, G2442, G2443, G2467, G2504, G2512, G2534, G2578, G2629, G2635, G2718, G2893, G3034. Orthologs of these sequences that are expected to function in a similar manner include G3380, G3381, G3383, G3392, G3393, G3430, G3431, G3444, G3445, G3446, G3447, G3448, G3449, G3450, G3490, G3515, G3516, G3517, G3518, G3519, G3520, G3524, G3643, G3644, G3645, G3646, G3647, G3649, G3651, G3656, G3659, G3660, G3661, G3717, G3718, G3735, G3736, G3737, G3739, G3794, G3841, G3843, G3844, G3845, G3846, G3848, G3852, G3856, G3857, G3858, G3864, G3865.

The invention also encompasses sequences that are complementary to the polynucleotides of the invention. The polynucleotides are also useful for screening libraries of molecules or compounds for specific binding and for creating transgenic plants having improved yield and/or fruit quality. Altering the expression levels of equivalogs of these sequences, including paralogs and orthologs in the Sequence Listing, and other orthologs that are structurally and sequentially similar to the former orthologs, has been shown and is expected to confer similar phenotypes, including improved biomass, yield and/or fruit quality in plants.

In some cases, exemplary polynucleotides encoding the polypeptides of the invention were identified in the Arabidopsis thaliana GenBank database using publicly available sequence analysis programs and parameters. Sequences initially identified were then further characterized to identify sequences comprising specified sequence strings corresponding to sequence motifs present in families of known transcription factors. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known transcription factors. Polynucleotide sequences meeting such criteria were confirmed as transcription factors.

Additional polynucleotides of the invention were identified by screening Arabidopsis thaliana and/or other plant cDNA libraries with probes corresponding to known transcription factors under low stringency hybridization conditions. Additional sequences, including full length coding sequences were subsequently recovered by the rapid amplification of cDNA ends (RACE) procedure, using a commercially available kit according to the manufacturer's instructions. Where necessary, multiple rounds of RACE are performed to isolate 5′ and 3′ ends. The full-length cDNA was then recovered by a routine end-to-end PCR using primers specific to the isolated 5′ and 3′ ends. Exemplary sequences are provided in the Sequence Listing.

The invention also entails an agronomic composition comprising a polynucleotide of the invention in conjunction with a suitable carrier and a method for altering a plant's trait using the composition.

Examples of specific polynucleotide and polypeptides of the invention, and equivalog sequences, along with descriptions of the gene families that comprise these polynucleotides and polypeptides, are provided below.

Table 1 shows a number of polypeptides of the invention shown to improve fruit or yield characteristics (SEQ ID NO: 2N, where N=1 to 82), paralogs of these sequences (SEQ ID NO: 2N, where N=83 to 148 or 416) and orthologs (SEQ ID NO: 2N, where N=150 to 201, 413 to 415, or 417 to 419), identified by SEQ ID NO; Identifier (e.g., Gene ID (GID) No); the transcription factor family to which the polypeptide belongs, and conserved domain amino acid coordinates of the polypeptide.

TABLE 1
Gene families and conserved domains

| | | Conserved | |
|---|---|---|---|
| | | Domains in | |
| Polypeptide | | Amino Acid | |
| SEQ ID NO: | GID | Coordinates | Family |
| 2 | G3 | 28-95 | AP2 |
| 4 | G22 | 88-152 | AP2 |
| 6 | G24 | 25-92 | AP2 |
| 8 | G47 | 10-75 | AP2 |
| 10 | G156 | 2-57 | MADS |
| 12 | G159 | 7-61 | MADS |
| 14 | G187 | 172-228 | WRKY |
| 16 | G190 | 110-169 | WRKY |
| 18 | G226 | 38-82 | MYB-related |
| 20 | G237 | 11-113 | MYB-(R1)R2R3 |
| 22 | G270 | 259-424 | AKR |
| 24 | G328 | 12-78 | Z-CO-like |
| 26 | G363 | 87-108 | Z-C2H2 |
| 28 | G383 | 77-102 | GATA/Zn |
| 30 | G435 | 4-67 | HB |
| 32 | G450 | 6-14, 78-89, | IAA |
| | | 112-128, 180-217 | |
| 34 | G522 | 10-165 | NAC |
| 36 | G551 | 73-133 | HB |
| 38 | G558 | 45-105 | bZIP |
| 40 | G567 | 210-270 | bZIP |
| 42 | G580 | 162-218 | bZIP |
| 44 | G635 | 239-323 | TH |
| 46 | G675 | 13-116 | MYB-(R1)R2R3 |
| 48 | G729 | 224-272 | GARP |
| 50 | G812 | 29-120 | HS |
| 52 | G843 | 60-119, 270-350 | MISC |
| 54 | G881 | 176-233 | WRKY |
| 56 | G937 | 197-246 | GARP |
| 58 | G989 | 121-186, 238-326, | SCR |
| | | 327-399 | |
| 60 | G1007 | 23-90 | AP2 |
| 62 | G1053 | 74-120 | bZIP |
| 64 | G1078 | 1-53, 440-550 | BZIPT2 |
| 66 | G1226 | 115-174 | HLH/MYC |
| 68 | G1273 | 163-218, 347-403 | WRKY |
| 70 | G1324 | 20-118 | MYB-(R1)R2R3 |
| 72 | G1328 | 14-119 | MYB-(R1)R2R3 |
| 74 | G1444 | 17-101 | GRF-like |
| 76 | G1462 | 14-273 | NAC |
| 78 | G1463 | 9-156 | NAC |
| 80 | G1481 | 5-27, 47-73 | Z-CO-like |
| 82 | G1504 | 193-206 | GATA/Zn |
| 84 | G1543 | 135-195 | HB |
| 86 | G1635 | 56-102 | MYB-related |
| 88 | G1638 | 27-77, 141-189 | MYB-related |
| 90 | G1640 | 14-115 | MYB-(R1)R2R3 |
| 92 | G1645 | 90-210 | MYB-(R1)R2R3 |
| 94 | G1650 | 284-334 | HLH/MYC |
| 96 | G1659 | 17-116 | DBP |
| 98 | G1752 | 83-151 | AP2 |
| 100 | G1755 | 71-133 | AP2 |
| 102 | G1784 | 60-248 | PMR |
| 104 | G1785 | 25-125 | MYB-(R1)R2R3 |
| 106 | G1791 | 10-74 | AP2 |
| 108 | G1808 | 140-200 | bZIP |
| 110 | G1809 | 136-196 | bZIP |
| 112 | G1815 | 65-170 | MYB-(R1)R2R3 |
| 114 | G1865 | 45-162 | GRF-like |
| 116 | G1884 | 43-71 | Z-Dof |
| 118 | G1895 | 58-100 | Z-Dof |
| 120 | G1897 | 34-62 | Z-Dof |
| 122 | G1903 | 134-180 | Z-Dof |
| 124 | G1909 | 23-51 | Z-Dof |
| 126 | G1935 | 1-57 | MADS |
| 128 | G1950 | 65-228 | AKR |
| 130 | G1954 | 187-259 | HLH/MYC |
| 132 | G1958 | 230-278 | GARP |
| 134 | G2052 | 7-158 | NAC |
| 136 | G2072 | 90-149 | bZIP |
| 138 | G2108 | 18-85 | AP2 |
| 140 | G2116 | 150-210 | bZIP |
| 142 | G2132 | 84-151 | AP2 |
| 144 | G2137 | 109-168 | WRKY |
| 146 | G2141 | 302-380 | HLH/MYC |
| 148 | G2145 | 166-243 | HLH/MYC |
| 150 | G2150 | 190-268 | HLH/MYC |
| 152 | G2157 | 82-102, 107-164 | AT-hook |
| 154 | G2294 | 32-100 | AP2 |
| 156 | G2296 | 85-145 | WRKY |
| 158 | G2313 | 111-159 | MYB-related |
| 160 | G2417 | 235-285 | GARP |
| 162 | G2425 | 12-119 | MYB-(R1)R2R3 |
| 164 | G2505 | 9-137 | NAC |
| 166 | G10 | 21-88 | AP2 |
| 168 | G12 | 27-94 | AP2 |
| 170 | G28 | 145-208 | AP2 |
| 172 | G30 | 16-80 | AP2 |
| 174 | G165 | 7-62 | MADS |
| 176 | G195 | 183-239 | WRKY |
| 178 | G198 | 14-117 | MYB-(R1)R2R3 |
| 180 | G225 | 36-80 | MYB-related |
| 182 | G248 | 264-332 | MYB-(R1)R2R3 |
| 184 | G448 | 11-20, 83-95, | IAA |
| | | 111-128, 180-214 | |
| 186 | G455 | 11-19, 84-95, | IAA |
| | | 126-142, 194-227 | |
| 188 | G456 | 7-14, 71-81, | IAA |
| | | 120-153, 185-221 | |
| 190 | G506 | 8-157 | NAC |
| 192 | G554 | 82-142 | bZIP |
| 194 | G555 | 38-110 | bZIP |
| 196 | G556 | 83-143 | bZIP |
| 198 | G568 | 215-265 | bZIP |
| 200 | G577 | 1-53, 356-466 | BZIPT2 |
| 202 | G578 | 36-96 | bZIP |
| 204 | G629 | 92-152 | bZIP |
| 206 | G682 | 33-77 | MYB-related |
| 208 | G730 | 169-217 | GARP |
| 210 | G761 | 10-156 | NAC |
| 212 | G798 | 19-47 | Z-Dof |
| 214 | G900 | 6-28, 48-74 | Z-CO-like |
| 216 | G986 | 146-203 | WRKY |
| 218 | G1006 | 113-177 | AP2 |
| 220 | G1040 | 109-158 | GARP |
| 222 | G1047 | 129-180 | bZIP |
| 224 | G1198 | 173-223 | bZIP |
| 226 | G1264 | 96-138 | Z-Dof |
| 228 | G1277 | 18-85 | AP2 |
| 230 | G1309 | 9-114 | MYB-(R1)R2R3 |
| 232 | G1354 | 7-157 | NAC |
| 234 | G1355 | 9-159 | NAC |
| 236 | G1379 | 18-85 | AP2 |
| 238 | G1453 | 13-160 | NAC |
| 240 | G1461 | 37-163 | NAC |
| 242 | G1464 | 12-160 | NAC |
| 244 | G1465 | 242-306 | NAC |
| 246 | G1754 | 69-136 | AP2 |
| 248 | G1766 | 10-153 | NAC |
| 250 | G1792 | 16-80 | AP2 |
| 252 | G1795 | 11-75 | AP2 |
| 254 | G1806 | 165-225 | bZIP |
| 256 | G1816 | 30-74 | MYB-related |
| 258 | G1846 | 16-83 | AP2 |
| 260 | G1917 | 153-179 | GATA/Zn |
| 262 | G2058 | 2-57 | MADS |
| 264 | G2067 | 40-102 | AP2 |
| 266 | G2115 | 47-113 | AP2 |
| 268 | G2133 | 10-77 | AP2 |
| 270 | G2148 | 130-268 | HLH/MYC |
| 272 | G2424 | 107-219 | MYB-(R1)R2R3 |
| 274 | G2436 | 16-111 | Z-CO-like |
| 276 | G2442 | 220-246 | GATA/Zn |
| 278 | G2443 | 20-86 | Z-CO-like |
| 280 | G2467 | 28-119 | HS |
| 282 | G2504 | 222-248 | GATA/Zn |
| 284 | G2512 | 79-147 | AP2 |
| 286 | G2534 | 10-157 | NAC |
| 288 | G2578 | 1-57 | MADS |
| 290 | G2629 | 85-154 | bZIP |
| 292 | G2635 | 8-161 | NAC |
| 294 | G2718 | 32-76 | MYB-related |
| 296 | G2893 | 19-120 | MYB-(R1)R2R3 |
| 298 | G3034 | 218-266 | GARP |
| 300 | G3380 | 18-82 | AP2 |
| 302 | G3381 | 14-78 | AP2 |
| 304 | G3383 | 9-73 | AP2 |
| 306 | G3392 | 32-76 | MYB-related |
| 308 | G3393 | 31-75 | MYB-related |
| 310 | G3430 | 109-173 | AP2 |
| 312 | G3431 | 31-75 | MYB-related |
| 314 | G3444 | 31-75 | MYB-related |
| 316 | G3445 | 25-69 | MYB-related |
| 318 | G3446 | 26-70 | MYB-related |
| 320 | G3447 | 26-70 | MYB-related |
| 322 | G3448 | 26-70 | MYB-related |
| 324 | G3449 | 26-70 | MYB-related |
| 326 | G3450 | 20-64 | MYB-related |
| 328 | G3490 | 60-120 | HB |
| 826 | G3510 | 74-134 | HB |
| 330 | G3515 | 11-75 | AP2 |
| 332 | G3516 | 6-70 | AP2 |
| 334 | G3517 | 13-77 | AP2 |
| 336 | G3518 | 13-77 | AP2 |
| 338 | G3519 | 13-77 | AP2 |
| 340 | G3520 | 14-78 | AP2 |
| 342 | G3524 | 60-120 | HB |
| 344 | G3643 | 13-78 | AP2 |
| 346 | G3644 | 52-122 | AP2 |
| 348 | G3645 | 10-75 | AP2 |
| 350 | G3646 | 10-77 | AP2 |
| 352 | G3647 | 13-78 | AP2 |
| 354 | G3649 | 15-87 | AP2 |
| 828 | G3650 | 75-139 | AP2 |
| 356 | G3651 | 60-130 | AP2 |
| 358 | G3656 | 23-86 | AP2 |
| 830 | G3657 | 47-109 | AP2 |
| 360 | G3659 | 130-194 | AP2 |
| 362 | G3660 | 119-183 | AP2 |
| 364 | G3661 | 126-190 | AP2 |
| 366 | G3717 | 130-194 | AP2 |
| 368 | G3718 | 139-203 | AP2 |
| 370 | G3735 | 23-87 | AP2 |
| 372 | G3736 | 12-76 | AP2 |
| 374 | G3737 | 8-72 | AP2 |
| 376 | G3739 | 13-77 | AP2 |
| 378 | G3794 | 6-70 | AP2 |
| 380 | G3841 | 102-166 | AP2 |
| 382 | G3843 | 130-194 | AP2 |
| 384 | G3844 | 141-205 | AP2 |
| 386 | G3845 | 101-165 | AP2 |
| 388 | G3846 | 95-159 | AP2 |
| 390 | G3848 | 149-213 | AP2 |
| 392 | G3852 | 102-167 | AP2 |
| 394 | G3856 | 140-204 | AP2 |
| 396 | G3857 | 98-162 | AP2 |
| 398 | G3858 | 108-172 | AP2 |
| 400 | G3864 | 127-191 | AP2 |
| 402 | G3865 | 125-189 | AP2 |
| 832 | G3930 | 33-77 | MYB-related |
| 834 | G4014 | 4-75 | Z-CO-like |
| 836 | G4015 | 8-79 | Z-CO-like |
| 838 | G4016 | 4-75 | Z-CO-like |

### Producing Polypeptides

The polynucleotides of the invention include sequences that encode transcription factors and transcription factor homolog polypeptides and sequences complementary thereto, as well as unique fragments of coding sequence, or sequence complementary thereto. Such polynucleotides can be, for example, DNA or RNA, the latter including mRNA, cRNA, synthetic RNA, genomic DNA, cDNA synthetic DNA, oligonucleotides, etc. The polynucleotides are either double-stranded or single-stranded, and include either, or both sense (i.e., coding) sequences and antisense (i.e., non-coding, complementary) sequences. The polynucleotides include the coding sequence of a transcription factor, or transcription factor homolog polypeptide, in isolation, in combination with additional coding sequences (e.g., a purification tag, a localization signal, as a fusion-protein, as a pre-protein, or the like), in combination with non-coding sequences (for example, introns or inteins, regulatory elements such as promoters, enhancers, terminators, and the like), and/or in a vector or host environment in which the polynucleotide encoding a transcription factor or transcription factor homolog polypeptide is an endogenous or exogenous gene.

A variety of methods exist for producing the polynucleotides of the invention. Procedures for identifying and isolating DNA clones are well known to those of skill in the art, and are described in, for example, Berger and Kimmel (1987); Sambrook et al. (1989) and Ausubel et al. (supplemented through 2000).

Alternatively, polynucleotides of the invention, can be produced by a variety of in vitro amplification methods adapted to the present invention by appropriate selection of specific or degenerate primers. Examples of protocols sufficient to direct persons of skill through in vitro amplification methods, including the polymerase chain reaction (PCR) the ligase chain reaction (LCR), Qβ-replicase amplification and other RNA polymerase mediated techniques (for example, NASBA), e.g., for the production of the homologous nucleic acids of the invention are found in Berger and Kimmel (1987), Sambrook (1989), and Ausubel (2000), as well as Mullis et al. (1990). Improved methods for cloning in vitro amplified nucleic acids are described in U.S. Pat. No. 5,426,039. Improved methods for amplifying large nucleic acids by PCR are summarized in Cheng et al. (1994) and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel (2000), Sambrook (1989) and Berger and Kimmel (1987).

Alternatively, polynucleotides and oligonucleotides of the invention can be assembled from fragments produced by solid-phase synthesis methods. Typically, fragments of up to approximately 100 bases are individually synthesized and then enzymatically or chemically ligated to produce a desired sequence, e.g., a polynucleotide encoding all or part of a transcription factor. For example, chemical synthesis using the phosphoramidite method is described, e.g., by Beaucage et al. (1981) and Matthes et al. (1984). According to such methods, oligonucleotides are synthesized, purified, annealed to their complementary strand, ligated and then optionally cloned into suitable vectors. And if so desired, the polynucleotides and polypeptides of the invention can be custom ordered from any of a number of commercial suppliers.

### Homologous Sequences

Sequences homologous, i.e., that share significant sequence identity or similarity, to those provided in the Sequence Listing, derived from Arabidopsis thaliana or from other plants of choice, are also an aspect of the invention. Homologous sequences can be derived from any plant including monocots and dicots and in particular agriculturally important plant species, including but not limited to, crops such as soybean, wheat, corn (maize), potato, cotton, rice, rape, oilseed rape (including canola), sunflower, alfalfa, clover, sugarcane, and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts, and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassava, turnip, radish, yam, and sweet potato; and beans. The homologous sequences may also be derived from woody species, such pine, poplar and eucalyptus, or mint or other labiates. In addition, homologous sequences may be derived from plants that are evolutionarily related to crop plants, but which may not have yet been used as crop plants. Examples include deadly nightshade (Atropa belladona), related to tomato; jimson weed (Datura strommium), related to peyote; and teosinte (Zea species), related to corn (maize).

### Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and functions. Orthologs are structurally related genes in different species that are derived by a speciation event. Paralogs are structurally related genes within a single species that are derived by a duplication event. Sequences that are sufficiently similar to one another will be appreciated by those of skill in the art and may be based upon percentage identity of the complete sequences, percentage identity of a conserved domain or sequence within the complete sequence, percentage similarity to the complete sequence, percentage similarity to a conserved domain or sequence within the complete sequence, and/or an arrangement of contiguous nucleotides or peptides particular to a conserved domain or complete sequence. Sequences that are sufficiently similar to one another will also bind in a similar manner to the same DNA binding sites of transcriptional regulatory elements using methods well known to those of skill in the art.

Paralogs typically cluster together or in the same clade (a group of similar genes) when a gene family phylogeny is analyzed using programs such as CLUSTAL (Thompson et al. (1994); Higgins et al. (1996)). Groups of similar genes can also be identified with pair-wise BLAST analysis (Feng and Doolittle (1987)). For example, a clade of very similar MADS domain transcription factors from Arabidopsis all share a common function in flowering time (Ratcliffe et al. (2001), and a group of very similar AP2 domain transcription factors from Arabidopsis are involved in tolerance of plants to freezing (Gilmour et al. (1998)). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount (2001)). Paralogous genes may retain similar functions of the encoded proteins. In such cases, paralogs can be used interchangeably with respect to certain embodiments of the instant invention (for example, transgenic expression of a coding sequence). An example of such highly related paralogs is the CBF family, with four well-defined members in Arabidopsis (CBF1, CBF2, CBF3 and GenBank accession number AB015478) and at least one ortholog in Brassica napus, bnCBF1, all of which control pathways involved in both freezing and drought stress (Gilmour et al. (1998); Jaglo et al. (1998)).

Speciation, the production of new species from a parental species, can also give rise to two or more genes with similar sequence. Because plants have common ancestors, many genes in any plant species will have a corresponding orthologous gene in another plant species. Once a phylogenic tree for a gene family of one species has been constructed using a program such as CLUSTAL (Thompson et al. (1994); Higgins et al. (1996) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence. Orthologous genes from different organisms have highly conserved functions, and very often essentially identical functions (Lee et al. (2002); Remm et al. (2001)).

Transcription factor gene sequences are conserved across diverse eukaryotic species lines (Goodrich et al. (1993); Lin et al. (1991); Sadowski et al. (1988)). Plants are no exception to this observation; diverse plant species possess transcription factors that have similar sequences and functions.

The following references represent a small sampling of the many studies that demonstrate that conserved transcription factor genes from diverse species are likely to function similarly (i.e., regulate similar target sequences and control the same traits), and that transcription factors may be transformed into diverse species to confer or improve traits.

(1) The Arabidopsis NPR1 gene regulates systemic acquired resistance (SAR; Cao et al. (1997)); over-expression of NPR1 leads to enhanced resistance in Arabidopsis. When either Arabidopsis NPR1 or the rice NPR1 ortholog was overexpressed in rice (which, as a monocot, is diverse from Arabidopsis), challenge with the rice bacterial blight pathogen Xanthomonas oryzae pv. Oryzae, the transgenic plants displayed enhanced resistance (Chern et al. (2001)). NPR1 acts through activation of expression of transcription factor genes, such as TGA2 (Fan and Dong (2002)).

(2) E2F genes are involved in transcription of plant genes for proliferating cell nuclear antigen (PCNA). Plant E2Fs share a high degree of similarity in amino acid sequence between monocots and dicots, and are even similar to the conserved domains of the animal E2Fs. functional similarity between plant and animal E2Fs. E2F transcription factors that regulate meristem development act through common cis-elements, and regulate related (PCNA) genes (Kosugi and Ohashi (2002)).

(3) The ABI5 gene (ABA insensitive 5) encodes a basic leucine zipper factor required for ABA response in the seed and vegetative tissues. Co-transformation experiments with ABI5 cDNA constructs in rice protoplasts resulted in specific transactivation of the ABA-inducible wheat, Arabidopsis, bean, and barley promoters. These results demonstrate that sequentially similar ABI5 transcription factors are key targets of a conserved ABA signaling pathway in diverse plants. (Gampala et al. (2001)).

(4) Sequences of three Arabidopsis GAMYB-like genes were obtained on the basis of sequence similarity to GAMYB genes from barley, rice, and L. temulentum. These three Arabidopsis genes were determined to encode transcription factors (AtMYB33, AtMYB65, and AtMYB101) and could substitute for a barley GAMYB and control alpha-amylase expression (Gocal et al. (2001)).

(5) The floral control gene LEAFY from Arabidopsis can dramatically accelerate flowering in numerous dicotyledonous plants. Constitutive expression of Arabidopsis LEAFY also caused early flowering in transgenic rice (a monocot), with a heading date that was 26-34 days earlier than that of wild-type plants. These observations indicate that floral regulatory genes from Arabidopsis are useful tools for heading date improvement in cereal crops (He et al. (2000)).

(6) Bioactive gibberellins (GAs) are essential endogenous regulators of plant growth. GA signaling tends to be conserved across the plant kingdom. GA signaling is mediated via GAI, a nuclear member of the GRAS family of plant transcription factors. Arabidopsis GAI has been shown to function in rice to inhibit gibberellin response pathways (Fu et al. (2001)).

(7) The Arabidopsis gene SUPERMAN (SUP), encodes a putative transcription factor that maintains the boundary between stamens and carpels. By over-expressing Arabidopsis SUP in rice, the effect of the gene's presence on whorl boundaries was shown to be conserved. This demonstrated that SUP is a conserved regulator of floral whorl boundaries and affects cell proliferation (Nandi et al. (2000)).

(8) Maize, petunia and Arabidopsis myb transcription factors that regulate flavonoid biosynthesis are very genetically similar and affect the same trait in their native species, therefore sequence and function of these myb transcription factors correlate with each other in these diverse species (Borevitz et al. (2000)).

(9) Wheat reduced height-1 (Rht-B1/Rht-D1) and maize dwarf-8 (d8) genes are orthologs of the Arabidopsis gibberellin insensitive (GAI) gene. Both of these genes have been used to produce dwarf grain varieties that have improved grain yield. These genes encode proteins that resemble nuclear transcription factors and contain an SH2-like domain, indicating that phosphotyrosine may participate in gibberellin signaling. Transgenic rice plants containing a mutant GAI allele from Arabidopsis have been shown to produce reduced responses to gibberellin and are dwarfed, indicating that mutant GAI orthologs could be used to increase yield in a wide range of crop species (Peng et al. (1999)).

Transcription factors that are homologous to the listed sequences will typically share at least about 70% amino acid sequence identity in the conserved domain. More closely related transcription factors can share at least about 79% or about 90% or about 95% or about 98% or more sequence identity with the listed sequences, or with the listed sequences but excluding or outside a known consensus sequence or consensus DNA-binding site, or with the listed sequences excluding one or all conserved domains. Factors that are most closely related to the listed sequences share, e.g., at least about 85%, about 90% or about 95% or more % sequence identity to the listed sequences, or to the listed sequences but excluding or outside a known consensus sequence or consensus DNA-binding site or outside one or all conserved domain. At the nucleotide level, the sequences will typically share at least about 40% nucleotide sequence identity, preferably at least about 50%, about 60%, about 70% or about 80% sequence identity, and more preferably about 85%, about 90%, about 95% or about 97% or more sequence identity to one or more of the listed sequences, or to a listed sequence but excluding or outside a known consensus sequence or consensus DNA-binding site, or outside one or all conserved domain. The degeneracy of the genetic code enables major variations in the nucleotide sequence of a polynucleotide while maintaining the amino acid sequence of the encoded protein. TH domains within the TH transcription factor family may exhibit a higher degree of sequence homology, such as at least 70% amino acid sequence identity including conservative substitutions, and preferably at least 80% sequence identity, and more preferably at least 85%, or at least about 86%, or at least about 87%, or at least about 88%, or at least about 90%, or at least about 95%, or at least about 98% sequence identity. Transcription factors that are homologous to the listed sequences should share at least 30%, or at least about 60%, or at least about 75%, or at least about 80%, or at least about 90%, or at least about 95% amino acid sequence identity over the entire length of the polypeptide or the homolog.

Percent identity can be determined electronically, e.g., by using the MEGALIGN program (DNASTAR, Inc. Madison, Wis.). The MEGALIGN program can create alignments between two or more sequences according to different methods, for example, the clustal method (see, for example, Higgins and Sharp (1988)). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Other techniques for alignment are described in Doolittle, ed. (1996). Preferably, an alignment program that permits gaps in the sequence is utilized to align the sequences. The Smith-Waterman is one type of algorithm that permits gaps in sequence alignments (see Shpaer (1997)). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (see, e.g., Hein (1990)). Identity between sequences can also be determined by other methods known in the art, e.g., by varying hybridization conditions (see US Patent Application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al. (1997)), PFAM, and other databases which contain previously identified and annotated motifs, sequences and gene functions. Methods that search for primary sequence patterns with secondary structure gap penalties (Smith et al. (1992) as well as algorithms such as Basic Local Alignment Search Tool (BLAST; Altschul (1993); Altschul et al. (1990)), BLOCKS (Henikoff and Henikoff (1991)), Hidden Markov Models (HMM; Eddy (1996); Sonnhammer et al. (1997)), and the like, can be used to manipulate and analyze polynucleotide and polypeptide sequences encoded by polynucleotides. These databases, algorithms and other methods are well known in the art and are described in Ausubel et al. (1997) and in Meyers (1995).

Another method for identifying or confirming that specific homologous sequences control the same function is by comparison of the transcript profile(s) obtained upon overexpression or knockout of two or more related transcription factors. Since transcript profiles are diagnostic for specific cellular states, one skilled in the art will appreciate that genes that have a highly similar transcript profile (e.g., with greater than 50% regulated transcripts in common, more preferably with greater than 70% regulated transcripts in common, most preferably with greater than 90% regulated transcripts in common) will have highly similar functions. Fowler and Thomashow (2002) have shown that three paralogous AP2 family genes (CBF1, CBF2 and CBF3), each of which is induced upon cold treatment, and each of which can condition improved freezing tolerance, have highly similar transcript profiles. Once a transcription factor has been shown to provide a specific function, its transcript profile becomes a diagnostic tool to determine whether putative paralogs or orthologs have the same function.

Furthermore, methods using manual alignment of sequences similar or homologous to one or more polynucleotide sequences or one or more polypeptides encoded by the polynucleotide sequences may be used to identify regions of similarity and TH domains. Such manual methods are well-known of those of skill in the art and can include, for example, comparisons of tertiary structure between a polypeptide sequence encoded by a polynucleotide which comprises a known function and a polypeptide sequence encoded by a polynucleotide sequence which has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

Orthologs and paralogs of presently disclosed transcription factors may be cloned using compositions provided by the present invention according to methods well known in the art. cDNAs can be cloned using mRNA from a plant cell or tissue that expresses one of the present transcription factors. Appropriate mRNA sources may be identified by interrogating Northern blots with probes designed from the present transcription factor sequences, after which a library is prepared from the mRNA obtained from a positive cell or tissue. Transcription factor-encoding cDNA is then isolated using, for example, PCR, using primers designed from a presently disclosed transcription factor gene sequence, or by probing with a partial or complete cDNA or with one or more sets of degenerate probes based on the disclosed sequences. The cDNA library may be used to transform plant cells. Expression of the cDNAs of interest is detected using, for example, methods disclosed herein such as microarrays, Northern blots, quantitative PCR, or any other technique for monitoring changes in expression. Genomic clones may be isolated using similar techniques to those.

### Identifying Polynucleotides or Nucleic Acids by Hybridization

Polynucleotides homologous to the sequences illustrated in the Sequence Listing and tables can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited below (e.g., Sambrook et al. (1989); Berger and Kimmel (1987); and Anderson and Young (1985)).

Encompassed by the invention are polynucleotide sequences that are capable of hybridizing to the claimed polynucleotide sequences, including any of the transcription factor polynucleotides within the Sequence Listing, and fragments thereof under various conditions of stringency (see, for example, Wahl and Berger (1987); and Kimmel (1987)). In addition to the nucleotide sequences in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al. (1989); Berger and Kimmel (1987) pp. 467-469; and Anderson and Young (1985).

Stability of DNA duplexes is affected by such factors as base composition, length, and degree of base pair mismatch Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

Tₘ(° C.)=81.5+16.6(log [Na+])+0.41 (% G+C)−0.62 (% formamide)−500/L (I) DNA-DNA

Tₘ(° C.)=79.8+18.5(log [Na+])+0.58 (% G+C)+0.12 (% G+C)²−0.5 (% formamide)−820/L (II) DNA-RNA

Tₘ(° C.)=79.8+18.5(log [Na+])+0.58 (% G+C)+0.12 (% G+C)²−0.35 (% formamide)−820/L (III) RNA-RNA

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young (1985)). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ-5° C. to Tₘ-20° C., moderate stringency at Tₘ-20° C. to Tₘ-35° C. and low stringency at Tₘ-35° C. to Tₘ-50° C. for duplex>150 base pairs. Hybridization may be performed at low to moderate stringency (25-50° C. below Tₘ), followed by post-hybridization washes at increasing stringencies. Maximum rates of hybridization in solution are determined empirically to occur at Tₘ-25° C. for DNA-DNA duplex and Tₘ-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present transcription factors include, for example:

6×SSC at 65° C.;

50% formamide, 4×SSC at 42° C.; or

0.5×SSC, 0.1% SDS at 65° C.;

with, for example, two wash steps of 10-30 minutes each. Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 min, or about 0.1×SSC, 0.1% SDS at 65° C. and washing twice for 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, US Patent Application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a transcription factor known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, for example, to SEQ ID NO: 2N-1, where N=1 to 201 or 413 to 419, and SEQ ID NO: 403-824, and fragments thereof under various conditions of stringency (see, e.g., Wahl and Berger (1987); Kimmel (1987)). Estimates of homology are provided by either DNA-DNA or DNA-RNA hybridization under conditions of stringency as is well understood by those skilled in the art (Hames and Higgins (1985). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

Identifying Polynucleotides or Nucleic Acids with Expression Libraries

In addition to hybridization methods, transcription factor homolog polypeptides can be obtained by screening an expression library using antibodies specific for one or more transcription factors. With the provision herein of the disclosed transcription factor, and transcription factor homolog nucleic acid sequences, the encoded polypeptide(s) can be expressed and purified in a heterologous expression system (e.g., E. coli) and used to raise antibodies (monoclonal or polyclonal) specific for the polypeptide(s) in question. Antibodies can also be raised against synthetic peptides derived from transcription factor, or transcription factor homolog, amino acid sequences. Methods of raising antibodies are well known in the art and are described in Harlow and Lane (1988). Such antibodies can then be used to screen an expression library produced from the plant from which it is desired to clone additional transcription factor homologs, using the methods described above. The selected cDNAs can be confirmed by sequencing and enzymatic activity.

### Sequence Variations

It will readily be appreciated by those of skill in the art, that any of a variety of polynucleotide sequences are capable of encoding the transcription factors and transcription factor homolog polypeptides of the invention. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (i.e., peptides having some degree of equivalent or similar biological activity) but differ in sequence from the sequence shown in the Sequence Listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms that may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Allelic variant refers to any of two or more alternative forms of a gene occupying the same chromosomal locus. Allelic variation arises naturally through mutation, and may result in phenotypic polymorphism within populations. Gene mutations can be silent (i.e., no change in the encoded polypeptide) or may encode polypeptides having altered amino acid sequence. The term allelic variant is also used herein to denote a protein encoded by an allelic variant of a gene. Splice variant refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Those skilled in the art would recognize that, for example, G1950, SEQ ID NO: 128, represents a single transcription factor; allelic variation and alternative splicing may be expected to occur. Allelic variants of SEQ ID NO: 127 can be cloned by probing cDNA or genomic libraries from different individual organisms according to standard procedures. Allelic variants of the DNA sequence shown in SEQ ID NO: 127, including those containing silent mutations and those in which mutations result in amino acid sequence changes, are within the scope of the present invention, as are proteins which are allelic variants of SEQ ID NO: 128. cDNAs generated from alternatively spliced mRNAs, which retain the properties of the transcription factor are included within the scope of the present invention, as are polypeptides encoded by such cDNAs and mRNAs. Allelic variants and splice variants of these sequences can be cloned by probing cDNA or genomic libraries from different individual organisms or tissues according to standard procedures known in the art (see U.S. Pat. No. 6,388,064).

Thus, in addition to the sequences set forth in the Sequence Listing, the invention also encompasses related nucleic acid molecules that include allelic or splice variants of the sequences of the invention, for example, SEQ ID NO: 2N-1, where N=1 to 201 or 413 to 419, or SEQ ID NO: 403 to 824, and include sequences that are complementary to any of the above nucleotide sequences. Related nucleic acid molecules also include nucleotide sequences encoding a polypeptide comprising a substitution, modification, addition and/or deletion of one or more amino acid residues compared to the polypeptide sequences of the invention, for example, SEQ ID NO: 2N, where N=1 to 201 or 413 to 419, or sequences encoded by SEQ ID NO: 403 to 824. Such related polypeptides may comprise, for example, additions and/or deletions of one or more N-linked or O-linked glycosylation sites, or an addition and/or a deletion of one or more cysteine residues.

For example, Table 2 illustrates, e.g., that the codons AGC, AGT, TCA, TCC, TCG, and TCT all encode the same amino acid: serine. Accordingly, at each position in the sequence where there is a codon encoding serine, any of the above trinucleotide sequences can be used without altering the encoded polypeptide.

TABLE 2

| Amino acid | | | Possible Codons |
|---|---|---|---|
| Alanine | Ala | A | GCA GCC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC AGT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, e.g., site-directed mutagenesis, available in the art. Accordingly, any and all such variations of a sequence selected from the above table are a feature of the invention.

In addition to silent variations, other conservative variations that alter one, or a few amino acid residues in the encoded polypeptide, can be made without altering the function of the polypeptide, these conservative variants are, likewise, a feature of the invention.

For example, substitutions, deletions and insertions introduced into the sequences provided in the Sequence Listing, are also envisioned by the invention. Such sequence modifications can be engineered into a sequence by site-directed mutagenesis (Wu (1993) or the other methods noted below. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, e.g., a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 3 when it is desired to maintain the activity of the protein. Table 3 shows amino acids which can be substituted for an amino acid in a protein and which are typically regarded as conservative substitutions.

TABLE 3

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Similar substitutions are those in which at least one residue in the amino acid sequence has been removed and a different residue inserted in its place. Such substitutions generally are made in accordance with the Table 4 when it is desired to maintain the activity of the protein. Table 4 shows amino acids which can be substituted for an amino acid in a protein and which are typically regarded as structural and functional substitutions. For example, a residue in column 1 of Table 4 may be substituted with a residue in column 2; in addition, a residue in column 2 of Table 4 may be substituted with the residue of column 1.

TABLE 4

| Residue | Similar Substitutions |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cys | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Gly; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 4 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

### Further Modifying Sequences of the Invention—Mutation/Forced Evolution

In addition to generating silent or conservative substitutions as noted, above, the present invention optionally includes methods of modifying the sequences of the Sequence Listing. In the methods, nucleic acid or protein modification methods are used to alter the given sequences to produce new sequences and/or to chemically or enzymatically modify given sequences to change the properties of the nucleic acids or proteins.

Thus, in one embodiment, given nucleic acid sequences are modified, e.g., according to standard mutagenesis or artificial evolution methods to produce modified sequences. The modified sequences may be created using purified natural polynucleotides isolated from any organism or may be synthesized from purified compositions and chemicals using chemical means well know to those of skill in the art. For example, Ausubel (2000), provides additional details on mutagenesis methods. Artificial forced evolution methods are described, for example, by Stemmer (1994a), Stemmer (1994b), and U.S. Pat. Nos. 5,811,238, 5,837,500, and 6,242,568. Methods for engineering synthetic transcription factors and other polypeptides are described, for example, by Zhang et al. (2000), Liu et al. (2001), and Isalan et al. (2001). Many other mutation and evolution methods are also available and expected to be within the skill of the practitioner.

Similarly, chemical or enzymatic alteration of expressed nucleic acids and polypeptides can be performed by standard methods. For example, sequence can be modified by addition of lipids, sugars, peptides, organic or inorganic compounds, by the inclusion of modified nucleotides or amino acids, or the like. For example, protein modification techniques are illustrated in Ausubel (2000). Further details on chemical and enzymatic modifications can be found herein. These modification methods can be used to modify any given sequence, or to modify any sequence produced by the various mutation and artificial evolution modification methods noted herein.

Accordingly, the invention provides for modification of any given nucleic acid by mutation, evolution, chemical or enzymatic modification, or other available methods, as well as for the products produced by practicing such methods, e.g., using the sequences herein as a starting substrate for the various modification approaches.

For example, optimized coding sequence containing codons preferred by a particular prokaryotic or eukaryotic host can be used e.g., to increase the rate of translation or to produce recombinant RNA transcripts having desirable properties, such as a longer half-life, as compared with transcripts produced using a non-optimized sequence. Translation stop codons can also be modified to reflect host preference. For example, preferred stop codons for Saccharomyces cerevisiae and mammals are TAA and TGA, respectively. The preferred stop codon for monocotyledonous plants is TGA, whereas insects and E. coli prefer to use TAA as the stop codon.

The polynucleotide sequences of the present invention can also be engineered in order to alter a coding sequence for a variety of reasons, including but not limited to, alterations which modify the sequence to facilitate cloning, processing and/or expression of the gene product. For example, alterations are optionally introduced using techniques which are well known in the art, e.g., site-directed mutagenesis, to insert new restriction sites, to alter glycosylation patterns, to change codon preference, to introduce splice sites, etc.

Furthermore, a fragment or domain derived from any of the polypeptides of the invention can be combined with domains derived from other transcription factors or synthetic domains to modify the biological activity of a transcription factor. For instance, a DNA-binding domain derived from a transcription factor of the invention can be combined with the activation domain of another transcription factor or with a synthetic activation domain. A transcription activation domain assists in initiating transcription from a DNA-binding site. Examples include the transcription activation region of VP16 or GAL4 (Moore et al. (1998); Aoyama et al. (1995)), peptides derived from bacterial sequences (Ma and Ptashne (1987)) and synthetic peptides (Giniger and Ptashne (1987)).

### Expression and Modification of Polypeptides

Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homolog.

The transgenic plants of the present invention comprising recombinant polynucleotide sequences are generally derived from parental plants, which may themselves be non-transformed (or non-transgenic) plants. These transgenic plants may either have a transcription factor gene “knocked out” (for example, with a genomic insertion by homologous recombination, an antisense or ribozyme construct) or expressed to a normal or wild-type extent. However, overexpressing transgenic “progeny” plants will exhibit greater mRNA levels, wherein the mRNA encodes a transcription factor, that is, a DNA-binding protein that is capable of binding to a DNA regulatory sequence and inducing transcription, and preferably, expression of a plant trait gene, such as a gene that improves plant and/or fruit quality and/or yield. Preferably, the mRNA expression level will be at least three-fold greater than that of the parental plant, or more preferably at least ten-fold greater mRNA levels compared to said parental plant, and most preferably at least fifty-fold greater compared to said parental plant.

### Vectors, Promoters, and Expression Systems

This section describes vectors, promoters, and expression systems that may be used with the present invention. Expression constructs that have been used to transform plants for testing in field trials are also described in Example III. The present invention includes recombinant constructs comprising one or more of the nucleic acid sequences herein. The constructs typically comprise a vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts that describe molecular biological techniques useful herein, including the use and production of vectors, promoters and many other relevant topics, include Berger and Kimmel (1987), Sambrook (1989) and Ausubel (2000). Any of the identified sequences can be incorporated into a cassette or vector, e.g., for expression in plants. A number of expression vectors suitable for stable transformation of plant cells or for the establishment of transgenic plants have been described including those described in Weissbach and Weissbach (1989) and Gelvin et al. (1990). Specific examples include those derived from a Ti plasmid of Agrobacterium tumefaciens, as well as those disclosed by Herrera-Estrella et al. (1983), Bevan (1984), and Klee (1985) for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou (1991) and corn (Gordon-Kamm (1990) can be produced. An immature embryo can also be a good target tissue for monocots for direct DNA delivery techniques by using the particle gun (Weeks et al. (1993); Vasil (1993a); Wan and Lemeaux (1994), and for Agrobacterium-mediated DNA transfer (Ishida et al. (1996)).

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

A potential utility for the transcription factor polynucleotides disclosed herein is the isolation of promoter elements from these genes that can be used to program expression in plants of any genes. Each transcription factor gene disclosed herein is expressed in a unique fashion, as determined by promoter elements located upstream of the start of translation, and additionally within an intron of the transcription factor gene or downstream of the termination codon of the gene. As is well known in the art, for a significant portion of genes, the promoter sequences are located entirely in the region directly upstream of the start of translation. In such cases, typically the promoter sequences are located within 2.0 KB of the start of translation, or within 1.5 KB of the start of translation, frequently within 1.0 KB of the start of translation, and sometimes within 0.5 KB of the start of translation.

The promoter sequences can be isolated according to methods known to one skilled in the art.

Examples of constitutive plant promoters which can be useful for expressing the transcription factor sequence include: the cauliflower mosaic virus (CaMV) 35S promoter, which confers constitutive, high-level expression in most plant tissues (see, e.g., Odell et al. (1985)); the nopaline synthase promoter (An et al. (1988)); and the octopine synthase promoter (Fromm et al. (1989)).

The transcription factors of the invention may be operably linked with a specific promoter that causes the transcription factor to be expressed in response to environmental, tissue-specific or temporal signals. A variety of plant gene promoters that regulate gene expression in response to environmental, hormonal, chemical, developmental signals, and in a tissue-active manner can be used for expression of a transcription factor sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like. Numerous known promoters have been characterized and can favorably be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988)), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli and Keller (1998)), flower-specific (Kaiser et al. (1995)), pollen (Baerson et al. (1994)), carpels (Ohl et al. (1990)), pollen and ovules (Baerson et al. (1993)), auxin-inducible promoters (such as that described in van der Kop et al. (999) or Baumann et al. (1999)), cytokinin-inducible promoter (Guevara-Garcia (1998)), promoters responsive to gibberellin (Shi et al. (1998), Willmott et al. (1998)) and the like. Additional promoters are those that elicit expression in response to heat (Ainley et al. (1993)), light (e.g., the pea rbcS-3A promoter, Kuhlemeier et al. (1989)), and the maize rbcS promoter, Schaffher and Sheen (1991)); wounding (e.g., wunI, Siebertz et al. (1989)); pathogens (such as the PR-1 promoter described in Buchel et al. (1999) and the PDF1.2 promoter described in Manners et al. (1998), and chemicals such as methyl jasmonate or salicylic acid (Gatz (1997)). In addition, the timing of the expression can be controlled by using promoters such as those acting at senescence (Gan and Amasino (1995)); or late seed development (Odell et al. (1994)).

Plant expression vectors can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

### Additional Expression Elements

Specific initiation signals can aid in efficient translation of coding sequences. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon can be separately provided. The initiation codon is provided in the correct reading frame to facilitate transcription. Exogenous transcriptional elements and initiation codons can be of various origins, both natural and synthetic. The efficiency of expression can be enhanced by the inclusion of enhancers appropriate to the cell system in use.

### Expression Hosts

The present invention also relates to host cells which are transduced with vectors of the invention, and the production of polypeptides of the invention (including fragments thereof) by recombinant techniques. Host cells are genetically engineered (i.e., nucleic acids are introduced, e.g., transduced, transformed or transfected) with the vectors of this invention, which may be, for example, a cloning vector or an expression vector comprising the relevant nucleic acids herein. The vector is optionally a plasmid, a viral particle, a phage, a naked nucleic acid, etc. The engineered host cells can be cultured in conventional nutrient media modified as appropriate for activating promoters, selecting transformants, or amplifying the relevant gene. The culture conditions, such as temperature, pH and the like, are those previously used with the host cell selected for expression, and will be apparent to those skilled in the art and in the references cited herein, including, Sambrook (1989) and Ausubel (2000).

The host cell can be a eukaryotic cell such as a yeast cell, or a plant cell, or the host cell can be a prokaryotic cell, such as a bacterial cell. Plant protoplasts are also suitable for some applications. For example, the DNA fragments are introduced into plant tissues, cultured plant cells or plant protoplasts by standard methods including electroporation (Fromm et al. (1985)), infection by viral vectors such as cauliflower mosaic virus (CaMV) (Hohn et al. (1982); U.S. Pat. No. 4,407,956), high velocity ballistic penetration by small particles with the nucleic acid either within the matrix of small beads or particles, or on the surface (Klein et al. (1987)), use of pollen as vector (WO 85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al. (1984); Fraley et al. (1983)).

The cell can include a nucleic acid of the invention that encodes a polypeptide, wherein the cell expresses a polypeptide of the invention. The cell can also include vector sequences, or the like. Furthermore, cells and transgenic plants that include any polypeptide or nucleic acid above or throughout this specification, e.g., produced by transduction of a vector of the invention, are an additional feature of the invention.

For long-term, high-yield production of recombinant proteins, stable expression can be used. Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding mature proteins of the invention can be designed with signal sequences which direct secretion of the mature polypeptides through a prokaryotic or eukaryotic cell membrane.

### Modified Amino Acid Residues

Polypeptides of the invention may contain one or more modified amino acid residues. The presence of modified amino acids may be advantageous in, for example, increasing polypeptide half-life, reducing polypeptide antigenicity or toxicity, increasing polypeptide storage stability, or the like. Amino acid residue(s) are modified, for example, co-translationally or post-translationally during recombinant production or modified by synthetic or chemical means.

Non-limiting examples of a modified amino acid residue include incorporation or other use of acetylated amino acids, glycosylated amino acids, sulfated amino acids, prenylated (e.g., farnesylated, geranylgeranylated) amino acids, PEG modified (e.g., “PEGylated”) amino acids, biotinylated amino acids, carboxylated amino acids, phosphorylated amino acids, etc. References adequate to guide one of skill in the modification of amino acid residues are replete throughout the literature.

The modified amino acid residues may prevent or increase affinity of the polypeptide for another molecule, including, but not limited to, polynucleotide, proteins, carbohydrates, lipids and lipid derivatives, and other organic or synthetic compounds.

### Identification of Additional Protein Factors

A transcription factor provided by the present invention can also be used to identify additional endogenous or exogenous molecules that can affect a phenotype or trait of interest. Such molecules include endogenous molecules that are acted upon either at a transcriptional level by a transcription factor of the invention to modify a phenotype as desired. For example, the transcription factors can be employed to identify one or more downstream genes that are subject to a regulatory effect of the transcription factor. In one approach, a transcription factor or transcription factor homolog of the invention is expressed in a host cell, e.g., a transgenic plant cell, tissue or explant, and expression products, either RNA or protein, of likely or random targets are monitored, e.g., by hybridization to a microarray of nucleic acid probes corresponding to genes expressed in a tissue or cell type of interest, by two-dimensional gel electrophoresis of protein products, or by any other method known in the art for assessing expression of gene products at the level of RNA or protein. Alternatively, a transcription factor of the invention can be used to identify promoter sequences (such as binding sites on DNA sequences) involved in the regulation of a downstream target. After identifying a promoter sequence, interactions between the transcription factor and the promoter sequence can be modified by changing specific nucleotides in the promoter sequence or specific amino acids in the transcription factor that interact with the promoter sequence to alter a plant trait. Typically, transcription factor DNA-binding sites are identified by gel shift assays. After identifying the promoter regions, the promoter region sequences can be employed in double-stranded DNA arrays to identify molecules that affect the interactions of the transcription factors with their promoters (Bulyk et al. (1999)).

The identified transcription factors are also useful to identify proteins that modify the activity of the transcription factor. Such modification can occur by covalent modification, such as by phosphorylation, or by protein-protein (homo or -heteropolymer) interactions. Any method suitable for detecting protein-protein interactions can be employed. Among the methods that can be employed are co-immunoprecipitation, cross-linking and co-purification through gradients or chromatographic columns, and the two-hybrid yeast system.

The two-hybrid system detects protein interactions in vivo and is described in Chien et al. (1991) and is commercially available from Clontech (Palo Alto, Calif.). In such a system, plasmids are constructed that encode two hybrid proteins: one consists of the DNA-binding domain of a transcription activator protein fused to the transcription factor polypeptide and the other consists of the transcription activator protein's activation domain fused to an unknown protein that is encoded by a cDNA that has been recombined into the plasmid as part of a cDNA library. The DNA-binding domain fusion plasmid and the cDNA library are transformed into a strain of the yeast Saccharomyces cerevisiae that contains a reporter gene (e.g., lacZ) whose regulatory region contains the transcription activator's binding site. Either hybrid protein alone cannot activate transcription of the reporter gene. Interaction of the two hybrid proteins reconstitutes the functional activator protein and results in expression of the reporter gene, which is detected by an assay for the reporter gene product. Then, the library plasmids responsible for reporter gene expression are isolated and sequenced to identify the proteins encoded by the library plasmids. After identifying proteins that interact with the transcription factors, assays for compounds that interfere with the transcription factor protein-protein interactions can be preformed.

### Subsequences

Also contemplated are uses of polynucleotides, also referred to herein as oligonucleotides, typically having at least 12 bases, preferably at least 50 bases, which hybridize under stringent conditions to a polynucleotide sequence described above. The polynucleotides may be used as probes, primers, sense and antisense agents, and the like, according to methods as noted above.

Subsequences of the polynucleotides of the invention, including polynucleotide fragments and oligonucleotides are useful as nucleic acid probes and primers. An oligonucleotide suitable for use as a probe or primer is at least about 15 nucleotides in length, more often at least about 18 nucleotides, often at least about 21 nucleotides, frequently at least about 30 nucleotides, or about 40 nucleotides, or more in length. A nucleic acid probe is useful in hybridization protocols, e.g., to identify additional polypeptide homologs of the invention, including protocols for microarray experiments. Primers can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods. See Sambrook (1989), and Ausubel (2000).

In addition, the invention includes an isolated or recombinant polypeptide including a subsequence of at least about 15 contiguous amino acids encoded by the recombinant or isolated polynucleotides of the invention. For example, such polypeptides, or domains or fragments thereof, can be used as immunogens, e.g., to produce antibodies specific for the polypeptide sequence, or as probes for detecting a sequence of interest. A subsequence can range in size from about 15 amino acids in length up to and including the full length of the polypeptide.

To be encompassed by the present invention, an expressed polypeptide which comprises such a polypeptide subsequence performs at least one biological function of the intact polypeptide in substantially the same manner, or to a similar extent, as does the intact polypeptide. For example, a polypeptide fragment can comprise a recognizable structural motif or functional domain such as a DNA binding domain that activates transcription, e.g., by binding to a specific DNA promoter region an activation domain, or a domain for protein-protein interactions.

### Production of Transgenic Plants

Modification of Traits

The polynucleotides of the invention are favorably employed to produce transgenic plants with various traits, or characteristics, that have been modified in a desirable manner, e.g., to improve the fruit quality characteristics of a plant. For example, alteration of expression levels or patterns (e.g., spatial or temporal expression patterns) of one or more of the transcription factors (or transcription factor homologs) of the invention, as compared with the levels of the same protein found in a wild-type plant, can be used to modify a plant's traits. An illustrative example of trait modification, improved characteristics, by altering expression levels of a particular transcription factor is described further in the Examples and the Sequence Listing.

Homologous Genes Introduced into Transgenic Plants.

Homologous genes that may be derived from any plant, or from any source whether natural, synthetic, semi-synthetic or recombinant, and that share significant sequence identity or similarity to those provided by the present invention, may be introduced into plants, for example, crop plants, to confer desirable or improved traits. Consequently, transgenic plants may be produced that comprise a recombinant expression vector or cassette with a promoter operably linked to one or more sequences homologous to presently disclosed sequences. The promoter may be, for example, a plant or viral promoter.

The invention thus provides for methods for preparing transgenic plants, and for modifying plant traits. These methods include introducing into a plant a recombinant expression vector or cassette comprising a functional promoter operably linked to one or more sequences homologous to presently disclosed sequences. Plants and kits for producing these plants that result from the application of these methods are also encompassed by the present invention.

Genes, Traits and Utilities that Affect Plant Characteristics

Plant transcription factors can modulate gene expression, and, in turn, be modulated by the environmental experience of a plant. Significant alterations in a plant's environment invariably result in a change in the plant's transcription factor gene expression pattern. Altered transcription factor expression patterns generally result in phenotypic changes in the plant. Transcription factor gene product(s) in transgenic plants then differ(s) in amounts or proportions from that found in wild-type or non-transformed plants, and those transcription factors likely represent polypeptides that are used to alter the response to the environmental change. By way of example, it is well accepted in the art that analytical methods based on altered expression patterns may be used to screen for phenotypic changes in a plant far more effectively than can be achieved using traditional methods.

Potential Applications of the Presently Disclosed Sequences that Improve Plant Yield and/or Fruit Yield or Quality

The genes identified by the experiment presently disclosed represent potential regulators of plant yield and/or fruit yield or quality. As such, these genes (or their orthologs and paralogs) can be applied to commercial species in order to produce higher yield and/or quality.

### Antisense and Co-Suppression

In addition to expression of the nucleic acids of the invention as gene replacement or plant phenotype modification nucleic acids, the nucleic acids are also useful for sense and anti-sense suppression of expression, e.g. to down-regulate expression of a nucleic acid of the invention, e.g. as a further mechanism for modulating plant phenotype. That is, the nucleic acids of the invention, or subsequences or anti-sense sequences thereof, can be used to block expression of naturally occurring homologous nucleic acids. A variety of sense and anti-sense technologies are known in the art, e.g. as set forth in Lichtenstein and Nellen (1997) Antisense Technology: A Practical Approach IRL Press at Oxford University Press, Oxford, U.K. Antisense regulation is also described in Crowley et al. (1985); Rosenberg et al. (1985); Preiss et al. (1985); Melton (1985); Izant and Weintraub (1985); and Kim and Wold (1985). Additional methods for antisense regulation are known in the art. Antisense regulation has been used to reduce or inhibit expression of plant genes in, for example in European Patent Publication No. 271988. Antisense RNA may be used to reduce gene expression to produce a visible or biochemical phenotypic change in a plant (Smith et al. (1988); Smith et al. (1990)). In general, sense or anti-sense sequences are introduced into a cell, where they are optionally amplified, e.g. by transcription. Such sequences include both simple oligonucleotide sequences and catalytic sequences such as ribozymes.

For example, a reduction or elimination of expression (i.e., a “knock-out”) of a transcription factor or transcription factor homolog polypeptide in a transgenic plant, e.g., to modify a plant trait, can be obtained by introducing an antisense construct corresponding to the polypeptide of interest as a cDNA. For antisense suppression, the transcription factor or homolog cDNA is arranged in reverse orientation (with respect to the coding sequence) relative to the promoter sequence in the expression vector. The introduced sequence need not be the full-length cDNA or gene, and need not be identical to the cDNA or gene found in the plant type to be transformed. Typically, the antisense sequence need only be capable of hybridizing to the target gene or RNA of interest. Thus, where the introduced sequence is of shorter length, a higher degree of homology to the endogenous transcription factor sequence will be needed for effective antisense suppression. While antisense sequences of various lengths can be utilized, preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous transcription factor gene in the plant cell.

Suppression of endogenous transcription factor gene expression can also be achieved using RNA interference, or RNAi. RNAi is a post-transcriptional, targeted gene-silencing technique that uses double-stranded RNA (dsRNA) to incite degradation of messenger RNA (mRNA) containing the same sequence as the dsRNA (Constans (2002)). Small interfering RNAs, or siRNAs are produced in at least two steps: an endogenous ribonuclease cleaves longer dsRNA into shorter, 21-23 nucleotide-long RNAs. The siRNA segments then mediate the degradation of the target mRNA (Zamore (2001). RNAi has been used for gene function determination in a manner similar to antisense oligonucleotides (Constans (2002)). Expression vectors that continually express siRNAs in transiently and stably transfected have been engineered to express small hairpin RNAs (shRNAs), which get processed in vivo into siRNAs-like molecules capable of carrying out gene-specific silencing (Brummelkamp et al. (2002), and Paddison, et al. (2002)). Post-transcriptional gene silencing by double-stranded RNA is discussed in further detail by Hammond et al. (2001), Fire et al. (1998) and Timmons and Fire (1998). Vectors in which RNA encoded by a transcription factor or transcription factor homolog cDNA is over-expressed can also be used to obtain co-suppression of a corresponding endogenous gene, e.g., in the manner described in U.S. Pat. No. 5,231,020 to Jorgensen. Such co-suppression (also termed sense suppression) does not require that the entire transcription factor cDNA be introduced into the plant cells, nor does it require that the introduced sequence be exactly identical to the endogenous transcription factor gene of interest. However, as with antisense suppression, the suppressive efficiency will be enhanced as specificity of hybridization is increased, e.g., as the introduced sequence is lengthened, and/or as the sequence similarity between the introduced sequence and the endogenous transcription factor gene is increased.

Vectors expressing an untranslatable form of the transcription factor mRNA, e.g., sequences comprising one or more stop codon, or nonsense mutation) can also be used to suppress expression of an endogenous transcription factor, thereby reducing or eliminating its activity and modifying one or more traits. Methods for producing such constructs are described in U.S. Pat. No. 5,583,021. Preferably, such constructs are made by introducing a premature stop codon into the transcription factor gene. Alternatively, a plant trait can be modified by gene silencing using double-strand RNA (Sharp (1999)). Another method for abolishing the expression of a gene is by insertion mutagenesis using the T-DNA of Agrobacterium tumefaciens. After generating the insertion mutants, the mutants can be screened to identify those containing the insertion in a transcription factor or transcription factor homolog gene. Plants containing a single transgene insertion event at the desired gene can be crossed to generate homozygous plants for the mutation. Such methods are well known to those of skill in the art (see for example Koncz et al. (1992a, 1992b)).

Alternatively, a plant phenotype can be altered by eliminating an endogenous gene, such as a transcription factor or transcription factor homolog, e.g., by homologous recombination (Kempin et al. (1997)).

A plant trait can also be modified by using the Cre-lox system (for example, as described in U.S. Pat. No. 5,658,772). A plant genome can be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

The polynucleotides and polypeptides of this invention can also be expressed in a plant in the absence of an expression cassette by manipulating the activity or expression level of the endogenous gene by other means, such as, for example, by ectopically expressing a gene by T-DNA activation tagging (Ichikawa et al. (1997); Kakimoto et al. (1996)). This method entails transforming a plant with a gene tag containing multiple transcriptional enhancers and once the tag has inserted into the genome, expression of a flanking gene coding sequence becomes deregulated. In another example, the transcriptional machinery in a plant can be modified so as to increase transcription levels of a polynucleotide of the invention (see, e.g., PCT Publications WO 96/06166 and WO 98/53057 which describe the modification of the DNA-binding specificity of zinc finger proteins by changing particular amino acids in the DNA-binding motif).

The transgenic plant can also include the machinery necessary for expressing or altering the activity of a polypeptide encoded by an endogenous gene, for example, by altering the phosphorylation state of the polypeptide to maintain it in an activated state.

Transgenic plants (or plant cells, or plant explants, or plant tissues) incorporating the polynucleotides of the invention and/or expressing the polypeptides of the invention can be produced by a variety of well established techniques as described above. Following construction of a vector, most typically an expression cassette, including a polynucleotide, e.g., encoding a transcription factor or transcription factor homolog, of the invention, standard techniques can be used to introduce the polynucleotide into a plant, a plant cell, a plant explant or a plant tissue of interest. Optionally, the plant cell, explant or tissue can be regenerated to produce a transgenic plant.

The plant can be any higher plant, including gymnosperms, monocotyledonous and dicotyledonous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984); Shimamoto et al. (1989); Fromm et al. (1990); and Vasil et al. (1990).

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumefaciens mediated transformation. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants showing a modified trait are identified using methods well known in the art that are specifically directed to improved fruit or yield characteristics. Methods that may be used are provided in Examples II through VI. The modified trait can be any of those traits described above. Additionally, to confirm that the modified trait is due to changes in expression levels or activity of the polypeptide or polynucleotide of the invention can be determined by analyzing mRNA expression using Northern blots, RT-PCR or microarrays, or protein expression using immunoblots or Western blots or gel shift assays.

### Integrated Systems—Sequence Identity

Additionally, the present invention may be an integrated system, computer or computer readable medium that comprises an instruction set for determining the identity of one or more sequences in a database. In addition, the instruction set can be used to generate or identify sequences that meet any specified criteria. Furthermore, the instruction set may be used to associate or link certain functional benefits, such improved characteristics, with one or more identified sequence.

For example, the instruction set can include, e.g., a sequence comparison or other alignment program, e.g., an available program such as, for example, the Wisconsin Package Version 10.0, such as BLAST, FASTA, PILEUP, FINDPATTERNS or the like (GCG, Madison, Wis.). Public sequence databases such as GenBank, EMBL, Swiss-Prot and PIR or private sequence databases such as PHYTOSEQ sequence database (Incyte Genomics, Wilmington, Del.) can be searched.

Alignment of sequences for comparison can be conducted by the local homology algorithm of Smith and Waterman (1981), by the homology alignment algorithm of Needleman and Wunsch (1970, by the search for similarity method of Pearson and Lipman (1988), or by computerized implementations of these algorithms. After alignment, sequence comparisons between two (or more) polynucleotides or polypeptides are typically performed by comparing sequences of the two sequences over a comparison window to identify and compare local regions of sequence similarity. The comparison window can be a segment of at least about 20 contiguous positions, usually about 50 to about 200, more usually about 100 to about 150 contiguous positions. A description of the method is provided in Ausubel (2000).

A variety of methods for determining sequence relationships can be used, including manual alignment and computer assisted sequence alignment and analysis. This later approach is a preferred approach in the present invention, due to the increased throughput afforded by computer assisted methods. As noted above, a variety of computer programs for performing sequence alignment are available, or can be produced by one of skill.

One example algorithm that is suitable for determining percent sequence identity and sequence similarity is the BLAST algorithm, which is described in Altschul et al. (1990). Software for performing BLAST analyses is publicly available, e.g., through the National Library of Medicine's National Center for Biotechnology Information (ncbi.nlm.nih; see at world wide web (www) National Institutes of Health US government (gov) website). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (2000)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix (see Henikoff and Henikoff (1992)). Unless otherwise indicated, “sequence identity” here refers to the % sequence identity generated from a tblastx using the NCBI version of the algorithm at the default settings using gapped alignments with the filter “off” (see, for example, NIH NLM NCBI website at ncbi.nlm.nih).

In addition to calculating percent sequence identity, the BLAST algorithm also performs a statistical analysis of the similarity between two sequences (see, e.g. Karlin and Altschul (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001. An additional example of a useful sequence alignment algorithm is PILEUP. PILEUP creates a multiple sequence alignment from a group of related sequences using progressive, pairwise alignments. The program can align, e.g., up to 300 sequences of a maximum length of 5,000 letters.

The integrated system, or computer typically includes a user input interface allowing a user to selectively view one or more sequence records corresponding to the one or more character strings, as well as an instruction set which aligns the one or more character strings with each other or with an additional character string to identify one or more region of sequence similarity. The system may include a link of one or more character strings with a particular phenotype or gene function. Typically, the system includes a user readable output element that displays an alignment produced by the alignment instruction set.

The methods of this invention can be implemented in a localized or distributed computing environment. In a distributed environment, the methods may be implemented on a single computer comprising multiple processors or on a multiplicity of computers. The computers can be linked, e.g. through a common bus, but more preferably the computer(s) are nodes on a network. The network can be a generalized or a dedicated local or wide-area network and, in certain preferred embodiments, the computers may be components of an intra-net or an internet.

Thus, the invention provides methods for identifying a sequence similar or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an inter or intra net) and a query is made against the sequence database using the relevant sequences herein and associated plant phenotypes or gene functions.

Any sequence herein can be entered into the database, before or after querying the database. This provides for both expansion of the database and, if done before the querying step, for insertion of control sequences into the database. The control sequences can be detected by the query to ensure the general integrity of both the database and the query. As noted, the query can be performed using a web browser based interface. For example, the database can be a centralized public database such as those noted herein, and the querying can be done from a remote terminal or computer across an internet or intranet.

Any sequence herein can be used to identify a similar, homologous, paralogous, or orthologous sequence in another plant. This provides means for identifying endogenous sequences in other plants that may be useful to alter a trait of progeny plants, which results from crossing two plants of different strain. For example, sequences that encode an ortholog of any of the sequences herein that naturally occur in a plant with a desired trait can be identified using the sequences disclosed herein. The plant is then crossed with a second plant of the same species but which does not have the desired trait to produce progeny which can then be used in further crossing experiments to produce the desired trait in the second plant. Therefore the resulting progeny plant contains no transgenes; expression of the endogenous sequence may also be regulated by treatment with a particular chemical or other means, such as EMR. Some examples of such compounds well known in the art include: ethylene; cytokinins; phenolic compounds, which stimulate the transcription of the genes needed for infection; specific monosaccharides and acidic environments which potentiate vir gene induction; acidic polysaccharides which induce one or more chromosomal genes; and opines; other mechanisms include light or dark treatment (for a review of examples of such treatments, see Winans (1992), Eyal et al. (1992), Chrispeels et al. (2000), or Piazza et al. (2002)).

Table 5 categorizes sequences within the National Center for Biotechnology Information (NCBI) UniGene database determined to be orthologous to many of the transcription factor sequences of the present invention. The column headings include the transcription factors listed by (a) the SEQ ID NO: of each Clade Identifier; (b) the Clade Identifier (the “reference” Arabidopsis Gene Identifier (GID) used to identify each clade); (c) the AGI Identifier for each Clade Identifier; (d) the UniGene identifier for each orthologous sequence identified in this study; (e) SEQ ID NO: of the ortholog found in the UniGene database (these public sequences are not provided in the Sequence Listing but are expected to function similarly to the respective Clade Identifiers based on sequence similarity, including similarity within the conserved domains); (f) the species in which the orthologs to the transcription factors are found; (g) the smallest sum probability relationship of the homologous sequence to Arabidopsis Clade Identifier sequence in a given row, determined by BLAST analysis, and (h) the percentage identity of the ortholog found in the UniGene database to the Clade Identifier.

TABLE 5
Orthologs of Representative Arabidopsis Transcription Factor Genes Identified Using BLAST
Analysis

| | | | | | | | % Identity |
|---|---|---|---|---|---|---|---|
| Clade | | AGI | | | | | of |
| Identifier | Clade | Identifier for | | Ortholog | | | Ortholog |
| SEQ ID | Identifier | Clade | UniGene | SEQ ID | | | to Clade |
| NO: | (GID) | Identifier | Identifier | NO: | Species | p-Value | Identifier |
| 1 | G3 | AT1G46768 | Gma_S4867812 | 437 | Glycine max | 8.00E−29 | 54% |
| 1 | G3 | AT1G46768 | Gma_S4919945 | 438 | Glycine max | 2.00E−27 | 59% |
| 1 | G3 | AT1G46768 | Lsa_S18816809 | 709 | Lactuca | 9.00E−12 | 53% |
| | | | | | sativa | | |
| 3 | G22 | AT2G44840 | Gma_S5146194 | 439 | Glycine max | 3.00E−30 | 58% |
| 3 | G22 | AT2G44840 | Hv_S8652 | 488 | Hordeum | 7.00E−08 | 49% |
| | | | | | vulgare | | |
| 3 | G22 | AT2G44840 | Lsa_S18782253 | 710 | Lactuca | 6.00E−27 | 65% |
| | | | | | sativa | | |
| 3 | G22 | AT2G44840 | Lco_S19325549 | 737 | Lotus | 2.00E−27 | 66% |
| | | | | | corniculatus | | |
| 3 | G22 | AT2G44840 | Lco_S19424678 | 738 | Lotus | 7.00E−14 | 40% |
| | | | | | corniculatus | | |
| 3 | G22 | AT2G44840 | Les_S5295747 | 574 | Lycopersicon | 1.00E−53 | 54% |
| | | | | | esculentum | | |
| 3 | G22 | AT2G44840 | SGN-UNIGENE- | 581 | Lycopersicon | 2.00E−53 | 54% |
| | | | 47863 | | esculentum | | |
| 3 | G22 | AT2G44840 | SGN-UNIGENE- | 582 | Lycopersicon | 1.00E−45 | 60% |
| | | | SINGLET-65809 | | esculentum | | |
| 3 | G22 | AT2G44840 | Mtr_S5317111 | 476 | Medicago | 2.00E−28 | 61% |
| | | | | | truncatula | | |
| 3 | G22 | AT2G44840 | Ppa_S17591179 | 807 | Physcomitrella | 3.00E−26 | 64% |
| | | | | | patens | | |
| 3 | G22 | AT2G44840 | Ppa_S17606123 | 808 | Physcomitrella | 2.00E−26 | 78% |
| | | | | | patens | | |
| 3 | G22 | AT2G44840 | Ppa_S17633322 | 809 | Physcomitrella | 7.00E−26 | 63% |
| | | | | | patens | | |
| 3 | G22 | AT2G44840 | Pta_S16845454 | 690 | Pinus taeda | 1.00E−26 | 55% |
| 3 | G22 | AT2G44840 | Stu_S18122190 | 783 | Solanum | 1.00E−54 | 54% |
| | | | | | tuberosum | | |
| 3 | G22 | AT2G44840 | Stu_S18128192 | 784 | Solanum | 1.00E−53 | 54% |
| | | | | | tuberosum | | |
| 3 | G22 | AT2G44840 | Vvi_S15422284 | 661 | Vitis vinifera | 6.00E−33 | 51% |
| 3 | G22 | AT2G44840 | Zm_S11434059 | 502 | Zea mays | 1.00E−06 | 48% |
| 5 | G24 | AT2G23340 | Gma_S5071803 | 440 | Glycine max | 3.00E−40 | 55% |
| 5 | G24 | AT2G23340 | Han_S18753000 | 704 | Helianthus | 2.00E−42 | 61% |
| | | | | | annuus | | |
| 5 | G24 | AT2G23340 | SGN-UNIGENE- | 583 | Lycopersicon | 1.00E−14 | 42% |
| | | | 49683 | | esculentum | | |
| 5 | G24 | AT2G23340 | SGN-UNIGENE- | 584 | Lycopersicon | 4.00E−41 | 53% |
| | | | 54594 | | esculentum | | |
| 5 | G24 | AT2G23340 | SGN-UNIGENE- | 585 | Lycopersicon | 1.00E−19 | 72% |
| | | | SINGLET-47313 | | esculentum | | |
| 5 | G24 | AT2G23340 | Os_S32369 | 403 | Oryza sativa | 1.00E−13 | 43% |
| 5 | G24 | AT2G23340 | Os_S80194 | 404 | Oryza sativa | 4.00E−08 | 59% |
| 5 | G24 | AT2G23340 | Stu_S18119664 | 785 | Solanum | 1.00E−23 | 75% |
| | | | | | tuberosum | | |
| 5 | G24 | AT2G23340 | Sbi_S19492185 | 761 | Sorghum | 2.00E−06 | 37% |
| | | | | | bicolor | | |
| 5 | G24 | AT2G23340 | Vvi_S15370190 | 662 | Vitis vinifera | 1.00E−38 | 52% |
| 5 | G24 | AT2G23340 | Vvi_S16806812 | 663 | Vitis vinifera | 6.00E−25 | 55% |
| 9 | G156 | AT5G23260 | SGN-UNIGENE- | 586 | Lycopersicon | 5.00E−40 | 49% |
| | | | 54690 | | esculentum | | |
| 13 | G187 | AT4G18170 | Zm_S11434549 | 503 | Zea mays | 4.00E−34 | 74% |
| 17 | G226 | AT2G30420 | Gma_S4892930 | 441 | Glycine max | 2.00E−06 | 72% |
| 17 | G226 | AT2G30420 | Gma_S4901946 | 442 | Glycine max | 0.004 | 76% |
| 17 | G226 | AT2G30420 | Ptp_S17966041 | 725 | Populus | 2.00E−12 | 54% |
| | | | | | tremula x | | |
| | | | | | Populus | | |
| | | | | | tremuloides | | |
| 17 | G226 | AT2G30420 | Ta_S45274 | 543 | Triticum | 3.00E−14 | 57% |
| | | | | | aestivum | | |
| 17 | G226 | AT2G30420 | Vvi_S15356289 | 664 | Vitis vinifera | 2.00E−30 | 76% |
| 17 | G226 | AT2G30420 | Vvi_S16820566 | 665 | Vitis vinifera | 3.00E−12 | 56% |
| 19 | G237 | AT4G25560 | Zm_S11529151 | 504 | Zea mays | 3.00E−13 | 69% |
| 21 | G270 | AT5G66055 | Gma_S4950212 | 443 | Glycine max | 3.00E−59 | 61% |
| 21 | G270 | AT5G66055 | Lsa_S18811068 | 711 | Lactuca | 1.00E−76 | 55% |
| | | | | | sativa | | |
| 21 | G270 | AT5G66055 | SGN-UNIGENE- | 587 | Lycopersicon | 9.00E−28 | 35% |
| | | | 51108 | | esculentum | | |
| 21 | G270 | AT5G66055 | SGN-UNIGENE- | 588 | Lycopersicon | 7.00E−19 | 34% |
| | | | 51109 | | esculentum | | |
| 21 | G270 | AT5G66055 | SGN-UNIGENE- | 589 | Lycopersicon | 1.00E−51 | 70% |
| | | | SINGLET-39801 | | esculentum | | |
| 21 | G270 | AT5G66055 | Stu_S14633069 | 787 | Solanum | 3.00E−42 | 71% |
| | | | | | tuberosum | | |
| 21 | G270 | AT5G66055 | Zm_S11522249 | 505 | Zea mays | 2.00E−57 | 63% |
| 23 | G328 | AT5G15850 | Gma_S4909503 | 444 | Glycine max | 6.00E−05 | 63% |
| 23 | G328 | AT5G15850 | Hv_S210900 | 489 | Hordeum | 1.00E−40 | 32% |
| | | | | | vulgare | | |
| 23 | G328 | AT5G15850 | Hv_S210901 | 490 | Hordeum | 1.00E−43 | 36% |
| | | | | | vulgare | | |
| 23 | G328 | AT5G15850 | SGN-UNIGENE- | 590 | Lycopersicon | 3.00E−58 | 50% |
| | | | 52452 | | esculentum | | |
| 23 | G328 | AT5G15850 | SGN-UNIGENE- | 591 | Lycopersicon | 6.00E−31 | 67% |
| | | | 58595 | | esculentum | | |
| 23 | G328 | AT5G15850 | Mtr_S5441621 | 477 | Medicago | 2.00E−40 | 64% |
| | | | | | truncatula | | |
| 23 | G328 | AT5G15850 | Os_S108164 | 407 | Oryza sativa | 4.00E−10 | 53% |
| 23 | G328 | AT5G15850 | Os_S60493 | 408 | Oryza sativa | 3.00E−47 | 37% |
| 23 | G328 | AT5G15850 | Os_S63686 | 409 | Oryza sativa | 2.00E−77 | 45% |
| 23 | G328 | AT5G15850 | Ppa_S17598269 | 811 | Physcomitrella | 9.00E−28 | 53% |
| | | | | | patens | | |
| 23 | G328 | AT5G15850 | Ppa_S17623794 | 812 | Physcomitrella | 9.00E−20 | 60% |
| | | | | | patens | | |
| 23 | G328 | AT5G15850 | Ptp_S17915054 | 726 | Populus | 3.00E−46 | 60% |
| | | | | | tremula x | | |
| | | | | | Populus | | |
| | | | | | tremuloides | | |
| 23 | G328 | AT5G15850 | Stu_S18109267 | 788 | Solanum | 3.00E−30 | 72% |
| | | | | | tuberosum | | |
| 23 | G328 | AT5G15850 | Ta_S344859 | 544 | Triticum | 0.55 | 33% |
| | | | | | aestivum | | |
| 23 | G328 | AT5G15850 | Ta_S378085 | 545 | Triticum | 4.00E−16 | 55% |
| | | | | | aestivum | | |
| 23 | G328 | AT5G15850 | Ta_S60632 | 546 | Triticum | 2.00E−12 | 59% |
| | | | | | aestivum | | |
| 23 | G328 | AT5G15850 | Vvi_S15370390 | 666 | Vitis vinifera | 5.00E−38 | 72% |
| 23 | G328 | AT5G15850 | Vvi_S16866787 | 667 | Vitis vinifera | 1.00E−57 | 57% |
| 23 | G328 | AT5G15850 | Zm_S11527431 | 506 | Zea mays | 4.00E−24 | 52% |
| 25 | G363 | AT1G66140 | Gma_S4865156 | 445 | Glycine max | 0.004 | 30% |
| 25 | G363 | AT1G66140 | Gma_S4916522 | 446 | Glycine max | 8.00E−21 | 45% |
| 25 | G363 | AT1G66140 | Gma_S5129767 | 447 | Glycine max | 1.00E−10 | 31% |
| 25 | G363 | AT1G66140 | Han_S18753949 | 705 | Helianthus | 4.00E−10 | 39% |
| | | | | | annuus | | |
| 25 | G363 | AT1G66140 | Lco_S19421621 | 739 | Lotus | 0.003 | 32% |
| | | | | | corniculatus | | |
| 25 | G363 | AT1G66140 | SGN-UNIGENE- | 592 | Lycopersicon | 1.00E−29 | 45% |
| | | | 50506 | | esculentum | | |
| 25 | G363 | AT1G66140 | SGN-UNIGENE- | 593 | Lycopersicon | 0.052 | 41% |
| | | | 50507 | | esculentum | | |
| 25 | G363 | AT1G66140 | Stu_S18124970 | 789 | Solanum | 2.00E−40 | 44% |
| | | | | | tuberosum | | |
| 25 | G363 | AT1G66140 | Stu_S18130146 | 790 | Solanum | 5.00E−43 | 44% |
| | | | | | tuberosum | | |
| 25 | G363 | AT1G66140 | Vvi_S16866946 | 668 | Vitis vinifera | 3.00E−17 | 33% |
| 25 | G363 | AT1G66140 | Vvi_S16868836 | 669 | Vitis vinifera | 1.00E−42 | 43% |
| 25 | G363 | AT1G66140 | Zm_S11443746 | 507 | Zea mays | 8.00E−23 | 42% |
| 29 | G435 | AT5G53980 | SGN-UNIGENE- | 594 | Lycopersicon | 1.00E−24 | 42% |
| | | | SINGLET-385221 | | esculentum | | |
| 31 | G450 | AT4G14550 | Gma_S4866223 | 448 | Glycine max | 3.00E−42 | 52% |
| 31 | G450 | AT4G14550 | Gma_S4868219 | 449 | Glycine max | 1.00E−44 | 41% |
| 31 | G450 | AT4G14550 | Gma_S4871358 | 450 | Glycine max | 0.01 | 94% |
| 31 | G450 | AT4G14550 | Gma_S4878791 | 451 | Glycine max | 2.00E−47 | 63% |
| 31 | G450 | AT4G14550 | Gma_S5052530 | 452 | Glycine max | 3.00E−21 | 62% |
| 31 | G450 | AT4G14550 | Gma_S5079574 | 453 | Glycine max | 4.00E−62 | 69% |
| 31 | G450 | AT4G14550 | Gma_S5146462 | 454 | Glycine max | 5.00E−36 | 55% |
| 31 | G450 | AT4G14550 | Gma_S5146870 | 455 | Glycine max | 4.00E−73 | 61% |
| 31 | G450 | AT4G14550 | Han_S18710127 | 706 | Helianthus | 2.00E−56 | 75% |
| | | | | | annuus | | |
| 31 | G450 | AT4G14550 | Hv_S5546 | 491 | Hordeum | 1.00E−11 | 69% |
| | | | | | vulgare | | |
| 31 | G450 | AT4G14550 | Hv_S65240 | 492 | Hordeum | 1.00E−36 | 45% |
| | | | | | vulgare | | |
| 31 | G450 | AT4G14550 | Hv_S68291 | 493 | Hordeum | 8.00E−52 | 67% |
| | | | | | vulgare | | |
| 31 | G450 | AT4G14550 | Hv_S69191 | 494 | Hordeum | 1.00E−55 | 55% |
| | | | | | vulgare | | |
| 31 | G450 | AT4G14550 | Lsa_S18800753 | 712 | Lactuca | 8.00E−19 | 88% |
| | | | | | sativa | | |
| 31 | G450 | AT4G14550 | Lsa_S18822784 | 713 | Lactuca | 8.00E−80 | 70% |
| | | | | | sativa | | |
| 31 | G450 | AT4G14550 | Lco_S19280850 | 740 | Lotus | 3.00E−30 | 48% |
| | | | | | corniculatus | | |
| 31 | G450 | AT4G14550 | Lco_S19282187 | 741 | Lotus | 2.00E−35 | 91% |
| | | | | | corniculatus | | |
| 31 | G450 | AT4G14550 | Lco_S19284100 | 742 | Lotus | 3.00E−41 | 58% |
| | | | | | corniculatus | | |
| 31 | G450 | AT4G14550 | Lco_S19307099 | 743 | Lotus | 2.00E−31 | 53% |
| | | | | | corniculatus | | |
| 31 | G450 | AT4G14550 | Lco_S19373911 | 744 | Lotus | 4.00E−29 | 84% |
| | | | | | corniculatus | | |
| 31 | G450 | AT4G14550 | Lco_S19399973 | 745 | Lotus | 5.00E−19 | 88% |
| | | | | | corniculatus | | |
| 31 | G450 | AT4G14550 | Lco_S19414267 | 746 | Lotus | 3.00E−13 | 67% |
| | | | | | corniculatus | | |
| 31 | G450 | AT4G14550 | Lco_S19457695 | 747 | Lotus | 5.00E−41 | 60% |
| | | | | | corniculatus | | |
| 31 | G450 | AT4G14550 | Lco_S19458479 | 748 | Lotus | 2.00E−05 | 87% |
| | | | | | corniculatus | | |
| 31 | G450 | AT4G14550 | Les_S5267807 | 575 | Lycopersicon | 5.00E−10 | 71% |
| | | | | | esculentum | | |
| 31 | G450 | AT4G14550 | Les_S5295354 | 576 | Lycopersicon | 8.00E−25 | 56% |
| | | | | | esculentum | | |
| 31 | G450 | AT4G14550 | Les_S5295355 | 577 | Lycopersicon | 4.00E−34 | 66% |
| | | | | | esculentum | | |
| 31 | G450 | AT4G14550 | Les_S5295425 | 578 | Lycopersicon | 5.00E−14 | 88% |
| | | | | | esculentum | | |
| 31 | G450 | AT4G14550 | SGN-UNIGENE- | 595 | Lycopersicon | 2.00E−82 | 64% |
| | | | 46256 | | esculentum | | |
| 31 | G450 | AT4G14550 | SGN-UNIGENE- | 596 | Lycopersicon | 4.00E−64 | 62% |
| | | | 46318 | | esculentum | | |
| 31 | G450 | AT4G14550 | SGN-UNIGENE- | 597 | Lycopersicon | 5.00E−54 | 50% |
| | | | 48967 | | esculentum | | |
| 31 | G450 | AT4G14550 | SGN-UNIGENE- | 598 | Lycopersicon | 0.056 | 71% |
| | | | 58998 | | esculentum | | |
| 31 | G450 | AT4G14550 | SGN-UNIGENE- | 599 | Lycopersicon | 7.00E−56 | 57% |
| | | | SINGLET-355280 | | esculentum | | |
| 31 | G450 | AT4G14550 | SGN-UNIGENE- | 600 | Lycopersicon | 2.00E−81 | 67% |
| | | | SINGLET-393131 | | esculentum | | |
| 31 | G450 | AT4G14550 | Mtr_S16420818 | 478 | Medicago | 6.00E−64 | 62% |
| | | | | | truncatula | | |
| 31 | G450 | AT4G14550 | Mtr_S5409604 | 479 | Medicago | 8.00E−36 | 87% |
| | | | | | truncatula | | |
| 31 | G450 | AT4G14550 | Mtr_S5443886 | 480 | Medicago | 3.00E−26 | 76% |
| | | | | | truncatula | | |
| 31 | G450 | AT4G14550 | Os_S106147 | 411 | Oryza sativa | 2.00E−09 | 73% |
| 31 | G450 | AT4G14550 | Os_S55790 | 413 | Oryza sativa | 7.00E−16 | 66% |
| 31 | G450 | AT4G14550 | Os_S83247 | 414 | Oryza sativa | 1.00E−59 | 54% |
| 31 | G450 | AT4G14550 | Ppa_S17639899 | 813 | Physcomitrella | 4.00E−32 | 42% |
| | | | | | patens | | |
| 31 | G450 | AT4G14550 | Ppa_S17639910 | 814 | Physcomitrella | 3.00E−32 | 42% |
| | | | | | patens | | |
| 31 | G450 | AT4G14550 | Pta_S16175974 | 692 | Pinus taeda | 2.00E−51 | 48% |
| 31 | G450 | AT4G14550 | Pta_S16175975 | 693 | Pinus taeda | 3.00E−53 | 47% |
| 31 | G450 | AT4G14550 | Pta_S16175977 | 694 | Pinus taeda | 2.00E−49 | 47% |
| 31 | G450 | AT4G14550 | Pta_S16792071 | 695 | Pinus taeda | 8.00E−27 | 83% |
| 31 | G450 | AT4G14550 | Ptp_S17971671 | 727 | Populus | 8.00E−87 | 68% |
| | | | | | tremula x | | |
| | | | | | Populus | | |
| | | | | | tremuloides | | |
| 31 | G450 | AT4G14550 | Ptp_S17971673 | 728 | Populus | 3.00E−75 | 56% |
| | | | | | tremula x | | |
| | | | | | Populus | | |
| | | | | | tremuloides | | |
| 31 | G450 | AT4G14550 | Ptp_S17971674 | 729 | Populus | 1.00E−84 | 63% |
| | | | | | tremula x | | |
| | | | | | Populus | | |
| | | | | | tremuloides | | |
| 31 | G450 | AT4G14550 | Sof_S17381655 | 773 | Saccharum | 5.00E−07 | 50% |
| | | | | | officinarum | | |
| 31 | G450 | AT4G14550 | Stu_S18110580 | 791 | Solanum | 8.00E−89 | 70% |
| | | | | | tuberosum | | |
| 31 | G450 | AT4G14550 | Stu_S18128606 | 792 | Solanum | 2.00E−82 | 67% |
| | | | | | tuberosum | | |
| 31 | G450 | AT4G14550 | Sbi_S19502140 | 763 | Sorghum | 2.00E−53 | 49% |
| | | | | | bicolor | | |
| 31 | G450 | AT4G14550 | Sbi_S19503070 | 764 | Sorghum | 3.00E−46 | 61% |
| | | | | | bicolor | | |
| 31 | G450 | AT4G14550 | Ta_S106537 | 547 | Triticum | 5.00E−33 | 59% |
| | | | | | aestivum | | |
| 31 | G450 | AT4G14550 | Ta_S214840 | 548 | Triticum | 7.00E−51 | 63% |
| | | | | | aestivum | | |
| 31 | G450 | AT4G14550 | Ta_S280029 | 549 | Triticum | 1.00E−22 | 39% |
| | | | | | aestivum | | |
| 31 | G450 | AT4G14550 | Ta_S300894 | 550 | Triticum | 3.00E−06 | 91% |
| | | | | | aestivum | | |
| 31 | G450 | AT4G14550 | Ta_S310132 | 552 | Triticum | 7.00E−23 | 80% |
| | | | | | aestivum | | |
| 31 | G450 | AT4G14550 | Ta_S321320 | 553 | Triticum | 2.00E−39 | 68% |
| | | | | | aestivum | | |
| 31 | G450 | AT4G14550 | Ta_S41569 | 554 | Triticum | 5.00E−50 | 67% |
| | | | | | aestivum | | |
| 31 | G450 | AT4G14550 | Ta_S51749 | 555 | Triticum | 1.00E−20 | 41% |
| | | | | | aestivum | | |
| 31 | G450 | AT4G14550 | Ta_S91137 | 556 | Triticum | 3.00E−10 | 80% |
| | | | | | aestivum | | |
| 31 | G450 | AT4G14550 | Vvi_S15400916 | 670 | Vitis vinifera | 1.00E−57 | 86% |
| 31 | G450 | AT4G14550 | Vvi_S15406370 | 671 | Vitis vinifera | 3.00E−09 | 86% |
| 31 | G450 | AT4G14550 | Vvi_S15428140 | 672 | Vitis vinifera | 5.00E−50 | 49% |
| 31 | G450 | AT4G14550 | Vvi_S16806965 | 673 | Vitis vinifera | 3.00E−43 | 75% |
| 31 | G450 | AT4G14550 | Vvi_S16871545 | 674 | Vitis vinifera | 1.00E−89 | 72% |
| 31 | G450 | AT4G14550 | Zm_S11324536 | 508 | Zea mays | 9.00E−31 | 41% |
| 31 | G450 | AT4G14550 | Zm_S11451126 | 510 | Zea mays | 2.00E−17 | 78% |
| 31 | G450 | AT4G14550 | Zm_S11451156 | 511 | Zea mays | 2.00E−46 | 56% |
| 31 | G450 | AT4G14550 | Zm_S11527890 | 512 | Zea mays | 2.00E−45 | 53% |
| 31 | G450 | AT4G14550 | Zm_S11528788 | 513 | Zea mays | 5.00E−77 | 59% |
| 33 | G522 | AT4G36160 | Lco_S19461175 | 749 | Lotus | 2.00E−04 | 31% |
| | | | | | corniculatus | | |
| 33 | G522 | AT4G36160 | SGN-UNIGENE- | 601 | Lycopersicon | 6.00E−80 | 60% |
| | | | SINGLET-397751 | | esculentum | | |
| 33 | G522 | AT4G36160 | Pta_S15762497 | 696 | Pinus taeda | 3.00E−30 | 76% |
| 33 | G522 | AT4G36160 | Pta_S15777524 | 697 | Pinus taeda | 1.00E−68 | 81% |
| 33 | G522 | AT4G36160 | Zm_S11327546 | 514 | Zea mays | 3.00E−07 | 34% |
| 37 | G558 | AT5G06950 | Gma_S4902665 | 456 | Glycine max | 3.00E−19 | 88% |
| 37 | G558 | AT5G06950 | Gma_S4911209 | 457 | Glycine max | 6.00E−65 | 82% |
| 37 | G558 | AT5G06950 | Gma_S4975330 | 458 | Glycine max | 2.00E−52 | 79% |
| 37 | G558 | AT5G06950 | Gma_S5146796 | 459 | Glycine max | 1.00E−139 | 69% |
| 37 | G558 | AT5G06950 | Hv_S227616 | 495 | Hordeum | 2.00E−42 | 84% |
| | | | | | vulgare | | |
| 37 | G558 | AT5G06950 | Hv_S27170 | 496 | Hordeum | 4.00E−52 | 51% |
| | | | | | vulgare | | |
| 37 | G558 | AT5G06950 | Lsa_S18776116 | 714 | Lactuca | 4.00E−82 | 64% |
| | | | | | sativa | | |
| 37 | G558 | AT5G06950 | Lsa_S18777336 | 715 | Lactuca | 8.00E−67 | 54% |
| | | | | | sativa | | |
| 37 | G558 | AT5G06950 | Lco_S19286074 | 750 | Lotus | 1.00E−18 | 84% |
| | | | | | corniculatus | | |
| 37 | G558 | AT5G06950 | Lco_S19343385 | 751 | Lotus | 2.00E−12 | 91% |
| | | | | | corniculatus | | |
| 37 | G558 | AT5G06950 | Les_S5295407 | 579 | Lycopersicon | 1.00E−120 | 59% |
| | | | | | esculentum | | |
| 37 | G558 | AT5G06950 | Les_S5295673 | 580 | Lycopersicon | 9.00E−99 | 75% |
| | | | | | esculentum | | |
| 37 | G558 | AT5G06950 | SGN-UNIGENE- | 602 | Lycopersicon | 3.00E−78 | 60% |
| | | | 46372 | | esculentum | | |
| 37 | G558 | AT5G06950 | SGN-UNIGENE- | 603 | Lycopersicon | 1.00E−134 | 75% |
| | | | 46373 | | esculentum | | |
| 37 | G558 | AT5G06950 | SGN-UNIGENE- | 604 | Lycopersicon | 1.00E−139 | 78% |
| | | | 47327 | | esculentum | | |
| 37 | G558 | AT5G06950 | SGN-UNIGENE- | 605 | Lycopersicon | 9.00E−51 | 76% |
| | | | 49500 | | esculentum | | |
| 37 | G558 | AT5G06950 | SGN-UNIGENE- | 606 | Lycopersicon | 4.00E−89 | 54% |
| | | | 50258 | | esculentum | | |
| 37 | G558 | AT5G06950 | SGN-UNIGENE- | 607 | Lycopersicon | 4.00E−06 | 76% |
| | | | 57605 | | esculentum | | |
| 37 | G558 | AT5G06950 | SGN-UNIGENE- | 608 | Lycopersicon | 3.00E−84 | 56% |
| | | | 57705 | | esculentum | | |
| 37 | G558 | AT5G06950 | SGN-UNIGENE- | 609 | Lycopersicon | 6.00E−97 | 69% |
| | | | 58538 | | esculentum | | |
| 37 | G558 | AT5G06950 | SGN-UNIGENE- | 611 | Lycopersicon | 6.00E−26 | 55% |
| | | | SINGLET-340722 | | esculentum | | |
| 37 | G558 | AT5G06950 | SGN-UNIGENE- | 612 | Lycopersicon | 2.00E−63 | 60% |
| | | | SINGLET-43282 | | esculentum | | |
| 37 | G558 | AT5G06950 | Mtr_S15185262 | 481 | Medicago | 2.00E−23 | 92% |
| | | | | | truncatula | | |
| 37 | G558 | AT5G06950 | Mtr_S5309116 | 482 | Medicago | 2.00E−84 | 70% |
| | | | | | truncatula | | |
| 37 | G558 | AT5G06950 | Mtr_S7091737 | 483 | Medicago | 9.00E−29 | 88% |
| | | | | | truncatula | | |
| 37 | G558 | AT5G06950 | Os_S83289 | 418 | Oryza sativa | 1.00E−144 | 78% |
| 37 | G558 | AT5G06950 | Os_S83290 | 419 | Oryza sativa | 1.00E−139 | 79% |
| 37 | G558 | AT5G06950 | Os_S83291 | 420 | Oryza sativa | 1.00E−139 | 75% |
| 37 | G558 | AT5G06950 | Os_S83292 | 421 | Oryza sativa | 1.00E−138 | 74% |
| 37 | G558 | AT5G06950 | Pta_S17047774 | 698 | Pinus taeda | 1.00E−56 | 64% |
| 37 | G558 | AT5G06950 | Pta_S17049082 | 699 | Pinus taeda | 5.00E−17 | 87% |
| 37 | G558 | AT5G06950 | Ptp_S17968122 | 730 | Populus | 6.00E−48 | 91% |
| | | | | | tremula x | | |
| | | | | | Populus | | |
| | | | | | tremuloides | | |
| 37 | G558 | AT5G06950 | Sof_S17339937 | 774 | Saccharum | 4.00E−74 | 32% |
| | | | | | officinarum | | |
| 37 | G558 | AT5G06950 | Sof_S17379632 | 775 | Saccharum | 3.00E−84 | 77% |
| | | | | | officinarum | | |
| 37 | G558 | AT5G06950 | Sof_S17473960 | 776 | Saccharum | 5.00E−92 | 80% |
| | | | | | officinarum | | |
| 37 | G558 | AT5G06950 | Stu_S14742290 | 793 | Solanum | 1.00E−125 | 62% |
| | | | | | tuberosum | | |
| 37 | G558 | AT5G06950 | Stu_S14742333 | 794 | Solanum | 1.00E−120 | 59% |
| | | | | | tuberosum | | |
| 37 | G558 | AT5G06950 | Stu_S18108323 | 795 | Solanum | 1.00E−17 | 68% |
| | | | | | tuberosum | | |
| 37 | G558 | AT5G06950 | Stu_S18130411 | 796 | Solanum | 1.00E−127 | 73% |
| | | | | | tuberosum | | |
| 37 | G558 | AT5G06950 | Stu_S18130846 | 797 | Solanum | 7.00E−88 | 54% |
| | | | | | tuberosum | | |
| 37 | G558 | AT5G06950 | Stu_S18131293 | 798 | Solanum | 6.00E−39 | 64% |
| | | | | | tuberosum | | |
| 37 | G558 | AT5G06950 | Sbi_S15655270 | 765 | Sorghum | 6.00E−22 | 77% |
| | | | | | bicolor | | |
| 37 | G558 | AT5G06950 | Sbi_S17497937 | 766 | Sorghum | 6.00E−30 | 67% |
| | | | | | bicolor | | |
| 37 | G558 | AT5G06950 | Sbi_S19492714 | 767 | Sorghum | 4.00E−27 | 67% |
| | | | | | bicolor | | |
| 37 | G558 | AT5G06950 | Sbi_S19493653 | 768 | Sorghum | 4.00E−39 | 65% |
| | | | | | bicolor | | |
| 37 | G558 | AT5G06950 | Ta_S115084 | 557 | Triticum | 1.00E−19 | 77% |
| | | | | | aestivum | | |
| 37 | G558 | AT5G06950 | Ta_S141705 | 558 | Triticum | 5.00E−10 | 90% |
| | | | | | aestivum | | |
| 37 | G558 | AT5G06950 | Ta_S66308 | 559 | Triticum | 1.00E−136 | 75% |
| | | | | | aestivum | | |
| 37 | G558 | AT5G06950 | Ta_S66461 | 560 | Triticum | 1.00E−142 | 77% |
| | | | | | aestivum | | |
| 37 | G558 | AT5G06950 | Vvi_S15429865 | 675 | Vitis vinifera | 2.00E−76 | 53% |
| 37 | G558 | AT5G06950 | Vvi_S16526894 | 676 | Vitis vinifera | 1.00E−80 | 81% |
| 37 | G558 | AT5G06950 | Zm_S11418176 | 515 | Zea mays | 1.00E−141 | 77% |
| 37 | G558 | AT5G06950 | Zm_S11418177 | 516 | Zea mays | 1.00E−138 | 76% |
| 37 | G558 | AT5G06950 | Zm_S11425511 | 517 | Zea mays | 5.00E−58 | 59% |
| 37 | G558 | AT5G06950 | Zm_S11432162 | 518 | Zea mays | 4.00E−29 | 67% |
| 39 | G567 | AT4G02640 | Os_S60616 | 422 | Oryza sativa | 3.00E−47 | 34% |
| 39 | G567 | AT4G02640 | Os_S64145 | 423 | Oryza sativa | 1.00E−37 | 33% |
| 39 | G567 | AT4G02640 | Stu_S18120365 | 799 | Solanum | 9.00E−45 | 37% |
| | | | | | tuberosum | | |
| 39 | G567 | AT4G02640 | Zm_S11417946 | 519 | Zea mays | 1.00E−46 | 34% |
| 39 | G567 | AT4G02640 | Zm_S11417974 | 520 | Zea mays | 2.00E−44 | 34% |
| 39 | G567 | AT4G02640 | Zm_S11418174 | 521 | Zea mays | 1.00E−31 | 30% |
| 41 | G580 | AT2G17770 | SGN-UNIGENE- | 613 | Lycopersicon | 1.00E−09 | 33% |
| | | | SINGLET-392194 | | esculentum | | |
| 43 | G635 | AT5G63420 | Lsa_S18814922 | 716 | Lactuca | 1.00E−110 | 78% |
| | | | | | sativa | | |
| 43 | G635 | AT5G63420 | Lco_S19346901 | 753 | Lotus | 2.00E−20 | 65% |
| | | | | | corniculatus | | |
| 43 | G635 | AT5G63420 | Mtr_S5399163 | 484 | Medicago | 8.00E−47 | 62% |
| | | | | | truncatula | | |
| 43 | G635 | AT5G63420 | Sof_S17305305 | 777 | Saccharum | 7.00E−98 | 79% |
| | | | | | officinarum | | |
| 43 | G635 | AT5G63420 | Zm_S11522393 | 522 | Zea mays | 2.00E−78 | 76% |
| 45 | G675 | AT1G34670 | Zm_S11529197 | 523 | Zea mays | 2.00E−18 | 93% |
| 47 | G729 | AT5G16560 | Gma_S4928741 | 460 | Glycine max | 3.00E−04 | 35% |
| 47 | G729 | AT5G16560 | Gma_S5129577 | 461 | Glycine max | 4.00E−04 | 27% |
| 47 | G729 | AT5G16560 | Lsa_S18816514 | 717 | Lactuca | 4.00E−45 | 37% |
| | | | | | sativa | | |
| 47 | G729 | AT5G16560 | Lco_S19334151 | 754 | Lotus | 3.00E−05 | 36% |
| | | | | | corniculatus | | |
| 47 | G729 | AT5G16560 | SGN-UNIGENE- | 615 | Lycopersicon | 2.00E−21 | 38% |
| | | | 54539 | | esculentum | | |
| 47 | G729 | AT5G16560 | SGN-UNIGENE- | 618 | Lycopersicon | 5.00E−33 | 61% |
| | | | SINGLET-39727 | | esculentum | | |
| 47 | G729 | AT5G16560 | SGN-UNIGENE- | 619 | Lycopersicon | 3.00E−19 | 38% |
| | | | SINGLET-40526 | | esculentum | | |
| 47 | G729 | AT5G16560 | Zm_S11478301 | 525 | Zea mays | 4.00E−27 | 50% |
| 49 | G812 | AT3G51910 | SGN-UNIGENE- | 620 | Lycopersicon | 7.00E−57 | 36% |
| | | | 45592 | | esculentum | | |
| 51 | G843 | AT3G07740 | Lsa_S18826577 | 718 | Lactuca | 4.00E−70 | 62% |
| | | | | | sativa | | |
| 51 | G843 | AT3G07740 | Os_S51420 | 425 | Oryza sativa | 2.00E−23 | 54% |
| 51 | G843 | AT3G07740 | Ppa_S17599742 | 815 | Physcomitrella | 7.00E−15 | 33% |
| | | | | | patens | | |
| 51 | G843 | AT3G07740 | Sbi_S14712583 | 769 | Sorghum | 2.00E−25 | 43% |
| | | | | | bicolor | | |
| 53 | G881 | AT4G31800 | Gma_S4999008 | 462 | Glycine max | 3.00E−27 | 56% |
| 53 | G881 | AT4G31800 | SGN-UNIGENE- | 621 | Lycopersicon | 3.00E−16 | 92% |
| | | | 45119 | | esculentum | | |
| 53 | G881 | AT4G31800 | SGN-UNIGENE- | 623 | Lycopersicon | 9.00E−39 | 56% |
| | | | SINGLET-440841 | | esculentum | | |
| 53 | G881 | AT4G31800 | Sof_S17309586 | 778 | Saccharum | 2.00E−04 | 56% |
| | | | | | officinarum | | |
| 53 | G881 | AT4G31800 | Ta_S141953 | 562 | Triticum | 3.00E−04 | 54% |
| | | | | | aestivum | | |
| 55 | G937 | AT1G49560 | Gma_S5129137 | 463 | Glycine max | 4.00E−20 | 54% |
| 55 | G937 | AT1G49560 | Lco_S19398752 | 755 | Lotus | 0.35 | 52% |
| | | | | | corniculatus | | |
| 55 | G937 | AT1G49560 | Vvi_S15431951 | 678 | Vitis vinifera | 2.00E−39 | 60% |
| 55 | G937 | AT1G49560 | Vvi_S16805106 | 679 | Vitis vinifera | 1.00E−16 | 50% |
| 55 | G937 | AT1G49560 | Zm_S11434591 | 526 | Zea mays | 1.00E−04 | 34% |
| 59 | G1007 | AT2G25820 | Pta_S16846031 | 700 | Pinus taeda | 5.00E−30 | 37% |
| 61 | G1053 | AT2G04038 | Ta_S121486 | 563 | Triticum | 4.00E−10 | 43% |
| | | | | | aestivum | | |
| 63 | G1078 | AT3G60320 | SGN-UNIGENE- | 625 | Lycopersicon | 5.00E−70 | 64% |
| | | | 54082 | | esculentum | | |
| 63 | G1078 | AT3G60320 | SGN-UNIGENE- | 626 | Lycopersicon | 2.00E−86 | 74% |
| | | | 57266 | | esculentum | | |
| 63 | G1078 | AT3G60320 | SGN-UNIGENE- | 627 | Lycopersicon | 1.00E−30 | 87% |
| | | | SINGLET-395949 | | esculentum | | |
| 63 | G1078 | AT3G60320 | Os_S66076 | 426 | Oryza sativa | 1.00E−999 | 47% |
| 63 | G1078 | AT3G60320 | Sbi_S15901323 | 770 | Sorghum | 1.00E−24 | 37% |
| | | | | | bicolor | | |
| 63 | G1078 | AT3G60320 | Vvi_S16868087 | 680 | Vitis vinifera | 3.00E−35 | 75% |
| 65 | G1226 | AT4G01460 | Zm_S11426582 | 527 | Zea mays | 0.047 | 51% |
| 67 | G1273 | AT2G37260 | Zm_S11425989 | 528 | Zea mays | 7.00E−23 | 67% |
| 69 | G1324 | AT1G68320 | Gma_S5011023 | 465 | Glycine max | 6.00E−18 | 63% |
| 69 | G1324 | AT1G68320 | Lsa_S18828897 | 719 | Lactuca | 2.00E−65 | 64% |
| | | | | | sativa | | |
| 69 | G1324 | AT1G68320 | Stu_S19063684 | 800 | Solanum | 2.00E−11 | 42% |
| | | | | | tuberosum | | |
| 69 | G1324 | AT1G68320 | Zm_S11529166 | 530 | Zea mays | 1.00E−18 | 86% |
| 69 | G1324 | AT1G68320 | Zm_S11529168 | 531 | Zea mays | 8.00E−16 | 76% |
| 71 | G1328 | AT4G05100 | SGN-UNIGENE- | 630 | Lycopersicon | 3.00E−74 | 81% |
| | | | SINGLET-39199 | | esculentum | | |
| 71 | G1328 | AT4G05100 | Stu_S19116842 | 801 | Solanum | 4.00E−10 | 34% |
| | | | | | tuberosum | | |
| 71 | G1328 | AT4G05100 | Zm_S11529155 | 533 | Zea mays | 1.00E−18 | 95% |
| 73 | G1444 | AT2G42040 | Gma_S4929057 | 467 | Glycine max | 1.00E−21 | 46% |
| 73 | G1444 | AT2G42040 | Ppa_S17595796 | 816 | Physcomitrella | 5.00E−04 | 53% |
| | | | | | patens | | |
| 73 | G1444 | AT2G42040 | Ppa_S17602854 | 817 | Physcomitrella | 3.00E−05 | 29% |
| | | | | | patens | | |
| 79 | G1481 | AT4G27310 | Gma_S5036787 | 468 | Glycine max | 3.00E−25 | 37% |
| 79 | G1481 | AT4G27310 | Lsa_S18813209 | 720 | Lactuca | 1.00E−37 | 46% |
| | | | | | sativa | | |
| 79 | G1481 | AT4G27310 | SGN-UNIGENE- | 632 | Lycopersicon | 5.00E−29 | 41% |
| | | | 49975 | | esculentum | | |
| 79 | G1481 | AT4G27310 | SGN-UNIGENE- | 633 | Lycopersicon | 4.00E−38 | 46% |
| | | | 52163 | | esculentum | | |
| 79 | G1481 | AT4G27310 | SGN-UNIGENE- | 635 | Lycopersicon | 1.00E−29 | 38% |
| | | | 54438 | | esculentum | | |
| 79 | G1481 | AT4G27310 | SGN-UNIGENE- | 636 | Lycopersicon | 5.00E−42 | 45% |
| | | | 57631 | | esculentum | | |
| 79 | G1481 | AT4G27310 | Stu_S18131013 | 802 | Solanum | 7.00E−41 | 44% |
| | | | | | tuberosum | | |
| 79 | G1481 | AT4G27310 | Vvi_S15383518 | 681 | Vitis vinifera | 4.00E−34 | 40% |
| 79 | G1481 | AT4G27310 | Vvi_S16870346 | 682 | Vitis vinifera | 4.00E−46 | 47% |
| 83 | G1543 | AT2G01430 | Os_S65512 | 428 | Oryza sativa | 1.00E−47 | 67% |
| 85 | G1635 | AT5G17300 | Gma_S4973270 | 470 | Glycine max | 4.00E−09 | 34% |
| 85 | G1635 | AT5G17300 | Gma_S5050105 | 471 | Glycine max | 2.00E−05 | 43% |
| 85 | G1635 | AT5G17300 | Vvi_S16870895 | 685 | Vitis vinifera | 1.00E−07 | 43% |
| 87 | G1638 | AT2G38090 | Lsa_S18802835 | 721 | Lactuca | 4.00E−56 | 48% |
| | | | | | sativa | | |
| 87 | G1638 | AT2G38090 | SGN-UNIGENE- | 637 | Lycopersicon | 2.00E−76 | 64% |
| | | | 53190 | | esculentum | | |
| 87 | G1638 | AT2G38090 | SGN-UNIGENE- | 638 | Lycopersicon | 4.00E−47 | 64% |
| | | | SINGLET-441055 | | esculentum | | |
| 87 | G1638 | AT2G38090 | Os_S31018 | 430 | Oryza sativa | 4.00E−31 | 48% |
| 87 | G1638 | AT2G38090 | Sbi_S19499592 | 771 | Sorghum | 8.00E−19 | 43% |
| | | | | | bicolor | | |
| 87 | G1638 | AT2G38090 | Zm_S11324534 | 534 | Zea mays | 4.00E−35 | 80% |
| 89 | G1640 | AT5G49330 | Lsa_S18786927 | 722 | Lactuca | 3.00E−52 | 58% |
| | | | | | sativa | | |
| 89 | G1640 | AT5G49330 | SGN-UNIGENE- | 639 | Lycopersicon | 3.00E−34 | 61% |
| | | | SINGLET-46216 | | esculentum | | |
| 89 | G1640 | AT5G49330 | Zm_S11529203 | 535 | Zea mays | 7.00E−15 | 74% |
| 91 | G1645 | AT1G26780 | SGN-UNIGENE- | 640 | Lycopersicon | 4.00E−61 | 92% |
| | | | SINGLET-14240 | | esculentum | | |
| 97 | G1752 | AT2G31230 | Hv_S20601 | 498 | Hordeum | 9.00E−15 | 35% |
| | | | | | vulgare | | |
| 99 | G1755 | AT2G40350 | SGN-UNIGENE- | 641 | Lycopersicon | 2.00E−07 | 28% |
| | | | 57946 | | esculentum | | |
| 107 | G1808 | AT4G37730 | Gma_S5132128 | 472 | Glycine max | 2.00E−11 | 34% |
| 107 | G1808 | AT4G37730 | SGN-UNIGENE- | 642 | Lycopersicon | 3.00E−29 | 40% |
| | | | 50805 | | esculentum | | |
| 117 | G1895 | AT1G26790 | Pta_S15747863 | 701 | Pinus taeda | 6.00E−08 | 49% |
| 119 | G1897 | AT5G66940 | Sof_S17450399 | 779 | Saccharum | 5.00E−25 | 78% |
| | | | | | officinarum | | |
| 121 | G1903 | AT1G69570 | Pta_S15747863 | 701 | Pinus taeda | 6.00E−08 | 49% |
| 123 | G1909 | AT1G07640 | SGN-UNIGENE- | 644 | Lycopersicon | 1.00E−30 | 53% |
| | | | 54382 | | esculentum | | |
| 123 | G1909 | AT1G07640 | Zm_S11443238 | 537 | Zea mays | 2.00E−05 | 39% |
| 125 | G1935 | AT1G77950 | SGN-UNIGENE- | 645 | Lycopersicon | 3.00E−18 | 30% |
| | | | 49757 | | esculentum | | |
| 125 | G1935 | AT1G77950 | SGN-UNIGENE- | 646 | Lycopersicon | 9.00E−13 | 41% |
| | | | 52060 | | esculentum | | |
| 125 | G1935 | AT1G77950 | SGN-UNIGENE- | 647 | Lycopersicon | 2.00E−24 | 52% |
| | | | SINGLET-16934 | | esculentum | | |
| 125 | G1935 | AT1G77950 | Ppa_S17639839 | 820 | Physcomitrella | 9.00E−31 | 41% |
| | | | | | patens | | |
| 125 | G1935 | AT1G77950 | Ppa_S17639840 | 821 | Physcomitrella | 8.00E−32 | 40% |
| | | | | | patens | | |
| 125 | G1935 | AT1G77950 | Ppa_S17639871 | 822 | Physcomitrella | 8.00E−32 | 39% |
| | | | | | patens | | |
| 125 | G1935 | AT1G77950 | Ppa_S17639872 | 823 | Physcomitrella | 6.00E−32 | 39% |
| | | | | | patens | | |
| 127 | G1950 | AT2G03430 | Lsa_S18777138 | 723 | Lactuca | 6.00E−80 | 64% |
| | | | | | sativa | | |
| 127 | G1950 | AT2G03430 | Lsa_S18831768 | 724 | Lactuca | 7.00E−13 | 30% |
| | | | | | sativa | | |
| 127 | G1950 | AT2G03430 | Lco_S19316645 | 758 | Lotus | 7.00E−24 | 76% |
| | | | | | corniculatus | | |
| 127 | G1950 | AT2G03430 | SGN-UNIGENE- | 648 | Lycopersicon | 3.00E−46 | 67% |
| | | | SINGLET-475671 | | esculentum | | |
| 127 | G1950 | AT2G03430 | SGN-UNIGENE- | 649 | Lycopersicon | 2.00E−17 | 36% |
| | | | SINGLET-56300 | | esculentum | | |
| 127 | G1950 | AT2G03430 | Mtr_S5402942 | 487 | Medicago | 7.00E−11 | 84% |
| | | | | | truncatula | | |
| 127 | G1950 | AT2G03430 | Ppa_S17636323 | 824 | Physcomitrella | 5.00E−13 | 35% |
| | | | | | patens | | |
| 127 | G1950 | AT2G03430 | Ta_S60643 | 565 | Triticum | 2.00E−50 | 68% |
| | | | | | aestivum | | |
| 127 | G1950 | AT2G03430 | Zm_S11413309 | 538 | Zea mays | 6.00E−35 | 72% |
| 129 | G1954 | AT3G24140 | SGN-UNIGENE- | 650 | Lycopersicon | 3.00E−18 | 51% |
| | | | SINGLET-53753 | | esculentum | | |
| 129 | G1954 | AT3G24140 | Pta_S16799286 | 702 | Pinus taeda | 1.00E−13 | 58% |
| 131 | G1958 | AT4G28610 | Gma_S5063433 | 473 | Glycine max | 3.00E−27 | 52% |
| 131 | G1958 | AT4G28610 | Gma_S5140349 | 474 | Glycine max | 1.00E−13 | 44% |
| 131 | G1958 | AT4G28610 | Hv_S114723 | 499 | Hordeum | 2.00E−11 | 51% |
| | | | | | vulgare | | |
| 131 | G1958 | AT4G28610 | SGN-UNIGENE- | 651 | Lycopersicon | 0.018 | 34% |
| | | | 57277 | | esculentum | | |
| 131 | G1958 | AT4G28610 | SGN-UNIGENE- | 652 | Lycopersicon | 1.00E−58 | 77% |
| | | | SINGLET-3690 | | esculentum | | |
| 131 | G1958 | AT4G28610 | SGN-UNIGENE- | 653 | Lycopersicon | 3.00E−48 | 43% |
| | | | SINGLET-38343 | | esculentum | | |
| 131 | G1958 | AT4G28610 | SGN-UNIGENE- | 654 | Lycopersicon | 2.00E−12 | 45% |
| | | | SINGLET-390838 | | esculentum | | |
| 131 | G1958 | AT4G28610 | SGN-UNIGENE- | 655 | Lycopersicon | 1.00E−10 | 32% |
| | | | SINGLET-57100 | | esculentum | | |
| 131 | G1958 | AT4G28610 | Ptp_S17904851 | 736 | Populus | 3.00E−12 | 84% |
| | | | | | tremula x | | |
| | | | | | Populus | | |
| | | | | | tremuloides | | |
| 131 | G1958 | AT4G28610 | Sof_S17303253 | 780 | Saccharum | 2.00E−55 | 60% |
| | | | | | officinarum | | |
| 131 | G1958 | AT4G28610 | Stu_S18126579 | 803 | Solanum | 1.00E−56 | 63% |
| | | | | | tuberosum | | |
| 131 | G1958 | AT4G28610 | Stu_S18135521 | 804 | Solanum | 9.00E−58 | 54% |
| | | | | | tuberosum | | |
| 131 | G1958 | AT4G28610 | Ta_S173982 | 566 | Triticum | 3.00E−25 | 37% |
| | | | | | aestivum | | |
| 131 | G1958 | AT4G28610 | Ta_S204555 | 567 | Triticum | 4.00E−59 | 48% |
| | | | | | aestivum | | |
| 131 | G1958 | AT4G28610 | Zm_S11333932 | 539 | Zea mays | 9.00E−32 | 57% |
| 133 | G2052 | AT5G46590 | SGN-UNIGENE- | 656 | Lycopersicon | 9.00E−47 | 87% |
| | | | 52489 | | esculentum | | |
| 133 | G2052 | AT5G46590 | SGN-UNIGENE- | 657 | Lycopersicon | 7.00E−58 | 73% |
| | | | 53237 | | esculentum | | |
| 133 | G2052 | AT5G46590 | Vvi_S15351555 | 688 | Vitis vinifera | 2.00E−10 | 34% |
| 139 | G2116 | AT1G06850 | Lco_S19325184 | 759 | Lotus | 4.00E−05 | 29% |
| | | | | | corniculatus | | |
| 139 | G2116 | AT1G06850 | SGN-UNIGENE- | 658 | Lycopersicon | 3.00E−06 | 37% |
| | | | SINGLET-8462 | | esculentum | | |
| 139 | G2116 | AT1G06850 | Zm_S11505224 | 540 | Zea mays | 5.00E−22 | 42% |
| 141 | G2132 | AT1G49120 | SGN-UNIGENE- | 659 | Lycopersicon | 5.00E−04 | 54% |
| | | | SINGLET-451192 | | esculentum | | |
| 145 | G2141 | AT1G68920 | SGN-UNIGENE- | 660 | Lycopersicon | 3.00E−16 | 37% |
| | | | 58219 | | esculentum | | |
| 145 | G2141 | AT1G68920 | Ta_S112420 | 569 | Triticum | 2.00E−16 | 71% |
| | | | | | aestivum | | |
| 147 | G2145 | AT1G27740 | Ta_S174040 | 570 | Triticum | 3.00E−40 | 64% |
| | | | | | aestivum | | |
| 149 | G2150 | AT3G23690 | Sbi_S19509323 | 772 | Sorghum | 3.00E−14 | 45% |
| | | | | | bicolor | | |
| 149 | G2150 | AT3G23690 | Ta_S118840 | 571 | Triticum | 3.00E−38 | 58% |
| | | | | | aestivum | | |
| 151 | G2157 | AT3G55560 | Gma_S4925445 | 475 | Glycine max | 2.00E−31 | 52% |
| 151 | G2157 | AT3G55560 | Han_S18724409 | 707 | Helianthus | 2.00E−08 | 30% |
| | | | | | annuus | | |
| 151 | G2157 | AT3G55560 | Stu_S18117799 | 805 | Solanum | 2.00E−70 | 50% |
| | | | | | tuberosum | | |
| 153 | G2294 | AT1G44830 | Lco_S19357424 | 760 | Lotus | 0.11 | 35% |
| | | | | | corniculatus | | |
| 153 | G2294 | AT1G44830 | Stu_S18109605 | 806 | Solanum | 2.00E−04 | 38% |
| | | | | | tuberosum | | |
| 153 | G2294 | AT1G44830 | Vvi_S15353048 | 689 | Vitis vinifera | 5.00E−07 | 36% |

Table 6 identifies the homologous relationships of sequences found in the Sequence Listing for which such a relationship has been identified. The column headings list: (a) the SEQ ID NO of each polynucleotide and polypeptide sequence; (b) the sequence identifier (i.e., the GID or UniGene identifier); (c) the biochemical nature of the sequence (i.e., polynucleotide (DNA) or protein (PRT)); (d) the species in which the given sequence in the first column is found; and (e) the paralogous or orthologous relationship to other sequences in the Sequence Listing.

TABLE 6
Homologous relationships found within the Sequence Listing

| SEQ ID | | DNA or | | |
|---|---|---|---|---|
| NO: | GID | PRT | Species | Relationship |
| 1 | G3 | DNA | Arabidopsis | Predicted polypeptide sequence is paralogous to G10 |
| | | | thaliana | |
| 2 | G3 | PRT | Arabidopsis | Paralogous to G10 |
| | | | thaliana | |
| 3 | G22 | DNA | Arabidopsis | Predicted polypeptide sequence is paralogous to G1006, |
| | | | thaliana | G28; orthologous to G3430, G3659, G3660, G3661, |
| | | | | G3717, G3718, G3841, G3843, G3844, G3845, G3846, |
| | | | | G3848, G3852, G3856, G3857, G3858, G3864, G3865 |
| 4 | G22 | PRT | Arabidopsis | Paralogous to G1006, G28; Orthologous to G3430, G3659, |
| | | | thaliana | G3660, G3661, G3717, G3718, G3841, G3843, G3844, |
| | | | | G3845, G3846, G3848, G3852, G3856, G3857, G3858, |
| | | | | G3864, G3865 |
| 5 | G24 | DNA | Arabidopsis | Predicted polypeptide sequence is paralogous to G12, |
| | | | thaliana | G1277, G1379; orthologous to G3656 |
| 6 | G24 | PRT | Arabidopsis | Paralogous to G12, G1277, G1379; Orthologous to G3656 |
| | | | thaliana | |
| 7 | G47 | DNA | Arabidopsis | Predicted polypeptide sequence is paralogous to G2133; |
| | | | thaliana | orthologous to G3643, G3644, G3645, G3646, G3647, |
| | | | | G3649, G3650, G3651 |
| 8 | G47 | PRT | Arabidopsis | Paralogous to G2133; Orthologous to G3643, G3644, |
| | | | thaliana | G3645, G3646, G3647, G3649, G3650, G3651 |
| 9 | G156 | DNA | Arabidopsis | |
| | | | thaliana | |
| 10 | G156 | PRT | Arabidopsis | |
| | | | thaliana | |
| 11 | G159 | DNA | Arabidopsis | Predicted polypeptide sequence is paralogous to G165 |
| | | | thaliana | |
| 12 | G159 | PRT | Arabidopsis | Paralogous to G165 |
| | | | thaliana | |
| 13 | G187 | DNA | Arabidopsis | Predicted polypeptide sequence is paralogous to G195 |
| | | | thaliana | |
| 14 | G187 | PRT | Arabidopsis | Paralogous to G195 |
| | | | thaliana | |
| 15 | G190 | DNA | Arabidopsis | |
| | | | thaliana | |
| 16 | G190 | PRT | Arabidopsis | |
| | | | thaliana | |
| 17 | G226 | DNA | Arabidopsis | Predicted polypeptide sequence is paralogous to G1816, |
| | | | thaliana | G225, G2718, G682, G3930; orthologous to G3392, |
| | | | | G3393, G3431, G3444, G3445, G3446, G3447, G3448, |
| | | | | G3449, G3450 |
| 18 | G226 | PRT | Arabidopsis | Paralogous to G1816, G225, G2718, G682, G3930; |
| | | | thaliana | Orthologous to G3392, G3393, G3431, G3444, G3445, |
| | | | | G3446, G3447, G3448, G3449, G3450 |
| 19 | G237 | DNA | Arabidopsis | Predicted polypeptide sequence is paralogous to G1309 |
| | | | thaliana | |
| 20 | G237 | PRT | Arabidopsis | Paralogous to G1309 |
| | | | thaliana | |
| 21 | G270 | DNA | Arabidopsis | |
| | | | thaliana | |
| 22 | G270 | PRT | Arabidopsis | |
| | | | thaliana | |
| 23 | G328 | DNA | Arabidopsis | Predicted polypeptide sequence is paralogous to G2436, |
| | | | thaliana | G2443 |
| 24 | G328 | PRT | Arabidopsis | Paralogous to G2436, G2443 |
| | | | thaliana | |
| 25 | G363 | DNA | Arabidopsis | |
| | | | thaliana | |
| 26 | G363 | PRT | Arabidopsis | |
| | | | thaliana | |
| 27 | G383 | DNA | Arabidopsis | Predicted polypeptide sequence is paralogous to G1917 |
| | | | thaliana | |
| 28 | G383 | PRT | Arabidopsis | Paralogous to G1917 |
| | | | thaliana | |
| 29 | G435 | DNA | Arabidopsis | |
| | | | thaliana | |
| 30 | G435 | PRT | Arabidopsis | |
| | | | thaliana | |
| 31 | G450 | DNA | Arabidopsis | Predicted polypeptide sequence is paralogous to G448, |
| | | | thaliana | G455, G456 |
| 32 | G450 | PRT | Arabidopsis | Paralogous to G448, G455, G456 |
| | | | thaliana | |
| 33 | G522 | DNA | Arabidopsis | Predicted polypeptide sequence is paralogous to G1354, |
| | | | thaliana | G1355, G1453, G1766, G2534, G761 |
| 34 | G522 | PRT | Arabidopsis | Paralogous to G1354, G1355, G1453, G1766, G2534, |
| | | | thaliana | G761 |
| 35 | G551 | DNA | Arabidopsis | |
| | | | thaliana | |
| 36 | G551 | PRT | Arabidopsis | |
| | | | thaliana | |
| 37 | G558 | DNA | Arabidopsis | Predicted polypeptide sequence is paralogous to G1198, |
| | | | thaliana | G1806, G554, G555, G556, G578, G629 |
| 38 | G558 | PRT | Arabidopsis | Paralogous to G1198, G1806, G554, G555, G556, G578, |
| | | | thaliana | G629 |
| 39 | G567 | DNA | Arabidopsis | |
| | | | thaliana | |
| 40 | G567 | PRT | Arabidopsis | |
| | | | thaliana | |
| 41 | G580 | DNA | Arabidopsis | Predicted polypeptide sequence is paralogous to G568 |
| | | | thaliana | |
| 42 | G580 | PRT | Arabidopsis | Paralogous to G568 |
| | | | thaliana | |
| 43 | G635 | DNA | Arabidopsis | |
| | | | thaliana | |
| 44 | G635 | PRT | Arabidopsis | |
| | | | thaliana | |
| 45 | G675 | DNA | Arabidopsis | |
| | | | thaliana | |
| 46 | G675 | PRT | Arabidopsis | |
| | | | thaliana | |
| 47 | G729 | DNA | Arabidopsis | Predicted polypeptide sequence is paralogous to G1040, |
| | | | thaliana | G3034, G730 |
| 48 | G729 | PRT | Arabidopsis | Paralogous to G1040, G3034, G730 |
| | | | thaliana | |
| 49 | G812 | DNA | Arabidopsis | Predicted polypeptide sequence is paralogous to G2467 |
| | | | thaliana | |
| 50 | G812 | PRT | Arabidopsis | Paralogous to G2467 |
| | | | thaliana | |
| 51 | G843 | DNA | Arabidopsis | |
| | | | thaliana | |
| 52 | G843 | PRT | Arabidopsis | |
| | | | thaliana | |
| 53 | G881 | DNA | Arabidopsis | Predicted polypeptide sequence is paralogous to G986 |
| | | | thaliana | |
| 54 | G881 | PRT | Arabidopsis | Paralogous to G986 |
| | | | thaliana | |
| 55 | G937 | DNA | Arabidopsis | |
| | | | thaliana | |
| 56 | G937 | PRT | Arabidopsis | |
| | | | thaliana | |
| 57 | G989 | DNA | Arabidopsis | |
| | | | thaliana | |
| 58 | G989 | PRT | Arabidopsis | |
| | | | thaliana | |
| 59 | G1007 | DNA | Arabidopsis | Predicted polypeptide sequence is paralogous to G1846 |
| | | | thaliana | |
| 60 | G1007 | PRT | Arabidopsis | Paralogous to G1846 |
| | | | thaliana | |
| 61 | G1053 | DNA | Arabidopsis | Predicted polypeptide sequence is paralogous to G2629 |
| | | | thaliana | |
| 62 | G1053 | PRT | Arabidopsis | Paralogous to G2629 |
| | | | thaliana | |
| 63 | G1078 | DNA | Arabidopsis | Predicted polypeptide sequence is paralogous to G577 |
| | | | thaliana | |
| 64 | G1078 | PRT | Arabidopsis | Paralogous to G577 |
| | | | thaliana | |
| 65 | G1226 | DNA | Arabidopsis | |
| | | | thaliana | |
| 66 | G1226 | PRT | Arabidopsis | |
| | | | thaliana | |
| 67 | G1273 | DNA | Arabidopsis | |
| | | | thaliana | |
| 68 | G1273 | PRT | Arabidopsis | |
| | | | thaliana | |
| 69 | G1324 | DNA | Arabidopsis | Predicted polypeptide sequence is paralogous to G2893 |
| | | | thaliana | |
| 70 | G1324 | PRT | Arabidopsis | Paralogous to G2893 |
| | | | thaliana | |
| 71 | G1328 | DNA | Arabidopsis | Predicted polypeptide sequence is paralogous to G198 |
| | | | thaliana | |
| 72 | G1328 | PRT | Arabidopsis | Paralogous to G198 |
| | | | thaliana | |
| 73 | G1444 | DNA | Arabidopsis | |
| | | | thaliana | |
| 74 | G1444 | PRT | Arabidopsis | |
| | | | thaliana | |
| 75 | G1462 | DNA | Arabidopsis | Predicted polypeptide sequence is paralogous to G1461, |
| | | | thaliana | G1463, G1464, G1465 |
| 76 | G1462 | PRT | Arabidopsis | Paralogous to G1461, G1463, G1464, G1465 |
| | | | thaliana | |
| 77 | G1463 | DNA | Arabidopsis | Predicted polypeptide sequence is paralogous to G1461, |
| | | | thaliana | G1462, G1464, G1465 |
| 78 | G1463 | PRT | Arabidopsis | Paralogous to G1461, G1462, G1464, G1465 |
| | | | thaliana | |
| 79 | G1481 | DNA | Arabidopsis | Predicted polypeptide sequence is paralogous to G900, |
| | | | thaliana | orthologous to G4014, G4015, G4016 |
| 80 | G1481 | PRT | Arabidopsis | Paralogous to G900; orthologous to G4014, G4015, G4016 |
| | | | thaliana | |
| 81 | G1504 | DNA | Arabidopsis | Predicted polypeptide sequence is paralogous to G2442, |
| | | | thaliana | G2504 |
| 82 | G1504 | PRT | Arabidopsis | Paralogous to G2442, G2504 |
| | | | thaliana | |
| 83 | G1543 | DNA | Arabidopsis | Predicted polypeptide sequence is orthologous to G3490, |
| | | | thaliana | G3510, G3524 |
| 84 | G1543 | PRT | Arabidopsis | Orthologous to G3490, G3510, G3524 |
| | | | thaliana | |
| 85 | G1635 | DNA | Arabidopsis | |
| | | | thaliana | |
| 86 | G1635 | PRT | Arabidopsis | |
| | | | thaliana | |
| 87 | G1638 | DNA | Arabidopsis | |
| | | | thaliana | |
| 88 | G1638 | PRT | Arabidopsis | |
| | | | thaliana | |
| 89 | G1640 | DNA | Arabidopsis | |
| | | | thaliana | |
| 90 | G1640 | PRT | Arabidopsis | |
| | | | thaliana | |
| 91 | G1645 | DNA | Arabidopsis | Predicted polypeptide sequence is paralogous to G2424 |
| | | | thaliana | |
| 92 | G1645 | PRT | Arabidopsis | Paralogous to G2424 |
| | | | thaliana | |
| 93 | G1650 | DNA | Arabidopsis | |
| | | | thaliana | |
| 94 | G1650 | PRT | Arabidopsis | |
| | | | thaliana | |
| 95 | G1659 | DNA | Arabidopsis | |
| | | | thaliana | |
| 96 | G1659 | PRT | Arabidopsis | |
| | | | thaliana | |
| 97 | G1752 | DNA | Arabidopsis | Predicted polypeptide sequence is paralogous to G2512 |
| | | | thaliana | |
| 98 | G1752 | PRT | Arabidopsis | Paralogous to G2512 |
| | | | thaliana | |
| 99 | G1755 | DNA | Arabidopsis | Predicted polypeptide sequence is paralogous to G1754 |
| | | | thaliana | |
| 100 | G1755 | PRT | Arabidopsis | Paralogous to G1754 |
| | | | thaliana | |
| 101 | G1784 | DNA | Arabidopsis | |
| | | | thaliana | |
| 102 | G1784 | PRT | Arabidopsis | |
| | | | thaliana | |
| 103 | G1785 | DNA | Arabidopsis | Predicted polypeptide sequence is paralogous to G248 |
| | | | thaliana | |
| 104 | G1785 | PRT | Arabidopsis | Paralogous to G248 |
| | | | thaliana | |
| 105 | G1791 | DNA | Arabidopsis | Predicted polypeptide sequence is paralogous to G1792, |
| | | | thaliana | G1795, G30; orthologous to G3380, G3381, G3383, |
| | | | | G3515, G3516, G3517, G3518, G3519, G3520, G3735, |
| | | | | G3736, G3737, G3794, G3739 |
| 106 | G1791 | PRT | Arabidopsis | Paralogous to G1792, G1795, G30; Orthologous to G3380, |
| | | | thaliana | G3381, G3383, G3515, G3516, G3517, G3518, G3519, |
| | | | | G3520, G3735, G3736, G3737, G3794, G3739 |
| 107 | G1808 | DNA | Arabidopsis | Predicted polypeptide sequence is paralogous to G1047 |
| | | | thaliana | |
| 108 | G1808 | PRT | Arabidopsis | Paralogous to G1047 |
| | | | thaliana | |
| 109 | G1809 | DNA | Arabidopsis | |
| | | | thaliana | |
| 110 | G1809 | PRT | Arabidopsis | |
| | | | thaliana | |
| 111 | G1815 | DNA | Arabidopsis | |
| | | | thaliana | |
| 112 | G1815 | PRT | Arabidopsis | |
| | | | thaliana | |
| 113 | G1865 | DNA | Arabidopsis | |
| | | | thaliana | |
| 114 | G1865 | PRT | Arabidopsis | |
| | | | thaliana | |
| 115 | G1884 | DNA | Arabidopsis | |
| | | | thaliana | |
| 116 | G1884 | PRT | Arabidopsis | |
| | | | thaliana | |
| 117 | G1895 | DNA | Arabidopsis | Predicted polypeptide sequence is paralogous to G1903 |
| | | | thaliana | |
| 118 | G1895 | PRT | Arabidopsis | Paralogous to G1903 |
| | | | thaliana | |
| 119 | G1897 | DNA | Arabidopsis | Predicted polypeptide sequence is paralogous to G798 |
| | | | thaliana | |
| 120 | G1897 | PRT | Arabidopsis | Paralogous to G798 |
| | | | thaliana | |
| 121 | G1903 | DNA | Arabidopsis | Predicted polypeptide sequence is paralogous to G1895 |
| | | | thaliana | |
| 122 | G1903 | PRT | Arabidopsis | Paralogous to G1895 |
| | | | thaliana | |
| 123 | G1909 | DNA | Arabidopsis | Predicted polypeptide sequence is paralogous to G1264 |
| | | | thaliana | |
| 124 | G1909 | PRT | Arabidopsis | Paralogous to G1264 |
| | | | thaliana | |
| 125 | G1935 | DNA | Arabidopsis | Predicted polypeptide sequence is paralogous to G2058, |
| | | | thaliana | G2578 |
| 126 | G1935 | PRT | Arabidopsis | Paralogous to G2058, G2578 |
| | | | thaliana | |
| 127 | G1950 | DNA | Arabidopsis | |
| | | | thaliana | |
| 128 | G1950 | PRT | Arabidopsis | |
| | | | thaliana | |
| 129 | G1954 | DNA | Arabidopsis | |
| | | | thaliana | |
| 130 | G1954 | PRT | Arabidopsis | |
| | | | thaliana | |
| 131 | G1958 | DNA | Arabidopsis | |
| | | | thaliana | |
| 132 | G1958 | PRT | Arabidopsis | |
| | | | thaliana | |
| 133 | G2052 | DNA | Arabidopsis | Predicted polypeptide sequence is paralogous to G506 |
| | | | thaliana | |
| 134 | G2052 | PRT | Arabidopsis | Paralogous to G506 |
| | | | thaliana | |
| 135 | G2072 | DNA | Arabidopsis | |
| | | | thaliana | |
| 136 | G2072 | PRT | Arabidopsis | |
| | | | thaliana | |
| 137 | G2108 | DNA | Arabidopsis | |
| | | | thaliana | |
| 138 | G2108 | PRT | Arabidopsis | |
| | | | thaliana | |
| 139 | G2116 | DNA | Arabidopsis | |
| | | | thaliana | |
| 140 | G2116 | PRT | Arabidopsis | |
| | | | thaliana | |
| 141 | G2132 | DNA | Arabidopsis | |
| | | | thaliana | |
| 142 | G2132 | PRT | Arabidopsis | |
| | | | thaliana | |
| 143 | G2137 | DNA | Arabidopsis | |
| | | | thaliana | |
| 144 | G2137 | PRT | Arabidopsis | |
| | | | thaliana | |
| 145 | G2141 | DNA | Arabidopsis | |
| | | | thaliana | |
| 146 | G2141 | PRT | Arabidopsis | |
| | | | thaliana | |
| 147 | G2145 | DNA | Arabidopsis | Predicted polypeptide sequence is paralogous to G2148 |
| | | | thaliana | |
| 148 | G2145 | PRT | Arabidopsis | Paralogous to G2148 |
| | | | thaliana | |
| 149 | G2150 | DNA | Arabidopsis | |
| | | | thaliana | |
| 150 | G2150 | PRT | Arabidopsis | |
| | | | thaliana | |
| 151 | G2157 | DNA | Arabidopsis | |
| | | | thaliana | |
| 152 | G2157 | PRT | Arabidopsis | |
| | | | thaliana | |
| 153 | G2294 | DNA | Arabidopsis | Predicted polypeptide sequence is paralogous to G2067, |
| | | | thaliana | G2115, orthologous to G3657 |
| 154 | G2294 | PRT | Arabidopsis | Paralogous to G2067, G2115; orthologous to G3657 |
| | | | thaliana | |
| 155 | G2296 | DNA | Arabidopsis | |
| | | | thaliana | |
| 156 | G2296 | PRT | Arabidopsis | |
| | | | thaliana | |
| 157 | G2313 | DNA | Arabidopsis | |
| | | | thaliana | |
| 158 | G2313 | PRT | Arabidopsis | |
| | | | thaliana | |
| 159 | G2417 | DNA | Arabidopsis | |
| | | | thaliana | |
| 160 | G2417 | PRT | Arabidopsis | |
| | | | thaliana | |
| 161 | G2425 | DNA | Arabidopsis | |
| | | | thaliana | |
| 162 | G2425 | PRT | Arabidopsis | |
| | | | thaliana | |
| 163 | G2505 | DNA | Arabidopsis | Predicted polypeptide sequence is paralogous to G2635 |
| | | | thaliana | |
| 164 | G2505 | PRT | Arabidopsis | Paralogous to G2635 |
| | | | thaliana | |
| 165 | G10 | DNA | Arabidopsis | Predicted polypeptide sequence is paralogous to G3 |
| | | | thaliana | |
| 166 | G10 | PRT | Arabidopsis | Paralogous to G3 |
| | | | thaliana | |
| 167 | G12 | DNA | Arabidopsis | Predicted polypeptide sequence is paralogous to G1277, |
| | | | thaliana | G1379, G24; orthologous to G3656 |
| 168 | G12 | PRT | Arabidopsis | Paralogous to G1277, G1379, G24; Orthologous to G3656 |
| | | | thaliana | |
| 169 | G28 | DNA | Arabidopsis | Predicted polypeptide sequence is paralogous to G22, |
| | | | thaliana | G1006; orthologous to G3430, G3659, G3660, G3661, |
| | | | | G3717, G3718, G3841, G3843, G3844, G3845, G3846, |
| | | | | G3848, G3852, G3856, G3857, G3858, G3864, G3865 |
| 170 | G28 | PRT | Arabidopsis | Paralogous to G22, G1006; Orthologous to G3430, G3659, |
| | | | thaliana | G3660, G3661, G3717, G3718, G3841, G3843, G3844, |
| | | | | G3845, G3846, G3848, G3852, G3856, G3857, G3858, |
| | | | | G3864, G3865 |
| 171 | G30 | DNA | Arabidopsis | Predicted polypeptide sequence is paralogous to G1791, |
| | | | thaliana | G1792, G1795; orthologous to G3380, G3381, G3383, |
| | | | | G3515, G3516, G3517, G3518, G3519, G3520, G3735, |
| | | | | G3736, G3737, G3794, G3739 |
| 172 | G30 | PRT | Arabidopsis | Paralogous to G1791, G1792, G1795; Orthologous to |
| | | | thaliana | G3380, G3381, G3383, G3515, G3516, G3517, G3518, |
| | | | | G3519, G3520, G3735, G3736, G3737, G3794, G3739 |
| 173 | G165 | DNA | Arabidopsis | Predicted polypeptide sequence is paralogous to G159 |
| | | | thaliana | |
| 174 | G165 | PRT | Arabidopsis | Paralogous to G159 |
| | | | thaliana | |
| 175 | G195 | DNA | Arabidopsis | Predicted polypeptide sequence is paralogous to G187 |
| | | | thaliana | |
| 176 | G195 | PRT | Arabidopsis | Paralogous to G187 |
| | | | thaliana | |
| 177 | G198 | DNA | Arabidopsis | Predicted polypeptide sequence is paralogous to G1328 |
| | | | thaliana | |
| 178 | G198 | PRT | Arabidopsis | Paralogous to G1328 |
| | | | thaliana | |
| 179 | G225 | DNA | Arabidopsis | Predicted polypeptide sequence is paralogous to G1816, |
| | | | thaliana | G226, G2718, G682, G3930; orthologous to G3392, |
| | | | | G3393, G3431, G3444, G3445, G3446, G3447, G3448, |
| | | | | G3449, G3450 |
| 180 | G225 | PRT | Arabidopsis | Paralogous to G1816, G226, G2718, G682, G3930; |
| | | | thaliana | Orthologous to G3392, G3393, G3431, G3444, G3445, |
| | | | | G3446, G3447, G3448, G3449, G3450 |
| 181 | G248 | DNA | Arabidopsis | Predicted polypeptide sequence is paralogous to G1785 |
| | | | thaliana | |
| 182 | G248 | PRT | Arabidopsis | Paralogous to G1785 |
| | | | thaliana | |
| 183 | G448 | DNA | Arabidopsis | Predicted polypeptide sequence is paralogous to G450, |
| | | | thaliana | G455, G456 |
| 184 | G448 | PRT | Arabidopsis | Paralogous to G450, G455, G456 |
| | | | thaliana | |
| 185 | G455 | DNA | Arabidopsis | Predicted polypeptide sequence is paralogous to G448, |
| | | | thaliana | G450, G456 |
| 186 | G455 | PRT | Arabidopsis | Paralogous to G448, G450, G456 |
| | | | thaliana | |
| 187 | G456 | DNA | Arabidopsis | Predicted polypeptide sequence is paralogous to G448, |
| | | | thaliana | G450, G455 |
| 188 | G456 | PRT | Arabidopsis | Paralogous to G448, G450, G455 |
| | | | thaliana | |
| 189 | G506 | DNA | Arabidopsis | Predicted polypeptide sequence is paralogous to G2052 |
| | | | thaliana | |
| 190 | G506 | PRT | Arabidopsis | Paralogous to G2052 |
| | | | thaliana | |
| 191 | G554 | DNA | Arabidopsis | Predicted polypeptide sequence is paralogous to G1198, |
| | | | thaliana | G1806, G555, G556, G558, G578, G629 |
| 192 | G554 | PRT | Arabidopsis | Paralogous to G1198, G1806, G555, G556, G558, G578, |
| | | | thaliana | G629 |
| 193 | G555 | DNA | Arabidopsis | Predicted polypeptide sequence is paralogous to G1198, |
| | | | thaliana | G1806, G554, G556, G558, G578, G629 |
| 194 | G555 | PRT | Arabidopsis | Paralogous to G1198, G1806, G554, G556, G558, G578, |
| | | | thaliana | G629 |
| 195 | G556 | DNA | Arabidopsis | Predicted polypeptide sequence is paralogous to G1198, |
| | | | thaliana | G1806, G554, G555, G558, G578, G629 |
| 196 | G556 | PRT | Arabidopsis | Paralogous to G1198, G1806, G554, G555, G558, G578, |
| | | | thaliana | G629 |
| 197 | G568 | DNA | Arabidopsis | Predicted polypeptide sequence is paralogous to G580 |
| | | | thaliana | |
| 198 | G568 | PRT | Arabidopsis | Paralogous to G580 |
| | | | thaliana | |
| 199 | G577 | DNA | Arabidopsis | Predicted polypeptide sequence is paralogous to G1078 |
| | | | thaliana | |
| 200 | G577 | PRT | Arabidopsis | Paralogous to G1078 |
| | | | thaliana | |
| 201 | G578 | DNA | Arabidopsis | Predicted polypeptide sequence is paralogous to G1198, |
| | | | thaliana | G1806, G554, G555, G556, G558, G629 |
| 202 | G578 | PRT | Arabidopsis | Paralogous to G1198, G1806, G554, G555, G556, G558, |
| | | | thaliana | G629 |
| 203 | G629 | DNA | Arabidopsis | Predicted polypeptide sequence is paralogous to G1198, |
| | | | thaliana | G1806, G554, G555, G556, G558, G578 |
| 204 | G629 | PRT | Arabidopsis | Paralogous to G1198, G1806, G554, G555, G556, G558, |
| | | | thaliana | G578 |
| 205 | G682 | DNA | Arabidopsis | Predicted polypeptide sequence is paralogous to G1816, |
| | | | thaliana | G225, G226, G2718, G3930; orthologous to G3392, |
| | | | | G3393, G3431, G3444, G3445, G3446, G3447, G3448, |
| | | | | G3449, G3450 |
| 206 | G682 | PRT | Arabidopsis | Paralogous to G1816, G225, G226, G2718, G3930; |
| | | | thaliana | Orthologous to G3392, G3393, G3431, G3444, G3445, |
| | | | | G3446, G3447, G3448, G3449, G3450 |
| 207 | G730 | DNA | Arabidopsis | Predicted polypeptide sequence is paralogous to G1040, |
| | | | thaliana | G3034, G729 |
| 208 | G730 | PRT | Arabidopsis | Paralogous to G1040, G3034, G729 |
| | | | thaliana | |
| 209 | G761 | DNA | Arabidopsis | Predicted polypeptide sequence is paralogous to G1354, |
| | | | thaliana | G1355, G1453, G1766, G2534, G522 |
| 210 | G761 | PRT | Arabidopsis | Paralogous to G1354, G1355, G1453, G1766, G2534, |
| | | | thaliana | G522 |
| 211 | G798 | DNA | Arabidopsis | Predicted polypeptide sequence is paralogous to G1897 |
| | | | thaliana | |
| 212 | G798 | PRT | Arabidopsis | Paralogous to G1897 |
| | | | thaliana | |
| 213 | G900 | DNA | Arabidopsis | Predicted polypeptide sequence is paralogous to G1481, |
| | | | thaliana | orthologous to G4014, G4015, G4016 |
| 214 | G900 | PRT | Arabidopsis | Paralogous to G1481; orthologous to G4014, G4015, |
| | | | thaliana | G4016 |
| 215 | G986 | DNA | Arabidopsis | Predicted polypeptide sequence is paralogous to G881 |
| | | | thaliana | |
| 216 | G986 | PRT | Arabidopsis | Paralogous to G881 |
| | | | thaliana | |
| 217 | G1006 | DNA | Arabidopsis | Predicted polypeptide sequence is paralogous to G22, G28; |
| | | | thaliana | orthologous to G3430, G3659, G3660, G3661, G3717, |
| | | | | G3718, G3841, G3843, G3844, G3845, G3846, G3848, |
| | | | | G3852, G3856, G3857, G3858, G3864, G3865 |
| 218 | G1006 | PRT | Arabidopsis | Paralogous to G22, G28; Orthologous to G3430, G3659, |
| | | | thaliana | G3660, G3661, G3717, G3718, G3841, G3843, G3844, |
| | | | | G3845, G3846, G3848, G3852, G3856, G3857, G3858, |
| | | | | G3864, G3865 |
| 219 | G1040 | DNA | Arabidopsis | Predicted polypeptide sequence is paralogous to G3034, |
| | | | thaliana | G729, G730 |
| 220 | G1040 | PRT | Arabidopsis | Paralogous to G3034, G729, G730 |
| | | | thaliana | |
| 221 | G1047 | DNA | Arabidopsis | Predicted polypeptide sequence is paralogous to G1808 |
| | | | thaliana | |
| 222 | G1047 | PRT | Arabidopsis | Paralogous to G1808 |
| | | | thaliana | |
| 223 | G1198 | DNA | Arabidopsis | Predicted polypeptide sequence is paralogous to G1806, |
| | | | thaliana | G554, G555, G556, G558, G578, G629 |
| 224 | G1198 | PRT | Arabidopsis | Paralogous to G1806, G554, G555, G556, G558, G578, |
| | | | thaliana | G629 |
| 225 | G1264 | DNA | Arabidopsis | Predicted polypeptide sequence is paralogous to G1909 |
| | | | thaliana | |
| 226 | G1264 | PRT | Arabidopsis | Paralogous to G1909 |
| | | | thaliana | |
| 227 | G1277 | DNA | Arabidopsis | Predicted polypeptide sequence is paralogous to G12, |
| | | | thaliana | G1379, G24; orthologous to G3656 |
| 228 | G1277 | PRT | Arabidopsis | Paralogous to G12, G1379, G24; Orthologous to G3656 |
| | | | thaliana | |
| 229 | G1309 | DNA | Arabidopsis | Predicted polypeptide sequence is paralogous to G237 |
| | | | thaliana | |
| 230 | G1309 | PRT | Arabidopsis | Paralogous to G237 |
| | | | thaliana | |
| 231 | G1354 | DNA | Arabidopsis | Predicted polypeptide sequence is paralogous to G1355, |
| | | | thaliana | G1453, G1766, G2534, G522, G761 |
| 232 | G1354 | PRT | Arabidopsis | Paralogous to G1355, G1453, G1766, G2534, G522, G761 |
| | | | thaliana | |
| 233 | G1355 | DNA | Arabidopsis | Predicted polypeptide sequence is paralogous to G1354, |
| | | | thaliana | G1453, G1766, G2534, G522, G761 |
| 234 | G1355 | PRT | Arabidopsis | Paralogous to G1354, G1453, G1766, G2534, G522, G761 |
| | | | thaliana | |
| 235 | G1379 | DNA | Arabidopsis | Predicted polypeptide sequence is paralogous to G12, |
| | | | thaliana | G1277, G24; orthologous to G3656 |
| 236 | G1379 | PRT | Arabidopsis | Paralogous to G12, G1277, G24; Orthologous to G3656 |
| | | | thaliana | |
| 237 | G1453 | DNA | Arabidopsis | Predicted polypeptide sequence is paralogous to G1354, |
| | | | thaliana | G1355, G1766, G2534, G522, G761 |
| 238 | G1453 | PRT | Arabidopsis | Paralogous to G1354, G1355, G1766, G2534, G522, G761 |
| | | | thaliana | |
| 239 | G1461 | DNA | Arabidopsis | Predicted polypeptide sequence is paralogous to G1462, |
| | | | thaliana | G1463, G1464, G1465 |
| 240 | G1461 | PRT | Arabidopsis | Paralogous to G1462, G1463, G1464, G1465 |
| | | | thaliana | |
| 241 | G1464 | DNA | Arabidopsis | Predicted polypeptide sequence is paralogous to G1461, |
| | | | thaliana | G1462, G1463, G1465 |
| 242 | G1464 | PRT | Arabidopsis | Paralogous to G1461, G1462, G1463, G1465 |
| | | | thaliana | |
| 243 | G1465 | DNA | Arabidopsis | Predicted polypeptide sequence is paralogous to G1461, |
| | | | thaliana | G1462, G1463, G1464 |
| 244 | G1465 | PRT | Arabidopsis | Paralogous to G1461, G1462, G1463, G1464 |
| | | | thaliana | |
| 245 | G1754 | DNA | Arabidopsis | Predicted polypeptide sequence is paralogous to G1755 |
| | | | thaliana | |
| 246 | G1754 | PRT | Arabidopsis | Paralogous to G1755 |
| | | | thaliana | |
| 247 | G1766 | DNA | Arabidopsis | Predicted polypeptide sequence is paralogous to G1354, |
| | | | thaliana | G1355, G1453, G2534, G522, G761 |
| 248 | G1766 | PRT | Arabidopsis | Paralogous to G1354, G1355, G1453, G2534, G522, G761 |
| | | | thaliana | |
| 249 | G1792 | DNA | Arabidopsis | Predicted polypeptide sequence is paralogous to G1791, |
| | | | thaliana | G1795, G30; orthologous to G3380, G3381, G3383, |
| | | | | G3515, G3516, G3517, G3518, G3519, G3520, G3735, |
| | | | | G3736, G3737, G3794, G3739 |
| 250 | G1792 | PRT | Arabidopsis | Paralogous to G1791, G1795, G30; Orthologous to G3380, |
| | | | thaliana | G3381, G3383, G3515, G3516, G3517, G3518, G3519, |
| | | | | G3520, G3735, G3736, G3737, G3794, G3739 |
| 251 | G1795 | DNA | Arabidopsis | Predicted polypeptide sequence is paralogous to G1791, |
| | | | thaliana | G1792, G30; orthologous to G3380, G3381, G3383, |
| | | | | G3515, G3516, G3517, G3518, G3519, G3520, G3735, |
| | | | | G3736, G3737, G3794, G3739 |
| 252 | G1795 | PRT | Arabidopsis | Paralogous to G1791, G1792, G30; Orthologous to G3380, |
| | | | thaliana | G3381, G3383, G3515, G3516, G3517, G3518, G3519, |
| | | | | G3520, G3735, G3736, G3737, G3794, G3739 |
| 253 | G1806 | DNA | Arabidopsis | Predicted polypeptide sequence is paralogous to G1198, |
| | | | thaliana | G554, G555, G556, G558, G578, G629 |
| 254 | G1806 | PRT | Arabidopsis | Paralogous to G1198, G554, G555, G556, G558, G578, |
| | | | thaliana | G629 |
| 255 | G1816 | DNA | Arabidopsis | Predicted polypeptide sequence is paralogous to G225, |
| | | | thaliana | G226, G2718, G682; orthologous to G3392, G3393, |
| | | | | G3431, G3444, G3445, G3446, G3447, G3448, G3449, |
| | | | | G3450 |
| 256 | G1816 | PRT | Arabidopsis | Paralogous to G225, G226, G2718, G682; Orthologous to |
| | | | thaliana | G3392, G3393, G3431, G3444, G3445, G3446, G3447, |
| | | | | G3448, G3449, G3450 |
| 257 | G1846 | DNA | Arabidopsis | Predicted polypeptide sequence is paralogous to G1007 |
| | | | thaliana | |
| 258 | G1846 | PRT | Arabidopsis | Paralogous to G1007 |
| | | | thaliana | |
| 259 | G1917 | DNA | Arabidopsis | Predicted polypeptide sequence is paralogous to G383 |
| | | | thaliana | |
| 260 | G1917 | PRT | Arabidopsis | Paralogous to G383 |
| | | | thaliana | |
| 261 | G2058 | DNA | Arabidopsis | Predicted polypeptide sequence is paralogous to G1935, |
| | | | thaliana | G2578 |
| 262 | G2058 | PRT | Arabidopsis | Paralogous to G1935, G2578 |
| | | | thaliana | |
| 263 | G2067 | DNA | Arabidopsis | Predicted polypeptide sequence is paralogous to G2115, |
| | | | thaliana | G2294, orthologous to G3657 |
| 264 | G2067 | PRT | Arabidopsis | Paralogous to G2115, G2294; orthologous to G3657 |
| | | | thaliana | |
| 265 | G2115 | DNA | Arabidopsis | Predicted polypeptide sequence is paralogous to G2067, |
| | | | thaliana | G2294, orthologous to G3657 |
| 266 | G2115 | PRT | Arabidopsis | Paralogous to G2067, G2294; orthologous to G3657 |
| | | | thaliana | |
| 267 | G2133 | DNA | Arabidopsis | Predicted polypeptide sequence is paralogous to G47; |
| | | | thaliana | orthologous to G3643, G3644, G3645, G3646, G3647, |
| | | | | G3649, G3650, G3651 |
| 268 | G2133 | PRT | Arabidopsis | Paralogous to G47; Orthologous to G3643, G3644, G3645, |
| | | | thaliana | G3646, G3647, G3649, G3650, G3651 |
| 269 | G2148 | DNA | Arabidopsis | Predicted polypeptide sequence is paralogous to G2145 |
| | | | thaliana | |
| 270 | G2148 | PRT | Arabidopsis | Paralogous to G2145 |
| | | | thaliana | |
| 271 | G2424 | DNA | Arabidopsis | Predicted polypeptide sequence is paralogous to G1645 |
| | | | thaliana | |
| 272 | G2424 | PRT | Arabidopsis | Paralogous to G1645 |
| | | | thaliana | |
| 273 | G2436 | DNA | Arabidopsis | Predicted polypeptide sequence is paralogous to G2443, |
| | | | thaliana | G328 |
| 274 | G2436 | PRT | Arabidopsis | Paralogous to G2443, G328 |
| | | | thaliana | |
| 275 | G2442 | DNA | Arabidopsis | Predicted polypeptide sequence is paralogous to G1504, |
| | | | thaliana | G2504 |
| 276 | G2442 | PRT | Arabidopsis | Paralogous to G1504, G2504 |
| | | | thaliana | |
| 277 | G2443 | DNA | Arabidopsis | Predicted polypeptide sequence is paralogous to G2436, |
| | | | thaliana | G328 |
| 278 | G2443 | PRT | Arabidopsis | Paralogous to G2436, G328 |
| | | | thaliana | |
| 279 | G2467 | DNA | Arabidopsis | Predicted polypeptide sequence is paralogous to G812 |
| | | | thaliana | |
| 280 | G2467 | PRT | Arabidopsis | Paralogous to G812 |
| | | | thaliana | |
| 281 | G2504 | DNA | Arabidopsis | Predicted polypeptide sequence is paralogous to G1504, |
| | | | thaliana | G2442 |
| 282 | G2504 | PRT | Arabidopsis | Paralogous to G1504, G2442 |
| | | | thaliana | |
| 283 | G2512 | DNA | Arabidopsis | Predicted polypeptide sequence is paralogous to G1752 |
| | | | thaliana | |
| 284 | G2512 | PRT | Arabidopsis | Paralogous to G1752 |
| | | | thaliana | |
| 285 | G2534 | DNA | Arabidopsis | Predicted polypeptide sequence is paralogous to G1354, |
| | | | thaliana | G1355, G1453, G1766, G522, G761 |
| 286 | G2534 | PRT | Arabidopsis | Paralogous to G1354, G1355, G1453, G1766, G522, G761 |
| | | | thaliana | |
| 287 | G2578 | DNA | Arabidopsis | Predicted polypeptide sequence is paralogous to G1935, |
| | | | thaliana | G2058 |
| 288 | G2578 | PRT | Arabidopsis | Paralogous to G1935, G2058 |
| | | | thaliana | |
| 289 | G2629 | DNA | Arabidopsis | Predicted polypeptide sequence is paralogous to G1053 |
| | | | thaliana | |
| 290 | G2629 | PRT | Arabidopsis | Paralogous to G1053 |
| | | | thaliana | |
| 291 | G2635 | DNA | Arabidopsis | Predicted polypeptide sequence is paralogous to G2505 |
| | | | thaliana | |
| 292 | G2635 | PRT | Arabidopsis | Paralogous to G2505 |
| | | | thaliana | |
| 293 | G2718 | DNA | Arabidopsis | Predicted polypeptide sequence is paralogous to G1816, |
| | | | thaliana | G225, G226, G682, G3930; orthologous to G3392, G3393, |
| | | | | G3431, G3444, G3445, G3446, G3447, G3448, G3449, |
| | | | | G3450 |
| 294 | G2718 | PRT | Arabidopsis | Paralogous to G1816, G225, G226, G682, G3930; |
| | | | thaliana | Orthologous to G3392, G3393, G3431, G3444, G3445, |
| | | | | G3446, G3447, G3448, G3449, G3450 |
| 295 | G2893 | DNA | Arabidopsis | Predicted polypeptide sequence is paralogous to G1324 |
| | | | thaliana | |
| 296 | G2893 | PRT | Arabidopsis | Paralogous to G1324 |
| | | | thaliana | |
| 297 | G3034 | DNA | Arabidopsis | Predicted polypeptide sequence is paralogous to G1040, |
| | | | thaliana | G729, G730 |
| 298 | G3034 | PRT | Arabidopsis | Paralogous to G1040, G729, G730 |
| | | | thaliana | |
| 299 | G3380 | DNA | Oryza sativa | Predicted polypeptide sequence is paralogous to G3381, |
| | | | (japonica | G3383, G3515, G3737; orthologous to G1791, G1792, |
| | | | cultivar-group) | G1795, G30, G3516, G3517, G3518, G3519, G3520, |
| | | | | G3735, G3736, G3794, G3739 |
| 300 | G3380 | PRT | Oryza sativa | Paralogous to G3381, G3383, G3515, G3737; Orthologous |
| | | | (japonica | to G1791, G1792, G1795, G30, G3516, G3517, G3518, |
| | | | cultivar-group) | G3519, G3520, G3735, G3736, G3794, G3739 |
| 301 | G3381 | DNA | Oryza sativa | Predicted polypeptide sequence is paralogous to G3380, |
| | | | (japonica | G3383, G3515, G3737; orthologous to G1791, G1792, |
| | | | cultivar-group) | G1795, G30, G3516, G3517, G3518, G3519, G3520, |
| | | | | G3735, G3736, G3794, G3739 |
| 302 | G3381 | PRT | Oryza sativa | Paralogous to G3380, G3383, G3515, G3737; Orthologous |
| | | | (japonica | to G1791, G1792, G1795, G30, G3516, G3517, G3518, |
| | | | cultivar-group) | G3519, G3520, G3735, G3736, G3794, G3739 |
| 303 | G3383 | DNA | Oryza sativa | Predicted polypeptide sequence is paralogous to G3380, |
| | | | (japonica | G3381, G3515, G3737; orthologous to G1791, G1792, |
| | | | cultivar-group) | G1795, G30, G3516, G3517, G3518, G3519, G3520, |
| | | | | G3735, G3736, G3794, G3739 |
| 304 | G3383 | PRT | Oryza sativa | Paralogous to G3380, G3381, G3515, G3737; Orthologous |
| | | | (japonica | to G1791, G1792, G1795, G30, G3516, G3517, G3518, |
| | | | cultivar-group) | G3519, G3520, G3735, G3736, G3794, G3739 |
| 305 | G3392 | DNA | Oryza sativa | Predicted polypeptide sequence is paralogous to G3393; |
| | | | (japonica | orthologous to G1816, G225, G226, G2718, G682, G3431, |
| | | | cultivar-group) | G3444, G3445, G3446, G3447, G3448, G3449, G3450, |
| | | | | G3930 |
| 306 | G3392 | PRT | Oryza sativa | Paralogous to G3393; Orthologous to G1816, G225, G226, |
| | | | (japonica | G2718, G682, G3431, G3444, G3445, G3446, G3447, |
| | | | cultivar-group) | G3448, G3449, G3450, G3930 |
| 307 | G3393 | DNA | Oryza sativa | Predicted polypeptide sequence is paralogous to G3392; |
| | | | (japonica | orthologous to G1816, G225, G226, G2718, G682, G3431, |
| | | | cultivar-group) | G3444, G3445, G3446, G3447, G3448, G3449, G3450, |
| | | | | G3930 |
| 308 | G3393 | PRT | Oryza sativa | Paralogous to G3392; Orthologous to G1816, G225, G226, |
| | | | (japonica | G2718, G682, G3431, G3444, G3445, G3446, G3447, |
| | | | cultivar-group) | G3448, G3449, G3450, G3930 |
| 309 | G3430 | DNA | Oryza sativa | Predicted polypeptide sequence is paralogous to G3848; |
| | | | (japonica | orthologous to G22, G1006, G28, G3659, G3660, G3661, |
| | | | cultivar-group) | G3717, G3718, G3841, G3843, G3844, G3845, G3846, |
| | | | | G3852, G3856, G3857, G3858, G3864, G3865 |
| 310 | G3430 | PRT | Oryza sativa | Paralogous to G3848; Orthologous to G22, G1006, G28, |
| | | | (japonica | G3659, G3660, G3661, G3717, G3718, G3841, G3843, |
| | | | cultivar-group) | G3844, G3845, G3846, G3852, G3856, G3857, G3858, |
| | | | | G3864, G3865 |
| 311 | G3431 | DNA | Zea mays | Predicted polypeptide sequence is paralogous to G3444; |
| | | | | orthologous to G1816, G225, G226, G2718, G682, G3392, |
| | | | | G3393, G3445, G3446, G3447, G3448, G3449, G3450, |
| | | | | G3930 |
| 312 | G3431 | PRT | Zea mays | Paralogous to G3444; Orthologous to G1816, G225, G226, |
| | | | | G2718, G682, G3392, G3393, G3445, G3446, G3447, |
| | | | | G3448, G3449, G3450, G3930 |
| 313 | G3444 | DNA | Zea mays | Predicted polypeptide sequence is paralogous to G3431; |
| | | | | orthologous to G1816, G225, G226, G2718, G682, G3392, |
| | | | | G3393, G3445, G3446, G3447, G3448, G3449, G3450, |
| | | | | G3930 |
| 314 | G3444 | PRT | Zea mays | Paralogous to G3431; Orthologous to G1816, G225, G226, |
| | | | | G2718, G682, G3392, G3393, G3445, G3446, G3447, |
| | | | | G3448, G3449, G3450, G3930 |
| 315 | G3445 | DNA | Glycine max | Predicted polypeptide sequence is paralogous to G3446, |
| | | | | G3447, G3448, G3449, G3450; orthologous to G1816, |
| | | | | G225, G226, G2718, G682, G3392, G3393, G3431, |
| | | | | G3444, G3930 |
| 316 | G3445 | PRT | Glycine max | Paralogous to G3446, G3447, G3448, G3449, G3450; |
| | | | | Orthologous to G1816, G225, G226, G2718, G682, G3392, |
| | | | | G3393, G3431, G3444, G3930 |
| 317 | G3446 | DNA | Glycine max | Predicted polypeptide sequence is paralogous to G3445, |
| | | | | G3447, G3448, G3449, G3450; orthologous to G1816, |
| | | | | G225, G226, G2718, G682, G3392, G3393, G3431, |
| | | | | G3444, G3930 |
| 318 | G3446 | PRT | Glycine max | Paralogous to G3445, G3447, G3448, G3449, G3450; |
| | | | | Orthologous to G1816, G225, G226, G2718, G682, G3392, |
| | | | | G3393, G3431, G3444, G3930 |
| 319 | G3447 | DNA | Glycine max | Predicted polypeptide sequence is paralogous to G3445, |
| | | | | G3446, G3448, G3449, G3450; orthologous to G1816, |
| | | | | G225, G226, G2718, G682, G3392, G3393, G3431, |
| | | | | G3444, G3930 |
| 320 | G3447 | PRT | Glycine max | Paralogous to G3445, G3446, G3448, G3449, G3450; |
| | | | | Orthologous to G1816, G225, G226, G2718, G682, G3392, |
| | | | | G3393, G3431, G3444, G3930 |
| 321 | G3448 | DNA | Glycine max | Predicted polypeptide sequence is paralogous to G3445, |
| | | | | G3446, G3447, G3449, G3450; orthologous to G1816, |
| | | | | G225, G226, G2718, G682, G3392, G3393, G3431, |
| | | | | G3444, G3930 |
| 322 | G3448 | PRT | Glycine max | Paralogous to G3445, G3446, G3447, G3449, G3450; |
| | | | | Orthologous to G1816, G225, G226, G2718, G682, G3392, |
| | | | | G3393, G3431, G3444, G3930 |
| 323 | G3449 | DNA | Glycine max | Predicted polypeptide sequence is paralogous to G3445, |
| | | | | G3446, G3447, G3448, G3450; orthologous to G1816, |
| | | | | G225, G226, G2718, G682, G3392, G3393, G3431, |
| | | | | G3444, G3930 |
| 324 | G3449 | PRT | Glycine max | Paralogous to G3445, G3446, G3447, G3448, G3450; |
| | | | | Orthologous to G1816, G225, G226, G2718, G682, G3392, |
| | | | | G3393, G3431, G3444, G3930 |
| 325 | G3450 | DNA | Glycine max | Predicted polypeptide sequence is paralogous to G3445, |
| | | | | G3446, G3447, G3448, G3449; orthologous to G1816, |
| | | | | G225, G226, G2718, G682, G3392, G3393, G3431, |
| | | | | G3444, G3930 |
| 326 | G3450 | PRT | Glycine max | Paralogous to G3445, G3446, G3447, G3448, G3449; |
| | | | | Orthologous to G1816, G225, G226, G2718, G682, G3392, |
| | | | | G3393, G3431, G3444, G3930 |
| 327 | G3490 | DNA | Zea mays | Predicted polypeptide sequence is orthologous to G1543, |
| | | | | G3510, G3524 |
| 328 | G3490 | PRT | Zea mays | Orthologous to G1543, G3510, G3524 |
| 825 | G3510 | DNA | Oryza sativa | Predicted polypeptide sequence is orthologous to G1543, |
| | | | (japonica | G3490, G3524 |
| | | | cultivar-group) | |
| 826 | G3510 | PRT | Oryza sativa | Orthologous to G1543, G3490, G3524 |
| | | | (japonica | |
| | | | cultivar-group) | |
| 329 | G3515 | DNA | Oryza sativa | Predicted polypeptide sequence is paralogous to G3380, |
| | | | (japonica | G3381, G3383, G3737; orthologous to G1791, G1792, |
| | | | cultivar-group) | G1795, G30, G3516, G3517, G3518, G3519, G3520, |
| | | | | G3735, G3736, G3794, G3739 |
| 330 | G3515 | PRT | Oryza sativa | Paralogous to G3380, G3381, G3383, G3737; Orthologous |
| | | | (japonica | to G1791, G1792, G1795, G30, G3516, G3517, G3518, |
| | | | cultivar-group) | G3519, G3520, G3735, G3736, G3794, G3739 |
| 331 | G3516 | DNA | Zea mays | Predicted polypeptide sequence is paralogous to G3517, |
| | | | | G3794, G3739; orthologous to G1791, G1792, G1795, |
| | | | | G30, G3380, G3381, G3383, G3515, G3518, G3519, |
| | | | | G3520, G3735, G3736, G3737 |
| 332 | G3516 | PRT | Zea mays | Paralogous to G3517, G3794, G3739; Orthologous to |
| | | | | G1791, G1792, G1795, G30, G3380, G3381, G3383, |
| | | | | G3515, G3518, G3519, G3520, G3735, G3736, G3737 |
| 333 | G3517 | DNA | Zea mays | Predicted polypeptide sequence is paralogous to G3516, |
| | | | | G3794, G3739; orthologous to G1791, G1792, G1795, |
| | | | | G30, G3380, G3381, G3383, G3515, G3518, G3519, |
| | | | | G3520, G3735, G3736, G3737 |
| 334 | G3517 | PRT | Zea mays | Paralogous to G3516, G3794, G3739; Orthologous to |
| | | | | G1791, G1792, G1795, G30, G3380, G3381, G3383, |
| | | | | G3515, G3518, G3519, G3520, G3735, G3736, G3737 |
| 335 | G3518 | DNA | Glycine max | Predicted polypeptide sequence is paralogous to G3519, |
| | | | | G3520; orthologous to G1791, G1792, G1795, G30, |
| | | | | G3380, G3381, G3383, G3515, G3516, G3517, G3735, |
| | | | | G3736, G3737, G3794, G3739 |
| 336 | G3518 | PRT | Glycine max | Paralogous to G3519, G3520; Orthologous to G1791, |
| | | | | G1792, G1795, G30, G3380, G3381, G3383, G3515, |
| | | | | G3516, G3517, G3735, G3736, G3737, G3794, G3739 |
| 337 | G3519 | DNA | Glycine max | Predicted polypeptide sequence is paralogous to G3518, |
| | | | | G3520; orthologous to G1791, G1792, G1795, G30, |
| | | | | G3380, G3381, G3383, G3515, G3516, G3517, G3735, |
| | | | | G3736, G3737, G3794, G3739 |
| 338 | G3519 | PRT | Glycine max | Paralogous to G3518, G3520; Orthologous to G1791, |
| | | | | G1792, G1795, G30, G3380, G3381, G3383, G3515, |
| | | | | G3516, G3517, G3735, G3736, G3737, G3794, G3739 |
| 339 | G3520 | DNA | Glycine max | Predicted polypeptide sequence is paralogous to G3518, |
| | | | | G3519; orthologous to G1791, G1792, G1795, G30, |
| | | | | G3380, G3381, G3383, G3515, G3516, G3517, G3735, |
| | | | | G3736, G3737, G3794, G3739 |
| 340 | G3520 | PRT | Glycine max | Paralogous to G3518, G3519; Orthologous to G1791, |
| | | | | G1792, G1795, G30, G3380, G3381, G3383, G3515, |
| | | | | G3516, G3517, G3735, G3736, G3737, G3794, G3739 |
| 341 | G3524 | DNA | Glycine max | Predicted polypeptide sequence is orthologous to G1543, |
| | | | | G3510, G3490 |
| 342 | G3524 | PRT | Glycine max | Orthologous to G1543, G3510, G3490 |
| 343 | G3643 | DNA | Glycine max | Predicted polypeptide sequence is orthologous to G2133, |
| | | | | G47, G3644, G3645, G3646, G3647, G3649, G3650, |
| | | | | G3651 |
| 344 | G3643 | PRT | Glycine max | Orthologous to G2133, G47, G3644, G3645, G3646, |
| | | | | G3647, G3649, G3650, G3651 |
| 345 | G3644 | DNA | Oryza sativa | Predicted polypeptide sequence is paralogous to G3649, |
| | | | (japonica | G3651; orthologous to G2133, G47, G3643, G3645, |
| | | | cultivar-group) | G3646, G3647, G3650 |
| 346 | G3644 | PRT | Oryza sativa | Paralogous to G3649, G3651; Orthologous to G2133, G47, |
| | | | (japonica | G3643, G3645, G3646, G3647, G3650 |
| | | | cultivar-group) | |
| 347 | G3645 | DNA | Brassica rapa | Predicted polypeptide sequence is orthologous to G2133, |
| | | | subsp. | G47, G3643, G3644, G3646, G3647, G3649, G3650, |
| | | | Pekinensis | G3651 |
| 348 | G3645 | PRT | Brassica rapa | Orthologous to G2133, G47, G3643, G3644, G3646, |
| | | | subsp. | G3647, G3649, G3650, G3651 |
| | | | Pekinensis | |
| 349 | G3646 | DNA | Brassica | Predicted polypeptide sequence is orthologous to G2133, |
| | | | oleracea | G47, G3643, G3644, G3645, G3647, G3649, G3650, |
| | | | | G3651 |
| 350 | G3646 | PRT | Brassica | Orthologous to G2133, G47, G3643, G3644, G3645, |
| | | | oleracea | G3647, G3649, G3650, G3651 |
| 351 | G3647 | DNA | Zinnia elegans | Predicted polypeptide sequence is orthologous to G2133, |
| | | | | G47, G3643, G3644, G3645, G3646, G3649, G3650, |
| | | | | G3651 |
| 352 | G3647 | PRT | Zinnia elegans | Orthologous to G2133, G47, G3643, G3644, G3645, |
| | | | | G3646, G3649, G3650, G3651 |
| 353 | G3649 | DNA | Oryza sativa | Predicted polypeptide sequence is paralogous to G3644, |
| | | | (japonica | G3651; orthologous to G2133, G47, G3643, G3645, |
| | | | cultivar-group) | G3646, G3647, G3650 |
| 354 | G3649 | PRT | Oryza sativa | Paralogous to G3644, G3651; Orthologous to G2133, G47, |
| | | | (japonica | G3643, G3645, G3646, G3647, G3650 |
| | | | cultivar-group) | |
| 827 | G3650 | DNA | Zea mays | Predicted polypeptide sequence is orthologous to G2133, |
| | | | | G47, G3643, G3644, G3645, G3646, G3647, G3649, |
| | | | | G3651 |
| 828 | G3650 | PRT | Zea mays | Orthologous to G2133, G47, G3643, G3644, G3645, |
| | | | | G3646, G3647, G3649, G3651 |
| 355 | G3651 | DNA | Oryza sativa | Predicted polypeptide sequence is paralogous to G3644, |
| | | | (japonica | G3649; orthologous to G2133, G47, G3643, G3645, |
| | | | cultivar-group) | G3646, G3647, G3650 |
| 356 | G3651 | PRT | Oryza sativa | Paralogous to G3644, G3649; Orthologous to G2133, G47, |
| | | | (japonica | G3643, G3645, G3646, G3647, G3650 |
| | | | cultivar-group) | |
| 357 | G3656 | DNA | Zea mays | Predicted polypeptide sequence is orthologous to G12, |
| | | | | G1277, G1379, G24 |
| 358 | G3656 | PRT | Zea mays | Orthologous to G12, G1277, G1379, G24 |
| 829 | G3657 | DNA | Oryza sativa | Predicted polypeptide sequence is orthologous to G2294, |
| | | | (japonica | G2067, G2115 |
| | | | cultivar-group) | |
| 830 | G3657 | PRT | Oryza sativa | Orthologous to G2294, G2067, G2115 |
| | | | (japonica | |
| | | | cultivar-group) | |
| 359 | G3659 | DNA | Brassica | Predicted polypeptide sequence is paralogous to G3660; |
| | | | oleracea | orthologous to G22, G1006, G28, G3430, G3661, G3717, |
| | | | | G3718, G3841, G3843, G3844, G3845, G3846, G3848, |
| | | | | G3852, G3856, G3857, G3858, G3864, G3865 |
| 360 | G3659 | PRT | Brassica | Paralogous to G3660; Orthologous to G22, G1006, G28, |
| | | | oleracea | G3430, G3661, G3717, G3718, G3841, G3843, G3844, |
| | | | | G3845, G3846, G3848, G3852, G3856, G3857, G3858, |
| | | | | G3864, G3865 |
| 361 | G3660 | DNA | Brassica | Predicted polypeptide sequence is paralogous to G3659; |
| | | | oleracea | orthologous to G22, G1006, G28, G3430, G3661, G3717, |
| | | | | G3718, G3841, G3843, G3844, G3845, G3846, G3848, |
| | | | | G3852, G3856, G3857, G3858, G3864, G3865 |
| 362 | G3660 | PRT | Brassica | Paralogous to G3659; Orthologous to G22, G1006, G28, |
| | | | oleracea | G3430, G3661, G3717, G3718, G3841, G3843, G3844, |
| | | | | G3845, G3846, G3848, G3852, G3856, G3857, G3858, |
| | | | | G3864, G3865 |
| 363 | G3661 | DNA | Zea mays | Predicted polypeptide sequence is paralogous to G3856; |
| | | | | orthologous to G22, G1006, G28, G3430, G3659, G3660, |
| | | | | G3717, G3718, G3841, G3843, G3844, G3845, G3846, |
| | | | | G3848, G3852, G3857, G3858, G3864, G3865 |
| 364 | G3661 | PRT | Zea mays | Paralogous to G3856; Orthologous to G22, G1006, G28, |
| | | | | G3430, G3659, G3660, G3717, G3718, G3841, G3843, |
| | | | | G3844, G3845, G3846, G3848, G3852, G3857, G3858, |
| | | | | G3864, G3865 |
| 365 | G3717 | DNA | Glycine max | Predicted polypeptide sequence is paralogous to G3718; |
| | | | | orthologous to G22, G1006, G28, G3430, G3659, G3660, |
| | | | | G3661, G3841, G3843, G3844, G3845, G3846, G3848, |
| | | | | G3852, G3856, G3857, G3858, G3864, G3865 |
| 366 | G3717 | PRT | Glycine max | Paralogous to G3718; Orthologous to G22, G1006, G28, |
| | | | | G3430, G3659, G3660, G3661, G3841, G3843, G3844, |
| | | | | G3845, G3846, G3848, G3852, G3856, G3857, G3858, |
| | | | | G3864, G3865 |
| 367 | G3718 | DNA | Glycine max | Predicted polypeptide sequence is paralogous to G3717; |
| | | | | orthologous to G22, G1006, G28, G3430, G3659, G3660, |
| | | | | G3661, G3841, G3843, G3844, G3845, G3846, G3848, |
| | | | | G3852, G3856, G3857, G3858, G3864, G3865 |
| 368 | G3718 | PRT | Glycine max | Paralogous to G3717; Orthologous to G22, G1006, G28, |
| | | | | G3430, G3659, G3660, G3661, G3841, G3843, G3844, |
| | | | | G3845, G3846, G3848, G3852, G3856, G3857, G3858, |
| | | | | G3864, G3865 |
| 369 | G3735 | DNA | Medicago | Predicted polypeptide sequence is orthologous to G1791, |
| | | | truncatula | G1792, G1795, G30, G3380, G3381, G3383, G3515, |
| | | | | G3516, G3517, G3518, G3519, G3520, G3736, G3737, |
| | | | | G3794, G3739 |
| 370 | G3735 | PRT | Medicago | Orthologous to G1791, G1792, G1795, G30, G3380, |
| | | | truncatula | G3381, G3383, G3515, G3516, G3517, G3518, G3519, |
| | | | | G3520, G3736, G3737, G3794, G3739 |
| 371 | G3736 | DNA | Triticum | Predicted polypeptide sequence is orthologous to G1791, |
| | | | aestivum | G1792, G1795, G30, G3380, G3381, G3383, G3515, |
| | | | | G3516, G3517, G3518, G3519, G3520, G3735, G3737, |
| | | | | G3794, G3739 |
| 372 | G3736 | PRT | Triticum | Orthologous to G1791, G1792, G1795, G30, G3380, |
| | | | aestivum | G3381, G3383, G3515, G3516, G3517, G3518, G3519, |
| | | | | G3520, G3735, G3737, G3794, G3739 |
| 373 | G3737 | DNA | Oryza sativa | Predicted polypeptide sequence is paralogous to G3380, |
| | | | (japonica | G3381, G3383, G3515; orthologous to G1791, G1792, |
| | | | cultivar-group) | G1795, G30, G3516, G3517, G3518, G3519, G3520, |
| | | | | G3735, G3736, G3794, G3739 |
| 374 | G3737 | PRT | Oryza sativa | Paralogous to G3380, G3381, G3383, G3515; Orthologous |
| | | | (japonica | to G1791, G1792, G1795, G30, G3516, G3517, G3518, |
| | | | cultivar-group) | G3519, G3520, G3735, G3736, G3794, G3739 |
| 375 | G3739 | DNA | Zea mays | Predicted polypeptide sequence is paralogous to G3516, |
| | | | | G3517, G3794; orthologous to G1791, G1792, G1795, |
| | | | | G30, G3380, G3381, G3383, G3515, G3518, G3519, |
| | | | | G3520, G3735, G3736, G3737 |
| 376 | G3739 | PRT | Zea mays | Paralogous to G3516, G3517, G3794; Orthologous to |
| | | | | G1791, G1792, G1795, G30, G3380, G3381, G3383, |
| | | | | G3515, G3518, G3519, G3520, G3735, G3736, G3737 |
| 377 | G3794 | DNA | Zea mays | Predicted polypeptide sequence is paralogous to G3516, |
| | | | | G3517, G3739; orthologous to G1791, G1792, G1795, |
| | | | | G30, G3380, G3381, G3383, G3515, G3518, G3519, |
| | | | | G3520, G3735, G3736, G3737 |
| 378 | G3794 | PRT | Zea mays | Paralogous to G3516, G3517, G3739; Orthologous to |
| | | | | G1791, G1792, G1795, G30, G3380, G3381, G3383, |
| | | | | G3515, G3518, G3519, G3520, G3735, G3736, G3737 |
| 379 | G3841 | DNA | Lycopersicon | Predicted polypeptide sequence is paralogous to G3843, |
| | | | esculentum | G3852; orthologous to G22, G1006, G28, G3430, G3659, |
| | | | | G3660, G3661, G3717, G3718, G3844, G3845, G3846, |
| | | | | G3848, G3856, G3857, G3858, G3864, G3865 |
| 380 | G3841 | PRT | Lycopersicon | Paralogous to G3843, G3852; Orthologous to G22, G1006, |
| | | | esculentum | G28, G3430, G3659, G3660, G3661, G3717, G3718, |
| | | | | G3844, G3845, G3846, G3848, G3856, G3857, G3858, |
| | | | | G3864, G3865 |
| 381 | G3843 | DNA | Lycopersicon | Predicted polypeptide sequence is paralogous to G3841, |
| | | | esculentum | G3852; orthologous to G22, G1006, G28, G3430, G3659, |
| | | | | G3660, G3661, G3717, G3718, G3844, G3845, G3846, |
| | | | | G3848, G3856, G3857, G3858, G3864, G3865 |
| 382 | G3843 | PRT | Lycopersicon | Paralogous to G3841, G3852; Orthologous to G22, G1006, |
| | | | esculentum | G28, G3430, G3659, G3660, G3661, G3717, G3718, |
| | | | | G3844, G3845, G3846, G3848, G3856, G3857, G3858, |
| | | | | G3864, G3865 |
| 383 | G3844 | DNA | Medicago | Predicted polypeptide sequence is orthologous to G22, |
| | | | truncatula | G1006, G28, G3430, G3659, G3660, G3661, G3717, |
| | | | | G3718, G3841, G3843, G3845, G3846, G3848, G3852, |
| | | | | G3856, G3857, G3858, G3864, G3865 |
| 384 | G3844 | PRT | Medicago | Orthologous to G22, G1006, G28, G3430, G3659, G3660, |
| | | | truncatula | G3661, G3717, G3718, G3841, G3843, G3845, G3846, |
| | | | | G3848, G3852, G3856, G3857, G3858, G3864, G3865 |
| 385 | G3845 | DNA | Nicotiana | Predicted polypeptide sequence is paralogous to G3846; |
| | | | tabacum | orthologous to G22, G1006, G28, G3430, G3659, G3660, |
| | | | | G3661, G3717, G3718, G3841, G3843, G3844, G3848, |
| | | | | G3852, G3856, G3857, G3858, G3864, G3865 |
| 386 | G3845 | PRT | Nicotiana | Paralogous to G3846; Orthologous to G22, G1006, G28, |
| | | | tabacum | G3430, G3659, G3660, G3661, G3717, G3718, G3841, |
| | | | | G3843, G3844, G3848, G3852, G3856, G3857, G3858, |
| | | | | G3864, G3865 |
| 387 | G3846 | DNA | Nicotiana | Predicted polypeptide sequence is paralogous to G3845; |
| | | | tabacum | orthologous to G22, G1006, G28, G3430, G3659, G3660, |
| | | | | G3661, G3717, G3718, G3841, G3843, G3844, G3848, |
| | | | | G3852, G3856, G3857, G3858, G3864, G3865 |
| 388 | G3846 | PRT | Nicotiana | Paralogous to G3845; Orthologous to G22, G1006, G28, |
| | | | tabacum | G3430, G3659, G3660, G3661, G3717, G3718, G3841, |
| | | | | G3843, G3844, G3848, G3852, G3856, G3857, G3858, |
| | | | | G3864, G3865 |
| 389 | G3848 | DNA | Oryza sativa | Predicted polypeptide sequence is paralogous to G3430; |
| | | | (japonica | orthologous to G22, G1006, G28, G3659, G3660, G3661, |
| | | | cultivar-group) | G3717, G3718, G3841, G3843, G3844, G3845, G3846, |
| | | | | G3852, G3856, G3857, G3858, G3864, G3865 |
| 390 | G3848 | PRT | Oryza sativa | Paralogous to G3430; Orthologous to G22, G1006, G28, |
| | | | (japonica | G3659, G3660, G3661, G3717, G3718, G3841, G3843, |
| | | | cultivar-group) | G3844, G3845, G3846, G3852, G3856, G3857, G3858, |
| | | | | G3864, G3865 |
| 391 | G3852 | DNA | Lycopersicon | Predicted polypeptide sequence is paralogous to G3841, |
| | | | esculentum | G3843; orthologous to G22, G1006, G28, G3430, G3659, |
| | | | | G3660, G3661, G3717, G3718, G3844, G3845, G3846, |
| | | | | G3848, G3856, G3857, G3858, G3864, G3865 |
| 392 | G3852 | PRT | Lycopersicon | Paralogous to G3841, G3843; Orthologous to G22, G1006, |
| | | | esculentum | G28, G3430, G3659, G3660, G3661, G3717, G3718, |
| | | | | G3844, G3845, G3846, G3848, G3856, G3857, G3858, |
| | | | | G3864, G3865 |
| 393 | G3856 | DNA | Zea mays | Predicted polypeptide sequence is paralogous to G3661; |
| | | | | orthologous to G22, G1006, G28, G3430, G3659, G3660, |
| | | | | G3717, G3718, G3841, G3843, G3844, G3845, G3846, |
| | | | | G3848, G3852, G3857, G3858, G3864, G3865 |
| 394 | G3856 | PRT | Zea mays | Paralogous to G3661; Orthologous to G22, G1006, G28, |
| | | | | G3430, G3659, G3660, G3717, G3718, G3841, G3843, |
| | | | | G3844, G3845, G3846, G3848, G3852, G3857, G3858, |
| | | | | G3864, G3865 |
| 395 | G3857 | DNA | Solanum | Predicted polypeptide sequence is paralogous to G3858; |
| | | | tuberosum | orthologous to G22, G1006, G28, G3430, G3659, G3660, |
| | | | | G3661, G3717, G3718, G3841, G3843, G3844, G3845, |
| | | | | G3846, G3848, G3852, G3856, G3864, G3865 |
| 396 | G3857 | PRT | Solanum | Paralogous to G3858; Orthologous to G22, G1006, G28, |
| | | | tuberosum | G3430, G3659, G3660, G3661, G3717, G3718, G3841, |
| | | | | G3843, G3844, G3845, G3846, G3848, G3852, G3856, |
| | | | | G3864, G3865 |
| 397 | G3858 | DNA | Solanum | Predicted polypeptide sequence is paralogous to G3857; |
| | | | tuberosum | orthologous to G22, G1006, G28, G3430, G3659, G3660, |
| | | | | G3661, G3717, G3718, G3841, G3843, G3844, G3845, |
| | | | | G3846, G3848, G3852, G3856, G3864, G3865 |
| 398 | G3858 | PRT | Solanum | Paralogous to G3857; Orthologous to G22, G1006, G28, |
| | | | tuberosum | G3430, G3659, G3660, G3661, G3717, G3718, G3841, |
| | | | | G3843, G3844, G3845, G3846, G3848, G3852, G3856, |
| | | | | G3864, G3865 |
| 399 | G3864 | DNA | Triticum | Predicted polypeptide sequence is paralogous to G3865; |
| | | | aestivum | orthologous to G22, G1006, G28, G3430, G3659, G3660, |
| | | | | G3661, G3717, G3718, G3841, G3843, G3844, G3845, |
| | | | | G3846, G3848, G3852, G3856, G3857, G3858 |
| 400 | G3864 | PRT | Triticum | Paralogous to G3865; Orthologous to G22, G1006, G28, |
| | | | aestivum | G3430, G3659, G3660, G3661, G3717, G3718, G3841, |
| | | | | G3843, G3844, G3845, G3846, G3848, G3852, G3856, |
| | | | | G3857, G3858 |
| 401 | G3865 | DNA | Triticum | Predicted polypeptide sequence is paralogous to G3864; |
| | | | aestivum | orthologous to G22, G1006, G28, G3430, G3659, G3660, |
| | | | | G3661, G3717, G3718, G3841, G3843, G3844, G3845, |
| | | | | G3846, G3848, G3852, G3856, G3857, G3858 |
| 402 | G3865 | PRT | Triticum | Paralogous to G3864; Orthologous to G22, G1006, G28, |
| | | | aestivum | G3430, G3659, G3660, G3661, G3717, G3718, G3841, |
| | | | | G3843, G3844, G3845, G3846, G3848, G3852, G3856, |
| | | | | G3857, G3858 |
| 831 | G3930 | DNA | Arabidopsis | Predicted polypeptide sequence is paralogous to G225, |
| | | | thaliana | G226, G1816, G2718, G682; orthologous to G3392, |
| | | | | G3393, G3431, G3444, G3445, G3446, G3447, G3448, |
| | | | | G3449, G3450 |
| 832 | G3930 | PRT | Arabidopsis | Paralogous to G225, G226, G1816, G2718, G682; |
| | | | thaliana | Orthologous to G3392, G3393, G3431, G3444, G3445, |
| | | | | G3446, G3447, G3448, G3449, G3450 |
| 833 | G4014 | DNA | Glycine max | Predicted polypeptide sequence is orthologous to G1481, |
| | | | | G900; paralogous to G4015, G4016 |
| 834 | G4014 | PRT | Glycine max | Orthologous to G1481, G900; paralogous to G4015, G4016 |
| 835 | G4015 | DNA | Glycine max | Predicted polypeptide sequence is orthologous to G1481, |
| | | | | G900; paralogous to G4014, G4016 |
| 836 | G4015 | PRT | Glycine max | Orthologous to G1481, G900; paralogous to G4014, G4016 |
| 837 | G4016 | DNA | Glycine max | Predicted polypeptide sequence is orthologous to G1481, |
| | | | | G900; paralogous to G4014, G4015 |
| 838 | G4016 | PRT | Glycine max | Orthologous to G1481, G900; paralogous to G4014, G4015 |

### Molecular Modeling

Another means that may be used to confirm the utility and function of transcription factor sequences that are orthologous or paralogous to presently disclosed transcription factors is through the use of molecular modeling software. Molecular modeling is routinely used to predict polypeptide structure, and a variety of protein structure modeling programs, such as “Insight II” (Accelrys, Inc.) are commercially available for this purpose. Modeling can thus be used to predict which residues of a polypeptide can be changed without altering function (U.S. Pat. No. 6,521,453). Thus, polypeptides that are sequentially similar can be shown to have a high likelihood of similar function by their structural similarity, which may, for example, be established by comparison of regions of superstructure. The relative tendencies of amino acids to form regions of superstructure (for example, helixes and β-sheets) are well established. For example, O'Neil et al. (1990) have discussed in detail the helix forming tendencies of amino acids. Tables of relative structure forming activity for amino acids can be used as substitution tables to predict which residues can be functionally substituted in a given region, for example, in DNA-binding domains of known transcription factors and equivalogs. Homologs that are likely to be functionally similar can then be identified.

Of particular interest is the structure of a transcription factor in the region of its conserved domain(s). Structural analyses may be performed by comparing the structure of the known transcription factor around its conserved domain with those of orthologs and paralogs. Analysis of a number of polypeptides within a transcription factor group or clade, including the functionally or sequentially similar polypeptides provided in the Sequence Listing, may also provide an understanding of structural elements required to regulate transcription within a given family.

### Methods for Increasing Plant Yield or Quality by Modifying Transcription Factor Expression

The present invention includes compositions and methods for increasing the yield and quality of a plant or its products, including those derived from a crop plant. These methods incorporate steps described in the Examples listed below, and may be achieved by inserting, in the 5′ to 3′ direction, a nucleic acid sequence of the invention into the genome of a plant cell: (i) a promoter that functions in the cell; and (ii) a nucleic acid sequence that is substantially identical to any of SEQ ID NO: 2N-1, where N=1 to 201 or 413 to 419, or SEQ ID NO: 403 to 824, where the promoter is operably linked to the nucleic acid sequence. A transformed plant may then be generated from the cell. One may either obtain seeds from that plant or its progeny, or propagate the transformed plant asexually. Alternatively, the transformed plant may be grow and harvested for plant products directly.

### EXAMPLES

It is to be understood that this invention is not limited to the particular devices, machines, materials and methods described. Although particular embodiments are described, equivalent embodiments may be used to practice the invention.

The invention, now being generally described, will be more readily understood by reference to the following examples, which are included merely for purposes of illustration of certain aspects and embodiments of the present invention and are not intended to limit the invention. It will be recognized by one of skill in the art that a transcription factor that is associated with a particular first trait may also be associated with at least one other, unrelated and inherent second trait which was not predicted by the first trait.

### Example I

### Isolation and Cloning of Full-Length Plant Transcription Factor cDNAs

Putative transcription factor sequences (genomic or ESTs) related to known transcription factors were identified in the Arabidopsis thaliana GenBank database using the tblastn sequence analysis program using default parameters and a P-value cutoff threshold of B4 or B5 or lower, depending on the length of the query sequence. Putative transcription factor sequence hits were then screened to identify those containing particular sequence strings. If the sequence hits contained such sequence strings, the sequences were confirmed as transcription factors.

Alternatively, Arabidopsis thaliana cDNA libraries derived from different tissues or treatments, or genomic libraries were screened to identify novel members of a transcription family using a low stringency hybridization approach. Probes were synthesized using gene specific primers in a standard PCR reaction (annealing temperature 60° C.) and labeled with ³²P dCTP using the High Prime DNA Labeling Kit (Roche Diagnostics Corp., Indianapolis, Ind.). Purified radiolabelled probes were added to filters immersed in Church hybridization medium (0.5 M NaPO₄ pH 7.0, 7% SDS, 1% w/v bovine serum albumin) and hybridized overnight at 60° C. with shaking. Filters were washed two times for 45 to 60 minutes with 1×SCC, 1% SDS at 60° C.

To identify additional sequence 5′ or 3′ of a partial cDNA sequence in a cDNA library, 5′ and 3′ rapid amplification of cDNA ends (RACE) was performed using the MARATHON cDNA amplification kit (Clontech, Palo Alto, Calif.). Generally, the method entailed first isolating poly(A) mRNA, performing first and second strand cDNA synthesis to generate double stranded cDNA, blunting cDNA ends, followed by ligation of the MARATHON Adaptor to the cDNA to form a library of adaptor-ligated ds cDNA.

Gene-specific primers were designed to be used along with adaptor specific primers for both 5′ and 3′ RACE reactions. Nested primers, rather than single primers, were used to increase PCR specificity. Using 5′ and 3′ RACE reactions, 5′ and 3′ RACE fragments were obtained, sequenced and cloned. The process can be repeated until 5′ and 3′ ends of the full-length gene were identified. Then the full-length cDNA was generated by PCR using primers specific to 5′ and 3′ ends of the gene by end-to-end PCR.

### Example II

### Strategy to Produce a Tomato Population Expressing all Transcription Factors Driven by Ten Promoters

Ten promoters were chosen to control the expression of transcription factors in tomato for the purpose of evaluating complex traits in fruit development. All ten are expressed in fruit tissues, although the temporal and spatial expression patterns in the fruit vary (Table 7). All of the promoters have been characterized in tomato using a LexA-GAL4 two-component activation system.

TABLE 7
Promoters used in the field study

| Promoter | General expression patterns | References |
|---|---|---|
| 35S (SEQ ID NO: 839) | Constitutive, high levels of expression in | Odell et al (1985) |
| | all throughout the plant and fruit | |
| SHOOT MERISTEMLESS | Expressed in meristematic tissues, | Long and Barton (1998) |
| (STM; SEQ ID NO: 840) | including apical meristems, cambium. | Long and Barton (2000) |
| | Low levels of expression also in some | |
| | differentiating tissues. In fruit, most | |
| | strongly expressed in vascular tissues and | |
| | endosperm. | |
| ASYMMETRIC LEAVES 1 | Expressed predominately in | Byrne et al (2000) |
| (AS1; SEQ ID NO: 841) | differentiating tissues. In fruit, most | Ori et al. (2000) |
| | strongly expressed in vascular tissues and | |
| | in endosperm. | |
| LIPID TRANSFER | In vegetative tissues, expression is | Thoma et al. (1994) |
| PROTEIN 1 (LTP1; SEQ ID | predominately in the epidermis. Low | |
| NO: 842) | levels of expression are also evident in | |
| | vascular tissue. In the fruit, expression is | |
| | strongest in the pith-like | |
| | columella/placental tissue. | |
| RIBULOSE-1,5- | Expression predominately in highly | Wanner and Gruissem |
| BISPHOSPHATE | photosynthetic vegetative tissues. Fruit | (1991) |
| CARBOXYLASE, SMALL | expression predominately in the pericarp. | |
| SUBUNIT 3 (RbcS-3; SEQ | | |
| ID NO: 843) | | |
| ROOT SYSTEM | Expression generally limited to roots. | Taylor and Scheuring |
| INDUCIBLE 1(RSI-1; SEQ | Also expressed in the vascular tissues of | (1994) |
| ID NO: 844) | the fruit. | |
| APETALA 1 (AP1; SEQ ID | Light expression in leaves increases with | Mandel et al. (1992a) |
| NO: 845) | maturation. Highest expression in flower | Hempel et al. (1997) |
| | primordia and flower organs. In fruits, | |
| | predominately in pith-like | |
| | columella/placental tissue. | |
| POLYGALACTURONASE | High expression throughout the fruit, | Nicholass et al.(1995) |
| (PG; SEQ ID NO: 846) | comparable to 35S. Strongest late in fruit | Montgomery et al. (1993) |
| | development. | |
| PHYTOENE DESATURASE | Moderate expression in fruit tissues. | Corona et al. (1996) |
| (PD; SEQ ID NO: 847) | | |
| CRUCIFERIN 1 (SEQ ID | Expressed at low levels in fruit vascular | Breen and Crouch (1992) |
| NO: 848) | tissue and columella. Seen and endosperm | Sjodahl et al. (1995) |
| | expression. | |

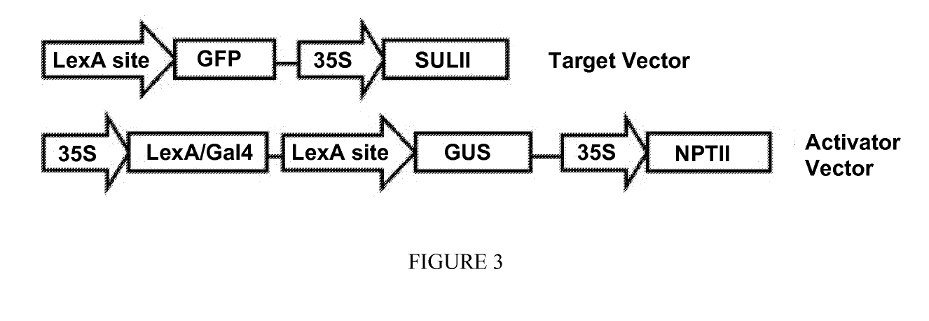

Transgenic tomato lines expressing all Arabidopsis transcription factors driven by ten tissue and/or developmentally regulated promoters relied on the use of a two-component system similar to that developed by Guyer et al. (1998) that uses the DNA binding domain of the yeast GAL4 transcriptional activator fused to the activation domains of the maize C1 or the herpes simplex virus VP16 transcriptional activators, respectively. Modifications used either the E. coli lactose repressor DNA binding domain (LacI) or the E. coli LexA DNA binding domain fused to the GAL4 activation domain. The LexA-based system was the most reliable in activating tissue-specific GFP expression in tomato and was used to generate the tomato population. A diagram of the test transformation vectors is shown in FIG. 3.

The full set of 1700 Arabidopsis transcription factor genes replaced the GFP gene in the target vector and the set of nine regulated promoters replaced the 35S promoter in the activator plasmid. Both families of vectors were used to transform tomato to yield one set of 1700 transgenic lines harboring 1700 different target vector constructs of transcription factor genes and a second population harboring the five different activator vector constructs of promoter-LexA/GAL4 fusions. Transgenic plants harboring the activator vector constructs of promoter-LexA/GAL4 fusions were screened to identify plants with appropriate and high level expression of GUS. In addition, five of each of the 1700 transgenic plants harboring the target vector constructs of transcription factor genes were grown and crossed with a 35 S activator line. F1 progeny were assayed to ensure that the transgene was capable of being activated by the LexA/GAL4 activator protein. The best plants constitutively expressing transcription factors were selected for subsequent crossing to the ten transgenic activator lines. Several of these initial lines have been evaluated and preliminary results of seedling traits indicate that similar phenotypes observed in Arabidopsis are also observed in tomato when the same transcription factor is constitutively overexpressed. Thus, each parental line harboring either a promoter-LexA/GAL4 activator or an activatable Arabidopsis transcription factors gene were pre-selected based on a functional assessment. These parental lines were used in sexual crosses to generate 17,000 F1 (hemizygous for the activator and target genes) lines representing the complete set of Arabidopsis transcription factors under the regulation of 10 developmentally-regulated promoters. The transgenic tomato population will be grown in the field for evaluation over a period of three years. The full population will consist of three individual plants from each of the 17000 lines grown in the field in the 2003-2005 seasons. Approximately 1400 of these lines were grown and evaluated.

### Example III

### Test Constructs

For the LacI system, the test construct was made in two steps. First, two intermediate constructs were generated. The first contained the LacI protein and gal4 activation domain, and the second contained the LacI operator and GFP. In the first construct, four fragments were generated separately and fused by overlap extension PCR. The four fragments included:
the 35S minimal promoter (SEQ ID NO: 849) and omega translation enhancer (SEQ ID NO: 850) (from construct SLJ4D4, Jones et al. (1992));
the E. coli LacI gene in which the translation initiation site is changed to ATG from GTG plus a Y to H mutation at position 17 (Lehming et al (1987));
the gal4 transcription activation domain (amino acids 768-881, from pGAD424, Clontech);
the E9 polyadenylation site (Fluhr et al (1986)).

To make the second intermediate construct, two copies of the LacI binding site and the 35S minimal promoter (SEQ ID NO: 849) and omega enhancer (SEQ ID NO: 850) were fused with a gene coding for GFP by overlap extension PCR. The system in which the LexA protein was used as the DNA binding domain was constructed in a similar fashion. The LexA protein was cloned from plasmid pLexA (Clontech), and the tandem of eight LexA operators was from plasmid p8op-lacZ (Clontech).

Inserts from the above two intermediate constructs were cloned together into a plant transformation vector that contained antibiotic resistance (e.g., sulfonamide resistance) markers. A multiple cloning site was added upstream of the region encoding the LacI (LexA)/gal4 fusion protein to facilitate cloning of promoter fragments. In order to test the functionality of the system, full 35S promoters were cloned upstream of the region encoding the LacI (LexA)/gal4 fusion protein to give the structures shown in FIG. 3. These were then transformed into Arabidopsis. As expected, GFP expression was identical to that of 35S/GFP control.

The Two-Component Multiplication System vectors have an activator vector and a target vector. The LexA version of these is shown in FIG. 3. The LacI versions are identical except that LacI replaces LexA portions. Both LacI and LexA DNA binding regions were tested in otherwise identical vectors. These regions were made from portions of the test vectors described above, using standard cloning methods. They were cloned into a binary vector that had been previously tested in tomato transformations. These vectors were then introduced into Arabidopsis and tomato plants to verify their functionality. The LexA-based system was determined to be the most reliable in activating tissue-specific GFP expression in tomato and was used to generate the tomato population.

A useful feature of the PTF Tool Kit vectors described in FIG. 3 is the use of two different resistance markers, one in the activator vector and another in the target vector. This greatly facilitates identifying the activator and target plant transcription factor genes in plants following crosses. The presence of both the activator and target in the same plant can be confirmed by resistance to both markers. Additionally, plants homozygous for one or both genes can be identified by scoring the segregation ratios of resistant progeny. These resistance markers are useful for making the technology easier to use for the breeder.

Another useful feature of the PTF Tool Kit activator vector described in FIG. 3 is the use of a target GFP construct to characterize the expression pattern of each of the 10 activator promoters. The Activator vector contains a construct consisting of multiple copies of the LexA (or LacI) binding sites and a TATA box upstream of the gene encoding the green fluorescence protein (GFP). This GFP reporter construct verifies that the activator gene is functional and that the promoter has the desired expression pattern before extensive plant crossing and characterizations proceed. The GFP reporter gene is also useful in plants derived from crossing the activator and target parents because it provides an easy method to detect the pattern of expression of expressed plant transcription factor genes.

### Example IV

### Tomato Transformation and Sulfonamide Selection

After the activator and target vectors were constructed, the vectors were used to transform Agrobacterium tumefaciens cells. Since the target vector comprised a polypeptide or interest (in the example given in FIG. 3, the polypeptide of interest was green fluorescent protein; other polypeptides of interest may include transcription factor polypeptides of the invention), it was expected that plants containing both vectors would be conferred with improved and useful traits. Methods for generating transformed plants with expression vectors are well known in the art; this Example also describes a novel method for transforming tomato plants with a sulfonamide selection marker. In this Example, tomato cotyledon explants were transformed with Agrobacterium cultures comprising target vectors having a sulfonamide selection marker.

### Seed Sterilization

T63 seeds were surface sterilized in a sterilization solution of 20% bleach (containing 6% sodium hypochlorite) for 20 minutes with constant stirring. Two drops of Tween 20 were added to the sterilization solution as a wetting agent. Seeds were rinsed five times with sterile distilled water, blotted dry with sterile filter paper and transferred to Sigma P4928 phytacons (25 seeds per phytacon) containing 84 ml of MSO medium (the formula for MS medium may be found in Murashige and Skoog (1962) Plant Physiol. 15: 473-497; MSO is supplemented as indicated in Table 8).

### Seed Germination and Explanting

Phytacons were placed in a growth room at 24° C. with a 16 hour photoperiod. Seedlings were grown for seven days.

Explanting plates were prepared by placing a 9 cm Whatman No. 2 filter paper onto a plate of 100 mm×25 mm Petri dish containing 25 ml of R1F medium. Tomato seedlings were cut and placed into a 100 mm×25 mm Petri dish containing a 9 cm Whatman No. 2 filter paper and 3 ml of distilled water. Explants were prepared by cutting cotyledons into three pieces. The two proximal pieces were transferred onto the explanting plate, and the distal section was discarded. One hundred twenty explants were placed on each plate. A control plate was also prepared that was not subjected to the Agrobacterium transformation procedure. Explants were kept in the dark at 24° C. for 24 hours.

### Agrobacterium Culture Preparation and Cocultivation

The stock of Agrobacterium tumefaciens cells for transformation were made as described by Nagel et al. (1990) FEMS Microbiol Letts. 67: 325-328. Agrobacterium strain ABI was grown in 250 ml LB medium (Sigma) overnight at 281 C with shaking until an absorbance over 1 cm at 600 nm (A₆₀₀) of 0.5 B 1.0 was reached. Cells were harvested by centrifugation at 4,000×g for 15 minutes at 4 C. Cells were then resuspended in 250 μl chilled buffer (1 mM HEPES, pH adjusted to 7.0 with KOH). Cells were centrifuged again as described above and resuspended in 125 μl chilled buffer. Cells were then centrifuged and resuspended two more times in the same HEPES buffer as described above at a volume of 100 μl and 750 μl, respectively. Resuspended cells were then distributed into 40 μl aliquots, quickly frozen in liquid nitrogen, and stored at −80° C.

Agrobacterium cells were transformed with vectors prepared as described above following the protocol described by Nagel et al. (1990) supra. For each DNA construct to be transformed, 50 to 100 ng DNA (generally resuspended in 10 mM Tris-HCl, 1 mM EDTA, pH 8.0) were mixed with 40 μl of Agrobacterium cells. The DNA/cell mixture was then transferred to a chilled cuvette with a 2 mm electrode gap and subject to a 2.5 kV charge dissipated at 25 μF and 200 μF using a Gene Pulser II apparatus (Bio-Rad, Hercules, Calif.). After electroporation, cells were immediately resuspended in 1.0 ml LB and allowed to recover without antibiotic selection for 2 B 4 hours at 28° C. in a shaking incubator. After recovery, cells were plated onto selective medium of LB broth containing 100 μg/ml spectinomycin (Sigma) and incubated for 24-48 hours at 28° C. Single colonies were then picked and inoculated in fresh medium. The presence of the vector construct was verified by PCR amplification and sequence analysis.

Agrobacteria were cultured in two sequential overnight cultures. On day 1, the agrobacteria containing the target vectors having the sulfonamide selection vector (FIG. 3) were grown in 25 ml of liquid 523 medium (Moore et al. (1988) in Schaad, ed., Laboratory Guide for the Identification of Plant Pathogenic Bacteria. APS Press, St. Paul, Minn.) plus 100 mg spectinomycin, 50 mg kanamycin, and 25 mg chloramphenicol per liter. On day 2, five ml of the first overnight suspension were added to 25 ml of AB medium to which is added 100 mg spectinomycin, 50 mg kanamycin, and 25 mg chloramphenicol per liter. Cultures were grown at 28° C. with constant shaking on a gyratory shaker. The second overnight suspension was centrifuged in a 38 ml sterile Oakridge tubes for 5 minutes at 8000 rpm in a Beckman JA20 rotor. The pellet was resuspended in 10 ml of MSO liquid medium containing 600 μm acetosyringone (for each 20 ml of MSO medium, 40 μl of 0.3 M stock acetosyringone were added). The Agrobacterium concentration was adjusted to an A₆₀₀ of 0.25.

Seven milliliters of this Agrobacterium suspension were added to each of explanting plates. After 20 minutes, the Agrobacterium suspension was aspirated and the explants were blotted dry three times with sterile filter paper. The plates were sealed with Parafilm and incubated in the dark at 21° C. for 48 hours.

### Regeneration

Cocultivated explants were transferred after 48 hours in the dark to 100 mm×25 mm Petri plates (20 explants per plate) containing 25 ml of R1SB10 medium (this medium and subsequently used media contained sulfadiazine, the sulfonamide antibiotic used to select transformants). Plates were kept in the dark for 72 hours and then placed in low light. After 14 days, the explants were transferred to fresh RZ1/2SB25 medium. After an additional 14 days, the regenerating tissues at the edge of the explants were excised away from the primary explants and were transferred onto fresh RZ1/2SB25 medium. After another 14 day interval, regenerating tissues were again transferred to fresh ROSB25 medium. After this period, the regenerating tissues were subsequently rotated between ROSB25 and RZ1/2SB25 media at two week intervals. The well defined shoots that appeared were excised and transferred to ROSB100 medium for rooting.

### Shoot Analysis

Once shoots were rooted on ROSB100 medium, small leaf pieces from the rooted shoots were sampled and analyzed with a polymerase chain reaction procedure (PCR) for the presence of the SulA gene. The PCR-positive shoots (T0) were then grown to maturity in the greenhouses. Some T0 plants were crossed to plants containing the CaMV 35S activator vector. The T0 self pollinated seeds were saved for later crosses to different activator promoters.

TABLE 8
Media Compositions (amounts per liter)

| | | | | | | | RZ1/ | | | | | |
|---|---|---|---|---|---|---|---|---|---|---|---|---|
| | MSO | | R1F | | R1SB10 | | 2SB25 | | ROSB25 | | ROSB100 | |
| Gibco MS Salts | 4.3 | g | 4.3 | g | 4.3 | g | 4.3 | g | 4.3 | g | 4.3 | g |
| RO Vitamins (100X) | 10 | ml | | | | | 5 | ml | 10 | ml | 10 | ml |
| R1 Vitamins (100X) | | | 10 | ml | 10 | ml | | | | | | |
| RZ Vitamins (100X) | | | | | | | 5 | ml | | | | |
| Glucose | 16.0 | g | 16.0 | g | 16.0 | g | 16.0 | g | 16.0 | g | 16.0 | g |
| Timentin ® | | | | | 100 | mg | | | | | | |
| Carbenicillin | | | | | | | 350 | mg | 350 | mg | 350 | mg |
| Noble Agar | 8 | | 11.5 | | 10.3 | | 10.45 | | 10.45 | | 10.45 | |
| MES | | | | | 0.6 | g | 0.6 | g | 0.6 | g | 0.6 | g |
| Sulfadiazine free acid | | | | | 1 | ml | 2.5 | ml | 2.5 | ml | 10 | ml |
| (10 mg/ml stock) | | | | | | | | | | | | |

| | | | | | | |
|---|---|---|---|---|---|---|
| pH | 5.7 | 5.7 | 5.7 | 5.7 | 5.7 | 5.7 |

TABLE 9
100x Vitamins (amounts per liter)

| | RO | | R1 | | RZ | |
|---|---|---|---|---|---|---|
| Nicotinic acid | 500 | mg | 500 | mg | 500 | mg |
| Thiamine HCl | 50 | mg | 50 | mg | 50 | mg |
| Pyridoxine HCl | 50 | mg | 50 | mg | 50 | mg |
| Myo-inositol | 20 | g | 20 | g | 20 | g |
| Glycine | 200 | mg | 200 | mg | 200 | mg |
| Zeatin | | | 0.65 | mg | 0.65 | mg |
| IAA | | | 1.0 | mg | | |

| | | | |
|---|---|---|---|
| pH | 5.7 | 5.7 | 5.7 |

TABLE 10
523 Medium (amounts per liter)

| Sucrose | 10 g |
|---|---|
| Casein Enzymatic Hydrolysate | 8 g |
| Yeast Extract | 4 g |
| K₂HPO₄ | 2 g |
| MgSO₄•7H₂O | 0.3 g |
| pH | 7.00 |

TABLE 11
AB Medium

| Part A | | | Part B (10X stock) | | |
|---|---|---|---|---|---|
| K₂HPO₄ | 3 | g | MgSO₄•7H₂O | 3 | g |
| NaH₂PO₄ | 1 | g | CaCl₂ | 0.1 | g |
| NH₄Cl | 1 | g | FeSO₄•H₂O | 0.025 | g |
| KCl | 0.15 | g | Glucose | 50 | g |

| | | |
|---|---|---|
| pH | 7.00 | 7.00 |

| | | | | |
|---|---|---|---|---|
| Volume | 900 | ml | 1000 | ml |

Prepared by autoclaving and mixing 900 ml Part A with 100 ml Part B.

### Example V

### Population Characterization and Measurements

After the crosses were made (to generate plants having both activator and target vectors), general characterization of the F1 population was performed in the field. General evaluation included photographs of seedling and adult plant morphology, photographs of leaf shape, open flower morphology and of mature green and ripe fruit. Vegetative plant size was measured by ruler at approximately two months after transplant. Plant volume was obtained by the multiplication of the three dimensions. In addition, observations were made to determine fruit number per plant. Three red-ripe fruit were harvested from each individual plant when possible and were used for the lycopene and Brix assays. Two weeks later, six fruits per promoter::gene grouping were harvested, with two fruits per plant harvested when possible. The fruits were pooled and seeds collected.

Measurement of soluble solids (“Brix”) was used to determine the amount of sugar in solution. For example, 18 degree Brix sugar solution contains 18% sugar (w/w basis). Brix was measured using a refractometer (which measures refractive index). Brix measurements were performed by the follow protocol:
1. Three red ripe fruit were harvest from each plant sampled.
2. Each sample of three fruit was weighed together
3. The three fruit were then quartered and blended together at high speed in a blender for approximately four minutes, until a fine puree was produced.
4. Two 40 ml aliquots were decanted from the pureed sampled into 50 ml polypropylene tubes.
5. Samples were then kept frozen at −20° C. until analysis
6. For analysis samples were thawed in warm water.
7. Approximately 15 ml of thawed tomato puree was filtered and placed onto the reading surface of a digital refractometer, and the reading recorded.

Source/sink activities. Source/sink activities were determined by screening for lines in which Arabidopsis transcription factors were driven by the RbcS-3 (leaf mesophyll expression), LTP1 (epidermis and vascular expression) and the PD (early fruit development) promoters. These promoters target source processes localized in photosynthetically active cells (RbcS-3), sink processes localized in developing fruit (PD) or transport processes active in vascular tissues (LTP1) that link source and sink activities. Leaf punches were collected within one hour of sunrise, in the seventh week after transplant, and stored in ethanol. The leaves were then stained with iodine, and plants with notably high or low levels of starch were noted.

Fruit ripening regulation. Screening for traits associated with fruit ripening focused on transgenic tomato lines in which Arabidopsis transcription factors are driven by the PD (early fruit development) and PG (fruit ripening) promoters. These promoters target fruit regulatory processes that lead to fruit maturation or which trigger ripening or components of the ripening process. In order to identify lines expressing transcription factors that impact ripening, fruits at 1 cm stage, a developmental time 7-10 days post anthesis and shortly after fruit set were tagged. Tagging occurred over a single two-day period per field trial at a time when plants are in the early fruiting stage to ensure tagging of one to two fruits per plant, and four to six fruits per line. Tagged fruit at the “breaker” stage on any given inspection were marked with a second colored and dated tag. Later inspections included monitoring of breaker-tagged fruit to identify any that have reached the full red ripe stage. To assess the regulation of components of the ripening process, fruit at the mature green and red ripe stage have been harvested and fruit texture analyzed by force necessary to compress equator of the fruit by 2 mm.

Post-harvest pathogen and other disease resistance. Screening for traits associated with post-harvest pathogen susceptibility and resistance focused on the lines in which Arabidopsis transcription factors are regulated by the fruit ripening promoter, PG. The PG promoter targets functions that are active in the later stages of ripening when the fruit are susceptible to necrotrophic pathogens. Mature green and red ripe fruit (two per line) were surface sterilized with 10% bleach and then wound inoculated with 10 ml droplets containing 10³ Botrytis cinerea or Alternaria alternata spores. A control site on each fruit was mock-inoculated with the water-0.05% Tween-80 solution used to suspend the spores. The titer of viable spores in the inoculating solution were determined by plating the samples on PDA plates. The inoculated fruit were held at 15° C. in humid storage boxes and lesion diameter measured daily. Resistance and susceptibility were scored as a percent of the spore-inoculated sites on each fruit that develop expanding necrotic lesions, and fruit from control non-transgenic lines were included.

### Example VI

### Screening CaMV 35S Activator Line Progeny with the Transcription Factor Target Lines to Identify Lines Expressing Plant Transcription Factors

The plant transcription factor target plants that were initially prepared lacked an activator gene to facilitate later crosses to various activator promoter lines. In order to find transformants that were adequately expressed in the presence of an activator, the plant transcription factor plants were crossed to the CaMV 35S promoter activator line and screened for transcription factor expression by RT-PCR. The mRNA was reverse transcribed into cDNA and the amount of product was measured by semi-quantitative PCR. The qualitative measurement was sufficient to distinguish high and low expressors.

Because the parental lines were each heterozygous for the transgenes, T1 hybrid progeny were sprayed with chlorsulfuron and cyanamide to find the 25% of the progeny containing both the activator (chlorsulfuron resistant) and target (cyanamide resistant) transgenes. Segregation ratios were measured and lines with abnormal ratios were discarded. Too high a ratio indicated multiple inserts, while too low a ratio indicated a variety of possible problems. The ideal inserts produced 50% resistant progeny. Progeny containing both inserts appeared at 25% because they also required the other herbicidal markers from the Activator parental line (50%×50%).

These T1 hybrid progeny were then screened in a 96 well format for plant transcription factor gene expression by RT-PCR to ensure expression of the target plant transcription factor gene, as certain chromosomal positions can be silent or very poorly expressed or the gene can be disrupted during the integration process. The 96 well format was also used for cDNA synthesis and PCR. This procedure involves the use of one primer in the transcribed portion of the vector and a second gene-specific primer.

Because both the activator and target genes are dominant in their effects, phenotypes were observable in hybrid progeny containing both genes. These TIPI plants were examined for visual phenotypes. However, more detailed analysis for increased color, high solids and disease resistance were also conducted once the best lines were identified and reproduced on a larger scale.

### Example VII

### Overexpression of Specific Promoter::Transcription Factor Combinations in Tomato Plants

Combined data obtained from the various promoter and gene combination in transformed tomato plants are shown in Table 12, with the minimum values, 25, 50 and 75 percentile values, and maximum values obtained for each of the three trait categories.

TABLE 12
Data ranges for fruit Brix, fruit lycopene, and two-month old vegetative plant size
measurements

| | | Percentile | | | | |
|---|---|---|---|---|---|---|
| | | Min | 25% | 50% | 75% | Max |
| Brix (g sugar/100 g sample) | Transformants | 3.5 | 5.18 | 5.56 | 5.91 | 8.37 |
| | Wild-type | 4.33 | 4.92 | 5.25 | 5.45 | 6.5 |
| Lycopene (ppm) | Transformants | 19.62 | 48.11 | 63.02 | 79.87 | 152.55 |
| | Wild-type | 36.45 | 44.57 | 55.75 | 73.2 | 94.65 |
| Volume (m³) | Transformants | 0.0005 | 0.122 | 0.179 | 0.231 | 0.675 |
| | Wild-type | 0.019 | 0.111 | 0.165 | 0.231 | 0.42 |

The data presented below for specific promoter::gene combinations in this Example include values with the highest significance for fruit Brix, fruit lycopene, or two-month old vegetative plant size measurements. Simple cutoff criteria were used to select these top “lead genes”—a gene and promoter combination rank in the top 95th percentile in any one measurement or if the same gene rank in the top 90th percentile under more than two promoters. The wild-type value at the 50% percentile in Table 12 was used as the control value for statistical purposes.

### G3 (SEQ ID NO: 1 and 2)

Published background information. G3 corresponds to RAP2.1, a gene first identified in a partial cDNA clone (Okamuro et al. (1997)). G3 is contained in BAC clone F2G19 (GenBank accession number AC083835; gene F2G19.32). Sakuma et al. (2002) categorized G3 into the A5 subgroup of the AP2 transcription factor family, with the A family related to the DREB and CBF genes. Fowler and Thomashow (2002) reported that G3 expression is enhanced in plants overexpressing CBF1, CBF2 or CBF3, and that the promoter region of G3 has two copies of the CCGAC core sequence of the CRT/DRE elements.

Discoveries in Arabidopsis. Overexpression of G3 under control of the 35S promoter produced very small plants with poor fertility. Overexpressors were also found to be sensitive to heat stress in a plate assay, exhibiting enhanced chlorosis following three days at 32° C. None of the stress challenge array background experiments revealed any regulation of G3 expression.

Discoveries in tomato. Lycopene content in fruit was greater than that in wild type controls, in plants expressing G3 under the RBCS3 promoter, with a rank in the 95th percentile among all measurements. In seedlings expressing G3 under the 35S promoter, size was reduced and an etiolated phenotype was evident. Plant size was also dramatically reduced upon overexpression of G3 with the 35S promoter in Arabidopsis.

TABLE 13
Data Summary for G3

| | Promoter summary: Avg. ± StD. (Count) | | |
|---|---|---|---|
| | Brix (g | | |
| Promoter | sugar/100 g sample) | Lycopene (ppm) | Volume (m³) |
| 35S | NA | NA | 0.18 ± 0.019 (3) |
| AP1 | 6.11 ± NA (1) | 93.77 ± NA (1) | 0.3 ± 0.046 (3) |
| Cruciferin | NA | NA | 0.11 ± NA (1) |
| RBCS3 | 4.88 ± NA (1) | 104.6 ± NA (1) | 0.25 ± 0.044 (3) |
| STM | 5.38 ± 0.367 (3) | 70.79 ± 29.746 (3) | 0.24 ± 0.044 (3) |

NA = not available
Avg. = average
StD. = standard deviation

### G22 (SEQ ID NO: 3 and 4)

Published background information. G22 has been identified in the sequence of BAC T13E15 (gene T13E15.5) by The Institute of Genomic Research (TIGR) as a “TINY transcription factor isolog”. Sakuma et al. (2002) categorized G22 into the B3 subgroup of the AP2 transcription factor family, with the B family containing ERF genes with a single AP2 domain.

Discoveries in Arabidopsis. Overexpression of G22 under control of the 35 S promoter produced plants with wild type morphology and development. Plants ectopically overexpressing G22 were slightly more tolerant to high NaCl containing media in a root growth assay compared to wild-type controls. G22 was found to be a stress-regulated gene in global transcript profiling experiments. Expression was repressed significantly in severe drought conditions, with expression repressed still during early recovery. In contrast, expression was significantly induced upon salt treatment, with induction increasing through eight hours. Treatments with cold and methyl jasmonate (MeJA) also induce expression.

Discoveries in tomato. Lycopene content in fruit was greater than that in wild type controls in plants expressing G22 under the RBCS3 promoter, with a rank in the 95th percentile among all measurements. Brix was higher than that in wild type in plants expressing G22 under the AP1 and STM promoters. Seedlings expressing G22 under the 35S promoter had curled leaves that were somewhat chlorotic.

Other related data. The paralogs of G22, G28 and G1006, were not tested in tomato in the present field study. In Arabidopsis, overexpression of G28, a G22 paralog, resulted in significant, multi-pathogen resistance in Arabidopsis.

TABLE 14
Data Summary for G22

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 7.29 ± 1.534 (2) | 90.4 ± 28.242 (2) | 0.22 ± 0.045 (3) |
| LTP1 | NA | NA | 0.19 ± 0.057 (2) |
| PD | 5.89 ± 0.487 (3) | 96.17 ± 1.623 (3) | 0.23 ± 0.056 (3) |
| PG | 5.34 ± NA (1) | 44.77 ± NA (1) | 0.2 ± 0.019 (3) |
| RBCS3 | 5.38 ± NA (1) | 102.29 ± NA (1) | 0.22 ± 0.098 (2) |
| STM | 6.34 ± 0.272 (3) | 85.29 ± 31.415 (3) | 0.25 ± 0.165 (3) |

### G24 (SEQ ID NO: 5 and 6)

Published background information. G24 corresponds to gene At2g23340 (AAB87098). Sakuma et al. (2002) categorized G24 into the A5 subgroup of the AP2 transcription factor family, with the A family related to the DREB and CBF genes.

Discoveries in Arabidopsis. Overexpression of G24 and its closely related paralog G12 under control of the 35S promoter both produced very small plants with necrotic patches on cotyledons. In the most severe cases, necrosis developed rapidly following germination, and the entire seedling turned black and died prior to the formation of true leaves. In 35S::G24 seedlings with a weaker phenotype, necrotic patches were visible on the cotyledons, but the plants survived transplantation to soil. At later stages of development, necrotic patches were no longer apparent on the leaves, but the plants were usually small, slower growing and poorly fertile in comparison to wild type controls. The leaves of older 35S::G24 plants were also observed to become yellow and senesce prematurely compared to wild type. Expression of G24 was modulated during stress responses. Expression was repressed during drought and abscisic acid (ABA) treatments, but induced after 4-8 hours treatment with mannitol, cold and salt stresses. Overexpression of CBF4 also enhanced expression of G24. In contrast, G12 was induced in roots transiently by ABA and MeJA treatments.

Discoveries in tomato. In plants expressing G24 under the AS1 and Cruciferin promoters, plant size was significantly greater than wild type controls, with a rank in the 95th percentile among all measurements. Interestingly, seedlings overexpressing G12 and G24 under the control of the 35S promoter were smaller than wild type controls. No paralog of G24 was tested in the field trial. In Arabidopsis, overexpression of G24 and its paralog G12 under control of the 35S promoter suggested that G12 and G24 participate in ethylene-regulated programmed cell death, based on the development of necrotic patches on cotyledons.

Other related data. The paralogs of G24-G12, G1277, and G1379—were not tested in tomato in the present field trial. In Arabidopsis, the G12 knockout mutant seedlings germinated in the dark on ACC-containing media (ethylene insensitivity assay) were more severely stunted than the wild-type controls. These results might indicate that G12 is involved in the ethylene signal transduction or response pathway, a process in which other proteins of the AP2/EREBP family are in fact implicated. G12 knockout (KO) mutant plants were wild type in morphology and development, and in all other physiological and biochemical analyses that were performed.

Constitutive expression of G1277 in Arabidopsis caused morphological alterations, including a reduction in plant size and curled leaves. These phenotypes were more apparent in the T1 than the T2 generation. T2 plants were wild type in all physiological and biochemical assays performed.

Overexpression of G1379 in Arabidopsis was severely detrimental. 35S::G1379 plants were extremely small compared to wild type controls at all stages of development. The most strongly affected individuals senesced and died at the vegetative stage, whereas transformants with a weaker phenotype produced very short inflorescence stems. The flowers from these plants often had poorly developed petals and stamens and set very little seed. Due to the tiny nature and sterility of 35S::G1379 plants, physiological and biochemical assays could not be performed.

TABLE 15
Data Summary for G24

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 5.5 ± 0.184 (2) | 56.06 ± 0.665 (2) | 0.09 ± 0.006 (3) |
| AS1 | 6.12 ± 0.667 (3) | 59.25 ± 13.098 (3) | 0.35 ± 0.095 (3) |
| Cruciferin | NA | NA | 0.4 ± 0.396 (2) |
| LTP1 | NA | NA | 0.12 ± NA (1) |
| PG | NA | NA | 0.18 ± 0.102 (3) |
| RBCS3 | 5.24 ± 0.255 (3) | 41.73 ± 2.181 (3) | 0.1 ± 0.006 (3) |
| STM | 5.69 ± 0.198 (2) | 45.75 ± 7.361 (2) | 0.09 ± 0.034 (3) |

### G47 (SEQ E) NO: 7 and 8)

Published background information. G47 corresponds to gene T22J18.2 (AAC25505). Sakuma et al. (2002) categorized G47 into the A5 subgroup of the AP2 transcription factor family, with the A family related to the DREB and CBF genes.

Discoveries in Arabidopsis. In seedlings expressing G47 under the 35S promoter, leaves had a brighter green color than wild types. Overexpression of G47 in Arabidopsis produced a substantial delay in flowering time and caused a marked change in shoot architecture. Interestingly, the inflorescences from these plants appeared thick and fleshy, had reduced apical dominance, and exhibited reduced internode elongation leading to a short compact stature. Stem sections from two lines were examined and found to be of wider diameter, and had large irregular vascular bundles containing a much greater number of xylem vessels than wild type. Furthermore some of the xylem vessels within the bundles appeared narrow and were possibly more lignified than were those of controls. G47 expression was significantly induced in roots by salt or cold stress treatments. Mannitol treatment produced a transient repression of expression. G47 overexpression in Arabidopsis has also been found to give enhanced drought tolerance.

Discoveries in tomato. Plant size was increased compared to that in wild type in G47 plants overexpressed under the LTP1 promoter. In seedlings expressing G47 under the 35S promoter, leaves had a brighter green color than wild types. Overexpression of G47 in Arabidopsis produced a substantial delay in flowering time and caused a marked change in shoot architecture. Interestingly, the inflorescences from these plants appeared thick and fleshy, had reduced apical dominance, and exhibited reduced internode elongation leading to a short compact stature. G47 stems had an increase in the number of xylem vessels, as well as increased lignin content.

Other related data. The paralog of G47, G2133, was not tested in tomato in the present field trial. In Arabidopsis, overexpression of G2133 caused a variety of alterations in plant growth and development: delayed flowering, altered inflorescence architecture, and a decrease in overall size and fertility.

TABLE 16
Data Summary for G47

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 5.51 ± 0.099 (2) | 49.21 ± 7.227 (2) | 0.29 ± 0.089 (2) |
| AS1 | 5.44 ± 0.255 (2) | 37.47 ± 14.552 (2) | 0.29 ± 0.067 (3) |
| LTP1 | 5.36 ± 0.488 (2) | 74.18 ± 29.663 (2) | 0.43 ± 0.185 (3) |
| PD | 5.96 ± 0.396 (3) | 57.73 ± 23.02 (3) | 0.32 ± 0.044 (3) |
| RBCS3 | NA | NA | 0.3 ± NA (1) |

### G156 (SEQ ID NO: 9 and 10)

Published background information. G156 corresponds to AT5G23260 and was initially assigned the name AGL32 by Alvarez-Buylla et al. (2000) during a survey of the MAD box gene family. The gene has subsequently been identified as TRANSPARENT TESTA16 (TT16) by Nesi et al. (2002), who determined that the gene has a role in regulating proanthocynidin biosynthesis in the inner-most cell layer of the seed coat. Additionally, (TT16) controls cell shape of the innermost cell layer of the seed coat. TT16 is also referenced in the literature by an alternative name: ARABIDOPSIS BSISTER (ABS).

Discoveries in Arabidopsis. G156 was analyzed during our Arabidopsis genomics program via both 35S::G156 lines and KO.G156 lines. Overexpression of the gene produced a variety of abnormalities in plant morphology; a pleiotropic phenotype commonly observed when MADS box proteins are overexpressed. Nevertheless, the KO.G156 phenotype provided a clear indication that the gene had a role in regulation of pigment production, since the seeds from KO.G156 plants were pale. This conclusion was subsequently confirmed by Nesi et al. (2002). It is also noteworthy that 35S::G156 lines performed better than wild type in a C/N sensing assay. This phenotype is likely related to the function of the gene in the control of flavonoid biosynthesis.

RT-PCR experiments revealed high levels of G156 expression in Arabidopsis embryo and silique tissues, which correlates with the potential role of the gene in seed coat. G156 has not been noted as significantly differentially expressed in any of the microarray studies to date.

Discoveries in tomato. In transgenic tomatoes expressing G156 under the regulation of the AP1, promoter, fruit lycopene levels from AP1::G156 plants were markedly higher than those found in wild-type controls. AP1::G156 tomato plants were also noted to have a compact morphology.

Other related data. We have not yet identified a paralog of G156 in Arabidopsis. Interestingly, during genomics screens, an Arabidopsis T-DNA insertion mutant for G156 exhibited pale seeds reminiscent of a transparent testa phenotype, suggesting that the gene could be a regulator of pigment production. Such a role was subsequently confirmed by Nesi et al. (2002) who identified the gene as TRANSPARENT TESTA 16.

TABLE 17
Data Summary for G156

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 6.05 ± NA (1) | 100.37 ± NA (1) | 0.14 ± 0.072 (3) |
| AS1 | 4.22 ± NA (1) | 58.47 ± NA (1) | 0.16 ± 0.069 (3) |
| Cruciferin | 5.39 ± 0.523 (2) | 75.72 ± 18.767 (2) | 0.29 ± 0.077 (3) |
| PD | 5.28 ± 0.049 (2) | 57.23 ± 8.761 (2) | 0.19 ± 0.008 (3) |
| PG | NA | NA | 0.2 ± 0.046 (3) |
| RBCS3 | 4.83 ± NA (1) | 71.95 ± NA (1) | 0.28 ± 0.113 (3) |
| STM | 4.84 ± NA (1) | 53.6 ± NA (1) | 0.27 ± 0.054 (3) |

### G159 (SEQ ID NO: 11 and 12)

Published background information: G159 corresponds to AT1G01530 and was assigned the name AGL28 by Alvarez-Buylla et al. (2000) during a survey of the MAD box gene family. G159 has a closely related paralog in the Arabidopsis genome, G165 (AT1G65360, AGL23).

Discoveries in Arabidopsis. G159 was analyzed during our Arabidopsis genomics program via 35S::G159 lines. Overexpression of the gene produced some abnormalities in plant growth and development (a pleiotropic phenotype commonly observed when MADS box proteins are overexpressed) but otherwise, no marked differences were observed compared to wild-type controls. A similar result was obtained from G165 overexpression in Arabidopsis.

RT-PCR experiments indicated that G159 and G165 were endogenously expressed at very low levels. Neither G159 nor G165 has been noted as significantly differentially expressed in any of the microarray studies performed to date.

Discoveries in tomato. Both fruit lycopene and soluble solid levels from LTP1::G159 fruits were markedly higher than those found in wild-type controls.

Other related data. The closely related paralog, G165, has not yet been analyzed in the tomato field trial. Overexpression of G165 in Arabidopsis produced a reduction in overall plant size.

TABLE 18
Data Summary for G159

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | NA | NA | 0.11 ± NA (1) |
| AS1 | 5.26 ± NA (1) | 57.29 ± NA (1) | 0.17 ± 0.042 (3) |
| Cruciferin | 5.41 ± 0.33 (3) | 48.91 ± 11.441 (3) | 0.25 ± 0.032 (3) |
| LTP1 | 6.41 ± NA (1) | 99.05 ± NA (1) | 0.2 ± 0.034 (3) |
| PD | 5.33 ± 0.127 (2) | 67.9 ± 35.56 (2) | 0.17 ± 0.024 (3) |
| PG | 5.74 ± 0.37 (3) | 69.73 ± 33.55 (3) | 0.25 ± 0.029 (3) |
| RBCS3 | 4.8 ± 0.071 (2) | 40.61 ± 7.658 (2) | 0.19 ± 0.017 (3) |
| STM | 5.43 ± 0.763 (3) | 46.37 ± 6.021 (3) | 0.21 ± 0.02 (3) |

### G187 (SEQ ID NO: 13 and 14)

Published background information. G187 corresponds to AtWRKY28 (At4g18170), for which there is no published literature beyond the general description of WRKY family members (Eulgem et al. (2000).

Discoveries in Arabidopsis. G187 is constitutively expressed. The function of G187 was analyzed using transgenic plants in which this gene was expressed under the control of the 35S promoter. G1187 T1 lines showed a variety of morphological alterations that included long and thin cotyledons at the seedling stage, and several flower abnormalities (for example, strap-like, sepaloid petals). These phenotypic alterations disappeared in the T2 generation, perhaps because of transgene silencing. Overexpression of G195, a G187 paralog, also produced similar deleterious effects. G187 overexpressing plants were indistinguishable from the corresponding wild-type controls in all the physiological and biochemical assays that were performed.

Discoveries in tomato. Transgenic tomatoes expressing G187 under the STM or RBCS3 promoter were analyzed for alteration in plant size, soluble solids and lycopene. The Brix levels under the STM promoter rank in the 95th percentile among all other measurements. Fruit-set in STM::G187 plants was delayed, and these plants did not produce mature fruit.

Other related data. G1198 is a paralog of G187 and was also tested in the field trial but no significant differences were detected in all assays performed. Several of the G187 paralogs were also overexpressed in Arabidopsis—some resulting in stunted plants while others had no phenotype.

TABLE 19
Data Summary for G187

| | Promoter summary: Avg. ± StD. (Count) | | |
|---|---|---|---|
| | | Lycopene | |
| Promoter | Brix (g sugar/100 g sample) | (ppm) | Volume (m³) |
| STM | 6.29 ± NA (1) | 55.21 ± NA (1) | 0.14 ± 0.04 (3) |

### G190 (SEQ ID NO: 15 and 16)

Published background information. G190 (At5g22570) corresponds to AtWRKY38 for which there is no published literature beyond the general description of WRKY family members (Eulgem et al. (2000).

Discoveries in Arabidopsis. The function of G190 was analyzed using transgenic plants in which this gene was expressed under the control of the 35S promoter. G190 overexpressing plants were morphologically wild type, and behaved like the corresponding controls in all physiological and biochemical assays that were performed. G190 was ubiquitously expressed, but at higher levels in roots and rosette leaves.

In a soil drought microarray experiment, G190 was found to be repressed in Arabidopsis leaves at multiple stages of drought stress. Repression levels correlated with the severity of drought, and expression began to recover after rewatering.

G190 was highly (up to 27-fold) induced by salicylic acid in both root and shoot tissue. Induction to a lesser extent was also observed with methyl jasmonate, sodium chloride and cold treatments.

Discoveries in tomato. The fruit lycopene levels of transgenic tomatoes expressing G190 under the STM promoter ranked in the 95th percentile among all lycopene measurements, and were higher than in any wild-type plant measured. Additionally, STM::G190 plants were noted to be larger and lower yielding, in terms of the number of fruit produced per plant, than wild type.

TABLE 20
Data Summary for G190

| | Promoter summary: Avg. ± StD. (Count) | | |
|---|---|---|---|
| | Brix | | |
| Promoter | (g sugar/100 g sample) | Lycopene (ppm) | Volume (m³) |
| 35S | 5.72 ± NA (1) | 72.2 ± NA (1) | 0.14 ± 0.047 (3) |
| AP1 | 6.01 ± NA (1) | 92.69 ± NA (1) | 0.15 ± 0.074 (3) |
| AS1 | 5.36 ± 0.206 (3) | 66.16 ± 14.14 (3) | 0.2 ± 0.034 (3) |
| RBCS3 | NA | NA | 0.16 ± 0.07 (3) |
| STM | 5.16 ± NA (1) | 98.31 ± NA (1) | 0.16 ± 0.088 (3) |

### G226 (SEQ ID NO: 17 and 18)

Published background information. G226 (At2g30420) was identified from the Arabidopsis BAC sequence AC002338, based on its sequence similarity within the conserved domain to other Myb family members in Arabidopsis.

Discoveries in Arabidopsis. Arabidopsis plants overexpressing G226 were more tolerant to low nitrogen and osmotic stress. They showed more root growth and more root hairs under conditions of nitrogen limitation compared to wild-type controls. Many plants were glabrous and also lacked anthocyanin production on stress conditions such as low nitrogen and high salt. In addition, one line showed higher amounts of seed protein, which could be a result of increased nitrogen uptake by these plants.

RT-PCR analysis of the endogenous levels of G226 indicated that the gene transcript was primarily found in leaf tissue. A cDNA array experiment supported this tissue distribution data by RT-PCR. G226 expression appeared to be repressed by soil drought treatment, as revealed by GeneChip microarray experiments. The gene itself was overexpressed 16-fold above wild type, however, very few changes in gene expression were observed. On the array, a chlorate/nitrate transporter was induced 2.7-fold over wild type, which could explain the low nitrogen tolerant phenotype of the plants and the increased amounts of seed protein in one of the lines. The same gene was spotted several times on the array and in all cases the gene showed induction, adding more validity to the data.

Discoveries in tomato. In transgenic tomatoes overexpressing G226 under the Cruciferin promoter, plant size was close to the highest wild type level and ranked in the 95th percentile among all size measurements.

Other related data: G226 paralogs include G1816, G225, G2718, and G682. Only G682 was tested in tomato in the tomato field trial, under the AP1, AS1, LTP1, RBCS3, and STM promoters. None of the promoters produced a positive hit in the three phenotypes discussed. Plants under the STM promoter were above average in size, but did not meet the 95th percentile cut off. Expressing G682 under the remaining promoters all resulted in plants that were smaller than average.

G682 and its paralogs have been studied extensively in Arabidopsis as part of the lead advancement drought program. During our earlier genomics program, members of the G682 clade were found to promote epidermal cell type alterations when overexpressed in Arabidopsis. These changes include both increased numbers of root hairs compared to wild type plants as well as a reduction in trichome number. In addition, overexpression lines for all members of the clade showed a reduction in anthocyanin accumulation in response to stress, enhanced tolerance to osmotic stress, and improved performance under nitrogen-limiting conditions. Information on gene function has been published for two of the genes in this clade, CAPRICE (CPC/G225) and TRYPTICHON (TRY/G1816). Mutations in CPC result in plants with very few root hairs and the overexpression of the gene causes an increase in the number of root hairs and a near trichome-less leaf phenotype, similar to results found by us (Wada (1997)). TRY has been shown to be involved in the lateral inhibition during epidermal cell specification in the leaf and root (Schellmann et al. (2002)). The model proposes that TRY (G11816) and CPC (G225) function as repressors of trichome and atrichoblast cell fate. TRY loss-of-function mutants form ectopic trichomes on the leaf surface. TRY gain-of-function mutants are glabrous and form ectopic root hairs.

Several orthologs were also tested in transgenic Arabidopsis. Plants overexpressing one of three soy orthologs (G3450, G3449, and G3448) were glabrous, had increased root hair density, and showed enhanced tolerance to low nitrogen. Overexpression of maize ortholog G3431 or rice ortholog G3393 gave a similar phenotype. Rice ortholog G3392 provided an even broader spectrum of stress tolerance in the plate-based assays.

TABLE 21
Data Summary for G226

| | Promoter summary: Avg. ± StD. (Count) | | |
|---|---|---|---|
| | Brix | | |
| Promoter | (g sugar/100 g sample) | Lycopene (ppm) | Volume (m³) |
| Cruciferin | 6.14 ± 0.064 (2) | 57.12 ± 5.827 (2) | 0.32 ± 0.066 (3) |
| PG | NA | NA | 0.16 ± 0.08 (2) |

### G237 (SEQ ID NO: 19 and 20)

Published background information. G237 (At4g25560) was identified from the Arabidopsis BAC sequence, AL022197, based on sequence homology to the conserved region of other members of the Myb family. The Myb consortium has named this gene AtMYB18 (Kranz et al. (1998)). Reverse-Northern data suggest that this gene is expressed at a low level in cauline leaves and may be slightly induced by cold.

Discoveries in Arabidopsis. The function of G237 was analyzed through its ectopic overexpression in Arabidopsis. Arabidopsis plants overexpressing G237 were small compared to wild-type controls and they displayed a variety of developmental abnormalities, particularly with respect to flower development. They also showed more disease spread after infection with the biotrophic fungal pathogen Erysiphe orontii compared to control plants. The transgenic plants did not have altered susceptibility to the necrotrophic fungal pathogen Fusarium oxysporum or the bacterial pathogen Pseudomonas syringae. RT-PCR analysis of endogenous levels of G237 only detected G237 transcript in root tissue. There was no induction of G237 transcript in leaf tissue in response to environmental stress treatments, based on RT-PCR and microarray analysis.

Discoveries in tomato. The fruit lycopene levels in transgenic tomatoes overexpressing G237 under the PD and PG promoter were higher than the highest wild type level and ranked in the 95th percentile among all lycopene measurements. Plant size under all promoters tested was smaller than average. Arabidopsis plants overexpressing G237 were small compared to wild-type controls and they displayed a variety of developmental abnormalities. They also showed more disease spread after infection with the biotrophic fungal pathogen Erysiphe orontii compared to control plants.

Other related data. G237 paralog G1309 was tested in transgenic tomatoes in the present field trial. Only volume measurements are available, and ectopic expression of G1309 did not result in a significant effect on plant size. In Arabidopsis, primary transformants of G1309 generally had smaller rosettes and shorter petioles than control plants in two plantings. However, this phenotype did not appear in the T2 generation. One line also showed a reproducible increase in mannose in leaves when compared with wild type. G237 was originally reported to have an increased percentage of arabinose and mannose but this did not repeat.

TABLE 22
Data Summary for G237

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.69 ± NA (1) | 36.31 ± NA (1) | 0.07 ± 0.01 (3) |
| AP1 | 5.53 ± 1.223 (2) | 72.33 ± 50.82 (2) | 0.07 ± 0.019 (3) |
| AS1 | 5.71 ± 0.113 (2) | 63.55 ± 33.969 (2) | 0.07 ± 0.044 (3) |
| Cruciferin | 5.1 ± NA (1) | 65.87 ± NA (1) | 0.1 ± 0.045 (3) |
| PD | 5.94 ± NA (1) | 106.1 ± NA (1) | 0.11 ± NA (1) |
| PG | 5.53 ± 0.157 (3) | 98.4 ± 22.843 (3) | 0.08 ± 0.007 (3) |
| STM | 5.65 ± 0.078 (2) | 69.31 ± 47.779 (2) | 0.09 ± 0.021 (3) |

### G270 (SEQ ID NO: 21 and 22)

Published background information. The sequence of G270 (At5g66055) was initially obtained from the Arabidopsis sequencing project, GenBank accession number AB01474.1 (GI:2924651). G1270 has no distinctive features other than the presence of a 33-amino acid repeated ankyrin element known for protein-protein interaction, in the C-terminus of the predicted protein. Amino acid sequence comparison shows similarity to Arabidopsis NPR1.

Discoveries in Arabidopsis. The analysis of the endogenous level of G270 transcripts by RT-PCR revealed constitutive expression in all tissues and biotic/abiotic treatments examined. Microarray analysis revealed a significant (p-value<0.01) reduction in G270 expression level in shoots of ABA treated plants (4 hr, 8 hr and 24 hr time points). The function of G270 was analyzed by ectopic overexpression in Arabidopsis. The characterization of G270 transgenic lines revealed no significant morphological, physiological or biochemical changes when compared to wild-type controls.

Discoveries in tomato. Transgenic tomatoes expressing G270 under the meristem (AS1) promoter were larger than wild type controls; ranking in the 95th percentile among all size measurements. In addition, morphological examination revealed that transgenic AS1-G270 tomato plants produced, in average, more green fruits than wild-type control plants. Under the cruciferin promoter, G270 expression resulted in larger fruits. 35S::G270 Arabidopsis plants were morphologically indistinguishable from wild-type plants. Those observations indicate that G270 may be an important regulator of plant biomass with a positive impact on overall fruit yield.

Other related data. The paralog of G270, G1280, was not tested in tomato in the present field trial. Similar to G270, transgenic 35S::G1280 Arabidopsis plants were indistinguishable from wild type controls.

TABLE 23
Data Summary for G270

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.67 ± NA (1) | 50.89 ± NA (1) | 0.18 ± 0.012 (3) |
| AP1 | NA | NA | 0.13 ± 0.029 (2) |
| AS1 | 4.96 ± 0.071 (2) | 37.92 ± 0.035 (2) | 0.34 ± 0.12 (2) |
| Cruciferin | 4.89 ± 0.247 (2) | 43.41 ± 16.461 (2) | 0.3 ± 0.112 (3) |
| PD | 5.61 ± NA (1) | 46.85 ± NA (1) | 0.25 ± 0.156 (3) |
| PG | 5.02 ± NA (1) | 25.37 ± NA (1) | 0.26 ± 0.028 (3) |
| RBCS3 | 5.59 ± NA (1) | 46.9 ± NA (1) | 0.21 ± 0.013 (2) |

### G328 (SEQ ID NO: 23 and 24)

Published background information. G328 was identified as COL-1 (CONSTANS LIKE-1, accession number Y10555) (1), and is a close homologue of the flowering time gene CONSTANS(CO). Both genes were found to form a tandem repeat on chromosome 5.

Ledger et al. (2001) showed that the circadian clock regulates expression of COL1 with a peak in transcript levels around dawn. Altered expression of COL1 in transgenic plants had little effect on flowering time. Analysis of circadian phenotypes in transgenic plants showed that over-expression of COL1 can shorten the period of two distinct circadian rhythms. Experiments with the highest COL1 over-expressing line indicate that its circadian defects are fluence rate-dependent, suggesting an effect on a light input pathway(s).

Discoveries in Arabidopsis. The function of this gene was analyzed using transgenic plants in which G328 was expressed under the control of the 35S promoter. The phenotype of these transgenic plants was wild type in all assays performed. Expression profiling assays using RT/PCR showed that the expression levels of G328 were slightly reduced in response to treatments with ABA, salt, drought and infection with Erysiphe. Microarray experiments indicate that G328 was induced by drought, cold, NaCl, mannitol, ABA, salicylic acid (SA), G481 overexpression, and G912 overexpression.

Discoveries in tomato. The fruit lycopene level under the LTP1 and STM promoters were above the highest wild type levels and ranked in the 95th percentile among all measurements.

Other related data. The paralogs of G328, G2436 and G2443, were not tested in tomato in the present field trial. No significant changes in lycopene, plant size, or Brix was detected in either LTP1::G1917 or STM::G1917 plants. Neither G2436 nor G2443 was analyzed in Arabidopsis.

TABLE 24
Data Summary for G328

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 5.65 ± NA (1) | 114.15 ± NA (1) | 0.21 ± 0.063 (2) |
| PG | 6.01 ± NA (1) | 102.46 ± NA (1) | 0.21 ± 0.02 (3) |
| RBCS3 | 5.65 ± 0.792 (3) | 71.77 ± 15.838 (3) | 0.2 ± 0.084 (3) |
| STM | 5.62 ± NA (1) | 65.16 ± NA (1) | 0.16 ± 0.023 (3) |

### G363 (SEQ ID NO: 25 and 26)

Published background information. G363 corresponds to ZFP4 (Tague and Goodman, 1995). ZPF4 was reported to be a member of a gene family with high expression in roots. A reduced level of expression was detected in stems. No other public information is available concerning the function of this gene.

Discoveries in Arabidopsis. As determined by RT-PCR, G363 was highly expressed in leaves, roots and shoots, and at lower levels in the other tissues tested. No expression of G363 was detected in the other tissues tested. The high expression detected in leaves is contrary to the lack of expression reported by Tague and Goodman (1995). G363 expression was also slightly induced in rosette leaves by auxin, ABA and cold treatments. Overexpression of G363 resulted in many primary transformants that were smaller than controls. Otherwise, all observed phenotypes in all assays were wild type.

G363 expression was induced by drought, ABA, SA, G1073 overexpression, G481 overexpression, G682 overexpression, and G912 overexpression.

Discoveries in tomato. The fruit lycopene level in transgenic tomato plants overexpressing G363 under the regulatory control of the LTP1 promoter was above the highest wild type levels and ranked in the 95th percentile among all measurements.

TABLE 25
Data Summary for G363

| | Promoter summary: Avg. ± StD. (Count) | | |
|---|---|---|---|
| | Brix | | |
| Promoter | (g sugar/100 g sample) | Lycopene (ppm) | Volume (m³) |
| LTP1 | 5 ± NA (1) | 105.08 ± NA (1) | 0.2 ± 0.039 (3) |

### G383 (SEQ ID NO: 27 and 28)

Published background information. G383 was identified as a gene in the sequence of chromosome 4, contig fragment No. 85 (Accession number AL161589), released by the European Union Arabidopsis sequencing project. No published information is available regarding the function(s) of G383.

Discoveries in Arabidopsis. The sequence of G383 was experimentally determined and the function of G383 was analyzed using transgenic plants in which G383 was expressed under the control of the 35S promoter. In roughly 50% of the T1 seedlings, increased amounts of anthocyanin in petioles and apical meristems was observed. However, this might be due to transplanting as this effect was not observed in the T2 seedlings. In all other morphological, physiological, or biochemical assays, plants overexpressing G383 appeared to be identical to controls.

G383 was expressed at low levels in flowers, rosette leaves, embryos and siliques by RT-PCR. No change in the expression of G383 was detected in response to the environmental stress-related conditions tested using RT-PCR. Microarray experiments indicated that G383 is induced by cold.

Discoveries in tomato. The fruit lycopene level in transgenic tomato plants overexpressing G383 under the regulatory control of the STM promoter was above the highest wild type levels and ranked in the 95th percentile among all measurements.

Other related data. A paralog of G383, G1917, tested in tomato in the present field trial. No significant changes in lycopene, plant size, or Brix was detected in either LTP1::G1917 or STM::G1917 plants. The function of G1917 was studied in Arabidopsis by knockout analysis. Plants homozygous for a T-DNA insertion in G1917 showed a significant increase in peak M39489 in the seed glucosinolate assay.

TABLE 26
Data Summary for G383

| | Promoter summary: Avg. ± StD. (Count) | | |
|---|---|---|---|
| | Brix | | |
| Promoter | (g sugar/100 g sample) | Lycopene (ppm) | Volume (m³) |
| 35S | 5.59 ± 0.764 (2) | 49.45 ± 5.197 (2) | 0.21 ± 0.073 (3) |
| LTP1 | 5.12 ± 1.103 (2) | 53.03 ± 0.792 (2) | 0.27 ± 0.044 (3) |
| PG | 6.12 ± 0.17 (2) | 84.78 ± 6.866 (2) | 0.3 ± 0.058 (3) |
| RBCS3 | 5.54 ± 0.112 (3) | 59.37 ± 9.826 (3) | 0.3 ± 0.035 (3) |
| STM | 5.76 ± 0.559 (2) | 99.38 ± 8.111 (2) | 0.27 ± 0.022 (3) |

### G435 (SEQ ID NO: 29 and 30)

Published background information. G435 corresponds to AT5G53980 and encodes a HD-ZIP class I HD protein.

Discoveries in Arabidopsis. Overexpression of G435 produced some alterations in morphology such as reduced size, delayed bolting, and altered seed shape. 35S::G435 Arabidopsis lines were also more shade tolerant in a screen under conditions deficient in red light.

RT-PCR experiments revealed that G435 is expressed in a wide range of Arabidopsis tissue types. Microarray experiments have subsequently revealed that expression of G435 is stress responsive. The gene was up-regulated in response to ACC, drought, mannitol, and salt and was repressed in response to cold treatments.

Discoveries in tomato. Lycopene levels in RBCS3::G435 fruits were markedly higher than those found in wild-type fruit.

TABLE 27
Data Summary for G435

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.55 ± 1.061 (2) | 63.11 ± 52.114 (2) | 0.15 ± 0.009 (3) |
| AP1 | 5.78 ± 0.227 (3) | 76.16 ± 12.648 (3) | 0.21 ± 0.039 (3) |
| AS1 | 5.56 ± 0.028 (2) | 72.47 ± 10.472 (2) | 0.16 ± 0.051 (3) |
| LTP1 | NA | NA | 0.27 ± 0.036 (3) |
| PG | 5.31 ± 0.721 (2) | 57.58 ± 5.918 (2) | 0.29 ± 0.209 (3) |
| RBCS3 | 6.05 ± NA (1) | 99.77 ± NA (1) | 0.18 ± 0.025 (3) |
| STM | 5.31 ± 0.834 (2) | 81.19 ± 7.022 (2) | 0.16 ± 0.014 (3) |

### G450 (SEQ ID NO: 31 and 32)

Published background information. G450 is IAA14, a member of the Aux/IAA class of small, short-lived nuclear proteins. Aux/IAA proteins function through heterodimerization with ARF transcriptional regulators, as well as homo- and heterodimerization with other IAA proteins. Most Aux/IAA proteins are thought to be negative regulators of ARF proteins, and are degraded in response to auxin. A gain-of-function mutant in IAA14, slr (solitary root), was found to abolish lateral root formation, reduce root hair formation, and impair gravitropic responses (Fukaki et al. (2002)).

Discoveries in Arabidopsis. Overexpression of G450 influenced leaf development, overall plant stature, and seed size, Some lines of 35S::G450 plants were slightly small and their leaves were often curled and twisted. Larger seeds were reported for two T2 lines; this phenotype could be related to lower fertility. 35S::G450 plants were wild type in all physiological and biochemical assays. Overexpression of G450 did not phenocopy the gain-of-function mutation slr. This is consistent with results obtained with other IAA family members such as axr3 (G448) and shy2 (G449).

Discoveries in tomato. Plants expressing G450 under the STM promoter scored in the 95th percentile for fruit lycopene and Brix.

Other related data. G448, G455 and G456 are G450 paralogs. None of these genes have been tested in field trials yet. The paralogs all produced either no phenotypic alterations in Arabidopsis, or only minor morphological alterations.

TABLE 28
Data Summary for G450

| | Promoter summary: Avg. ± StD. (Count) | | |
|---|---|---|---|
| | Brix | | |
| Promoter | (g sugar/100 g sample) | Lycopene (ppm) | Volume (m³) |
| 35S | NA | NA | 0.16 ± 0.016 (3) |
| AP1 | 5.96 ± NA (1) | 87.02 ± NA (1) | 0.2 ± 0.075 (3) |
| AS1 | 4.52 ± NA (1) | 41.2 ± NA (1) | 0.16 ± 0.063 (3) |
| LTP1 | 5.52 ± NA (1) | 41.7 ± NA (1) | 0.2 ± 0.052 (3) |
| PD | NA | NA | 0.17 ± 0.091 (3) |
| RBCS3 | NA | NA | 0.21 ± 0.039 (3) |
| STM | 6.28 ± NA (1) | 109.97 ± NA (1) | 0.16 ± 0.037 (3) |

### G522 (SEQ ID NO: 33 and 34)

Published background information. G522 was first identified in the sequence of the BAC clone F23E13, GenBank accession number AL022141, released by the Arabidopsis Genome initiative. It also corresponds to the AGI locus of AT4G36160. A comprehensive analysis of NAC family transcription factors was recently published by Ooka et al. (2003) where G522 was identified as ANAC076.

Discoveries in Arabidopsis. The function of G522 was analyzed using transgenic plants in which G522 was expressed under the control of the 35S promoter. The phenotype of these transgenic plants was wild-type in all assays performed. RT-PCR analysis was used to determine the endogenous levels of G522 in a variety of issues and under a variety of environmental stress-related conditions. G522 is primarily expressed in flowers and at low levels in shoots and roots. RT-PCR data also indicates an induction of G522 transcript accumulation upon auxin treatment.

Discoveries in tomato. Transgenic tomatoes expressing G522 under the regulation of both 35S and AP1 promoters showed a significant increase in soluble solids levels.

Other related data. Putative paralogs of G522 have been identified by us. These consist of: G1354, G1355, G1453, G1766, G2534 and G761. The most closely related paralog (G1355) exhibited a decrease in seed oil in one line and no obvious effects on growth and development. However all other paralogs, when overexpressed in Arabidopsis exhibited gross to mild alteration in growth and development.

TABLE 29
Data Summary for G522

| | Promoter summary: Avg. ± StD. (Count) | | |
|---|---|---|---|
| | Brix | | |
| Promoter | (g sugar/100 g sample) | Lycopene (ppm) | Volume (m³) |
| 35S | 6.8 ± NA (1) | 35.69 ± NA (1) | 0.06 ± 0.001 (2) |
| AP1 | 6.41 ± NA (1) | 56.55 ± NA (1) | 0.1 ± 0.037 (3) |
| AS1 | NA | NA | 0.06 ± 0.012 (3) |
| PG | 5.76 ± NA (1) | 56.42 ± NA (1) | 0.08 ± 0.018 (3) |
| RBCS3 | NA | NA | 0.04 ± 0.013 (3) |
| STM | 6.1 ± NA (1) | 72.33 ± NA (1) | 0.06 ± 0.027 (2) |

### G551 (SEQ ID NO: 35 and 36)

Published background information. G551 corresponds to AT5G03790 and encodes a HD-ZIP class I HD protein.

Discoveries in Arabidopsis. G551 was analyzed during our Arabidopsis genomics program. The function of G551 was assessed by analysis of transgenic Arabidopsis lines in which the cDNA was constitutively expressed from the 35S CaMV promoter. Overexpression of G551 produced a range of effects on morphology, including changes in leaf and cotyledon shape, coloration, and a reduction in overall plant size, and fertility. However, these phenotypes were somewhat variable between different transformants. In particular, the most severely affected lines were very small, dark green, in some cases had serrated leaves, and in some cases flowered early.

RT-PCR experiments revealed that G551 is expressed at moderately high levels in a range of tissue types. However, G551 has not been found to be significantly differentially expressed in any of the conditions examined in microarray studies performed to date.

Discoveries in tomato. Transgenic tomatoes expressing G551 under the regulation of each of the 35S, AP1, Cruciferin, LTP1, RBCS3, and STM promoters were analyzed for alterations in plant size, soluble solids and lycopene. Soluble solid levels in STM::G551 fruits were markedly higher than those found in wild-type controls.

TABLE 30
Data Summary for G551

| | Promoter summary: Avg. ± StD. (Count) | | |
|---|---|---|---|
| | Brix | | |
| Promoter | (g sugar/100 g sample) | Lycopene (ppm) | Volume (m³) |
| 35S | NA | NA | 0.18 ± 0.026 (3) |
| AP1 | NA | NA | 0.07 ± 0.042 (2) |
| Cruciferin | 5.54 ± NA (1) | 30.11 ± NA (1) | 0.1 ± 0.092 (3) |
| LTP1 | 5.8 ± NA (1) | 69.57 ± NA (1) | 0.1 ± 0.01 (3) |
| RBCS3 | 5.36 ± 0.262 (2) | 55.22 ± 3.083 (2) | 0.14 ± 0.008 (2) |
| STM | 6.58 ± NA (1) | 60.31 ± NA (1) | 0.08 ± 0.026 (3) |

### G558 (SEQ ID NO: 37 and 38)

Published background information. G558 is the Arabidopsis transcription factor TGA2 (de Pater S, et al, 1996) or AHBP-1b (Kawata T, et al. 1992). TGA2 was shown by the two hybrid system to interact with NPR1—a key component of the SA-regulated pathogenesis-related gene expression and disease resistance pathways in plants (Zhang Y, et al 1999). Furthermore, gel shift analysis showed TGA2 can bind to the PR1 promoter (Zhang Y, et al 1999). In vitro, binding activity of TGA2 can be abolished by a dominant negative mutant of TGA1a from tobacco (Miao Z H, et al 1995) and it is constitutively expressed in roots, shoots, leaves and flowers, and expressed at lower levels in siliques (de Pater S, et al, 1996).

Discoveries in Arabidopsis. Determination of endogenous levels of G558 by RT-PCR indicates that this gene is expressed in all tissues tested. G558 is significantly repressed in cold and salt stress and marginally induced by Erysiphe and salicylic acid. G558 overexpressing lines were subject to gene expression profiling experiments using a 7000 element cDNA array. These experiments showed that G558 is highly overexpressed (at least 15-fold) in rosette leaves of overexpressing plants, and that several known genes are induced. These genes encode: GST, phospholipase D, PGP224 (also strongly induced by Erysiphe), PR1, berberine bridge enzyme (the bridge enzyme of antimicrobial benzophenanthridine alkaloid biosynthesis which is methyl jasmonate-inducible), polygalacturonase, WAK 1 PGP224 (also strongly induced by Erysiphe), pathogen-inducible protein CXc750, tryptophan synthase, tyrosine transaminase and an antifungal protein. Almost all of the top induced genes in G558 overexpressing lines are related to disease, and most of these have been shown to be induced or repressed in response to Erysiphe or Fusarium infection. Thus genes involved in the defense response appeared to be induced in plants overexpressing G558 T2 plants expressing G558 were noted as having poor fertility and were slightly earlier flowering in comparison to wild type. Published data demonstrate that G558 interacts with NPR1 (3). We have shown that G558 was marginally inducible with Erysiphe and salicylic acid and that when G558 was overexpressed, genes involved in the defense response appeared to be induced. These data indicate that G558 is an important component of the defense response. However, overexpression of G558 does not appear to cause plants to be more resistant to disease, suggesting that its expression alone is not sufficient to mount a full defense response. G558 is also repressed by cold treatment, raising the possibility that G558 may be responsible for making Arabidopsis more susceptible to some pathogens at lower temperatures.

Discoveries in tomato. The respective fruit lycopene level under the AS1 promoter and Brix level under the STM promoter were close to the highest wild type levels and ranked in the 95th percentile among all measurements. Under the AP1 promoter, plant size is also significantly more than the wild type controls. Its paralog G1198 was also tested in a field trial but no significant differences were detected in all assays performed. Several of its paralogs were also overexpressed in Arabidopsis—some resulting in stunted plants while others having no phenotype.

Other related data. G558 paralogs include G1198 G1806 G554 G555 G556 G578 and G629. Only G1198 was tested in tomato in the field trial. No significant differences were detected in all assays performed with G1198 in tomato. In Arabidopsis, overexpression of G1198 and G1806 was deleterious and overexpression of G578 was lethal. In contrast, overexpression of G554, G555, G556 and G629 did not result in any observable

TABLE 31
Data Summary for G558

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.76 ± NA (1) | 43.48 ± NA (1) | 0.28 ± 0.075 (3) |
| AP1 | 6.18 ± 0.189 (3) | 75.2 ± 22.272 (3) | 0.32 ± 0.056 (3) |
| AS1 | 6.31 ± NA (1) | 98.75 ± NA (1) | 0.2 ± 0.104 (3) |
| STM | 6.39 ± 0.417 (2) | 92.88 ± 3.479 (2) | 0.17 ± 0.042 (2) |

### G567 (SEQ ID NO: 39 and 40)

Published background information. G567 was discovered as a bZIP gene in BAC T10P11, accession number AC002330, released by the Arabidopsis genome initiative. There is no published information regarding the function of G567.

Discoveries in Arabidopsis. The annotation of G567 in BAC AC002330 was experimentally confirmed and the function of G567 was analyzed using transgenic plants in which G567 was expressed under the control of the 35S promoter. Seedlings overexpressing G567 had slowly opening cotyledons and very short roots when grown on MS plates containing glucose. These plants were otherwise wild type. G567 could be involved in sugar sensing or metabolism during germination. G567 appeared to be constitutively expressed, and induced in leaves in a variety of conditions.

Discoveries in tomato. The fruit Brix level under the AP1 promoter was close to the highest wild type level and ranked above the 95th percentile among all Brix measurements. Arabidopsis seedlings overexpressing G567 had slowly opening cotyledons and very short roots when grown on MS plates containing glucose but were otherwise wild type.

TABLE 32
Data Summary for G567

| | Promoter summary: | | |
|---|---|---|---|
| | Avg. ± StD. (Count) | | |
| | Brix (g sugar/100 g | | |
| Promoter | sample) | Lycopene (ppm) | Volume (m³) |
| AP1 | 6.31 ± 0.368 (2) | 71.1 ± 13.195 (2) | 0.17 ± 0.024 (3) |
| AS1 | 5.8 ± 0.375 (2) | 89.39 ± 10.479 (2) | 0.18 ± 0.055 (3) |
| LTP1 | 5.87 ± NA (1) | 81.33 ± NA (1) | 0.26 ± 0.106 (3) |
| PD | 5.83 ± NA (1) | 81.02 ± NA (1) | 0.17 ± 0.072 (3) |
| RBCS3 | 5.6 ± 0.035 (2) | 61.79 ± 13.096 (2) | 0.25 ± 0.029 (3) |
| STM | NA | NA | 0.2 ± NA (1) |

### G580 (SEQ ID NO: 41 and 42)

Published background information. G580 was identified in the sequence of BAC T17A5, GenBank accession number AF024504, released by the Arabidopsis Genome Initiative. The annotation of G580 in BAC AF024504 was experimentally confirmed.

Discoveries in Arabidopsis. The function of this gene was analyzed using transgenic plants in which G580 was expressed under the control of the 35S promoter. 35S::G580 plants displayed a variety of morphological phenotypes in the T1 generation when compared to controls. These overexpressor plants were small and spindly, had altered flower and silique development, and had reduced and inflorescence internode length. G580 overexpressors were otherwise physiologically and biochemically wild-type, although phenotypes caused by G580 may be attenuated in the T2 generation.

G580 appeared to be preferentially expressed in roots and flowers but was otherwise constitutive. Microarray analysis revealed no significant (p-value<0.01) change in G580 expression in all conditions examined.

Discoveries in tomato. The PG::G580 lines had poor fruit set, thus limiting the analysis to plant size. The fruit Brix level under the STM promoter was higher than the highest wild type level and ranked above the 95th percentile among all Brix measurements. Fruit lycopene levels under both the 35S and STM promoters were higher than the highest wild type level and ranked above the 95th percentile among all lycopene measurements. Lycopene level in Cruc::G580 fruit was also above controls (above 75th percentile). Arabidopsis plants overexpressing G580 displayed a variety of morphological phenotypes in the T1 generation when compared to controls. These overexpressor plants were small and spindly, had altered flower and silique development, and had reduced and inflorescence internode length. These data indicate that G580 may be an important regulator affecting lycopene and soluble solids in tomato fruit.

Other related data. G568 is a paralog of G580, however, this gene was not tested in the field trial. Arabidopsis plants overexpressing G568 displayed a variety of morphological phenotypes when compared to control plants but were otherwise biochemically and physiologically wild-type. These morphological phenotypes included narrow leaves, a darker green coloration, and bushy, spindly, poorly fertile shoots, dwarfing and flowering time alteration.

TABLE 33
Data Summary for G580

| | Promoter summary: | | |
|---|---|---|---|
| | Avg. ± StD. (Count) | | |
| | Brix (g sugar/100 g | | |
| Promoter | sample) | Lycopene (ppm) | Volume (m³) |
| 35S | 5.38 ± NA (1) | 111.92 ± NA (1) | 0.19 ± 0.04 (3) |
| Cruciferin | 4.6 ± NA (1) | 84.25 ± NA (1) | 0.26 ± 0.085 (2) |
| PG | NA | NA | 0.08 ± 0.011 (3) |
| STM | 6.7 ± 0.474 (2) | 106.67 ± 22.832 (2) | 0.16 ± 0.07 (3) |

### G635 (SEQ IUD NO: 43 and 44)

Published background information. 0635 corresponds to AT5G63420. This gene encodes a protein with similarities to the TH family of transcription factors. However, the locus is annotated at TAIR as encoding a metallo-beta-lactamase protein and is classified as having a potential role in chloroplast metabolism. G635 does not appear to have any closely related paralogs.

Discoveries in Arabidopsis. The function of this gene was analyzed using transgenic plants in which G635 was expressed under the control of the 35S promoter. 35S::G635 Arabidopsis lines generally appeared wild-type, but about 15% of the lines exhibited a very striking variegated phenotype in which sectors of white chlorotic tissues were observed on the leaves and stems. Such a phenotype implicated the gene in the regulation of pigmentation or chloroplast biogenesis. Interestingly, the lines that showed these effects had very low levels of transgene expression, suggesting that the phenotype might be the result of co-suppression or some related gene silencing type phenomenon. The morphological effects observed were consistent with the TAIR annotation of the locus being involved in chloroplast metabolism.

In some initial biochemical analyses performed on 35S::G635 Arabidopsis plants, one of three (non-chlorotic) lines tested showed an alteration in leaf insoluble sugar composition and had an increase in galactose levels. However, this phenotype was not observed in an initial repeat of the experiment; further repeats and examination of a larger number of lines would therefore be required to confirm or discount the effect. In addition to the effects above, G635 lines (non-chlorotic) showed enhanced performance in a first round C/N sensing screen. However, this result still awaits confirmation in repeat experiments.

RT-PCR experiments revealed that G635 was expressed at in a range of Arabidopsis tissue types. Microarray experiments performed revealed that G635 was significantly repressed in response to ABA, SA and NaCl.

Discoveries in tomato. The 35S, AP1, AS1 PG and RBCS3::G635 lines had poor fruit set, thus limiting the analysis to plant size. Both lycopene and soluble solid levels in PD::G635 fruits were markedly higher than those found in wild-type controls; ranking in the 95th percentile of all measurements. The results of Arabidopsis genomics studies performed and the annotation at TAIR suggest that the gene might have an endogenous role in the regulation of pigmentation or chloroplast biogenesis/metabolism. These data indicate that G635 may be an important regulator affecting lycopene and soluble solids in tomato fruit.

TABLE 34
Data Summary for G635

| | Promoter summary: | | |
|---|---|---|---|
| | Avg. ± StD. (Count) | | |
| | Brix | | |
| Promoter | (g sugar/100 g sample) | Lycopene (ppm) | Volume (m³) |
| 35S | NA | NA | 0.22 ± 0.013 (2) |
| AP1 | NA | NA | 0.2 ± 0.045 (3) |
| AS1 | NA | NA | 0.15 ± 0.14 (3) |
| PD | 6.85 ± NA (1) | 108.82 ± NA (1) | 0.22 ± 0.044 (3) |
| PG | NA | NA | 0.17 ± 0.031 (3) |
| RBCS3 | NA | NA | 0.27 ± NA (1) |

### G675 (SEQ ID NO: 45 and 46)

Published background information. G675 (At1 g34670) was discovered by its identification from an Arabidopsis EST based on its similarity to other proteins containing a conserved Myb motif. Subsequently, Kranz et al. (1998) published a partial cDNA sequence corresponding to G675, naming it AtMYB93. Reverse-Northern data suggest that this gene could be induced slightly by the plant growth regulators ABA and IAA, and a low level of expression was detected in roots but no other plant parts tested (Kranz et al. (1998)).

Discoveries in Arabidopsis. In Arabidopsis, a line homozygous for a T-DNA insertion in G675 as well as transgenic plants expressing G675 under the control of the 35S promoter were used to determine the function of this gene. The phenotype of the knockout mutant and overexpressing transgenic plants was wild-type in all assays performed.

A line homozygous for a T-DNA insertion in G675 as well as transgenic plants expressing G675 under the control of the 35S promoter were used to determine the function of this gene. The phenotype of the knockout mutant and overexpressing transgenic plants was wild-type in all assays performed. RT-PCR analysis of the endogenous levels of G675 suggested the gene was expressed at low levels in root and silique tissues, and at slightly higher levels in embryos and germinating seeds. No induction of G675 was detected in response to stress-related treatments, as determined by RT-PCR. Microarray analysis showed that G675 is induced in roots by ABA, mannitol, and NaCl; it is also induced briefly in the shoot by SA, potentially implicating it in the drought response pathways, although physiology assays did not show an altered response to osmotic or drought stress in the transgenic lines.

Discoveries in tomato. LTP1::G675 lines had poor fruit set, thus limiting the analysis to plant size. Under the regulatory control of AS1, RBCS3, and STM promoters, fruit lycopene levels were higher than the highest wild type level and ranked in the 95th percentile among all lycopene measurements. All three of these promoters are active in tomato fruits. 35S::G675 fruits also showed higher lycopene level than controls (above 75th percentile). In addition, plant size under the 35S and AP1 promoters ranked in the 95th percentile among all measurements. Additionally, STM- and AP1-G675 transgenic plants produced small fruits. These data indicate that G675 may be an important regulator affecting fruit lycopene and plant biomass.

TABLE 35
Data Summary for G675

| | Promoter summary: | | |
|---|---|---|---|
| | Avg. ± StD. (Count) | | |
| | Brix (g sugar/100 g | | |
| Promoter | sample) | Lycopene (ppm) | Volume (m³) |
| 35S | 5.23 ± 0.433 (3) | 50.09 ± 6.992 (3) | 0.33 ± 0.093 (3) |
| AP1 | 5.58 ± 1.082 (2) | 90.1 ± 2.729 (2) | 0.33 ± 0.129 (3) |
| AS1 | 6.22 ± 0.467 (2) | 97.58 ± 12.841 (2) | 0.2 ± 0.027 (3) |
| Cruciferin | 5.68 ± 0.676 (3) | 63.04 ± 2.741 (3) | 0.27 ± 0.05 (3) |
| LTP1 | NA | NA | 0.31 ± 0.036 (3) |
| PD | 4.47 ± NA (1) | 38.59 ± NA (1) | 0.27 ± 0.103 (3) |
| PG | 5.41 ± 0.325 (2) | 41.41 ± 6.498 (2) | 0.25 ± 0.035 (3) |
| RBCS3 | 6.18 ± NA (1) | 103 ± NA (1) | 0.26 ± 0.115 (2) |
| STM | 4.32 ± NA (1) | 101.65 ± NA (1) | 0.21 ± 0.002 (3) |

### G729 (SEQ ID NO: 47 and 48)

Published background information. G729 corresponds to KANADI (KAN1), a regulator of abaxial/adaxial polarity (Kerstetter et al. (2001), Eshed et al. (2001)). Further published work (Eshed et al. (2001)) describes a clade of four KANADI genes, and shows that KAN1 and KAN2 (G3034) act redundantly to promote abaxial cell fates. Plants carrying mutations in both kan1 and kan2 showed severe morphological abnormalities that are interpreted as adaxialization of abaxial structures. Plants overexpressing KAN1, KAN2, or KAN3 (G730) under the 35S promoter generally arrested at the cotyledon stage, while only a small minority survived to produce leaves. Overexpressing KAN1, KAN2, or KAN3 under the AS1 promoter, which does not drive expression in the meristem, caused abaxialization of adaxial structures.

Discoveries in Arabidopsis. Subtle morphological changes were noted for the G729 knockout: the first pair of true leaves stood upright, though rosette stage plants looked normal, and older plants had slightly shorter siliques and rounder cauline leaves than control (WS-0) plants. Upon further examination of the silique phenotype, we found that many KO.G729 flowers possessed an additional one or two vestigial carpels fused to either side of the replum of main carpel. In some flowers, these extra carpels were very small and filamentous, in other cases they were more extensively developed. These results were consistent with the published phenotype of KANADI knockouts (Kerstetter et al. (2001); Eshed et al. (2001)). Overexpression of G729 under the 35S promoter produced highly abnormal plants or complete lethality, also consistent with published data (Eshed et al. (2001).

G729 was expressed at low levels throughout the plant with higher levels of expression in embryos and siliques, and it is not induced by any condition tested. Microarray analysis revealed no significant change (p-value<0.01) in G729 expression in all conditions examined.

Discoveries in tomato. Tomato plants overexpressing G729 under the cruciferin and PG promoters scored in the 95th percentile for plant size. These plants generally exhibited higher lycopene content than controls as well. The cruciferin and PG promoters are both active in tomato seedlings, as well as in fruits and seeds.

LTP1::G729 lines were are also significantly larger than controls. The PG::G729 plants were noted to have heavy fruit set, indicating that the increase in plant volume did not represent production of vegetative mass at the expense of fruit set. This result was somewhat surprising, given the published role of the KANADI genes in regulation of abaxial/adaxial polarity. It is possible that the action of these genes is through regulation of differential growth, and low level expression causes a non-specific growth increase.

Other related data. G730, G1040, and G3034 are paralogs of G729. None of these genes have been tested in the ATP field trials yet. G730 (KAN3) and G3034 (KAN2) are also implicated in determination of abaxial polarity in Arabidopsis (Eshed et al. (2001).

TABLE 36
Data Summary for G729

| | Promoter summary: Avg. ± StD. (Count) | | |
|---|---|---|---|
| | Brix | | |
| Promoter | (g sugar/100 g sample) | Lycopene (ppm) | Volume (m³) |
| 35S | 5.41 ± 0.373 (3) | 49.25 ± 5.438 (3) | 0.3 ± 0.04 (3) |
| Cruciferin | 5.57 ± 0.07 (3) | 79.11 ± 6.816 (3) | 0.41 ± 0.042 (3) |
| PG | 5.61 ± 0.845 (3) | 64.85 ± 35.15 (3) | 0.36 ± 0.039 (3) |

### G812 (SEQ ID NO: 49 and 50)

Published background information. The sequence of G812 (At3g511910) was initially obtained from the Arabidopsis sequencing project, GenBank accession number AL049711.3 (GI:6807566), based on sequence similarity to the heat shock transcription factors. G812 is a member of the class-A HSFs (Nover (1996)) characterized by an extended HR-A/B oligomerization domain.

Discoveries in Arabidopsis. The function of this gene was analyzed using transgenic plants in which G812 was expressed under the control of the 35S promoter. 35S::G812 Arabidopsis plants showed better tolerance to infection with the necrotrophic fungal pathogen Botrytis cinerea when compared to wild-type control plants. T1 transgenic plants were generally smaller than wild type and somewhat spindly.

G812 transcripts in wild type Arabidopsis were below detectable level in all tissues and biotic/abiotic treatments examined. Microarray analysis revealed a significant (p-value<0.01), but transient reduction (8 hr time point) in G812 expression level in root of cold-treated (4° C.) plants. Similarly, we observed transient induction of G812 in root, 0.5 hr after treatment with ABA. No changes in G812 expression were observed in response to other biotic and abiotic treatments.

Discoveries in tomato. LTP1::G812 lines had poor fruit set, thus limiting the analysis to plant size. Transgenic tomato plants expressing G812 under the seed (cruciferin) and fruit (PD) promoters were larger than wild type control; ranking among the 95th percentile of all volumetric measurements. Similarly, but to a lesser extent, LTP1, RBSCS3 and STM lines were larger than controls (90th percentile). All transgenic tomato seedlings expressing G812, regardless of the promoter, were more tolerant to extended drought conditions. This indicated that the transgenic G812 tomatoes were better adapted to water limiting conditions, resulting in increased fitness in the field and greater size. Constitutive ectopic expression of G812 resulted in moderate pleiotropic effects. Seedlings were etiolated and mature plants somewhat smaller than wild type. The same phenotypes were observed in 35S::G1560 tomato seedlings. G812 and G1560 are from the same phylogenetic clade and may be functionally redundant.

Transgenic 35S::G812 Arabidopsis plants were smaller than wild type, spindly and more tolerant to infection with the necrotrophic fungal pathogen Botrytis cinerea. This observation suggested that the increased fitness of G812 transgenic tomatoes in field-grown condition may be related to better tolerance to biotic and/or abiotic stresses.

Other related data. The paralog of G812, G2467, was not tested in field trial. Transgenic 35S::G2467 Arabidopsis plants were generally smaller than wild type, and formed rather thin inflorescence stems that carried flowers that sometimes displayed abnormal, poorly developed organs. Preliminary characterization tomato seedlings ectopically expressing G1560 revealed similar etiolated and drought tolerance phenotypes.

TABLE 37
Data Summary for G812

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.75 ± NA (1) | 55.24 ± NA (1) | 0.13 ± 0.044 (3) |
| Cruciferin | 5.96 ± 0.177 (2) | 50.38 ± 2.383 (2) | 0.35 ± 0.166 (3) |
| LTP1 | NA | NA | 0.29 ± 0.193 (3) |
| PD | 5.43 ± 0.198 (2) | 66.04 ± 21.666 (2) | 0.45 ± 0.152 (3) |
| RBCS3 | 5.87 ± 0.241 (3) | 95.29 ± 11.821 (3) | 0.27 ± 0.11 (3) |
| STM | 6.15 ± 0.156 (2) | 79.87 ± 5.254 (2) | 0.3 ± 0.094 (3) |

### G843 (SEQ ID NO: 51 and 52)

Published background information. The sequence of G843 (At3g07740) was initially obtained from the Arabidopsis sequencing project, GenBank accession number AC009176.5 (GI: 12408710), based on sequence similarity to the yeast transcriptional activator ADA2 (GI: 6320656). The Arabidopsis genome encodes two ADA2 proteins, G843 is designated as the transcriptional adaptor ADA2a. In yeast ADA2 proteins are part of the GCN5 multi-component complex of histone acetyltransferase. The paralog is G285 (ADA2b).

Discoveries in Arabidopsis. The function of G843 was analyzed through its ectopic overexpression in Arabidopsis. The characterization of 35S::G843 transgenic lines revealed no significant morphological, physiological or biochemical changes when compared to wild-type controls.

The analysis of the endogenous level of G843 transcripts by RT-PCR revealed a constitutive expression in all tissues and a moderate induction in response to auxin and heat shock treatment. Microarray analysis revealed no significant (p-value<0.01) alteration in G843 expression in all conditions examined.

Discoveries in tomato. In plants expressing G843 under the leaf (RBCS3), flower (AP1) and the fruit (PG) promoters, soluble solids (Brix measurement) in fruit was greater than that in wild type controls; ranking in the 95th percentile among all measurements. The RBCS3 and AP1 promoters are active in tomato fruits. Lycopene level in mature fruit of plants expressing G843 under the constitutive (35S) and the flower (AP1) promoters was higher than wild type controls; also ranking in the 95th percentile of all lycopene measurements. Expression of G843 under the seed (cruciferin) and meristem (STM) promoters negatively impacted fruit yield and maturation. These observations suggested that G843 may be an important regulator affecting soluble solids and lycopene in ripening tomato fruits. Overexpression of G843 resulted in no other significant pleiotropic effects on growth and development in transgenic tomato plants.

Other related data. The paralog of G843, G285, was not tested in field trial. Similar to G843, transgenic 35S::G285 Arabidopsis plants were indistinguishable from wild type controls.

TABLE 38
Data Summary for G843

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.75 ± NA (1) | 97.32 ± NA (1) | 0.27 ± 0.104 (3) |
| AP1 | 6.59 ± NA (1) | 100.95 ± NA (1) | 0.19 ± 0.097 (3) |
| AS1 | 5.82 ± 0.453 (2) | 68.63 ± 52.51 (2) | 0.16 ± 0.021 (3) |
| Cruciferin | 5.36 ± 0.29 (2) | 68.13 ± 17.763 (2) | 0.18 ± 0.032 (3) |
| PG | 6.26 ± NA (1) | 67.67 ± NA (1) | 0.28 ± 0.014 (3) |
| RBCS3 | 6.61 ± NA (1) | 65.64 ± NA (1) | 0.21 ± 0.01 (3) |
| STM | 5.76 ± NA (1) | 74.27 ± NA (1) | 0.19 ± 0.012 (2) |

### G881 (SEQ ID NO: 53 and 54)

Published background information. G881 (At4g31800) corresponds to AtWRKY18. There is no published literature beyond the general description of WRKY family members (Eulgem et al. (2000)).

Discoveries in Arabidopsis. The function of this gene was analyzed using transgenic plants in which G881 was expressed under the control of the 35S promoter. While one line of 35S::G881 plants showed a very marginal early flowering phenotype, all other lines were wild type morphologically. Arabidopsis 35S::G881 overexpressors were more susceptible to infection with the fungal pathogens Erysiphe orontii and Botrytis cinerea. These results, together with the fact that many WRKY family proteins are known to be involved in the disease signaling, implicate G881 in the disease response.

G881 is ubiquitously expressed, but appeared to be significantly induced in response to salicylic acid treatment. Additionally, in a soil drought microarray experiment, G881 was found to be repressed in Arabidopsis leaves during moderate drought stress, as well as after rewatering. G881 was highly (up to ˜14-fold) induced by salicylic acid in both root and shoot tissue. Induction was also observed in response to methyl jasmonate. Interestingly, in response to mannitol, cold or sodium chloride treatments, G881 was repressed at early timepoints (e.g., 0.5 hr and 1 hr), but induced to high levels at later timepoints (e.g., 4 and 8 hr).

Discoveries in tomato. Transgenic tomatoes expressing G881 under the AP1, LTP1, RBCS3 or STM promoters were analyzed for alteration in plant size, soluble solids and lycopene. The Cruciferin, PD and PG::G881 lines had poor fruit set, thus limiting the analysis to plant size. The fruit lycopene levels under the STM promoter rank in the 95th percentile among all lycopene measurements, and were higher than in any wild-type plant measured. Additionally, STM::G881 plants did not produce any ripe fruit. Arabidopsis 35 S:: These data indicate that G881 may be an important regulator affecting lycopene level in tomato fruit, with a negative impact on fruit maturation.

Other related data. G986 is a paralog of G881, however, this gene was not tested in the field trial. The function of 35S::G986 was analyzed in transgenic Arabidopsis and resulting plants were indistinguishable from wild-type controls in all assays performed. G986 was found to be ubiquitously expressed in all tissues tested.

TABLE 39
Data Summary for G881

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 5.71 ± 0.629 (2) | 70.06 ± 24.918 (2) | 0.08 ± 0.015 (3) |
| Cruciferin | NA | NA | 0.06 ± 0.026 (3) |
| LTP1 | 5.61 ± NA (1) | 74.7 ± NA (1) | 0.07 ± 0.004 (2) |
| PD | NA | NA | 0.03 ± 0.003 (2) |
| PG | NA | NA | 0.09 ± 0.004 (3) |
| RBCS3 | 5.29 ± 0.198 (2) | 70.69 ± 30.172 (2) | 0.09 ± 0.027 (2) |
| STM | 4.85 ± NA (1) | 108.85 ± NA (1) | 0.08 ± 0.046 (3) |

### G937 (SEQ ID NO: 55 and 56)

Published background information. G937 was identified in the sequence of BAC F14J22, GenBank accession number AC011807, released by the Arabidopsis Genome Initiative.

Discoveries in Arabidopsis. The function of this gene was analyzed using transgenic plants in which G937 was expressed under the control of the 35S promoter. The majority of 35S::G937 primary transformants were smaller than wild type, slightly slow developing, and produced thin inflorescence sterns that carried relatively few siliques. In later analysis, G937 was found to have a phenotype in a C/N sensing assay. Anthocyanin accumulation was slightly less than that observed in control wild-type seedlings in one of three lines tested. Thus, G937 might have a role in the response to nutrient limitation.

In our microarray analyses, G937 was found to be induced during drought stress and by sodium chloride treatment, and repressed by ABA treatment.

Discoveries in tomato. Plants expressing G937 under the PG promoter were in the 95th percentile for plant size. Analysis of G937 function and expression in Arabidopsis suggests that this gene plays a role in response to nutrient and drought stress. Therefore, the increased fitness of G937 transgenic tomatoes in field-grown condition may be related to drought tolerance and/or better nutrient utilization.

In contrast, AP1::G937 plants were noted to be compact and bear small fruit, although the plant volume measurements were within the normal range.

TABLE 40
Data Summary for G937

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.4 ± 0.327 (3) | 43.81 ± 22.048 (3) | 0.24 ± 0.061 (3) |
| AP1 | 5.77 ± NA (1) | 84.56 ± NA (1) | 0.3 ± 0.045 (2) |
| AS1 | 6 ± 0.146 (3) | 57.23 ± 17.205 (3) | 0.24 ± 0.051 (3) |
| PG | 5.07 ± 0.231 (3) | 44.18 ± 21.243 (3) | 0.33 ± 0.027 (3) |

### G989 (SEQ ID NO: 57 and 58)

Published background information. G989 corresponds to a predicted SCARECROW (SCR) gene regulator-like protein in annotated P1 clone MJC20 (AB017067), from chromosome 5 of Arabidopsis (Kaneko, et al. (1998)). This gene is a member of the SCARECROW branch of the SCR (or GRAS) phylogenetic tree, and it is closely related to SCR (Bolle, 2004). SCARECROW is involved in meristem maintenance and development, and has also been proposed to be involved in auxin regulation (Sabatini et al. (1999)).

Discoveries in Arabidopsis. The function of G989 was analyzed using transgenic plants in which G989 was expressed under the control of the 35S promoter. Plants overexpressing G989 were somewhat early flowering. The phenotype of the transgenic plants was wild type in all other assays performed.

G989 appeared to be expressed at highest levels in embryo tissue, and at low levels in all other tissues tested. Expression of G989 appeared to be induced in response to treatment with auxin, ABA, heat and drought, and to a lesser extent in response to salt treatment and osmotic stress. G989 was also shown to be up-regulated 3× in the leaves of drought-stressed plants in microarray experiments.

Discoveries in tomato. The size of the Cruciferin::G989 and STM::G989 tomato plants was markedly higher than of wild-type controls; ranking in the 95th percentile of all volumetric measurements. LTP1::G989 plants were also larger than wild type, but were not above the 95th percentile. All three of these promoters are associated with relatively low levels of expression in vegetative tomatoes. This indicates that low levels of G989 are effective in increasing biomass under field conditions.

Expression analyses indicated that G989 may be involved in stress response pathways.

Other relevant data: Bolle have suggested that G989 may also be involved in meristem/growth pathways Bolle (2004). One hypothesis is that G989, when expressed at relatively low levels and under adverse field conditions, may function to promote plant/meristem growth.

We have not yet identified a paralog of G989 in Arabidopsis. Our data showing induction of 0989 by stress treatments may indicate that G989 functions via stress pathways. Published information on the SCR family indicates that this family of genes function to promote meristem growth and development. Taken together, it is possible that G989 provides a link between stress response and the promotion of growth/biomass, and may promote plant growth in the periodically stressful environments common in the field.

TABLE 41
Data Summary for G989

| | Promoter summary: Avg. ± StD. (Count) | | |
|---|---|---|---|
| | Brix (g sugar/ | | |
| Promoter | 100 g sample) | Lycopene (ppm) | Volume (m³) |
| Cruciferin | 5.37 ± 0.368 (3) | 51.51 ± 17.663 (3) | 0.32 ± 0.015 (3) |
| LTP1 | 5.65 ± 0.318 (2) | 70.19 ± 8.726 (2) | 0.3 ± 0.057 (3) |
| STM | 5.41 ± NA (1) | 79.5 ± NA (1) | 0.32 ± NA (1) |

### G1007 (SEQ ID NO: 59 and 60)

Published background information. G1007 corresponds to gene At2g25820 (GenBank accession number AAC42248). Sakuma et al. (2002) categorized G1007 into the A4 subgroup of the AP2 transcription factor family, with the A family related to the DREB and CBF genes.

Discoveries in Arabidopsis. The function of this gene was analyzed using transgenic plants in which G1007 was expressed under the control of the 35S promoter. Overexpression of G1007 under control of the 35S promoter produced very small plants with poor fertility. Many plants arrested development in the vegetative phase and senesced without producing an inflorescence. Those lines that did bolt formed very spindly shoots bearing small poorly fertile flowers.

Global transcript profiling under a variety of stress conditions revealed repression of G1007 expression under severe drought only, with repression maintained but reduced during early recovery from drought. G1007 transcripts were below detectable level in all tissues examined by RT-PCR.

Discoveries in tomato. 35S::G1007 lines had poor fruit set, thus limiting the analysis to plant size. Lycopene content in fruit and Brix were greater than that in wild type controls in plants expressing G1007 under the AP1 promoter, with a rank in the 95th percentile among all measurements. In addition, Brix was also higher in G1007 overexpressors under the Cruciferin promoter. Plant size in Arabidopsis and tomato seedlings were also dramatically reduced upon overexpression of G1007 under the constitutive 35S promoter. In the most severe phenotypes, Arabidopsis plants senesced without producing an inflorescence. These data indicate that G1007 may be an important regulator affecting lycopene and soluble solids in tomato fruit.

Other related data. G1836 is a paralog of G1007, however, this gene was not tested in the field trial. Overexpression of G1846 in Arabidopsis caused significant growth defects. In general, transformants were smaller, and the reduced size of the inflorescences resulted in only a low seed yield.

TABLE 42
Data Summary for G1007

| | Promoter summary: Avg. ± StD. (Count) | | |
|---|---|---|---|
| | Brix | | |
| Promoter | (g sugar/100 g sample) | Lycopene (ppm) | Volume (m³) |
| 35S | NA | NA | 0.18 ± NA (1) |
| AP1 | 6.42 ± NA (1) | 100.75 ± NA (1) | 0.17 ± 0.092 (3) |
| Cruciferin | 6.67 ± NA (1) | 26.35 ± NA (1) | 0.16 ± 0.023 (3) |

### G1053 (SEQ ID NO: 61 and 62)

Published background information. G1053 was identified in the sequence of BAC T7123, GenBank accession number U89959, released by the Arabidopsis Genome Initiative.

Discoveries in Arabidopsis. The boundaries of G1053 in BAC T7123 were experimentally determined and the function of G1053 was analyzed using transgenic plants in which this gene was expressed under the control of the 35S promoter. G1053 overexpressing lines appeared to be small, slow growing and displayed curled leaves and spindly stems.

G1053 expression seemed to be restricted to shoots and siliques. Microarray analysis revealed no significant change (p-value<0.01) in G1053 expression in all conditions examined.

Discoveries in tomato. 35S, AS1, LTP1, PG and RCBS3::G1053 lines had poor fruit set, thus limiting the analysis to plant size. Soluble solids under the Cruciferin promoter was higher than the highest wild type level and ranked in the 95th percentile among all Brix measurements. In addition, under the AP1 promoter, plants were larger wild type controls in the field and ranked in the 95th percentile among all volumetric measurements. In Arabidopsis, G1053 expression seemed to be restricted to shoots and siliques. G1053 overexpressing Arabidopsis lines were small, slow growing and had curled leaves and spindly stems. These data indicate that G1053 may be an important regulator affecting plant biomass and soluble solids in tomato fruit.

Other related data. The paralog of G1053, G2629, was not tested in field trial. In Arabidopsis, overexpression of G2629 produced no consistent effects on Arabidopsis morphology or physiology in all assays performed.

TABLE 43
Data Summary for G1053

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | NA | NA | 0.25 ± 0.083 (3) |
| AP1 | 5.56 ± 1.075 (2) | 69.94 ± 0.502 (2) | 0.46 ± 0.178 (3) |
| AS1 | NA | NA | 0.36 ± 0.12 (3) |
| Cruciferin | 6.55 ± NA (1) | 53.48 ± NA (1) | 0.2 ± NA (1) |
| LTP1 | NA | NA | 0.24 ± 0.102 (3) |
| PG | NA | NA | 0.27 ± 0.006 (3) |
| RBCS3 | NA | NA | 0.22 ± 0.097 (3) |
| STM | 6.16 ± 0.085 (2) | 94.98 ± 12.084 (2) | 0.28 ± 0.09 (3) |

### G1078 (SEQ ID NO: 63 and 64)

Published background information. G1078 is the published bZIPt2 cDNA described by Lu and Ferl (1995).

Discoveries in Arabidopsis. The function of G1078 was analyzed using transgenic plants in which G1078 was expressed under the control of the 35S promoter. The phenotype of these transgenic plants was wild type in all assays performed. G1078 appeared to be constitutively expressed in all tissues and environmental conditions tested by RT-PCR. However, GeneChip experiment indicated the G1078 is repressed by most abiotic stress treatments, including drought, ABA, and mannitol.

Discoveries in tomato. Cruciferin, PG and STM::G1078 lines had poor fruit set, thus limiting the analysis to plant size. Fruit lycopene level under the RBCS3 promoter was higher than the highest wild type and ranked in the 95th percentile among all measurements. Expression of G1078 under the AP1 and STM promoters result in plants with longer vegetative period. Arabidopsis 35S::G1078 transgenic plants were wild type phenotype in all assays performed. These data indicated that G1078 may be an important regulator affecting lycopene in ripening tomato fruit.

Other related data. The paralog of G1078, G577, was not tested in tomato in the present field trial. Overexpression of G577 in Arabidopsis produced a range of effects on growth and development, including slight smallness and slower growth, dark green leaves with elevated levels of anthocyanins and wrinkled curled leaves that formed yellow patches. It is possible that G577 is a regulator of anthocyanins in Arabidopsis.

TABLE 44
Data Summary for G1078

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 5.59 ± 0.495 (2) | 76.07 ± 9.136 (2) | 0.26 ± 0.043 (3) |
| Cruciferin | NA | NA | 0.14 ± 0.032 (2) |
| PG | NA | NA | 0.17 ± 0.088 (3) |
| RBCS3 | 5.97 ± 0.359 (3) | 105.46 ± 8.59 (3) | 0.23 ± 0.075 (3) |
| STM | NA | NA | 0.22 ± 0.048 (3) |

### G1226 (SEQ ID NO: 65 and 66)

Published background information. G1226 corresponds to AtbHLH057, as described by Heim et al., (2003) and Toledo-Ortiz et al. (2003), which describe the Arabidopsis bHLH gene family.

Discoveries in Arabidopsis. Overexpression of G1226 under control of the 35S promoter in Arabidopsis conferred an earlier flowering phenotype and a statistically significant elevation in seed oil content.

In a series of stress challenge array background experiments, G1226 was found to be induced during recovery from drought treatment, and repressed in shoots of plants treated with ABA, SA or cold. RT-PCR analysis indicates that G1226 is constitutively expressed in all tissues, except in root where it is undetected.

Discoveries in tomato. 35S and PG::G1226 lines had poor fruit set, thus limiting the analysis to plant size. Lycopene content in fruit was greater than that in wild type controls in plants expressing G1226 under the RBCS3 promoter, with a rank in the 95th percentile among all measurements. These data indicate that G1226 may be an important regulator affecting lycopene in ripening tomato fruit.

TABLE 45
Data Summary for G1226

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | NA | NA | 0.14 ± 0.02 (3) |
| Cruciferin | 5.32 ± 1.111 (3) | 65.88 ± 32.849 (3) | 0.25 ± 0.05 (3) |
| PG | NA | NA | 0.2 ± 0.043 (3) |
| RBCS3 | 5.69 ± 0.113 (2) | 102.73 ± 25.095 (2) | 0.27 ± 0.023 (3) |

### G1273 (SEQ ID NO: 67 and 68)

Published background information. G1273 (At2g37260, AtWRKY44) corresponds TRANSPARENT TESTA GLABRA2 (TTG2; Johnson et al. (2002)). From the work of Johnson et al., it is known that TTG2 is involved in trichome development and tanin/mucilage production in seed coat tissue. TTG2 is strongly expressed in trichomes throughout their development, in the endothelium of developing seeds (in which tannin is later generated) and subsequently in other layers of the seed coat, as well as in the atrichoblasts of developing roots. TTG2 acts downstream of the trichome initiation genes TTG1 and GLABROUS1. In the seed coat, TTG2 expression requires TTG1 function in the production of tannin. In ttg2 mutants, synthesis of tannins, but not anthocyanins is disrupted. Therefore, the authors speculate that TTG2 regulates the expression of gene(s) involved in the tannin biosynthetic pathway after the leucoanthocyanidin branch point.

Discoveries in Arabidopsis. G1273 was found to be expressed in a variety of tissues (eaves, flowers, embryo, silique, germinating seedling) at apparently low levels. Additionally, in a soil drought microarray experiment, G1273 was found to be induced 4.6-fold (p<0.01) in the leaf tissue of plants exposed to moderate drought conditions.

The function of G1273 was studied using transgenic plants in which the gene was expressed under the control of the 35S promoter. No consistent morphological alterations were detected in G1273 overexpressing plants. G1273 transgenic lines behave similarly to wild-type controls in all physiological and biochemical assays performed.

Discoveries in tomato. PG::G1273 lines had poor fruit set thus, limiting the analysis to plant size. The fruit lycopene levels of G1273 overexpressors under the control of the AP1 promoter ranked in the 95th percentile among all lycopene measurements, and were higher than in any wild-type plant measured. These data indicate that G1273 may be an important regulator affecting lycopene in ripening tomato fruit.

TABLE 46
Data Summary for G1273

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.55 ± 0.75 (2) | 36.78 ± 14.913 (2) | 0.27 ± 0.033 (3) |
| AP1 | 5.94 ± NA (1) | 110.56 ± NA (1) | 0.21 ± NA (1) |
| Cruciferin | 5.62 ± 0.113 (2) | 51.61 ± 12.113 (2) | 0.22 ± 0.047 (3) |
| PD | 5.87 ± 0.46 (2) | 59.13 ± 44.774 (2) | 0.22 ± 0.01 (3) |
| PG | NA | NA | 0.18 ± 0.062 (3) |
| STM | 5.55 ± 0.276 (3) | 75.44 ± 17.32 (3) | 0.24 ± 0.051 (3) |

### G1324 (SEQ II) NO: 69 and 70)

Published background information. The full-length cDNA sequence of G1324 (At1g68320) was discovered from a partial published clone corresponding to AtMYB62. Reverse-Northern data suggest that this gene is expressed at low levels in siliques (Kranz et al. (1998)).

Discoveries in Arabidopsis. As determined by RT-PCR, G1324 is expressed in flowers, siliques and seedlings. No expression of G1324 was detected in the other tissues tested. G1324 expression is not induced under any environmental stress-related treatment tested, based on RT-PCR and microarray analysis.

The function of G1324 was analyzed using transgenic plants in which the gene was expressed under the control of the 35S promoter. The phenotype of these transgenic plants was wild type in all assays performed. Morphological analysis showed that the primary transformants of G1324 were small, dark green, and late flowering. However, these phenotypes were apparently unstable, as T2 lines 1, 6, and 9 were scored as wild type.

Discoveries in tomato. The fruit lycopene level under the PG promoter was higher than the highest wild type level and ranked in the 95th percentile among all lycopene measurements. In Arabidopsis, 35S::G1324 transgenic plants were wild type in all assays performed. These data indicated that G1324 may be an important regulator affecting lycopene in ripening tomato fruit.

Other related data. The paralog of G1324, G2893, was not tested in tomato in the present field trial. In Arabidopsis, transgenic plants overexpressing G2893 were generally small, slightly dark green, and produced flowers with a variety of abnormalities in organ identity, organ number, and organ fusions. Due to the small size and poor fertility of some T2 lines, insufficient material was available for a complete set of biochemical assays. 35S::G2893 plants were wild type in the physiology assays performed.

TABLE 47
Data Summary for G1324

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.03 ± 0.777 (3) | 76.73 ± 12.19 (3) | 0.07 ± 0.016 (3) |
| AP1 | 5.86 ± 0.304 (2) | 70.34 ± 51.47 (2) | 0.09 ± 0.026 (3) |
| AS1 | 5.39 ± NA (1) | 74.16 ± NA (1) | 0.08 ± 0.028 (3) |
| Cruciferin | 5.34 ± 0.503 (3) | 55.36 ± 5.078 (3) | 0.1 ± 0.031 (3) |
| LTP1 | 5.79 ± 0.219 (2) | 57.58 ± 7.828 (2) | 0.1 ± 0.034 (2) |
| PD | 5.76 ± 0.82 (2) | 60.83 ± 5.148 (2) | 0.12 ± 0.001 (2) |
| PG | 5.52 ± NA (1) | 112.42 ± NA (1) | 0.08 ± 0.049 (2) |

### G1328 (SEQ ID NO: 71 and 72)

Published background information. The full-length cDNA sequence of G1328 (At4g05100) was determined from a partial published clone corresponding to MYB74. Reverse-Northern data suggest that this gene is detected in mature leaves, cauline leaves, and siliques; it appeared to be induced in mature leaves in response to drought treatment, and in etiolated seedlings in response to light (Kranz et al. (1998)). The promoter sequence of G1328 has been reported to contain ABRE, CE1, and W box cis-elements, which are known to be involved in stress responses (Denekamp and Smeekens, 2003).

Discoveries in Arabidopsis. The function of G1328 was analyzed using transgenic plants in which the gene was expressed under the control of the 35S promoter. Arabidopsis plants overexpressing G1328 in primary transformants displayed a phenotype of numerous secondary inflorescence meristems that produced extra leaves and short secondary bolts. However, this phenotype was unstable in the T2 generation. The phenotype of these transgenic plants was wild type in all physiological assays performed.

RT-PCR analysis suggests that endogenous G1328 transcripts were found at very low levels in roots, embryos, seedlings and siliques. Microarray experiments showed that G1328 transcript accumulation was induced by ABA, drought, and osmotic stress treatments; it was also slightly induced in the G912 overexpressing lines.

Discoveries in tomato. 35S and RBCS3::G1328 lines had poor fruit set, thus limiting the analysis to plant size. Under the RBCS3 promoter, overall plant size ranked in the 95th percentile among all measurements. These data indicate that G1328 may be an important regulator affecting plant biomass in tomato.

Other related data. The paralog of G1328, G198, was not tested in tomato in the present field trial. In Arabidopsis, the phenotype of G198 overexpressors was wild-type for all assays performed. The morphological phenotype of G198 overexpressors suggests this gene could function in flowering time. G198 as a similar expression pattern as G1328 (mainly induced by drought, ABA, and osmotic stress treatments), as determined by RT-PCR and microarray analysis.

TABLE 48
Data Summary for G1328

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | NA | NA | 0.18 ± 0.083 (2) |
| AP1 | 5.41 ± 0.049 (2) | 57.34 ± 30.561 (2) | 0.27 ± 0.059 (3) |
| AS1 | 5.24 ± 0.064 (2) | 81.69 ± 1.435 (2) | 0.25 ± 0.051 (3) |
| RBCS3 | NA | NA | 0.32 ± NA (1) |

### G1444 (SEQ ID NO: 73 and 74)

Published background information. The sequence of G1444 (At2g42040) was initially obtained from the Arabidopsis sequencing project, GenBank accession number U90439.3 (GI: 20198316), based on sequence similarity to the rice Growth-regulating-factor1 (GRF1, GI: 6573149; Knaap et al. (2000)). Nine of the ten members of the Arabidopsis atGRF family were recently published by Kim et al. (2003). Their analysis of the gene family did not include G1444, a phylogenetically distant member of the atGRF family with the characteristic WRC domain. Detailed characterization of 35S::atGRF1 and 35S::atGRF2 overexpressor in Arabidopsis revealed a significant increased in leaf/cotyledon surface area, 35-135% greater than in wild type control, and delayed shoot development (Kim et al, 2003). In the triple grf1 (G1439), grf2 (G1868), grf3 (G2334) mutants the opposite phenotype was observed in addition to delayed leaf development and fusion of cotyledon.

Discoveries in Arabidopsis. The function of G1444 was analyzed by ectopic overexpression in Arabidopsis. The characterization of G1444 transgenic lines revealed no significant morphological, physiological or biochemical changes when compared to wild-type controls.

The analysis of the endogenous level of G1444 transcripts by RT-PCR revealed low, constitutive expression in all tissues tested. Microarray analysis revealed a significant (p-value<0.01) reduction in G1444 expression level in leaves of soil-drought treated plants. No changes in G1444 expression were observed in response to other biotic and abiotic treatments.

Discoveries in tomato. In plants expressing G1444 under the leaf (LTP1) promoter, soluble solids (Brix measurement) in fruit was greater than that in wild type controls; ranking in the 95th percentile among all measurements. Transgenic tomato plants expressing G1444 under the constitutive (35S), meristem (AS1) and green-tissue (RBCS3) promoters were larger than wild type controls; ranking among the 95th percentile of all measurements. Supporting this phenotype, LTP1 and PD lines were both larger than controls (90th percentile). Transgenic tomato plants expressing G1444 under the meristem (STM) promoter also displayed smaller fruits.

Other related data. There is no close paralog for G1444. However, the size-related phenotype in tomato is supported by observation made in transgenic Arabidopsis constitutively overexpression a number of genes of the GRF-like family. Transgenic Arabidopsis overexpressing G1439 (atGRF1), G1868 (atGRF2), G1863, G2334 and G1865 have all shown alteration in leaf shape and coloration. They also are delayed in the onset of flowering.

TABLE 49
Data Summary for G1444

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.98 ± 0.794 (3) | 43.79 ± 6.021 (3) | 0.33 ± 0.015 (3) |
| AP1 | 5.81 ± NA (1) | 58.89 ± NA (1) | 0.25 ± NA (1) |
| AS1 | 5.45 ± 0.411 (3) | 45.23 ± 21.765 (3) | 0.32 ± 0.098 (3) |
| LTP1 | 6.63 ± 0.262 (2) | 56.77 ± 23.78 (2) | 0.3 ± 0.026 (3) |
| PD | 5.31 ± 0.601 (3) | 57.66 ± 10.019 (3) | 0.29 ± 0.084 (3) |
| RBCS3 | 5.45 ± NA (1) | 37.46 ± NA (1) | 0.32 ± 0.005 (2) |
| STM | 5.5 ± NA (1) | 49.65 ± NA (1) | 0.21 ± 0.187 (3) |

### G1462 (SEQ ID NO: 75 and 76)

Published background information. G1462 was identified in the sequence of BAC T13D8, GenBank accession number AC004473, released by the Arabidopsis Genome Initiative. It also corresponds to the AGI locus of At1g60300. A comprehensive analysis of NAC family transcription factors was recently published by Ooka et al. (2003) but did not include G1462. G1462 and G1463 are both tightly clustered to three other genes (G1461, G1464, and G1465) in a phylogenetic alignment and most likely arose through tandem gene duplication events.

Discoveries in Arabidopsis. The complete sequence of G1462 was determined. The function of this gene was analyzed using transgenic plants in which G1462 was expressed under the control of the promoter. The phenotype of these transgenic plants was wild-type in all assays performed.

G1462 transcript can be detected at very low levels in flower tissue only. The expression of G1462 in leaf does not respond to any environmental conditions tested.

Discoveries in tomato. Soluble solids and lycopene levels of plants overexpressing G1462 under the regulation of the AP1 promoter were significantly above wild type levels and in the 95th percentile of all measurements. A closely related paralog of G1462, G1463, demonstrated a significant increase in plant size when expressed from STM and RBCS3 promoters. These data indicate that G1462 may be an important regulator affecting size, lycopene and soluble solids in tomato.

Other related data. G1462 is highly related to four other putative paralogs. Included in these are G1461, G1463, G1464 and G1465. All genes within the G1462 clade are tightly clustered on chromosome number one suggesting that they may have originated through tandem gene duplication events. G1465 is most related to G1462 in a phylogenetic analysis and displayed alterations in compositions of leaf fatty acids in the phase I genomics screen. In addition, G1463 showed premature senescence. RT-PCR analysis of the endogenous levels of G1464 in leaves indicates that this gene could be induced by ABA, auxin, cold, drought, and salt.

TABLE 50
Data Summary for G1462

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 6.36 ± NA (1) | 97.53 ± NA (1) | 0.22 ± 0.086 (3) |
| Cruciferin | 5.91 ± 0.424 (2) | 76.09 ± 11.342 (2) | 0.25 ± 0.064 (3) |

### G1463 (SEQ ID NO: 77 and 78)

Published background information. G2052 was identified in the sequence of BAC clone:F10E10, GenBank accession number AB028605, released by the Arabidopsis Genome Initiative. It also corresponds to the AGI locus of AT1G60380. A comprehensive analysis of NAC family transcription factors was recently published by Ooka et al. (2003) but did not include G1463. G1463 and G1462 are both tightly clustered to three other genes (G1461, G1464, and G1465) in a phylogenetic alignment and most likely arose through tandem gene duplication events.

Discoveries in Arabidopsis. The function of G1463 was analyzed using transgenic plants in which the gene was expressed under the control of the 35S promoter. In later stage plants, overexpression of G1463 resulted in premature senescence of rosette leaves. Under continuous light conditions, the most severely affected plants started to senesce approximately 10 days earlier than wild-type controls, at around 30 days after sowing. Additionally, 35S::G1463 plants formed slightly thin inflorescence stems and showed a relatively low seed yield.

G1463 expression was analyzed by transcriptional profiling using microarrays. In experiments where Arabidopsis seedlings (ecotype col) were treated with a panel of stresses, G1463 transcript levels were significantly repressed in response to ABA, Methyl Jasmonate, NaCl and Cold. Although both shoot and root tissues were assayed, G1463 expression was only differentially regulated in the roots.

Discoveries in tomato. LTP1 and PG::G1463 lines had poor fruit set, thus limiting the analysis to plant size. Under the regulation of the both STM and RBCS3 promoters, significant increases in G1463-overexpressing plant size were observed. Tomato seedlings expressing G1463 under the constitutive 35S promoter were smaller than wild type controls.

A closely related paralog of G1463, G1462, revealed a significant increase in soluble solids and lycopene when expressed from the AP1 promoter.

Other related data. G1463 is highly related to four other putative paralogs. Included in these are G1461, G1462, G1464 and G1465. All genes within the G1463 clade are tightly clustered on chromosome number one suggesting that they may have originated through tandem gene duplication events. G1464 is most related to G1463 in a phylogenetic analysis. G1465 displayed alterations in compositions of leaf fatty acids in the phase I genomics screen. RT-PCR analysis of the endogenous levels of G1464 in leaves indicates that this gene could be induced by ABA, auxin, cold, drought, and salt. This transcriptional response of G1464 shows strikingly similar characteristics to G1463 transcriptional profiling in our microarray studies, suggesting that there may be some overlap in function between the two genes.

TABLE 51
Data Summary for G2425

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.79 ± NA (1) | 63.32 ± NA (1) | 0.22 ± 0.055 (3) |
| AP1 | 5.92 ± 0.417 (2) | 85.42 ± 20.195 (2) | 0.27 ± 0.064 (3) |
| AS1 | 5.19 ± NA (1) | 60.53 ± NA (1) | 0.21 ± 0.045 (3) |
| Cruciferin | 4.45 ± NA (1) | 35.72 ± NA (1) | 0.23 ± 0.022 (3) |
| LTP1 | NA | NA | 0.14 ± 0.055 (3) |
| PD | NA | NA | 0.25 ± 0.019 (3) |
| PG | 5.03 ± 0.382 (2) | 48.08 ± 9.108 (2) | 0.2 ± 0.027 (3) |
| RBCS3 | 5.05 ± 0.042 (2) | 44.77 ± 7.87 (2) | 0.5 ± 0.079 (3) |
| STM | 4.85 ± 1.073 (3) | 56.2 ± 9.72 (3) | 0.38 ± 0.162 (3) |

### G1481 (SEQ ID NO: 79 and 80)

Published background information. G1481 was identified as a gene in the sequence of the P1 clone M4I22 (Accession Number AL030978), released by the European Union Arabidopsis Sequencing Project.

Discoveries in Arabidopsis. The sequence of G1481 was experimentally determined, and the function of this gene was analyzed using transgenic plants in which G1481 was expressed under the control of the 35S promoter. 35S::G1481 plants appeared identical to controls in all assays examined.

RT-PCR analysis indicated G1481 was expressed in all tissues except shoots. G1481 was expressed at higher levels in embryonic tissue. G1481 was not significantly induced by any treatment examined using RT-PCR. Microarray experiments indicated that G1481 was induced by drought and cold.

Discoveries in tomato. The fruit Brix level under the RBCS3 promoter was higher than the highest wild type level and ranked in the 95th percentile among all Brix measurements. STM::G1481 fruits also showed higher soluble solids than controls (above 75th percentile). These data indicate that G1481 may be an important regulator affecting soluble solids in tomato fruit.

Other related data. The paralog of G1481, G900, was tested in tomato in the present field trial. Overexpression of G900 under the 35S promoter in Arabidopsis produced a range of effects on growth and development, including small, slow growing plants with rather narrow dark green leaves. Later, these plants developed somewhat thin inflorescence stems and had a relatively low seed yield. Overexpression of G900 in tomato under the STM promoter also produced small plants.

TABLE 52
Data Summary for G1481

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.63 ± 0.556 (3) | 53.18 ± 2.615 (3) | 0.2 ± 0.029 (3) |
| AP1 | 5.18 ± 0.329 (3) | 71.23 ± 10.794 (3) | 0.22 ± 0.05 (3) |
| LTP1 | 5.56 ± 0.332 (2) | 66.16 ± 6.901 (2) | 0.19 ± 0.025 (3) |
| PD | 5.24 ± 0.458 (3) | 63.34 ± 0.875 (3) | 0.19 ± 0.019 (3) |
| RBCS3 | 6.6 ± NA (1) | 81.03 ± NA (1) | 0.15 ± 0.069 (3) |
| STM | 6.27 ± 0.573 (2) | 78.78 ± 2.864 (2) | 0.18 ± 0.048 (3) |

### G1504 (SEQ ID NO: 81 and 82)

Published background information. G1504 was identified as a gene in the sequence of BAC AC006283, released by the Arabidopsis Genome Initiative.

Discoveries in Arabidopsis. The sequence of G1504 was experimentally determined and the function of G1504 was analyzed using transgenic plants in which G1504 was expressed under the control of the 35S promoter. Plants overexpressing G1504 appeared to be identical to controls in all assays.

RT-PCR analysis indicates that G1504 is expressed in flowers and embryos and may be slightly induced in leaves by cold, drought and osmotic stresses. This observation is not supported by microarray analysis, which shows no significant changes (p-value<0.01) in G1505 expression levels.

Discoveries in tomato. The AS1::G1504 lines had poor fruit set, thus limiting the analysis to plant size. Under the STM promoter, plant size ranked in the 95th percentile among all measurements. Overexpression of G1504 under the AS1 promoter produced only green fruit; no red fruit were obtained. Fruits of AP1::G1504 tomato plants split before maturity. These data indicate that G1504 may be an important regulator affecting plant biomass and/or fruit development.

Other related data. Two paralogs of G1504, G2442 and G2504 were not tested in tomato in the present field trial. Both 35S::G2504 and 35S::2442 plants showed no consistent differences to wild-type in all morphological and physiological analyses that were performed.

TABLE 53
Data Summary for G1504

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 4.6 ± NA (1) | 84.73 ± NA (1) | 0.19 ± 0.049 (3) |
| AS1 | NA | NA | 0.23 ± 0.034 (3) |
| RBCS3 | 5.75 ± 0.711 (3) | 67.18 ± 16.545 (3) | 0.2 ± 0.044 (3) |
| STM | 5.5 ± 0.085 (3) | 66.59 ± 20.772 (3) | 0.33 ± 0.053 (3) |

### G1543 (SEQ ID NO: 83 and 84)

Published background information. G1543 corresponds to AT2G01430 and encodes a HD-ZIP class II HD protein. The gene is annotated as ATHB-17 at the TAIR site.

Discoveries in Arabidopsis. G1543 was analyzed during our Arabidopsis genomics program; overexpression of the gene produced short compact architecture, a dark coloration and an increase in leaf chlorophyll and carotenoid levels. Notably, RT-PCR experiments revealed that G1543 expression is up-regulated in response to auxin applications. The morphological phenotype, along with the expression data, might implicate G1543 as a component of a growth or developmental response to auxin. Subsequently, G1543 was found to be significantly up-regulated in response to ABA and NaCl, during microarray studies, suggesting that the gene might have a role in response pathways to abiotic stress.

Discoveries in tomato. A notable increase in biomass, as determined by measurements of plant volume, was observed in LTP1::G1543 and PG::G1543 tomato lines relative to wild type. Overall fruit-set for LTP1::G1543 and PG::G1543 was low, and thus increases in vegetative biomass may be an indirect result of a decrease in fruit-set.

Other related data. G1543 was recognized to be of particular interest during Arabidopsis studies, since 35S::G1543 lines exhibited a dark green coloration and a compact architecture. Biochemical assays reflected the changes in leaf color noted during morphological analysis; increased levels of leaf chlorophylls and carotenoids were detected in the 35S::G1543 lines. In many crops for which the vegetative portion of the plant comprises the product, increased biomass would improve yield.

There are no highly related paralogs to G1543 in the Arabidopsis genome but we have identified potential orthologs in soy, rice, and maize. These sequences include G3524 (SEQ ID NO: 341 and 342, conserved domain coordinates 60-120, conserved domain 88% identical to the conserved domain of G1543), G3490 (SEQ ID NO: 327 and 328, conserved domain coordinates 60-120, conserved domain 80% identical to the conserved domain of G1543), and G3510 (SEQ ID NO: 825 and 826, conserved domain coordinates 74-134, conserved domain 80% identical to the conserved domain of G1543).

TABLE 54
Data Summary for G1543

| | Promoter summary: Avg. ± StD. (Count) | | |
|---|---|---|---|
| | Brix | | |
| Promoter | (g sugar/100 g sample) | Lycopene (ppm) | Volume (m³) |
| AS1 | 5.18 ± NA (1) | 86.09 ± NA (1) | 0.3 ± 0.036 (3) |
| Cruciferin | 5.48 ± NA (1) | 83.05 ± NA (1) | 0.17 ± 0.097 (3) |
| LTP1 | NA | NA | 0.34 ± 0.102 (3) |
| PG | 4.44 ± NA (1) | 68.52 ± NA (1) | 0.32 ± 0.063 (3) |
| STM | 4.66 ± NA (1) | 60 ± NA (1) | 0.21 ± 0.045 (3) |

### G1635 (SEQ ID NO: 85 and 86)

Published background information. G1635 (At5g17300) was identified in the sequence of BAC MKP11 (GenBank accession number AB005238), released by the Arabidopsis Genome Initiative.

Discoveries in Arabidopsis. The function of this gene was analyzed using transgenic plants in which G1635 was expressed under the control of the 35S promoter. Overexpression of G1635 in transgenic Arabidopsis caused numerous morphological changes, including reduced apical dominance, reduced bolt elongation, narrow rosette leaves, and poor fertility. The phenotype of these transgenic plants was wild-type in all biochemical and physiological assays performed. G1635 is expressed in all tissues of soil-grown plants tested by RT-PCR. Microarray analysis revealed that G1635 is induced by drought, ABA, mannitol, and cold treatments.

Discoveries in tomato. The fruit Brix levels under the LTP1 and PG promoters were close to the highest wild type level and ranked in the 95th percentile among all Brix measurements. In addition, under the AP1 and PD promoters, plant size ranked in the 95th percentile among all plant size measurements. The fruit lycopene level under the STM promoter was higher than the highest wild type level and ranked in the 95th percentile among all lycopene measurements. These tomato plants appeared bushier, possibly due to an increase in lateral branching. Significantly, the large plant size in the AP1::G1635 and PD::G1635 was correlated with a very high fruitset. This indicates a synergy between plant biomass and fruit-set in these lines. Similarly, the high lycopene phenotype of the STM::G1635 plants was also correlated with good fruitset.

TABLE 55
Data Summary for G1635

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | NA | NA | 0.21 ± 0.019 (3) |
| AP1 | 5.64 ± 0.457 (3) | 53.34 ± 21.227 (3) | 0.32 ± 0.068 (3) |
| AS1 | 5.23 ± NA (1) | 58.77 ± NA (1) | 0.27 ± 0.145 (3) |
| Cruciferin | 5.55 ± NA (1) | 55.73 ± NA (1) | 0.23 ± 0.135 (3) |
| LTP1 | 6.31 ± NA (1) | 90.87 ± NA (1) | 0.2 ± 0.016 (3) |
| PD | 4.76 ± 0.522 (3) | 55.56 ± 13.367 (3) | 0.33 ± 0.203 (3) |
| PG | 6.3 ± NA (1) | 73.78 ± NA (1) | 0.21 ± 0.012 (3) |
| RBCS3 | 5.46 ± 0.29 (2) | 73.81 ± 17.501 (2) | 0.27 ± 0.041 (3) |
| STM | 5.62 ± 0.629 (2) | 121.53 ± 11.795 (2) | 0.28 ± 0.073 (3) |

### G1638 (SEQ ID NO: 87 and 88)

Published background information. G1638 (At2g38090) was identified in the sequence of BAC F16M14 (GenBank accession number AC003028), released by the Arabidopsis Genome Initiative.

Discoveries in Arabidopsis. The complete sequence of G1638 was expressed in Arabidopsis under the control of the 35S promoter. The phenotype of transgenic Arabidopsis plants overexpressing G1638 was wild-type in all assays performed. G1638 is moderately expressed in all tissues and under all conditions tested in RT-PCR experiments. Microarray experiments revealed no induction or repression patterns related to stress or hormone treatment, or in any of the transcription factor overexpressing lines.

Discoveries in tomato. The fruit lycopene level in PG::G1638 plants was higher than the highest wild type level and ranked in the 95th percentile among all lycopene measurements.

TABLE 56
Data Summary for G1638

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | NA | NA | 0.16 ± 0.038 (3) |
| Cruciferin | 4.59 ± NA (1) | 43.54 ± NA (1) | 0.29 ± 0.023 (3) |
| LTP1 | NA | NA | 0.16 ± 0.015 (3) |
| PD | 5.29 ± 0.382 (2) | 53.51 ± 6.378 (2) | 0.27 ± 0.094 (3) |
| PG | 5.86 ± 0.141 (2) | 119.22 ± 7.446 (2) | 0.23 ± 0.002 (2) |
| STM | 5.17 ± NA (1) | 58.99 ± NA (1) | 0.28 ± 0.119 (2) |

### G1640 (SEQ ID NO: 89 and 90)

Published background information. G1640 (At5g49330) was identified in the sequence of BAC K21P3 (GenBank accession number AB016872), released by the Arabidopsis Genome Initiative. This gene has since been given the name AtMYB111 by Stracke et. al. (2001).

Discoveries in Arabidopsis. The function of this gene was analyzed using transgenic plants in which G1640 was expressed under the control of the 35S promoter. The transgenic plants were morphologically indistinguishable from wild-type plants. They were wild-type in all physiological assays performed. Biochemical analysis suggests that overexpression of G1640 in Arabidopsis results in an increase in seed oil content and a decrease in seed protein content, at least in one of the three lines analyzed. This result should be repeated on additional lines and in additional seed lots.

As determined by RT-PCR, G1640 was expressed in leaves, flowers, embryos and siliques. No expression of G1640 was detected in the other tissues tested nor was the gene induced in rosette leaves by any stress-related treatment, as determined by RT-PCR. Microarray analysis showed that G1640 may be induced by cold treatment and slightly repressed by ABA.

Discoveries in tomato. The plant size under the PG promoter was close to the highest wild type level and ranked in the 95th percentile among all biomass measurements. PG::G1640 plants had low fruit-set.

TABLE 57
Data Summary for G1640

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.48 ± NA (1) | 69.86 ± NA (1) | 0.23 ± 0.177 (3) |
| AS1 | 6.19 ± 0.481 (2) | 67.68 ± 12.735 (2) | 0.34 ± 0.126 (3) |
| Cruciferin | 6.08 ± 0.539 (3) | 94.61 ± 22.549 (3) | 0.29 ± 0.097 (3) |
| PG | NA | NA | 0.28 ± 0.098 (3) |

### G1645 (SEQ ID NO: 91 and 92)

Published background information. G1645 (At1g26780) is a member of the (R1)R2R3 subfamily of MYB transcription factors. G1645 was identified in the sequence of BAC T24P13 (GenBank accession number AC006535), released by the Arabidopsis Genome Initiative. This gene has since been given the name AtMYB117 by Stracke et. al. (2001).

Discoveries in Arabidopsis. The function of G1645 was analyzed using transgenic Arabidopsis plants in which the gene was expressed under the control of the 35S promoter. Overexpression of G1645 produced marked changes in Arabidopsis leaf, flower, and shoot development. These effects were observed, to varying extents, in the majority of 35S::G1645 primary transformants.

At early stages, many 35S::G1645 T1 lines appeared slightly small and most had rather rounded leaves. However, later, as the leaves expanded, in many cases they became misshapen and highly contorted. Furthermore, some of the lines grew slowly and bolted markedly later than control plants. Following the switch to flowering, 35S::G1645 inflorescences often showed aberrant growth patterns, and had a reduction in apical dominance. Additionally, the flowers were frequently abnormal and had organs missing, reduced in size, or contorted. Pollen production also appeared poor in some instances. Due to these deficiencies, the fertility of many of the 35S::G1645 lines was low and only small numbers of seeds were produced.

Since 35S::G1645 primary transformants were obtained at a late stage in the research program, and many of the T1 lines developed slowly, therefore physiological assays were performed on the individual lines only. Overexpression of G1645 resulted in a low germination efficiency during a 32° C. heat stress assay.

As determined by RT-PCR, G1645 is expressed in flowers, embryos, germinating seeds, and siliques. No expression of G1645 was detected in the other tissues tested. G1645 expression appeared to be repressed in rosette leaves infected with Erysiphe orontii. No significant increases or decreases in G1645 expression were detected in any of the microarray experiments.

Discoveries in tomato. The fruit Brix level under the PG promoter was close to the highest wild type level and ranked in the 95th percentile among all Brix measurements. However, the high Brix measurements in PG::G1645 plants were correlated with a very low fruit-set.

Other related data. The paralog of G1645, G2424, was not tested in tomato in the present field trial. Similar to G1645 overexpression, constitutive expression of G2424 produced a spectrum of developmental abnormalities and poor fertility in Arabidopsis. An increase in leaf stigmastanol was observed in two independent T2 lines.

TABLE 58
Data Summary for G1645

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.44 ± NA (1) | 46.17 ± NA (1) | 0.13 ± 0.044 (3) |
| AP1 | 5.42 ± 0.474 (2) | 71.97 ± 12.028 (2) | 0.29 ± 0.046 (2) |
| AS1 | NA | NA | 0.07 ± NA (1) |
| Cruciferin | NA | NA | 0.18 ± 0 (2) |
| LTP1 | 5.27 ± 0.339 (2) | 83.72 ± 4.78 (2) | 0.17 ± 0.011 (2) |
| PD | 4.92 ± 0.247 (2) | 47.86 ± 17.197 (2) | 0.16 ± 0.027 (2) |
| PG | 6.33 ± NA (1) | 66.65 ± NA (1) | 0.21 ± 0.012 (2) |
| STM | 5.1 ± NA (1) | 77.38 ± NA (1) | 0.17 ± NA (1) |

### G1650 (SEQ ID NO: 93 and 94)

Published background information. G1650 has been identified in the sequence of a BAC clone from chromosome 4 (BAC clone F16A16, gene F16A16.100, GenBank accession number AL035353). Heim et al. (2003) and Toledo-Ortiz et al. (2003) identified G1650 as AtbHLH023.

Discoveries in Arabidopsis. Overexpressors of G1650 under control of the 35S promoter had normal morphological and physiological characteristics.

None of the stress challenge array background experiments revealed any regulation of G1650 expression.

Discoveries in tomato. Plant volume was greater than that in wild type controls in plants expressing G1650 under the AP1 promoter, with a rank in the 95th percentile among all measurements. Brix was greater than that in wild type controls in plants expressing G1650 under the LTP1 promoter, with a rank in the 95th percentile among all measurements.

TABLE 59
Data Summary for G1650

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.62 ± NA (1) | 50.61 ± NA (1) | 0.18 ± 0.063 (3) |
| AP1 | 5.93 ± NA (1) | 52.21 ± NA (1) | 0.32 ± 0.19 (3) |
| AS1 | 5.49 ± 0.608 (3) | 53.74 ± 8.962 (3) | 0.29 ± 0.02 (3) |
| Cruciferin | 5.35 ± 0.618 (3) | 46.03 ± 23.883 (3) | 0.26 ± 0.043 (3) |
| LTP1 | 6.38 ± 0.142 (3) | 84.95 ± 22.889 (3) | 0.19 ± 0.061 (3) |
| PD | 4.79 ± NA (1) | 47.07 ± NA (1) | 0.27 ± 0.034 (3) |
| PG | 5.39 ± NA (1) | 35.24 ± NA (1) | 0.15 ± 0.05 (3) |
| RBCS3 | 5.69 ± 0.085 (2) | 81.27 ± 1.704 (2) | 0.27 ± 0.023 (3) |
| STM | 5.43 ± 0.401 (3) | 66.19 ± 18.96 (3) | 0.31 ± 0.15 (3) |

### G1659 (SEQ ID NO: 95 and 96)

Published background information: The sequence of G1659 (AT4G00670) was obtained from Arabidopsis genomic sequencing project, GenBank accession number AF058919, based on its sequence similarity within the conserved domain to other DBP related proteins in Arabidopsis. To date, there is no published information regarding the functions of this gene.

Discoveries in Arabidopsis. The function of G1659 was studied in Arabidopsis using transgenic plants in which the gene was expressed under the control of the 35S promoter. 35S::G1659 plants were wild-type in morphology and development, as well as in the physiological and biochemical analyses that were performed.

RT-PCR analysis of G1659 shows expression at low to moderate levels throughout the plant and is induced by auxin, ABA, heat, salt and drought. In a soil drought microarray experiment, G1659 was found to be repressed in Arabidopsis leaves at multiple stages of drought stress. Repression levels correlated with the severity of drought, and expression began to recover after rewatering. In a microarray study of ABA treated plants G1659 was found to be up regulated in shoots but down regulated in roots. G1659 was also found to be repressed in roots in the salicylic acid (400 μM), stress avg. mannitol (400 mM), and stress avg. NaCl (200 mM) microarray experiments.

Discoveries in tomato. Lycopene content in fruit was greater than in wild type controls, in plants expressing G1659 under the control of the Cruciferin, AS1, and STM promoters, and ranked in the 90th percentile among all measurements.

Transgenic plants expressing G1659 under the control of the Cruciferin, AS1, and STM promoters also showed morphological differences to controls. Plants expressing G1659 with the Cruciferin and STM promoters were noted to have a heavy late fruitset. Plants expressing G1659 under the control of the AS1 promoter, however, had a very heavy fruit-set that was not delayed. The combination of high lycopene with heavy fruit-set seen with different promoters in combination with G1659 is highly desirable.

TABLE 60
Data Summary for G1659

| | Promoter summary: Avg. ± StD. (Count) | | |
|---|---|---|---|
| | Brix (g sugar/ | | |
| Promoter | 100 g sample) | Lycopene (ppm) | Volume (m³) |
| AP1 | 5.82 ± 0.423 (3) | 70.69 ± 4.675 (3) | 0.2 ± 0.047 (3) |
| AS1 | 5.71 ± 0.126 (3) | 91.49 ± 10.288 (3) | 0.17 ± 0.022 (3) |
| Cruciferin | 5.86 ± 0.417 (2) | 90.41 ± 10.932 (2) | 0.16 ± 0.029 (3) |
| LTP1 | NA | NA | 0.17 ± 0 (2) |
| PD | 5.14 ± 0.675 (3) | 66.74 ± 14.982 (3) | 0.27 ± 0.044 (3) |
| PG | 5.36 ± 0.092 (2) | 42.91 ± 1.245 (2) | 0.19 ± 0.012 (2) |
| STM | 5.36 ± NA (1) | 90.45 ± NA (1) | 0.13 ± 0.02 (3) |

### G1752 (SEQ ID NO: 97 and 98)

Published background information. G1752, also designated AtERF15, corresponds to gene At2g31230 (AAD20668). Sakuma et al. (2002) categorized G1752 into the B3 subgroup of the AP2 transcription factor family, with the B family having only a single AP2 domain. G1752 is closely related to ERF1 (G1266), whose overexpression has been shown to confer multi-pathogen resistance on Arabidopsis (Berrocal-Lobo et al. (2002)).

Discoveries in Arabidopsis. The majority of 35S::G1752 Arabidopsis transformants were extremely small, with curled dark leaves, and were slow growing compared to controls. The most severely affected individuals arrested development at an early stage, and failed to flower.

In a series of microarray experiments with hormone and stress treatments, G1752 was found to be up-regulated by ACC treatment in roots after 24 hours, and repressed dramatically by drought treatment in leaves.

Discoveries in tomato. Plant size was greater than that in wild type controls in plants expressing G1752 under the 35S, Cruciferin and PG promoters, with a rank in the 95th percentile among all measurements. Increased plant size in the Cruciferin::G1752 plants was correlated with a good fruit-set. In contrast, seedlings expressing G1752 under the 35S promoter had reduced size and wrinkled leaves. Plant size was also dramatically reduced upon overexpression of G1752 with the 35S promoter in Arabidopsis.

Other related data. G2512, the paralog of G1752 was not in the field trial.

TABLE 61
Data Summary for G1752

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 4.86 ± 0.255 (3) | 31.17 ± 12.577 (3) | 0.33 ± 0.031 (3) |
| AP1 | 5.45 ± 0.389 (2) | 56.07 ± 22.019 (2) | 0.29 ± 0.045 (3) |
| AS1 | 5.68 ± NA (1) | 68.27 ± NA (1) | 0.23 ± NA (1) |
| Cruciferin | 5.43 ± 0.633 (3) | 38.33 ± 3.143 (3) | 0.39 ± 0.076 (3) |
| PG | 5.6 ± 0.904 (3) | 81.6 ± 4.384 (3) | 0.33 ± 0.101 (3) |
| RBCS3 | 4.86 ± 0.495 (2) | 67.34 ± 32.294 (2) | 0.23 ± 0.01 (3) |
| STM | NA | NA | 0.2 ± 0.044 (3) |

### G1755 (SEQ ID NO: 99 and 100)

Published background information. G1755 was identified in the sequence of BAC T3G21; it corresponds to gene At2g40350 (GenBank PID AAD25670). Sakuma et al. (2002) categorized G1755 into the AZ subgroup of the AP2 transcription factor family, with the A family related to the DREB and CBF genes, and G1755 relatively closely related to the DREB2 group.

Discoveries in Arabidopsis. Overexpression of G1755 under control of the 35S promoter in Arabidopsis resulted in plants that had normal morphology at all developmental stages and normal physiological responses in all assays.

In a series of microarray experiments with hormone and stress treatments, G1755 was not found to be regulated.

Discoveries in tomato. Plant volume was greater than that in wild type controls in plants expressing G1755 under the PD and PG promoters, with a rank in the 95th percentile among all measurements. Brix was greater than that in wild type controls in plants expressing G1755 under the AP1 and PD promoters, with a rank in the 95th percentile among all measurements. Lycopene content was greater than that in wild type controls in plants expressing G1755 under the PD promoter, with a rank in the 95th percentile among all measurements. Overexpression of G1755 under the 35S promoter in seedlings yielded plants with reduced size and darker green leaves. Overexpression of G1755 with the 35S promoter in Arabidopsis produced plants with normal morphology and physiology. The ability of G1755 to impact Brix, lycopene and volume, with all three affected by overexpression with the phytoene desaturase promoter, may have significant commercial value.

The increase in Brix levels in the AP1::G1755 plants was correlated with good fruit-set. However the increased volume seen in the PG::G1755 plants was associated with low fruit-set.

Other related data. G1754, a paralog of G1755 was not in the field trial.

TABLE 62
Data Summary for G1755

| | Promoter summary: Avg. ± StD. (Count) | | |
|---|---|---|---|
| | Brix (g sugar/ | | |
| Promoter | 100 g sample) | Lycopene (ppm) | Volume (m³) |
| 35S | 5.62 ± 0.304 (2) | 56.16 ± 16.603 (2) | 0.23 ± 0.059 (3) |
| AP1 | 6.67 ± 0.3 (3) | 86.05 ± 58.789 (3) | 0.22 ± 0.069 (3) |
| AS1 | 5.62 ± NA (1) | 65.76 ± NA (1) | 0.11 ± 0.076 (3) |
| Cruciferin | 5.91 ± 0.475 (3) | 64.32 ± 34.528 (3) | 0.18 ± 0.051 (3) |
| LTP1 | NA | NA | 0.18 ± 0.047 (2) |
| PD | 6.65 ± 0.375 (2) | 102.03 ± 6.201 (2) | 0.33 ± 0.026 (3) |
| PG | 5.61 ± 0.247 (2) | 54.75 ± 6.753 (2) | 0.32 ± 0.13 (3) |

### G1784 (SEQ ID NO: 101 and 102)

Published background information. G1784 (At2g02030) is a member of the putative myb-related gene family. G1784 was identified as part of BAC F14H20 (GenBank accession number AC006532), released by the Arabidopsis Genome sequencing project.

Discoveries in Arabidopsis. The function of this gene was analyzed using transgenic plants in which G1784 was expressed under the control of the 35S promoter. The phenotype of these transgenic plants was wild-type in all assays performed. G1784 appears to be expressed primarily in germinating seeds. The expression of G1784 is not induced in rosette leaves by any stress-related treatments tested, based on RT-PCR and microarray analyses.

Discoveries in tomato. The fruit Brix level under the Cruciferin promoter was close to the highest wild type level and ranked in the 95th percentile among all Brix measurements. The LTP1 promoter also produced an above average Brix level, but not in the 95th percentile.

TABLE 63
Data Summary for G1784

| | Promoter summary: Avg. ± StD. (Count) | | |
|---|---|---|---|
| | Brix (g sugar/ | | |
| Promoter | 100 g sample) | Lycopene (ppm) | Volume (m³) |
| Cruciferin | 6.36 ± 0.467 (2) | 85.65 ± 19.361 (2) | 0.2 ± 0.062 (3) |
| LTP1 | 6.13 ± NA (1) | 46.02 ± NA (1) | 0.22 ± 0.046 (3) |
| PG | NA | NA | 0.15 ± 0.084 (3) |
| RBCS3 | 4.52 ± 0.841 (2) | 76.23 ± 18.307 (2) | 0.12 ± 0.013 (3) |
| STM | 5.53 ± 0.576 (3) | 54.55 ± 22.338 (3) | 0.18 ± 0.017 (3) |

### G1785 (SEQ ID NO: 103 and 104)

Published background information. G1785 corresponds to gene AT2g25230, and it has also been described as AtMYB100 (Stracke et al. (2001)).

Discoveries in Arabidopsis. G1785 was studied in a knockout mutant (T-DNA insertion) and overexpressing lines in Arabidopsis. For both the knockout and the overexpressing lines, there were no consistent differences in morphology compared to wild-type controls and the plants were wild-type in the physiological analyses that were performed. RT-PCR analysis of the endogenous levels of G1785 indicates that this gene is primarily expressed in embryos. No expression is detected in leaf tissue under any stress-related condition tested, as determined by RT-PCR and microarray experiments.

Overexpression of G248 in Arabidopsis was found to confer greater sensitivity to disease, particularly following infection by Botrytis cinerea.

Discoveries in tomato. The fruit Brix level under the STM promoter was very close to the highest wild type level and ranked in the 95th percentile among all Brix measurements. The volume of these plants was smaller than average.

Other related data. The paralog of G1785, G248, was not tested in tomato in the present field trial.

TABLE 64
Data Summary for G1785

| | Promoter summary: Avg. ± StD. (Count) | | |
|---|---|---|---|
| | Brix | | |
| Promoter | (g sugar/100 g sample) | Lycopene (ppm) | Volume (m³) |
| AP1 | 5.67 ± 0.116 (3) | 42.98 ± 5.376 (3) | 0.11 ± 0.02 (3) |
| Cruciferin | 5.62 ± 0.177 (2) | 76.19 ± 10.09 (2) | 0.17 ± 0.037 (3) |
| PD | NA | NA | 0.12 ± 0.049 (3) |
| STM | 6.44 ± NA (1) | 42.91 ± NA (1) | 0.09 ± 0.03 (3) |

### G1791 (SEQ ID NO: 105 and 106)

Published background information. G1791 corresponds to gene K14B15.13 (BAA95735). Sakuma et al. (2002) categorized G1791 into the B3 subgroup of the AP2 transcription factor family, with the B family containing one AP2 DNA binding domain.

Discoveries in Arabidopsis. Overexpression of G1791 severely retarded growth and development. This phenotype was 100% penetrant across 35 independent T1 lines. 35S::G1791 plants were extremely tiny, slow growing, and formed dark green leaves. All lines were completely sterile and many arrested growth without initiating flower buds. In other lines, a few vestigial flower buds were noted, but very little inflorescence extension occurred, and these structures senesced without producing seed.

None of the stress challenge array background experiments revealed any regulation of G1791 expression.

Discoveries in tomato. Brix level in fruit was greater than that in wild type controls in plants expressing G1791 under the PG promoter, with a rank in the 95th percentile among all measurements. Fruit-set for PG::G1791 plants was low, and the potential relationship of this low fruit set on Brix measurements remains to be determined.

Plant size was dramatically reduced upon overexpression of G1791 with the 35S promoter in Arabidopsis. G1791 is a paralog of G1792, and both of these genes have been found to confer disease resistance on Arabidopsis overexpressors. The interaction between Brix and disease resistance bears further investigation, in terms of the basis for Brix increase in these lines, as alterations in cell wall synthesis, which could be related to an increased Brix, have been linked with disease resistance (e.g., Ellis et al. (2002)).

Other related data. G1791 paralog of G1792, and both of these genes have been found to confer disease resistance on Arabidopsis overexpressors. The interaction between Brix and disease resistance bears further investigation, in terms of the basis for Brix increase in these lines, as alterations in cell wall synthesis, which could be related to an increased Brix, have been linked with disease resistance (e.g., Ellis et al. (2002)). G1791 was not analyzed in the present field trial ATP field trial.

TABLE 65
Data Summary for G1791

| | Promoter summary: Avg. ± StD. (Count) | | |
|---|---|---|---|
| | Brix | | |
| Promoter | (g sugar/100 g sample) | Lycopene (ppm) | Volume (m³) |
| Cruciferin | 5.19 ± 0.601 (2) | 35.89 ± 9.899 (2) | 0.19 ± 0.087 (3) |
| LTP1 | 5.11 ± NA (1) | 76.79 ± NA (1) | 0.13 ± 0.057 (3) |
| PG | 6.48 ± NA (1) | 83.06 ± NA (1) | 0.14 ± 0.064 (2) |
| RBCS3 | 5.36 ± 0.134 (2) | 59.25 ± 7.913 (2) | 0.17 ± 0.041 (3) |

### G1808 (SEQ ID NO: 107 and 108)

Published background information. G1808 (At4g37730) was identified as part of the BAC clone T28119, GenBank accession number AL035709 (nid=4490717). G1808 is equivalent to AtbZIP7, a member of subgroup S (Jakoby et al. (2002)). Some genes of bZIP subgroup S contain 5′-upstream ORFs (uORFs) that are involved in post-transcriptional repression by sucrose. No published information on the function of G1808 is available.

Discoveries in Arabidopsis. G1808 appears to be constitutively expressed in all tissues and environmental conditions tested. However, gene chip experiment showed that G1808 is induced by drought, ABA, JA and SA. The annotation of G1808 in BAC ATT28I19 was experimentally determined. A line homozygous for a T-DNA insertion in G1808 was initially used to determine the function of this gene. The T-DNA insertion of G1808 is approximately 140 nucleotides after the ATG in coding sequence and therefore is likely to result in a null mutation. The phenotype of these transgenic plants was wild-type in all assays performed. Subsequently, the function of G1808 was studied by overexpression of the genomic DNA for the gene under control of the 35S promoter in transgenic plants. Overexpression of G1808 resulted in major growth abnormalities including reduced size, and changes in flower development. G1808 overexpressing lines showed reduced seedling size and vigor in the cold germination assay. Based on the germination controls this was not due to an overall reduced seedling germination and growth. The same phenotype was observed for overexpression of G2070, another bZIP transcription factor, suggesting redundancy of gene function.

Arabidopsis lines overexpressing G1047, a paralog of G1808, were more tolerant to infection with a moderate dose of the fungal pathogen Fusarium oxyporum.

Discoveries in tomato. The fruit Brix level under the RBCS3 promoter was close to the highest wild type level and ranked above the 95th percentile among all Brix measurements. The paralog of G1808, G1047, was not tested in tomato in the present field trial.

Other related data. The paralog of G1808, G1047, was not tested in tomato in the present field trial. In Arabidopsis, lines with overexpression of G1047 were more tolerant to infection with a moderate dose of the fungal pathogen Fusarium oxysporum.

TABLE 66
Data Summary for G1808

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | 6.13 ± NA (1) | 91.06 ± NA (1) | 0.16 ± 0.066 (3) |
| AS1 | 5.87 ± 0.468 (3) | 83.56 ± 11.824 (3) | 0.2 ± 0.011 (3) |
| LTP1 | 5.66 ± NA (1) | 59.03 ± NA (1) | 0.17 ± 0.042 (3) |
| RBCS3 | 6.42 ± 0.12 (2) | 80.44 ± 31.176 (2) | 0.2 ± 0.062 (3) |

### G1809 (SEQ ID NO: 109 and 110)

Published background information. G1809 was identified in the sequence of BAC MKP6, GenBank accession number AB022219, released by the Arabidopsis Genome Initiative.

Discoveries in Arabidopsis. The function of this gene was analyzed using transgenic plants in which G1809 was expressed under the control of the 35S promoter. The phenotype of these transgenic plants was wild-type in all assays performed. G1809 appears to be constitutively expressed in all tissues and environmental conditions tested.

Discoveries in tomato. The fruit Brix level under the LTP1 promoter is higher than the highest wild type level and ranked above the 95th percentile among all Brix measurements. There are no apparent paralogs of G1808. Arabidopsis lines overexpressing G1809 produced wild-type phenotypes in all assays performed.

TABLE 67
Data Summary for G1809

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | 5.65 ± NA (1) | 37 ± NA (1) | 0.28 ± 0.025 (3) |
| Cruciferin | 4.87 ± NA (1) | 59.1 ± NA (1) | 0.25 ± 0.04 (3) |
| LTP1 | 6.51 ± NA (1) | 87.11 ± NA (1) | 0.25 ± 0.042 (3) |
| PG | 6.19 ± NA (1) | 84.97 ± NA (1) | 0.22 ± 0.08 (3) |

### G1815 (SEQ ID NO: 111 and 112)

Published background information. G1815 (At3g29020) was identified in the sequence of TAC clone:K5K13 (GenBank accession number AB025615), released by the Arabidopsis Genome Initiative, and is also referred to as AtYB110 (Stracke et al, 2001).

Discoveries in Arabidopsis. The function of G1815 was analyzed using transgenic Arabidopsis plants in which the gene was expressed under the control of the 35S promoter. The phenotype of the 35S::G1815 transgenics was wild-type in morphology, and wild-type with respect to their response to biochemical and physiological analyses.

RT-PCR analysis of the endogenous levels of G1815 indicates that this gene is expressed at low levels mainly in flower tissue. In leaf tissue, G1815 is induced in response to a variety of stress-related conditions, as detected by RT-PCR. Microarray analysis did not show any significant changes in G1815 expression due to the stress treatments, hormone treatments, or overexpression of any of the tested transcription factors.

Discoveries in tomato. In tomatoes overexpressing G1815 under the control of the 35S promoter, plant size was close to the highest wild type level and ranked in the 95th percentile among all volume measurements. The leaf edges of these plants were curled. In Arabidopsis, the phenotype of the 35S::G1815 transgenics was wild-type in morphology, and wild-type with respect to their response to biochemical and physiological analyses.

TABLE 68
Data Summary for G18155

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| | 5.43 ± 0.512 (3) | 60.35 ± 16.104 (3) | 0.35 ± 0.14 (3) |
| AP1 | NA | NA | 0.17 ± 0.042 (2) |
| AS1 | NA | NA | 0.18 ± 0.05 (3) |
| Cruciferin | 5.86 ± 0.163 (2) | 41.7 ± 13.343 (2) | 0.2 ± 0.028 (3) |
| PD | 5.47 ± 0.538 (3) | 55.35 ± 24.251 (3) | 0.18 ± 0.045 (3) |
| PG | 5.43 ± 0.778 (2) | 70.44 ± 1.365 (2) | 0.19 ± 0.059 (2) |
| STM | 5.79 ± 0.46 (3) | 65.75 ± 4.052 (3) | 0.2 ± 0.05 (3) |

### G1865 (SEQ ID NO: 113 and 114)

Published background information. The sequence of G1865 (At2g06200) was initially obtained from the Arabidopsis sequencing project, GenBank accession number AC006413 (GI:20197765), based on sequence similarity to the rice Growth-regulating-factor1 (GRF1, GI: 6573149; Knaap et al. (2000)). Nine of the ten members of the Arabidopsis AtGRF family were recently published by Kim et al. (2003)), including G1865 referred as AtGRF6. Their functional analysis of the gene family did not include G1865.

Discoveries in Arabidopsis. The function of G1865 was analyzed through its ectopic overexpression in plants. The analysis of the endogenous level of G1865 transcripts by RT-PCR revealed a predominant expression in roots, flowers, embryo and siliques, with very little expression in shoots and rosette leaves, in agreement with northern blot analysis (Kim et al. (2003)). In addition, G1865 expression was repressed in response to cold, heat and in interaction with Fusarium oxysporum and Erysiphe orontii. Microarray analysis revealed no significant (p-value<0.01) in G1865. The function of G865 was analyzed by ectopic overexpression in Arabidopsis. 35S::G1865 transgenic Arabidopsis displayed rounded, dark green leaves, with short petioles, and were smaller than controls at early stages of development. Overexpression of G1865 markedly delayed the onset of flowering. Several lines exhibited such effects and all showed a distinct delay in bolting, producing a greatly increased number leaves; the most extreme individuals formed visible flower buds around a month after wild type (continuous light conditions), by which time rosette leaves had become rather large and contorted.

Discoveries in tomato. Transgenic tomatoes expressing G1865 under the seed (cruciferin) promoter were significantly larger than wild type controls; ranking among the 95th percentile of all volumetric measurements. Similarly, but to a lesser extent, overexpression of G1865 under the meristem (AS1) and flower (AP1) promoters results in transgenic tomato plants larger than wild-type (90th percentile). Transgenic AP1::G1865 tomato plants also produced many more fruits than wild-type control plants.

35S::G1865 transgenic Arabidopsis displayed rounded, dark green leaves, with short petioles, and were smaller than controls at early stages of development. Overexpression of G1865 markedly delayed the onset of flowering.

Other related data. The phenotype observed in 35S::G1865 plants is similar to results obtained by Knaap et al. (2000) when overexpressing the rice Os-GRF1 in Arabidopsis. Transgenic plants showed a comparable late bolting phenotype that could be partially rescued by external application of gibberellic acid to the plant. This result suggests that G1865 is a functional ortholog of the rice Os-GRF1 in Arabidopsis, but has significant differences in expression pattern. The Os-GRF1 is found to be specifically expressed in intercalary meristem of deepwater rice, while G1865 is expressed in all tissues except shoots and rosette leaves where expression in almost absent. G1865 may play an important role in GA-response, and in regulation of cell elongation.

TABLE 69
Data Summary for G1865

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| AP1 | 5.32 ± 0.855 (3) | 96.35 ± 21.847 (3) | 0.29 ± 0.021 (3) |
| AS1 | 5.11 ± NA (1) | 75.58 ± NA (1) | 0.27 ± 0.025 (3) |
| Cruciferin | 4.74 ± NA (1) | 54.71 ± NA (1) | 0.32 ± 0.049 (3) |

### G1884 (SEQ ID NO: 115 and 116)

Published background information. G1884 was identified as a gene in the sequence of BAC clone F20D10 (Accession Number AL035538), released by the European Union Arabidopsis Sequencing Project. A partial sequence of G1884 is found in the sequence of the EST FB026h08F (Accession Number AV531601), which was obtained from a cDNA library derived from Arabidopsis flower buds. No further information is available concerning the function of this gene.

Discoveries in Arabidopsis. The sequence of G1884 was experimentally determined and the function of this gene was analyzed using transgenic plants in which G1884 was expressed under the control of the 35S promoter. Overexpression of G1884 produced deleterious effects on Arabidopsis growth and development. No transformants were obtained during the first two selection attempts on T0 seeds, suggesting that the gene might have lethal effects. However, a small number of transformants were finally obtained from a third and fourth batch of T0 seed (RT-PCR confirmed that these lines displayed high levels of G1884 overexpression). These 35S::G1884 plants were uniformly much smaller than wild-type controls throughout development. Following the switch to flowering, the inflorescences from these lines were very poorly developed and produced very few, if any, seeds. RT-PCR analysis indicates that G1884 is expressed at low levels in flowers and rosette leaves, and at higher levels in embryos and siliques, which suggests a role for this gene in embryo or early seedling development and is slightly induced by osmotic stress. Microarray analysis indicates that G1884 is induced by SA.

Discoveries in tomato. The fruit lycopene level under the LTP1 promoter was above the highest wild type levels and ranked in the 95th percentile among all measurements.

TABLE 70
Data Summary for G1884

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| AP1 | 5.33 ± 0.191 (2) | 66.69 ± 37.342 (2) | 0.18 ± 0.124 (3) |
| AS1 | 5.64 ± 0.41 (2) | 68.84 ± 2.468 (2) | 0.24 ± 0.075 (2) |
| Cruciferin | 5.95 ± NA (1) | 53.32 ± NA (1) | 0.16 ± 0.015 (3) |
| LTP1 | 6.2 ± 0.184 (2) | 108.76 ± 6.746 (2) | 0.15 ± 0.027 (2) |
| PD | 5 ± 0.548 (3) | 60.24 ± 5.295 (3) | 0.21 ± 0.112 (3) |
| RBCS3 | 5.36 ± NA (1) | 39.89 ± NA (1) | 0.14 ± 0.159 (2) |
| STM | 5.18 ± 0.354 (2) | 57.2 ± 9.504 (2) | 0.19 ± 0.018 (2) |

### G1895 (SEQ ID NO: 117 and 118)

Published background information. G1895 was identified as a gene in the sequence of the BAC T24P13 (Accession Number AC006535), released by the Arabidopsis thaliana Genome Center. No further published information about the function of G1895 is available.

Discoveries in Arabidopsis. The function of G1895 was analyzed using transgenic plants in which G1895 was expressed under the control of the 35S promoter. Overexpression of G1895 delayed the onset of flowering in Arabidopsis by around 2-3 weeks under continuous light conditions, although this phenotype was observed only at low frequency. In all other physiological and biochemical assays, 35S::G1895 plants appeared identical to controls. RT-PCR analysis indicates G1895 was expressed in all tissues and the highest levels of expression were found in flowers, rosette leaves, and embryos. In rosette leaves using RT-PCR, G1895 appears to be induced by auxin, ABA, and by cold stress. Microarray analysis confirmed the induction of G1895 by cold stress.

Discoveries in tomato. Under the AP1 and AS1 promoters, plant size ranked in the 95th percentile among all plant size measurements. The AP1::G1895 and AS1::G1895 plants had good fruit-set, although this trait was somewhat variable.

Other related data. A paralog of G1895, G1903, was tested in the tomato field trials in the present field trial. Significant changes in plant size (greater than the 95th percentile, was observed in LTP1::1903 and Cruciferin::G1903 tomato plants.

TABLE 71
Data Summary for G1895

| | Promoter summary: Avg. ± StD. (Count) | | |
|---|---|---|---|
| Promoter | Brix (g sugar/ | | |
| | 100 g sample) | Lycopene (ppm) | Volume (m^{³}) |
| 35S | 5.2 ± 0.339 (2) | 66.19 ± 28.617 (2) | 0.1 ± 0.037 (3) |
| AP1 | 4.62 ± NA (1) | 29.5 ± NA (1) | 0.37 ± 0.097 (3) |
| AS1 | 4.91 ± NA (1) | 37.91 ± NA (1) | 0.34 ± NA (1) |

### G1897 (SEQ ID NO: 119 and 120)

Published background information. G1897 was identified as a gene in the sequence of the TAC clone K8A10 (Accession Number AB026640), released by the Kazusa DNA Research Institute (Chiba, Japan). No further published information about the function of G1897 is available.

Discoveries in Arabidopsis. The function of G1897 was analyzed using transgenic plants in which G1897 was expressed under the control of the 35S promoter. Overexpression of G1897 produced marked effects on leaf and floral organ development. 35S::G1897 transformants formed narrow, dark-green rossette and cauline leaves. Additionally, most lines were rather small and slow developing compared to wild type. Following the switch to flowering, inflorescences often displayed short internodes and carried flowers with various abnormalities. Interestingly, perianth organs showed equivalent effects to those observed in leaves, and were typically rather long and narrow. By contrast, stamens were rather short; silique formation was very poor, presumably as a result of this defect. 35S::G1897 plants also appeared to have delayed abscission of floral organs, and delayed senescence compared to wild type. Such features were likely a consequence of the overall low fertility and poor seed.

In addition, overexpression of G1897 in Arabidopsis resulted in an increase in seed glucosinolates M39491 and M39493 in T2 lines 2 and 3. Otherwise, overexpression of G1897 in Arabidopsis did not result in any altered phenotypes in any of the physiological or biochemical assays.

G1897 expression was detected in flowers, embryos, and siliques, and to a lesser degree in seedlings. The expression of G1897 appears to be reduced in response to Erysiphe infection.

Discoveries in tomato. Under the cruciferin promoter, plant size ranked in the 95th percentile in plant size. These plants also had good fruit-set.

Other related data. A paralog of G1897, G798, was not tested in tomato in the present field trial. Overexpression of g1897 under various promoters in tomato caused the production of small plants or small fruit. For example, AP1::G1897 tomato plants were small, while AS1::G1897 tomato plants had small green fruit.

TABLE 72
Data Summary for G1897

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | 5.3 ± 0.188 (3) | 50.93 ± 3.285 (3) | 0.31 ± 0.085 (3) |
| AP1 | 5.29 ± 0.615 (2) | 42.75 ± 0.969 (2) | 0.23 ± 0.029 (3) |
| AS1 | 5.91 ± NA (1) | 59.8 ± NA (1) | 0.22 ± 0.046 (3) |
| Cruciferin | 4.93 ± 0.269 (2) | 74.18 ± 1.81 (2) | 0.32 ± 0.024 (3) |
| LTP1 | 4.88 ± 1.124 (2) | 68.86 ± 25.053 (2) | 0.21 ± 0.07 (3) |
| PG | 5.67 ± 0.269 (2) | 41.89 ± 8.648 (2) | 0.14 ± 0.079 (3) |
| RBCS3 | 5.66 ± 0.14 (3) | 59.43 ± 17.173 (3) | 0.3 ± 0.027 (3) |

### G1903 (SEQ ID NO: 121 and 122)

Published background information. G1903 was identified from the Arabidopsis genomic sequence, GenBank accession number AC021046, based on its sequence similarity within the conserved domain to other DOF related proteins in Arabidopsis. To date, there is no published information regarding the function of this gene.

Discoveries in Arabidopsis. The function of this gene was analyzed using transgenic plants in which G1903 was expressed under the control of the 35S promoter. Two lines (5 and 7) showed a significant decrease in seed protein content and an increase in seed oil content (though the increase was slightly below our significance cutoffs) as assayed by NIR, otherwise the phenotype of these transgenic plants was wild-type in all other assays performed.

Gene expression profiling using RT/PCR shows that G1903 is expressed predominantly in flowers, however it is almost undetected in roots and seedlings. Furthermore, there is no significant effect on expression levels of G1903 after exposure to environmental stress conditions. However, microarray analysis indicates that G1903 is induced by cold stress.

Discoveries in tomato. The fruit lycopene levels for LTP1::G1903 plants were above the highest wild type levels and ranked in the 95th percentile among all measurements. Under the cruciferin and LTP1 promoters, plant size is also significantly greater than the wild-type controls, and cruciferin::G1903 plants also had a heavy fruit-set.

A G1903 paralog, G1895, was also tested in the field trial. Under the cruciferin promoter, the size of G1895 overexpressors was significantly greater than wild type controls.

Other related data. Its paralog G1895 was also tested in the field trial. Under the cruciferin promoter, plant size was significantly more than wild type controls.

### Table 73. Data Summary for G1903

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| | 5.53 ± 0.5 (3) | 58.95 ± 6.98 (3) | 0.29 ± 0.076 (3) |
| AP1 | NA | NA | 0.23 ± 0.057 (3) |
| Cruciferin | 5.02 ± 0.61 (3) | 68.79 ± 10.74 (3) | 0.33 ± 0.125 (3) |
| LTP1 | 6.12 ± NA (1) | 98.26 ± NA (1) | 0.4 ± 0.033 (3) |
| PG | NA | NA | 0.25 ± 0.06 (3) |
| STM | 5.34 ± 0.247 (2) | 45.66 ± 1.259 (2) | 0.3 ± 0.127 (3) |

### G1909 (SEQ ID NO: 123 and 124)

Published background information. G1909 is equivalent to the Arabidopsis OBP2 gene (Accession Number AF155816) (Kang H G, Singh K B, 2000). OBP2 was shown by Northern blots to be highly expressed in leaves and roots, and at lower levels in stems and flowers. In roots, OBP2 was induced by auxin and salicylic acid. No further published information about the function of G1909 is available.

Discoveries in Arabidopsis. The function of G1909 was analyzed using transgenic plants in which G1909 was expressed under the control of the 35S promoter. 35S::G1909 plants appeared identical to controls morphologically and physiologically. In one line (#2), overexpression of G1909 resulted in a marginal decreased in seed protein content as measured by NIR.

G1909 is expressed in all tissues of Arabidopsis, and its expression in rosette leaves appears to be relatively unchanged in response to the environmental stress-related conditions tested using RT-PCR. Microarray analysis indicated that G1909 is induced by drought, cold, mannitol, ABA, and MeJA.

Discoveries in tomato. In transgenic tomatoes overexpressing G1909 under the regulatory control of the cruciferin promoter, plant size ranked in the 95th percentile among all plant size measurements.

Other related data. Overexpression of G1909 under various promoters in tomato caused the production of small plants or small fruit. For example, AP1::G1909 tomato plants were small, while AS1::G1909 tomato plants had small green fruit. Cruciferin::G1909 plants also had compact, small fruit. G1264, a paralog of G1909 was not in the field trial.

TABLE 74
Data Summary for G1909

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| AP1 | 5.44 ± NA (1) | 50.69 ± NA (1) | 0.21 ± 0.025 (3) |
| AS1 | NA | NA | 0.22 ± 0.05 (2) |
| Cruciferin | 6.05 ± 0.445 (2) | 84.4 ± 5.841 (2) | 0.33 ± 0.049 (2) |
| PG | 5.26 ± NA (1) | 37.57 ± NA (1) | 0.28 ± 0.146 (3) |

### G1935 (SEQ ID NO: 125 and 126)

Published background information. G1935 corresponds to AT1G77950. G1935 has two potential paralogs in the Arabidopsis genome, G2058 (AT1G77980, AGL66) and G2578 (AT1G22130).

Discoveries in Arabidopsis. G1935 was analyzed during our Arabidopsis genomics program via 35S::G1935 lines. Overexpression of G1935 in Arabidopsis produced no consistent differences in phenotype compared to wild type. However, it was noted that some of the 35S::G1935 lines were reduced in size and showed accelerated flowering. 35S::G2058 Arabidopsis lines were also analyzed by overexpression during our genomics program and exhibited a wild-type phenotype. Analysis of G2578 was not completed at that time.

RT-PCR experiments indicated that G1935 was expressed at high levels in siliques. G2058 expression was not detectable in a range of tissues examined by RT-PCR and it was concluded that the gene is expressed either at very low levels or in a highly cell-specific or condition-specific pattern.

Neither G1935 nor G2058 nor G2578 has been found significantly differentially expressed in response to conditions examined in the microarray studies performed to date.

Discoveries in tomato. Brix levels from LTP1::G1935 fruits were markedly higher than those found in wild-type control fruit.

Other related data. The closely related paralogs G2058 and G2578 have not yet been analyzed in the tomato field trial.

TABLE 75
Data Summary for G1935

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| AP1 | 5.5 ± 0.238 (3) | 82 ± 22.814 (3) | 0.26 ± 0.051 (3) |
| LTP1 | 6.49 ± 0.204 (3) | 53 ± 25.048 (3) | 0.21 ± 0.023 (3) |
| PD | 5.34 ± 0.127 (2) | 81.25 ± 31.346 (2) | 0.24 ± 0.103 (3) |
| RBCS3 | 5.87 ± NA (1) | 77.13 ± NA (1) | 0.18 ± 0.041 (3) |
| STM | 5.98 ± 0.148 (2) | 83.34 ± 14.651 (2) | 0.29 ± 0.107 (3) |

### G1950 (SEQ ID NO: 127 and 128)

Published background information. The sequence of G1950 (At2g03430) was initially obtained from the Arabidopsis sequencing project, GenBank accession number AC006284.4 (GI:20197736). G1950 has no distinctive features other than the presence of a 33-amino acid repeated ankyrin element known for protein-protein interaction, in the C-terminus of the predicted protein. Amino acid sequence comparison shows similarity to Arabidopsis NPR1.

Discoveries in Arabidopsis. The analysis of the endogenous level of G1950 transcripts by RT-PCR revealed specific expression in embryos, siliques and germinating seeds. G1950 expression is induced upon auxin treatment, which suggests that G1950 may play an important role in seed/embryo development or other processes specific to seeds (stress-related or desiccation-related). Microarray analysis revealed no significant (p-value<0.01) alteration in G1950 expression in all conditions examined. The function of G1950 was analyzed by ectopic overexpression in Arabidopsis. Plants overexpressing G1950 were more tolerant to infection with the necrotrophic fungal pathogen Botrytis cinerea when compared to wild type control. This phenotype was confirmed using mixed and individual transgenic Arabidopsis lines. G1950 transgenic Arabidopsis plants were morphologically indistinguishable from wild-type plants, and showed no biochemical changes in comparison to wild type control.

Discoveries in tomato. Transgenic plants expressing G1950 under the AP1, LTP1, PD and PG promoters have significantly (76-130%) increased plant size compared with wild type controls, ranking in the 95th percentile among all volumetric measurements. Similarly, 35S::G1950 transgenic tomatoes ranked in the 90th percentile for plant volume. This is particularly notable for the AP1 and PD promoters, as enhanced volume was not at the expense of fruit yield, since fruit set with these promoters was above average. 35S::G1950 Arabidopsis were morphologically indistinguishable from wild-type plants and more tolerant to Botrytis cinerea, suggesting increased fitness of G1950 transgenic tomatoes in field-grown conditions. This phenotype may be related to better tolerance to stress and/or pathogens.

Other related data. We have not yet identified a paralog of G1950 in Arabidopsis. Structural similarities with the Arabidopsis NPR1 suggest that G1950 may have a function related to NPR I in regulating transcriptional activity in response to pathogen ingress.

TABLE 76
Data Summary for G1950

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | 5.76 ± 1.054 (2) | 75.5 ± 24.805 (2) | 0.29 ± 0.159 (3) |
| AP1 | 5.42 ± 0.435 (3) | 86.72 ± 9.687 (3) | 0.42 ± 0.085 (3) |
| Cruciferin | NA | NA | 0.21 ± NA (1) |
| LTP1 | 5.51 ± 0.548 (3) | 89.77 ± 25.386 (3) | 0.32 ± 0.127 (3) |
| PD | 5.26 ± 0.535 (3) | 89.65 ± 13.85 (3) | 0.36 ± 0.145 (2) |
| PG | 5.67 ± 0.658 (2) | 84.35 ± 33.531 (2) | 0.32 ± 0.043(3) |
| RBCS3 | 5.55 ± 0.29 (2) | 72.16 ± 19.141 (2) | 0.21 ± 0.109(3) |
| STM | 5.68 ± 0.976 (2) | 89.85 ± 28.899 (2) | 0.27 ± 0.074(3) |

### G1954 (SEQ ID NO: 129 and 130)

Published background information. The sequence of G1954 was obtained from GenBank accession number AB028621, based on its sequence similarity within the conserved domain to other bHLH related proteins in Arabidopsis. G1954 corresponds to AtbHLH097, as described by Heim et al. (2003) and Toledo-Ortiz et al. (2003), which describe the Arabidopsis bHLH gene family.

Discoveries in Arabidopsis. Overexpression of G1954 under control of the 35S promoter was lethal in Arabidopsis. The transformation frequency obtained with the 35S::G1954 transgene was very low, suggesting that the gene might be lethal at high levels of activity. Zero transformants were isolated from the first two batches of T0 seed sown to kanamycin selection plates (normally we obtain 15-120 T1 plants from each batch). A single tiny transformant was eventually obtained from a third batch of T0 seed, but this plant died at an early stage without setting seeds. A final batch of T0 seed was then selected; no transformants were visible at seven days after sowing, but the plates were incubated for a further seven days. At that point, four very small, late germinating, putative transformants were apparent; these plants displayed very rudimentary development and were too tiny for transplantation to soil. To verify that such plants overexpressed the transgene they were pooled together for RNA extraction; RT-PCR experiments confirmed that G1954 was overexpressed at high levels.

In a series of microarray experiments with hormone and stress treatments, G1954 expression was not found to be regulated.

Discoveries in tomato. Brix content in fruit was greater than that in wild type controls in plants expressing G1954 under the AP1 promoter, with a rank in the 95th percentile among all measurements. However, there were no ripe fruit when samples were collected, due to a late-fruiting phenotype in the AP1-regulated lines.

TABLE 77
Data Summary for G1954

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | NA | NA | 0.14 ± 0.058 (2) |
| AP1 | 6.47 ± 0.262 (2) | 69.7 ± 6.35 (2) | 0.25 ± 0.027 (3) |
| Cruciferin | 5.52 ± NA (1) | 72.41 ± NA (1) | 0.27 ± NA (1) |
| RBCS3 | 5.81 ± NA (1) | 44.61 ± NA (1) | 0.21 ± NA (1) |
| STM | 4.63 ± NA (1) | 72.13 ± NA (1) | 0.2 ± 0.023 (2) |

### G1958 (SEQ ID NO: 131 and 132)

Published background information. G1958 was initially identified in the sequence of BAC T5F17, GenBank accession number AL049917, released by the Arabidopsis Genome Initiative. Subsequently, G1958 was published as PHR1. Mutants in PHR1 show reduced growth under conditions of phosphate starvation and fail to induce genes normally regulated by low phosphate concentration (Rubio et al. (2001)).

Discoveries in Arabidopsis. During our genomics program, we studied both lines homozygous for a T-DNA insertion in G1958 and lines expressing G1958 under the control of the 35S promoter. The knockout plants showed a reduction in root growth on plates, but otherwise appeared wild type. The reduced root growth was accentuated when seedlings were transferred to stress conditions, indicating that it may be environmentally influenced. No consistent differences were observed between 35S::G1958 lines and wild-type controls in any of the assays. Despite the published data indicating a function for G1958 in adaptation to phosphate starvation, overexpression of G1958 did not improve growth on low phosphate in our plate assay. G1958 was not induced in any of our microarray analyses to date, but low nutrient conditions have not been examined.

Discoveries in tomato. Plants expressing G1958 under three different promoters (35S, AS1 and cruciferin) produced significantly increased plant size at two months. It is possible that this increase is related to the published function of G1958 in regulation of a phosphate starvation response. If plants in the field are somewhat limited for phosphate, up-regulation of phosphorus intake or recycling may increase size. The result that plant volume increased when G1958 was driven under the cruciferin promoter (a seed promoter) may seem surprising; however, this promoter does show some expression in seedlings. Conversely, plants expressing G1958 under the STM promoter were noted to be “compact”. Meristematic expression of this gene may be deleterious.

TABLE 78
Data Summary for G1958

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | 5.73 ± NA (1) | 80.07 ± NA (1) | 0.33 ± 0.156 (3) |
| AS1 | 5.97 ± 0.582 (3) | 75.96 ± 5.821 (3) | 0.4 ± 0.029 (3) |
| Cruciferin | 6.05 ± 0.13 (3) | 85 ± 17.886 (3) | 0.41 ± 0.087 (3) |
| PG | NA | NA | 0.17 ± 0.071 (3) |
| STM | 5.8 ± 0.424 (2) | 61.45 ± 8.754 (2) | 0.28 ± 0.191 (3) |

### G2052 (SEQ ID NO: 133 and 134)

Published background information. G2052 was identified in the sequence of BAC T13D8 with accession number AC004473 released by the Arabidopsis Genome Initiative. It also corresponds to the AGI locus of AT5G46590. A comprehensive analysis of NAC family transcription factors was recently published by Ooka et al. (2003) where G2052 was identified as ANAC096.

Discoveries in Arabidopsis. The function of G2052 was analyzed using transgenic plants in which the gene was expressed under the control of the 35S promoter. The phenotype of the 35S::G2052 transgenics was wild type in morphology, and wild type with respect to their response to biochemical and physiological analyses. RT-PCR analysis of the endogenous levels of G2052 indicates that this gene is expressed at moderate levels in most tissues. Microarrays of eight-week-old Arabidopsis (ecotype col) plants exposed to drought stress and allowed to recover were performed. Plants in the drought recovery stage were found to produce G2052 transcript above four fold that of untreated plants.

Discoveries in tomato. Transgenic tomatoes expressing G2052 under the regulation of 35S, AP1, AS1, Cruciferin, LTP1, PD and PG promoters were analyzed for alterations in plant size, soluble solids and lycopene. Under the regulation of three out seven promoters (AP1, LTP1, PD) significant increases in plant size were observed. It is particularly notable that in lines overexpressing G2052 with the AP1 promoter, increased plant size was also associated with increased fruit set.

Other related data. G2052 has one paralog in Arabidopsis, G506, which was also included in the present field trial. G506 transgenic lines did not score in the 95th percentile for any trait.

TABLE 79
Data Summary for G2052

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | 5.44 ± 0.151 (3) | 70.12 ± 18.895 (3) | 0.25 ± 0.06 (3) |
| AP1 | 5.43 ± 0.372 (3) | 66.48 ± 18.905 (3) | 0.36 ± 0.038 (3) |
| AS1 | 5.27 ± 0.569 (3) | 69.74 ± 25.614 (3) | 0.25 ± 0.035 (3) |
| Cruciferin | 5.6 ± 0.336 (3) | 52.97 ± 10.726 (3) | 0.32 ± 0.021 (3) |
| LTP1 | 6.03 ± NA (1) | 76.26 ± NA (1) | 0.34 ± NA (1) |
| PD | 4.3 ± 0.643 (2) | 67.69 ± 6.06 (2) | 0.34 ± 0.109 (3) |
| PG | 5.48 ± 0.834 (3) | 81.23 ± 13.142 (3) | 0.3 ± 0.127 (3) |

### G2072 (SEQ ID NO: 135 and 136)

Published background information. G2072 was discovered as a gene in BAC F1504, accession number AC007887, released by the Arabidopsis genome initiative. There is no published information regarding the function of G2072.

Discoveries in Arabidopsis. The boundaries of G2072 were determined and the function of this gene was analyzed using transgenic plants in which G2072 was expressed under the control of the 35 S promoter. The phenotype of these transgenic plants was wild type in all assays performed. G2072 expression appeared to be flower specific and not induced by any of the environmental conditions tested.

Discoveries in tomato. The fruit lycopene level under the AS1 promoter was higher than the highest wild type level and ranked above the 95th percentile among all lycopene measurements, and was higher than the highest wild type level. Arabidopsis lines overexpressing G2072 produced wild-type phenotypes in all assays performed.

TABLE 80
Data Summary for G2072

| | Promoter summary: Avg. ± StD. (Count) | | |
|---|---|---|---|
| | Brix (g sugar/ | | |
| Promoter | 100 g sample) | Lycopene (ppm) | Volume (m^{³}) |
| 35S | 4.85 ± 0.629 (2) | 76.78 ± 12.82 (2) | 0.13 ± 0.072 (3) |
| AP1 | 5.26 ± NA (1) | 73.92 ± NA (1) | 0.14 ± 0.008 (3) |
| AS1 | 5.66 ± NA (1) | 104.79 ± NA (1) | 0.17 ± 0.038 (3) |
| LTP1 | 5.71 ± NA (1) | 40.6 ± NA (1) | 0.08 ± 0.012 (3) |
| PG | NA | NA | 0.18 ± NA (1) |

### G2108 (SEQ ID NO: 137 and 138)

Published background information. G2108 was identified in the sequence of BAC clone F13K23 (AC012187, gene F13K23.14). Sakuma et al. (2002) categorized G2108 into the B1 subgroup of the AP2 transcription factor family, with the B family having only a single ERF domain.

Discoveries in Arabidopsis. Overexpression of G2108 under control of the 35S promoter produced plants with alterations in plant growth and development. 35S::G2108 plants had a more compact inflorescence structure than wild type; internodes were short and an increased number of cauline leaf nodes were apparent on both the primary and higher order shoots. Apical dominance was also reduced, and a number of shoots borne from the axils of rosette leaves attained the same length as the primary inflorescence. The plants with altered shoot morphology also produced siliques that were rather wide and flat compared to those of wild type. In addition to the alterations in inflorescence structure, many of the individuals in the replant populations were noted to have rather curled leaves. Global transcript profiling under a variety of stress conditions revealed no conditions in which G2108 expression was modified compared to standard growth conditions. Qualitative RT-PCR indicated that G2108 is induced following auxin treatment.

Discoveries in tomato. Lycopene content and Brix content in fruit were greater than that in wild type controls in plants expressing G2108 under the PG promoter, with a rank in the 95th percentile among all measurements. Arabidopsis plants overexpressing G2108 under the 35S promoter had more compact inflorescences, twisted and curled leaves, and flattened siliques. The curling of leaves was reminiscent of epinasty, which can be induced by auxin treatment. Fruit development is also promoted by auxin treatment, suggesting the hypothesis that the effect of G2108 ectopic expression in fruit under the PG promoter may have its effects through modulation of certain auxin responses.

TABLE 81
Data Summary for G2108

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | 5.09 ± NA (1) | 69.22 ± NA (1) | 0.16 ± 0.093 (3) |
| AS1 | 5.58 ± 0.665 (2) | 58.41 ± 0.127 (2) | 0.18 ± 0.034 (3) |
| Cruciferin | 6.06 ± NA (1) | 87.55 ± NA (1) | 0.17 ± 0.024 (3) |
| LTP1 | 5.77 ± 0.085 (3) | 40.41 ± 3.103 (3) | 0.18 ± 0.072 (3) |
| PD | 4.55 ± 1.485 (2) | 32.83 ± 18.675 (2) | 0.21 ± 0.027 (3) |
| PG | 6.58 ± NA (1) | 105.17 ± NA (1) | 0.13 ± 0.008 (3) |

### G2116 (SEQ ID NO: 139 and 140)

Published background information. G2116 was identified in the sequence of BAC F4H5, GenBank accession number AC011001, released by the Arabidopsis Genome Initiative. There is no published information regarding the function of G2116.

Discoveries in Arabidopsis. The annotation of G2116 in BAC AC011001 was experimentally determined. The function of this gene was analyzed using transgenic plants in which G2116 was expressed under the control of the 35S promoter. The phenotype of these transgenic plants was wild type in all assays performed. G2116 appeared to be constitutively expressed in all tissues and environmental conditions tested.

Discoveries in tomato. In transgenic tomatoes overexpressing G2116 under the regulatory control of the PG promoter, the fruit lycopene level was higher than the highest wild type level and ranked above the 95th percentile among all lycopene measurements.

TABLE 82
Data Summary for G2116

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | 6.18 ± NA (1) | 94 ± NA (1) | 0.09 ± 0.014 (2) |
| AP1 | 4.91 ± NA (1) | 56.06 ± NA (1) | 0.1 ± 0.015 (2) |
| AS1 | 5.49 ± NA (1) | 45.85 ± NA (1) | 0.1 ± 0.035 (3) |
| Cruciferin | 5.4 ± 0.188 (3) | 73.02 ± 31.149 (3) | 0.14 ± 0.023 (3) |
| PG | 5.37 ± 0.735 (2) | 103.61 ± 35.44 (2) | 0.13 ± 0.032 (3) |

### G2132 (SEQ ID NO: 141 and 142)

Published background information. G2132 was identified in the sequence of BAC clone F27J15 (AC016041, gene F27J15.11). Sakuma et al. (2002) categorized G2132 into the B6 subgroup of the AP2 transcription factor family, with the B family having only a single ERF domain.

Discoveries in Arabidopsis. Overexpressors of G2132 under control of the 35S promoter were slightly small, slower developing, sometimes had pale patches on leaves, and showed reductions in seed yield.

None of the stress challenge array background experiments revealed any regulation of G2132 expression.

Discoveries in tomato. Brix content in fruit was greater than that in wild type controls in plants expressing G2132 under the PG promoter, with a rank in the 95th percentile among all measurements. However, there were no ripe fruit when samples were collected, due to a late-fruiting phenotype in the PG-regulated lines.

TABLE 83
Data Summary for G2132

| | Promoter summary: Avg. ± StD. (Count) | | |
|---|---|---|---|
| | Brix (g sugar/ | Lycopene | Volume |
| Promoter | 100 g sample) | (ppm) | (m³) |
| AP1 | 5.94 ± 0.87 (2) | 75.38 ± 16.278 (2) | 0.27 ± 0.051 (3) |
| AS1 | NA | NA | 0.15 ± 0.041 (3) |
| Cruciferin | NA | NA | 0.2 ± 0.02 (3) |
| PD | NA | NA | 0.19 ± 0.093 (3) |
| PG | 6.43 ± NA (1) | 92.6 ± NA (1) | 0.21 ± 0.037 (2) |

### G2137 (SEQ ID NO: 143 and 144)

Published background information. G2137 corresponds to AtWRKY9 (At1g68150), for which there is no published literature beyond the general description of WRKY family members (Eulgem et al. (2000)).

Discoveries in Arabidopsis. The function of G2137 was studied using transgenic plants in which the gene was expressed under the control of the 35S promoter. 35S::G2137 plants were wild type in morphology and development, as well as in the physiological and biochemical analyses that were performed.

G2137 expression is detected at higher levels in root tissue, and can also be detected in leaf, embryo, and seedling tissue samples. G2137 expression is not ectopically induced by any of the conditions tested, except perhaps by auxin treatment.

In an Arabidopsis microarray experiment, G2137 was found to be five-fold induced (p<0.01) after treatment (0.5 hr) with salicylic acid.

Discoveries in tomato. Transgenic tomatoes expressing G2137 under the AP1, Cruciferin, LTP1, PG, RBCS3 or STM promoters were analyzed for alteration in plant size, soluble solids and lycopene. The Brix levels of STM::G2137 overexpressing tomato plants ranked in the 95th percentile among all other measurements. STM::G2137 overexpressors were noted to be smaller than wild type, and to produce small fruit, consistent with reported observations that fruit size and Brix are frequently inversely related.

TABLE 84
Data Summary for G2137

| | Promoter summary: Avg. ± StD. (Count) | | |
|---|---|---|---|
| | Brix (g sugar/ | Lycopene | Volume |
| Promoter | 100 g sample) | (ppm) | (m³) |
| AP1 | 5.47 ± 0.311 (3) | 44.7 ± 5.315 (3) | 0.18 ± 0.031 (3) |
| Cruciferin | 5.46 ± 0.141 (2) | 42.2 ± 16.589 (2) | 0.2 ± 0.055 (3) |
| LTP1 | 5.09 ± 0.919 (2) | 46.84 ± 0.311 (2) | 0.11 ± 0.063 (3) |
| PG | 4.67 ± NA (1) | 36.06 ± NA (1) | 0.16 ± 0.054 (3) |
| RBCS3 | 5.36 ± 0.12 (3) | 56.45 ± 16.584 (3) | 0.18 ± 0.016 (3) |
| STM | 6.32 ± NA (1) | 84.07 ± NA (1) | 0.14 ± 0.107 (3) |

### G2141 (SEQ ID NO: 145 and 146)

Published background information. The sequence of G2141 was obtained from GenBank accession number AC011665, corresponding to gene T6L1.10, based on its sequence similarity within the conserved domain to other bHLH related proteins in Arabidopsis. G2141 corresponds to AtbHLH049, as described by Heim et al. (2003) and Toledo-Ortiz et al. (2003), which describe the Arabidopsis bHLH gene family.

Discoveries in Arabidopsis. Overexpression of G2141 under control of the 35S promoter in Arabidopsis resulted in plants with elongated cotyledons. Later in development, the majority of these plants appeared wild type, but a number of lines were smaller than controls. Additionally, 3/18 T1 plants (#1, 3 and 12) displayed somewhat flat broad leaves.

In a series of microarray experiments with hormone and stress treatments, G2141 expression was not found to be regulated.

Discoveries in tomato. Brix and lycopene content in fruit was greater than that in wild type controls in plants expressing G2141 under the PG promoter, with a rank in the 95th percentile among all measurements.

TABLE 85
Data Summary for G2141

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | NA | NA | 0.14 ± 0.033 (3) |
| AP1 | 6 ± 0.696 (3) | 58.44 ± 13.932 (3) | 0.13 ± 0.006 (3) |
| LTP1 | 5.88 ± NA (1) | 64.97 ± NA (1) | 0.18 ± 0.04 (3) |
| PG | 6.88 ± NA (1) | 98.78 ± NA (1) | 0.09 ± 0.016 (3) |
| STM | NA | NA | 0.15 ± NA (1) |

### G2145 (SEQ ID NO: 147 and 148)

Published background information. The sequence of G2145 was obtained from GenBank accession number AC012375, based on its sequence similarity within the conserved domain to other bHLH related proteins in Arabidopsis. G2145 corresponds to AtbHLH054, as described by Heim et al. (2003) and Toledo-Ortiz et al. (2003), which describe the Arabidopsis bHLH gene family.

Discoveries in Arabidopsis. Overexpression of G2145 under control of the 35S promoter in Arabidopsis resulted in plants that were distinctly smaller than wild-type at all developmental stages, produced rather curled dark green leaves, and generated thin inflorescences that yielded relatively few seeds.

In a series of microarray experiments with hormone and stress treatments, G2145 expression was found to be up-regulated by cold treatment in roots. Expression of G2145 was also up-regulated in 35S::G682 transgenic in roots. Qualitative RT-PCR experiments indicated that G2145 was expressed root-preferentially.

Discoveries in tomato. Lycopene content in fruit was greater than that in wild type controls in plants expressing G2145 under the PG promoter, with a rank in the 95th percentile among all measurements. In seedlings expressing G2145 under the 35S promoter, leaves had paler green color than in wild type controls. Overexpression of G2145 with the 35S promoter in Arabidopsis produced small plants with contorted, dark green leaves and poor fertility.

Other related data. We have identified one paralog of G2145, G2148, which was not included in the present field trial.

TABLE 86
Data Summary for G2145

| | Promoter summary: Avg. ± StD. (Count) | | |
|---|---|---|---|
| | Brix (g sugar/ | Lycopene | Volume |
| Promoter | 100 g sample) | (ppm) | (m³) |
| AP1 | NA | NA | 0.05 ± 0.039 (3) |
| LTP1 | NA | NA | 0.11 ± 0.015 (3) |
| RBCS3 | 5.83 ± NA (1) | 103.06 ± NA (1) | 0.12 ± 0.032 (3) |
| STM | 4.55 ± NA (1) | 70.84 ± NA (1) | 0.03 ± 0.014 (3) |

### G2150 (SEQ ID NO: 149 and 150)

Published background information. The sequence of G2150 was obtained from GenBank accession number AP000377, corresponding to gene MYM9.3 (13AB01846), based on its sequence similarity within the conserved domain to other bHLH related proteins in Arabidopsis. G2150 corresponds to AtbHLH077, as described by Heim et al. (2003) and Toledo-Ortiz et al. (2003), which describe the Arabidopsis bHLH gene family.

Discoveries in Arabidopsis. Overexpression of G2150 under control of the 35S promoter in Arabidopsis resulted in plants with normal appearance and physiology.

In a series of microarray experiments with hormone and stress treatments, G2150 expression was not found to be regulated.

Discoveries in tomato. Brix content in fruit was greater than that in wild type controls in plants expressing G2150 under the LTP1 promoter, with a rank in the 95th percentile among all measurements. In seedlings expressing G2150 under the 35S promoter, leaves were chlorotic and stems were elongate (etiolated appearance). Overexpression of G2150 with the 35S promoter in Arabidopsis produced plants with normal appearance and physiology.

TABLE 87
Data Summary for G2150

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | 5.45 ± NA (1) | 91.64 ± NA (1) | 0.08 ± 0.061 (3) |
| AP1 | 5.93 ± 0.37 (3) | 85.46 ± 32.407 (3) | 0.19 ± 0.018 (3) |
| AS1 | 6.28 ± 0.134 (2) | 70.95 ± 37.265 (2) | 0.2 ± 0.042 (3) |
| LTP1 | 6.37 ± 0.226 (2) | 81.49 ± 12.544 (2) | 0.1 ± 0.042 (3) |
| RBCS3 | 5.4 ± NA (1) | 70.51 ± NA (1) | 0.12 ± NA (1) |
| STM | 5.85 ± 0.276 (2) | 67.88 ± 18.144 (2) | 0.14 ± 0.046 (3) |

### G2157 (SEQ ID NO: 151 and 152)

Published background information. The sequence of G2157 was obtained from Arabidopsis genomic sequencing project, GenBank accession number AL132975, based on its sequence similarity within the conserved domain to other AT-hook related proteins in Arabidopsis. G2157 corresponds to gene T22E16.220 (CAB75914).

Discoveries in Arabidopsis. The complete sequence of G2157 was determined. G2157 is expressed at low to moderate levels throughout the plant. It shows induction by Fusarium infection and possibly by auxin. The function of this gene was analyzed using transgenic plants in which G2157 was expressed under the control of the 35S promoter.

Overexpression of G2157 produced distinct changes in leaf development and severely reduced overall plant size and fertility. The most strongly affected 35S::G2157 primary transformants were tiny, slow growing, and developed small dark green leaves that were often curled, contorted, or had serrated margins. A number of these plants arrested growth at a vegetative stage and failed to flower. Lines with a more moderate phenotype produced thin inflorescence stems; the flowers borne on these structures were frequently sterile and failed to open or had poorly formed stamens. Due to such defects, the vast majority of T1 plants produced very few seeds. The progeny of three T1 lines showing a moderately severe phenotype were examined; all three T2 populations, however, displayed wild-type morphology, suggesting that activity of the transgene had been reduced between the generations.

G2157 expression has been assayed using microarrays. Assays in which severe drought conditions were applied to 6-week-old Arabidopsis plants resulted in the increase of G2157 transcript approximately two fold above wild type plants.

Discoveries in tomato. Under the regulation of AP1, LTP and STM a significant increase in G2157 overexpressor plant size was observed. Results with the AP1 and STM promoters were particularly notable as the increased plant size was also associated with increased fruit set in these lines.

G2157 is closely related to a subfamily of transcription factors well characterized in their ability to confer drought tolerance and to increase organ size. Genes within this subfamily have also exhibited deleterious morphological effects as in the overexpression of G2157 in Arabidopsis. It has been hypothesized that targeted expression of genes in this subfamily could increase the efficacy or penetrance of desirable phenotypes.

In our overexpression studies of G1073 (G2157 related), different promoters were used to optimize desired phenotypes. In this analysis, we discovered that localized expression via a promoter specific to young leaf and stem primordia (SUC2) was more effective than a promoter (RbcS3) lacking expression in meristematic tissue. In tomato, a similar result was obtained by expressing G2157 in meristematic and primordial tissues via the STM and AP1 promoters, respectively. G2157 has also been identified as being significantly induced under severe drought conditions. These results provide strong evidence that G2157, when expressed in localized tissues in tomatoes, mechanistically functions in a similar fashion to its closely related putative paralogs in the G1073 clade.

Other related data. In a phylogenetic analysis of AT-hook proteins, G2157 falls within the G1073 clade of transcription factor polypeptides, a subfamily characterized as being involved in regulation of abiotic stress responses, organ size and overall plant size. This clade contains a sizable number of genes from monocot and dicot species that have been shown to increase organ size when overexpressed.

TABLE 88
Data Summary for G2157

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | 4.83 ± 0.272 (3) | 51.17 ± 11.663 (3) | 0.31 ± 0.087 (3) |
| AP1 | 6.14 ± 0.43 (3) | 78.05 ± 12.231 (3) | 0.33 ± 0.068 (3) |
| AS1 | 5.94 ± 0.242 (3) | 80.99 ± 27.876 (3) | 0.18 ± 0.035 (3) |
| Cruciferin | 5.08 ± 0.219 (2) | 69.16 ± 9.737 (2) | 0.29 ± 0.054 (3) |
| LTP1 | 5.5 ± 0.321 (3) | 87.62 ± 15.783 (3) | 0.33 ± 0.054 (3) |
| PD | 5.84 ± 0.255 (2) | 67.94 ± 35.751 (2) | 0.31 ± 0.049 (3) |
| PG | 5.43 ± 0.099 (2) | 70.38 ± 24.947 (2) | 0.23 ± 0.1 (3) |
| RBCS3 | 5.7 ± 0.862 (3) | 75.57 ± 4.603 (3) | 0.23 ± 0.168 (3) |
| STM | 5.5 ± 0.163 (2) | 64.78 ± 17.388 (2) | 0.36 ± 0.114 (2) |

### G2294 (SEQ ID NO: 153 and 154)

Published background information. G2294 corresponds to gene T12C22.10 (AAF78266). Sakuma et al. (2002) categorized G2294 into the A5 subgroup of the AP2 transcription factor family, with the A family related to the DREB and CBF genes.

Discoveries in Arabidopsis. Overexpression of G2294 under control of the 35S promoter produced plants that were markedly smaller than wild-type controls. The most severely affected T1 plant died without flowering, whilst the others formed short, thin, inflorescences that carried small, poorly-fertile flowers, and set few seeds. In a series of microarray experiments with hormone and stress treatments, G2294 was found to be up-regulated by ACC treatment in shoots after 4-8 hours, induced in roots by cold treatment from 0.5 up through 8 hours following treatment, and induced in roots 4-8 hours following salt treatment.

Discoveries in tomato. Lycopene and Brix content in fruit were greater than that in wild type controls in plants expressing G2294 under the LTP1 promoter, with a rank in the 95th percentile among all measurements (but this result was obtained with only a single fruit sample). Brix level and plant size were greater than that in wild type controls in plants expressing G2294 under the 35S promoter, with a rank in the 95th percentile among all measurements. In seedlings expressing G2294 under the 35S promoter, size was normal but leaves were narrow and curled downward. Plant size was also significantly reduced upon overexpression of G2294 with the 35S promoter in Arabidopsis.

Other related data. We have identified two paralogs of G2294 in Arabidopsis, G2067 and G2115. These genes were not included in the present field trial.

TABLE 89
Data Summary for G2294

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | 6.31 ± 0.453 (3) | 71.9 ± 9.018 (3) | 0.32 ± 0.078 (3) |
| AS1 | 5.76 ± 0.969 (2) | 62.41 ± 11.985 (2) | 0.16 ± 0.098 (3) |
| LTP1 | 6.31 ± NA (1) | 127.71 ± NA (1) | 0.22 ± 0.047 (3) |
| RBCS3 | 5.49 ± 0.357 (3) | 73.09 ± 4.85 (3) | 0.29 ± 0.045 (3) |
| STM | 5.88 ± 0.845 (3) | 72.51 ± 7.079 (3) | 0.23 ± 0.053 (3) |

### G2296 (SEQ ID NO: 155 and 156)

Published background information. G2296 corresponds to AtWRKY66 (At1 g80590), for which there is no published literature beyond the general description of WRKY family members (Eulgem et al. (2000)).

Discoveries in Arabidopsis. The function of G2296 was studied using transgenic plants in which the gene was expressed under the control of the 35S promoter. 35S::G2296 plants were wild type in morphology and development, as well as in the physiological and biochemical analyses that were performed.

G2296 expression was detected in a variety of tissues, and the gene was strongly induced by salicylic acid in root tissue (up to 8-fold).

Discoveries in tomato. Plants expressing Cruciferin::G2296 were noted to be very large, and to be generally delayed in fruit maturation. The Brix level of transgenic tomatoes expressing G2296 under control of the Cruciferin promoter ranked in the 95th percentile among all Brix measurements and was higher than in any wild-type plant measured. A single plant expressing Cruciferin::G2296 produced no fruit, as did plants overexpressing G2296 with the AP1 or AS1 promoters.

TABLE 90
Data Summary for G2296

| | Promoter summary: Avg. ± StD. (Count) | | |
|---|---|---|---|
| | Brix (g sugar/ | Lycopene | Volume |
| Promoter | 100 g sample) | (ppm) | (m³) |
| AP1 | NA | NA | 0.11 ± 0.018 (3) |
| AS1 | 6.24 ± NA (1) | 50.62 ± NA (1) | 0.07 ± 0.008 (3) |
| Cruciferin | 6.73 ± NA (1) | 50.74 ± NA (1) | 0.1 ± 0.078 (3) |
| PG | NA | NA | 0.17 ± 0.072 (3) |
| RBCS3 | 5.95 ± 0.191 (3) | 91.18 ± 35.404 (3) | 0.21 ± 0.044 (3) |
| STM | 6.02 ± NA (1) | 42.39 ± NA (1) | 0.07 ± 0.016 (2) |

### G2313 (SEQ ID NO: 157 and 158)

Published background information. G2313 (At3g10590) was identified in the sequence of BAC F13M14 (GenBank accession number AC011560), released by the Arabidopsis Genome Initiative.

Discoveries in Arabidopsis. The function of this gene was analyzed using transgenic Arabidopsis plants in which G2313 was expressed under the control of the 35S promoter. Analysis of primary 35S::G2313 transformants indicates that overexpression of this gene in Arabidopsis has detrimental effects for plant growth and development. However, these lines displayed a wild-type morphology in the next generation, possibly due to silencing of the transgene. T2 generation plants were wild type in all biochemical and physiological assays performed. As determined by RT-PCR, G2313 is highly expressed in flower, embryo, and silique. Very low levels of G-313 expression were also detected in other tissue with the exception of germinating seeds. G2313 was also induced slightly by SA, auxin, ABA, osmotic stress and heat stress treatments, as determined by RT-PCR. G2313 was not found to be significantly induced or repressed in any of our GeneChip microarray experiments.

Discoveries in tomato. The fruit lycopene level under the AS1 promoter was higher than the highest wild type level and ranked in the 95th percentile among all lycopene measurements. Analysis of primary 35S::G2313 transformants indicated that overexpression of this gene in Arabidopsis had detrimental effects for plant growth and development. However, these lines displayed a wild-type morphology in the next generation, possibly due to silencing of the transgene. T2 generation plants were wild type in all biochemical and physiological assays performed.

TABLE 91
Data Summary for G2313

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | 4.87 ± 0.398 (3) | 34.51 ± 9.183 (3) | 0.15 ± 0.053 (3) |
| AP1 | 5.28 ± 0.58 (2) | 45.68 ± 21.793 (2) | 0.19 ± 0.009 (3) |
| AS1 | 5.35 ± 0.509 (2) | 100.96 ± 17.522 (2) | 0.15 ± 0.014 (3) |
| STM | NA | NA | 0.14 ± 0.019 (2) |

### G2417 (SEQ ID NO: 159 and 160)

Published background information. G2417 was identified in the sequence of chromosome 2, GenBank accession number AC00656, released by the Arabidopsis Genome Initiative. No further published or public information is available about G2417.

Discoveries in Arabidopsis. The function of G2417 was analyzed using transgenic plants in which this gene was expressed under the control of the 35S promoter. The phenotype of these transgenic plants was wild type in all morphological, physiological, and biochemical assays performed. G2417 is ubiquitously expressed, and it is not induced or repressed by any condition tested by RT-PCR or microarray analysis.

Discoveries in tomato. Plants expressing G2417 under the LTP1 promoter were in the 95th percentile of fruit lycopene measurements.

TABLE 92
Data Summary for G2417

| | Promoter summary: Avg. ± StD. (Count) | | |
|---|---|---|---|
| | Brix (g sugar/ | Lycopene | Volume |
| Promoter | 100 g sample) | (ppm) | (m³) |
| AP1 | 5.91 ± 0.12 (2) | 61.53 ± 1.322 (2) | 0.27 ± 0.022 (3) |
| AS1 | NA | NA | 0.15 ± 0.066 (3) |
| Cruciferin | 5.35 ± 0.283 (2) | 47 ± 18.604 (2) | 0.24 ± 0.014 (3) |
| LTP1 | 5.74 ± NA (1) | 114.96 ± NA (1) | 0.2 ± 0.056 (3) |
| PD | NA | NA | 0.18 ± 0.034 (3) |
| PG | 5.45 ± NA (1) | 63.04 ± NA (1) | 0.25 ± 0.076 (3) |
| STM | 5.42 ± 0.643 (2) | 53.45 ± 8.294 (2) | 0.17 ± 0.055 (3) |

### G2425 (SEQ ID NO: 161 and 162)

Published background information. G2425 corresponds to gene At1 g74430 and is also referred to as AtMYB95 (Stracke et al. (2001)).

Discoveries in Arabidopsis. The function of G2425 was analyzed using transgenic Arabidopsis plants in which the gene was expressed under the control of the 35S promoter. The phenotype of the 35S::G2425 transgenic plants was wild type in morphology and development, as well as in the different physiological and biochemical analyses that were performed.

RT-PCR analysis of the endogenous levels of G2425 indicates that this gene is expressed ubiquitously and that it may be induced by ABA and auxin treatments. Microarray analysis shows that G2425 is repressed by drought stress, induced by methyl jasmonate, and may be induced by ABA.

Discoveries in tomato. The size of tomato plants overexpressing G2425 under the AP1 and PD promoters ranked in the 95th percentile among all plant size measurements. In addition, under the LTP1 promoter, the fruit Brix level was very close to the highest wild-type level and ranked in the 95th percentile among all Brix measurements.

TABLE 93
Data Summary for G2425

| | Promoter summary: Avg. ± StD. (Count) | | |
|---|---|---|---|
| | Brix (g sugar/ | Lycopene | Volume |
| Promoter | 100 g sample) | (ppm) | (m³) |
| 35S | 5.53 ± NA (1) | 56.39 ± NA (1) | 0.25 ± 0.042 (3) |
| AP1 | 5.03 ± 0.615 (3) | 68 ± 28.893 (3) | 0.32 ± 0.01 (3) |
| AS1 | 4.62 ± NA (1) | 50.49 ± NA (1) | 0.25 ± 0.059 (3) |
| Cruciferin | 6.1 ± 0.401 (3) | 55.05 ± 2.412 (3) | 0.26 ± 0.027 (3) |
| LTP1 | 6.32 ± NA (1) | 49.06 ± NA (1) | 0.21 ± 0.032 (3) |
| PD | 5.51 ± 0.611 (3) | 46.7 ± 15.531 (3) | 0.33 ± 0.052 (3) |
| PG | NA | NA | 0.15 ± 0.049 (3) |

### G2505 (SEQ ID NO: 163 and 164)

Published background information. G2505 was identified in the sequence of contig fragment No. 29, GenBank accession number AL161517, released by the Arabidopsis Genome Initiative. It also corresponds to the AGI locus of AT4G10350. A comprehensive analysis of NAC family transcription factors was recently published by Ooka et al. (2003) where G2052 was identified as ANAC070.

Discoveries in Arabidopsis. Analysis of the function of G2505 was attempted through the generation transgenic plants in which the gene was expressed under the control of the 35S promoter. However, despite numerous repeated attempts, we were only able to obtain a few 35S::G2505 transformants; thus, overexpression of this gene likely caused lethality during embryo or early seedling development. In addition to the deleterious effects of this gene when overexpressed, a few lines that were obtained were distinctly small and dark in coloration. Only two of these lines produced sufficient seed for physiology assays to be performed. Both of those lines displayed enhanced performance in a severe drought assay. In a phylogenetic analysis, G2635 was determined to the most similar to G2505. We have not identified functional data for G2635. Microarray data did not show any significant transcriptional differences to wild type in all experimental conditions assayed.

Discoveries in tomato. Under the regulation of the RBCS3 promoter, a significant increase in lycopene levels in G2505 overexpressors was observed.

Other related data. We have identified one paralog of G2505 in Arabidopsis, G2635, which was not included in the present field trial.

TABLE 94
Data Summary for G2505

| | Promoter summary: Avg. ± StD. (Count) | | |
|---|---|---|---|
| | Brix (g sugar/ | Lycopene | Volume |
| Promoter | 100 g sample) | (ppm) | (m³) |
| AP1 | 4.72 ± 0.233 (2) | 81.77 ± 16.44 (2) | 0.23 ± 0.024 (3) |
| AS1 | NA | NA | 0.2 ± 0.035 (3) |
| Cruciferin | 5.69 ± NA (1) | 82.83 ± NA (1) | 0.29 ± NA (1) |
| LTP1 | NA | NA | 0.22 ± 0.01 (3) |
| PD | NA | NA | 0.13 ± 0.038 (3) |
| RBCS3 | 5.29 ± NA (1) | 99.52 ± NA (1) | 0.24 ± 0.03 (3) |
| STM | NA | NA | 0.23 ± 0.039 (3) |

### Example VII

### Summary of Results

Using the methods described in the above Examples, we identified a number of Arabidopsis sequences that resulted in higher fruit Brix, higher fruit lycopene, and enhanced plant size, respectively, when expressed in tomato. A summary of the sequences that resulted in higher fruit Brix, higher fruit lycopene, and enhanced plant size is presented in Tables 95, 96 and 97. In the tables, a G0D may be repeated if two or more replicates fell within the 95th percentile.

TABLE 95
Experimental values for soluble solids
(Brix) in or above 95% percentile

| | | Measured Brix |
|---|---|---|
| GID | Promoter | (g sugar/100 g sample) |
| G22 | AP1 | 7.29 |
| G2141 | PG | 6.88 |
| G635 | PD | 6.85 |
| G522 | 35S | 6.8 |
| G2296 | Cruciferin | 6.73 |
| G580 | STM | 6.7 |
| G1007 | Cruciferin | 6.67 |
| G1755 | AP1 | 6.67 |
| G1755 | PD | 6.66 |
| G1444 | LTP1 | 6.63 |
| G843 | RBCS3 | 6.61 |
| G1481 | RBCS3 | 6.6 |
| G843 | AP1 | 6.59 |
| G551 | STM | 6.58 |
| G2108 | PG | 6.58 |
| G1053 | Cruciferin | 6.55 |
| G1809 | LTP1 | 6.51 |
| G1935 | LTP1 | 6.49 |
| G1791 | PG | 6.48 |
| G1954 | AP1 | 6.47 |
| G1785 | STM | 6.44 |
| G2132 | PG | 6.43 |
| G1808 | RBCS3 | 6.42 |
| G1007 | AP1 | 6.42 |
| G522 | AP1 | 6.41 |
| G159 | LTP1 | 6.41 |
| G558 | STM | 6.39 |
| G1650 | LTP1 | 6.38 |
| G2150 | LTP1 | 6.37 |
| G1784 | Cruciferin | 6.36 |
| G1462 | AP1 | 6.36 |
| G22 | STM | 6.34 |
| G1645 | PG | 6.33 |
| G2425 | LTP1 | 6.32 |
| G2137 | STM | 6.32 |
| G567 | AP1 | 6.31 |
| G558 | AS1 | 6.31 |
| G2294 | LTP1 | 6.31 |
| G1635 | LTP1 | 6.31 |
| G2294 | 35S | 6.31 |
| G1635 | PG | 6.3 |
| G187 | STM | 6.29 |
| G450 | STM | 6.28 |

TABLE 96
Experimental values for lycopene in or above 95% percentile

| | | Measured Lycopene |
|---|---|---|
| GID | Promoter | (PPM) |
| G2294 | LTP1 | 127.71 |
| G1635 | STM | 121.53 |
| G1638 | PG | 119.22 |
| G2417 | LTP1 | 114.96 |
| G328 | AP1 | 114.15 |
| G1324 | PG | 112.42 |
| G580 | 35S | 111.92 |
| G1273 | AP1 | 110.56 |
| G450 | STM | 109.97 |
| G881 | STM | 108.85 |
| G635 | PD | 108.82 |
| G1884 | LTP1 | 108.76 |
| G580 | STM | 106.67 |
| G237 | PD | 106.1 |
| G1078 | RBCS3 | 105.46 |
| G2108 | PG | 105.17 |
| G363 | LTP1 | 105.08 |
| G2072 | AS1 | 104.79 |
| G3 | RBCS3 | 104.6 |
| G2116 | PG | 103.61 |
| G2145 | RBCS3 | 103.06 |
| G675 | RBCS3 | 103 |
| G1226 | RBCS3 | 102.73 |
| G328 | PG | 102.46 |
| G22 | RBCS3 | 102.29 |
| G1755 | PD | 102.03 |
| G675 | STM | 101.65 |
| G2313 | AS1 | 100.96 |
| G843 | AP1 | 100.95 |
| G1007 | AP1 | 100.75 |
| G156 | AP1 | 100.37 |
| G435 | RBCS3 | 99.77 |
| G2505 | RBCS3 | 99.52 |
| G383 | STM | 99.38 |
| G159 | LTP1 | 99.05 |
| G2141 | PG | 98.78 |
| G558 | AS1 | 98.75 |
| G237 | PG | 98.4 |
| G190 | STM | 98.31 |
| G1903 | LTP1 | 98.26 |
| G675 | AS1 | 97.58 |
| G1462 | AP1 | 97.53 |
| G843 | 35S | 97.32 |

TABLE 97
Experimental values for plant volume in or above 95% percentile

| GID | Promoter | Measured Volume (m³) |
|---|---|---|
| G1463 | RBCS3 | 0.5 |
| G1053 | AP1 | 0.46 |
| G812 | PD | 0.45 |
| G47 | LTP1 | 0.43 |
| G1950 | AP1 | 0.42 |
| G729 | Cruciferin | 0.41 |
| G1958 | Cruciferin | 0.41 |
| G1958 | AS1 | 0.4 |
| G1903 | LTP1 | 0.4 |
| G24 | Cruciferin | 0.4 |
| G1752 | Cruciferin | 0.39 |
| G1463 | STM | 0.38 |
| G1895 | AP1 | 0.37 |
| G2157 | STM | 0.36 |
| G2052 | AP1 | 0.36 |
| G1053 | AS1 | 0.36 |
| G729 | PG | 0.36 |
| G1950 | PD | 0.36 |
| G812 | Cruciferin | 0.35 |
| G1815 | 35S | 0.35 |
| G24 | AS1 | 0.35 |
| G1895 | AS1 | 0.34 |
| G1543 | LTP1 | 0.34 |
| G2052 | PD | 0.34 |
| G1640 | AS1 | 0.34 |
| G2052 | LTP1 | 0.34 |
| G270 | AS1 | 0.34 |
| G2425 | PD | 0.33 |
| G675 | 35S | 0.33 |
| G1903 | Cruciferin | 0.33 |
| G1504 | STM | 0.33 |
| G1755 | PD | 0.33 |
| G1635 | PD | 0.33 |
| G1444 | 35S | 0.33 |
| G2157 | AP1 | 0.33 |
| G1752 | 35S | 0.33 |
| G675 | AP1 | 0.33 |
| G1909 | Cruciferin | 0.33 |
| G1958 | 35S | 0.33 |
| G1752 | PG | 0.33 |
| G2157 | LTP1 | 0.33 |
| G937 | PG | 0.33 |
| G2425 | AP1 | 0.32 |
| G989 | STM | 0.32 |
| G989 | Cruciferin | 0.32 |
| G1755 | PG | 0.32 |
| G1865 | Cruciferin | 0.32 |
| G1950 | LTP1 | 0.32 |
| G1950 | PG | 0.32 |
| G1328 | RBCS3 | 0.32 |
| G1650 | AP1 | 0.32 |
| G558 | AP1 | 0.32 |
| G1635 | AP1 | 0.32 |
| G1897 | Cruciferin | 0.32 |
| G1444 | AS1 | 0.32 |
| G1543 | PG | 0.32 |
| G226 | Cruciferin | 0.32 |
| G2294 | 35S | 0.32 |

Of particular interest, seven genes (G558, G843, G1007, G1755, G22, G2294, and G522) showed high Brix levels when overexpressed with more than one promoter; five genes (G580, G237, G675, G843, and G328) resulted in high fruit lycopene when overexpressed with more than one promoter; while eighteen genes (G989, G1053, G1635, G675, G1444, G1950, G812, G1958, G729, G1752, G1755, G24, G1543, G1463, G2052, G2157, G1895, and G1903) resulted in larger vegetative plant size when overexpressed with more than one promoter. It is noteworthy that plants overexpressing G1950 under four different promoters rank in the top 95th percentile in size measurement while plants overexpressing G1958, G1752, G2052, or G2157 under three different promoters showed an increase in plant size. A few examples are discussed below.

G1950 (AKR family) is structurally related to NPR1, and thus may have a similar function in disease resistance. The enhanced size observed with AP1, LTP1, PD and PG promoters (in addition, the 35S::G1950 gene gave rise to increased size at 90th percentile) may be due to resistance to plant diseases in the field. It is also possible that enhanced expression of G1950 fosters enhanced growth, compared to wild-type controls, under stressful conditions that include biotic and abiotic stresses. Interestingly, Arabidopsis growth was unaffected in 35S::G1950 plants.

G1958 (GARP family) is known to be involved in regulation of a response to phosphate limitation. Over-expression of G1958 with 35S, AS1 and cruciferin promoters resulted in increased plant size, suggesting that phosphate levels in the field conditions were limiting and the improved response contributed to enhanced plant growth.

Plant size was also significantly increased with G2157 (AT-hook family) under the control of either the AP1, LTP1 and STM promoters. Plant size was also above the median with every other promoter tested, with the exception of the AS1 promoter (which has the median value). These results are consistent with increased plant growth associated with overexpression of a set of related AT-hook genes. Interestingly, in Arabidopsis, overexpression with the 35S promoter yielded significantly stunted plants with contorted leaves. This is consistent with possible involvement of auxin pathways (and perhaps an epinastic leaf response) in increased plant size. Other related AT-hook genes in Arabidopsis have been found to give mostly dwarfed transgenic plants, with occasional lines larger than wild type controls. These data support the role of AT-hook genes in the control of overall plant biomass.

Several genes may cause increases in plant size by conferring drought tolerance to plants in the field. For example, G675 expression under three different promoters (35S, AP1, and LTP1) ranked in the 95th percentile for size. This observation is supported by the Cruciferin promoter, PD, and PG promoters—all ranked above 75th percentile. Interestingly, G675 is also a lycopene hit under three different promoters (AS1, RBCS3, and STM), suggesting a relationship between the two traits. G675 is induced in roots by osmotic stress and ABA in Arabidopsis and it is possible it may be involved in general abiotic stress tolerance. G989 (related to SCR) also has produced increases in plant size under three promoters (Cruciferin and STM, 95 percentile; and LTP1, 90th percentile). G989 expression is induced by auxin, heat, drought, salt, osmotic stress. Others that have increased plant size such as G812 under multiple promoters (Cruciferin and PD, 95th percentile; LTP1, RBCS3, and STM, above 90th percentile) have shown drought tolerance directly when expressed under the 35S promoter.

Increased plant size can also be a result of effects on plant development. In the case of G1444 (GRF family), overexpression resulted in increased plant size under three different promoters (35S, AS1, and RBCS3). Ectopic expression in Arabidopsis of a large majority of the genes belonging to the GRF family results in a morphological phenotype analogous to that in tomato, i.e., increased leaf/cotyledon surface area and delayed flowering.

In some cases plant size was positively correlated with fruit yield. Examples include G226 under the Cruciferin promoter and G558 under the AP1 promoter, where both plant size and fruit yield were near the top. We have found that G226 confers drought tolerance and enhanced nitrogen utilization.

We have also identified genes that resulted in increases in Brix and lycopene with good or increased fruit yield. For example, expression of G22 under both the AP1 and STM promoters have resulted in high Brix levels while the yield of all five plants was excellent. G22 expression has been found to be responsive to a number of stress conditions in Arabidopsis. G1659 (DBP family) also induced increased lycopene when expressed under the control of the Cruciferin, AS1, and STM promoters. Cruciferin::G1659 and STM::G1659 plants were also noted to have a heavy, but somewhat late fruit-set. However, AS1::G1659 plants had a very heavy fruit-set that was not delayed developmentally.

Brix levels were increased by the expression of G1755 (AP2 family) under control of the AP1 and PD promoters, with a rank in the 95th percentile among all measurements. Lycopene content and plant size was also found to be in the 95th percentile of the PD::G1755 plants. The ability of G1755 to impact Brix, lycopene and plant size may prove to be commercially significant.

G1635 (MYB related) expression was correlated with high lycopene, large plant size and good fruit-set, when expressed under control of the STM promoter. Additionally, large size was also correlated with very high fruit-set in AP1::G1635 and PD::G1635 plants. These tomato plants appeared bushier, possibly due to an increase in lateral branching. A similar reduced apical dominance phenotype was previously documented in Arabidopsis. Finally, the fruit Brix levels for G1635 expressed under the LTP1 and PG promoters were close to the highest wild type level and ranked in the 95th percentile among all Brix measurements.

### Example IX

### Introduction of Polynucleotides into Dicotyledonous Plants and Cereal Plants

Transcription factor sequences listed in the Sequence Listing recombined into expression vectors, such as pMEN20 or pMEN65, may be transformed into a plant for the purpose of modifying plant traits. It is now routine to produce transgenic plants using most dicot plants (see Weissbach and Weissbach, (1989) supra; Gelvin et al. (1990); Herrera-Estrella et al. (1983); Bevan (1984); and Klee (1985)). Methods for analysis of traits are routine in the art and examples are disclosed above.

The cloning vectors of the invention may also be introduced into a variety of cereal plants. Cereal plants such as, but not limited to, corn, wheat, rice, sorghum, or barley, may also be transformed with the present polynucleotide sequences in pMEN20 or pMEN65 expression vectors for the purpose of modifying plant traits. For example, pMEN020 may be modified to replace the NptII coding region with the BAR gene of Streptomyces hygroscopicus that confers resistance to phosphinothricin. The KpnI and BglII sites of the Bar gene are removed by site-directed mutagenesis with silent codon changes.

The cloning vector may be introduced into a variety of cereal plants by means well known in the art such as, for example, direct DNA transfer or Agrobacterium tumefaciens-mediated transformation. It is now routine to produce transgenic plants of most cereal crops (Vasil (1994)) such as corn, wheat, rice, sorghum (Cassas et al. (1993)), and barley (Wan and Lemeaux (1994)). DNA transfer methods such as the microprojectile can be used for corn (Fromm et al. (1990); Gordon-Kamm et al. (1990); Ishida (1990)), wheat (Vasil et al. (1992); Vasil et al. (1993b); Weeks et al. (1993)), and rice (Christou (1991); Hiei et al. (1994); Aldemita and Hodges (1996); and Hiei et al. (1997)). For most cereal plants, embryogenic cells derived from immature scutellum tissues are the preferred cellular targets for transformation (Hiei et al. (1997); Vasil (1994)).

Vectors according to the present invention may be transformed into corn embryogenic cells derived from immature scutellar tissue by using microprojectile bombardment, with the A 88XB73 genotype as the preferred genotype (Fromm et al. (1990); Gordon-Kamm et al. (1990)). After microprojectile bombardment the tissues are selected on phosphinothricin to identify the transgenic embryogenic cells (Gordon-Kamm et al. (1990)). Transgenic plants are regenerated by standard corn regeneration techniques (Fromm et al. (1990); Gordon-Kamm et al. (1990)).

The vectors prepared as described above can also be used to produce transgenic wheat and rice plants (Christou (1991); Hiei et al. (1994); Aldemita and Hodges (1996); and Hiei et al. (1997)) that coordinately express genes of interest by following standard transformation protocols known to those skilled in the art for rice and wheat (Vasil et al. (1992); Vasil et al. (1993); and Weeks et al. (1993)), where the bar gene is used as the selectable marker.

### Example X

### Genes that Confer Significant Improvements to Diverse Plant Species

The function of specific orthologs of the sequences of the invention may be further characterized and incorporated into crop plants. The ectopic overexpression of these orthologs may be regulated using constitutive, inducible, or tissue specific regulatory elements. Genes that have been examined and have been shown to modify plant traits (including increasing lycopene, soluble solids and disease tolerance) encode orthologs of the transcription factor polypeptides found in the Sequence Listing, including, for example, G3380, G3381, G3383, G3392, G3393, G3430, G3431, G3444, G3445, G3446, G3447, G3448, G3449, G3450, G3490, G3515, G3516, G3517, G3518, G3519, G3520, G3524, G3643, G3644, G3645, G3646, G3647, G3649, G3651, G3656, G3659, G3660, G3661, G3717, G3718, G3735, G3736, G3737, G3739, G3794, G3841, G3843, G3844, G3845, G3846, G3848, G3852, G3856, G3857, G3858, G3864, and G3865. In addition to these sequences, it is expected that related polynucleotide sequences encoding polypeptides found in the Sequence Listing can also induce altered traits, including increasing lycopene, soluble solids and disease tolerance, when transformed into a considerable variety of plants of different species, and including dicots and monocots. The polynucleotide and polypeptide sequences derived from monocots (e.g., the rice sequences) may be used to transform both monocot and dicot plants, and those derived from dicots (e.g., the Arabidopsis and soy genes) may be used to transform either group, although it is expected that some of these sequences will function best if the gene is transformed into a plant from the same group as that from which the sequence is derived.

Transgenic plants are subjected to assays to measure plant volume, lycopene, soluble solids, disease tolerance, and fruit set according to the methods disclosed in the above Examples.

These experiments demonstrate that a significant number the transcription factor polypeptide sequences of the invention can be identified and shown to increased volume, lycopene, soluble solids and disease tolerance. It is expected that the same methods may be applied to identify and eventually make use of other members of the clades of the present transcription factor polypeptides, with the transcription factor polypeptides deriving from a diverse range of species.

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the Claims. Modifications that become apparent from the foregoing description and accompanying figures fall within the scope of the following Claims.

### REFERENCES CITED

Aldemita and Hodges (1996) Planta 199:612-617
Ainley et al. (1993) Plant Mol. Biol. 22: 13-23
Altschul et al. (1990) J. Mol. Biol. 215: 403-410
Altschul (1993) J. Mol. Evol. 36: 290-300
Alvarez-Buylla et al. (2000) Proc. Natl. Acad. Sci. USA 97: 5328-5333
Ammirato et al., eds., (1984) Handbook of Plant Cell Culture—Crop Species, Macmillan Publ. Co., New York, N. Y.
An et al. (1988) Plant Physiol. 88: 547-552
Anderson and Young (1985) “Quantitative Filter Hybridisation.” In: Hames and Higgins, ed., Nucleic Acid Hybridisation, A Practical Approach. Oxford, IRL Press, 73-111
Angiosperm Phylogeny Group (1998) Ann. Missouri Bot. Gard. 84: 1-49
Aoyama et al. (1995) Plant Cell 7: 1773-1785
Assmann (2002) Plant Cell 14: S355-S373
Ausubel et al. (1997) Short Protocols in Molecular Biology, John Wiley & Sons, New York, N.Y., unit 7.7
Ausubel et al., eds. (1998) Current Protocols in Molecular Biology, Greene Publishing Associates, Inc. and John Wiley & Sons, Inc., (supplemented through 2000) (“Ausubel”)
Baerson et al. (1993) Plant Mol. Biol. 22: 255-267
Baerson et al. (1994) Plant Mol. Biol. 26: 1947-1959
Bairoch et al. (1997) Nucleic Acids Res. 25: 217-221
Bartley and Scolnik (1995) Plant Cell 7: 1027-1038
Baumann et al., (1999) Plant Cell 11: 323-334
Beaucage et al. (1981) Tetrahedron Letters 22: 1859-1869
Berger and Kimmel (1987) Guide to Molecular Cloning Techniques, Methods in Enzymology, vol. 152 Academic Press, Inc., San Diego, Calif. (“Berger and Kimmel”)
Berrocal-Lobo et al. (2002) Plant J. 29: 23-32
Bevan (1984) Nucleic Acids Res. 12: 8711-8721
Bhattacharjee et al. (2001) Proc Natl. Acad. Sci., USA, 98: 13790-13795
Bolle (2003) Planta 218: 683-692
Borevitz et al. (2000) Plant Cell 12: 2383-2394
Boss and Thomas (2002) Nature, 416: 847-850
Breen and Crouch (1992) Plant Mol. Biol. 19:1049-1055
Bruce et al. (2000) Plant Cell, 12: 65-79
Buchel et al. (1999) Plant Mol. Biol. 40: 387-396
Bulyk et al. (1999) Nature Biotechnol. 17: 573-577
Brummelkamp et al. (2002) Science 296:550-553
Byrne et al (2000) Nature 408: 967-971
Cassas et al. (1993) Proc. Natl. Acad. Sci. 90: 11212-11216
Cao et al. (1997) Cell 88: 57-63
Chase et al. (1993) Ann. Missouri Bot. Gard. 80: 528-580
Cheng et al. (1994) Nature 369: 684-685
Chern et al. (2001)Plant J. 27: 101-113
Chien et al. (1991) Proc. Natl. Acad. Sci. 88: 9578-9582
Chrispeels et al. (2000) Plant Mol. Biol. 42: 279-290
Christou (1991) Bio/Technology 9: 957-962
Constans (2002) The Scientist 16: 36
Corona et al. (1996) Plant J. 9: 505-512
Coupland (1995) Nature 377: 482-483
Crowley et al. (1985) Cell 43: 633-641
Cunningham and Gantt (1998) Annu. Rev. Plant Physiol. Plant Mol. Biol. 49: 557-583
Daly et al. (2001) Plant Physiol. 127: 1328-1333
de Pater et al (1996) Mol. Gen. Genet. 250: 237-239
Denekamp and Smeekens (2003) Plant Physiol. 132: 1415-1423
Doolittle, ed., (1996) Methods Enzymol., vol. 266, “Computer Methods for Macromolecular Sequence Analysis”, Academic Press, Inc., San Diego, Calif., USA
Di Laurenzio et al. (1996) Cell 86:423-433
Eddy (1996) Curr. Opin. Str. Biol. 6: 361-365
Ellis et al. (2002) Plant Cell 14: 1557-1566
Eulgem et al. (2000) Trends Plant Sci. 5: 199-206
Eyal et al. (1992) Plant Mol. Biol. 19: 589-599
Fan and Dong (2002) Plant Cell 14: 1377-1389
Feng and Doolittle (1987) J. Mol. Evol. 25: 351-360
Fire et al. (1998) Nature 391: 806-811
Fluhr et al (1986) EMBO J. 5: 2063-2071
Foley et al. (1993) Plant J. 3: 669-679
Fowler and Thomashow (2002) Plant Cell 14: 1675-1690
Fraley et al. (1983) Proc. Natl. Acad. Sci. 80: 48034807
Frary et al. (2000) Science 289: 85-88
Fraser et al. (1994) Plant Physiol. 105: 405-413
Fraser et al. (2002) Proc. Natl. Acad. Sci. USA 99: 1092-1097
Fridman et al. (2002) Mol. Genet. Genomics 66: 821-826
Fromm et al. (1985) Proc. Natl. Acad. Sci. 82: 5824-5828
Fromm et al. (1989) Plant Cell 1: 977-984
Fromm et al. (1990) Bio/Technol. 8: 833-839
Fu et al. (2001) Plant Cell 13: 1791-1802
Fukaki et al. (2002) Plant J. 29: 153-168
Gampala et al. (2001) J. Biol. Chem. 277: 1689-1694
Gan and Amasino (1995) Science 270: 1986-1988)
Gatz (1997) Annu. Rev. Plant Physiol. Plant Mol. Biol. 48: 89-108
Gelvin et al. (1990) Plant Molecular Biology Manual, Kluwer Academic Publishers
Giniger and Ptashne (1987) Nature 330: 670-672
Giovannoni (2001) Annu. Rev. Plant Physiol. Plant Mol. Biol. 52: 725-749
Gilmour et al. (1998) Plant J. 16: 433-442
Gocal et al. (2001) Plant Physiol. 127:1682-1693
Goodrich et al. (1993) Cell 75: 519-530
Gordon-Kamm (1990) Plant Cell 2: 603-618
Guevara-Garcia (1998) Plant Mol. Biol. 38: 743-753
Guyer et al. (1998) Genetics 149: 633639
Hames and Higgins, eds. (1985) Nucleic Acid Hybridisation: A Practical Approach, IRL Press, Oxford, U. K.
Hammond et al. (2001) Nature Rev Gen 2: 110-119
Harlow and Lane (1988), Antibodies: A Laboratory Manual, Cold Spring Harbor Laboratory, New York
He et al. (2000) Transgenic Res. 9: 223-227
Heim et al. (2003) Mol. Biol. Evol. 20: 735-747
Hein (1990) Methods Enzymol. 183: 626-645
Hempel et al. (1997) Development 124: 3845-3853
Henikoff and Henikoff (991) Nucleic Acids Res. 19: 6565-6572
Henikoff and Henikoff (1992) Proc. Natl. Acad. Sci. 89: 10915-10919
Herrera-Estrella et al. (1983) Nature 303: 209
Hiei et al. (1994) Plant J. 6:271-282
Hiei et al. (1997) Plant Mol. Biol. 35:205-218
Higgins and Sharp (1988) Gene 73: 237-244
Higgins et al. (1996) Methods Enzymol. 266: 383-402
Hohn et al. (1982) Molecular Biology of Plant Tumors Academic Press, New York, N.Y., pp. 549-560
Horsch et al. (1984) Science 233: 496-498
Ichikawa et al. (1997) Nature 390 698-701
Isaacson et al. (2002) Plant Cell 14: 333-342
Isalan et al. (2001) Nature Biotechnol. 19: 656-660
Ishida (1990) Nature Biotechnol 14:745-750
Ishida et al. (1996) Nature Biotechnol. 14: 745-750
Izant and Weintraub (1985) Science 229: 345-352
Jakoby et al. (2002) Trends Plant Sci. 7: 106-111
Jaglo et al. (1998) Plant Physiol 127: 910-917
Jaglo et al. (2001) Plant Physiol. 127: 910-917
Jones et al. (1992) Transgenic Res. 1: 285-297
Kaiser et al. (1995) Plant Mol. Biol. 28: 231-243
Kakimoto et al. (1996) Science 274: 982-985
Kaneko et al. (1999) DNA Res. 6: 183-195
Kang et al. (2000) Plant J. 21: 329-339
Karlin and Altschul (1993) Proc. Natl. Acad. Sci. 90: 5873-5787
Kashima et al. (1985) Nature 313:402-404
Kawata et al. (1992) Nucleic Acids Res. 20: 1141
Kempin et al. (1997) Nature 389: 802-803
Kerstetter (2001) Nature 411: 706-709
Kim and Wold (1985) Cell 42: 129-138
Kim et al. (2001) Plant J. 25: 247-259
Kim et al. (2003) Plant J. 36: 94-104
Kimmel (1987) Methods Enzymol. 152: 507-511
Klann et al. (1996) Plant Physiol. 112: 1321-1330
Klee (1985) Bio/Technology 3: 637-642
Klein et al. (1987) Nature 327: 70-73
Knaap et al. (2000) Plant Physiol. 122: 695-704
Koncz et al. (1992a) Methods in Arabidopsis Research, World Scientific, River Edge, N.J.
Koncz et al. (1992b) Plant Molec. Biol. 20: 963-976
Kop et al. (1999) Plant Mol. Biol. 39: 979-990
Kosugi and Ohashi (2002) Plant J. 29: 45-59
Kranz et al. (1998)Plant J. 16:263-276
Ku et al. (2000) Proc. Natl. Acad. Sci. 97: 9121-9126
Kuhlemeier et al. (1989) Plant Cell 1: 471-478
Kyozuka and Shimamoto (2002) Plant Cell Physiol. 43: 130-135
Ledger et al. (2001) Plant J. 26: 15-22
Lee (1998) Proc. Natl. Acad. Sci. USA 95: 2001-2004
Lee et al. (2002) Genome Res. 12: 493-502
Lehming et al (1987) EMBO J. 6: 3145-3153
Lichtenthaler (1999) Annu. Rev. Plant. Physiol. Plant. Mol. Biol. 50: 47-65
Lichtenthaler et al. (1997) FEBS Lett. 400: 271-274
Lin et al. (1991) Nature 353: 569-571
Liu et al. (2001) J. Biol. Chem. 276: 11323-11334
Liu et al. (2002) Proc. Natl. Acad. Sci. USA 99: 13302-13306
Long and Barton (1998) Development 125: 3027-3035
Long and Barton (2000) Dev. Biol. 218: 341-353
Lu and Ferl (1995) Plant Physiol. 109: 723
Ma and Ptashne (1987) Cell 51: 113-119
Mandel et al. (1992a) Nature 360: 273-277
Mandel et al. (1992b) Cell 71: 133-143
Manners et al. (1998) Plant Mol. Biol. 38: 1071-1080
Matthes et al. (1984) EMBO J. 3: 801-805
Mehta et al. (2002) Nature Biotechnol. 20: 613-618
Melton (1985) Proc. Natl. Acad. Sci. 82: 144-148
Meyers (1995) Molecular Biology and Biotechnology, Wiley VCH, New York, N.Y., p 856-853
Miao et al. (1995) Plant J. 7: 887-896
Montgomery et al. (1993) Plant Cell 5: 1049-1062
Moore et al. (1998) Proc. Natl. Acad. Sci. 95: 376-381
Moore et al. (2002) J. Exp. Bot. 53: 2023-2030
Mount (2001) in Bioinformatics: Sequence and Genome Analysis Cold Spring Harbor Laboratory Press, Cold Spring Harbor, N.Y., page 543
Müller et al. (2001) Plant J. 28: 169-179
Mullis et al. (1990) PCR Protocols A Guide to Methods and Applications (Innis et al. eds) Academic Press Inc. San Diego, Calif.
Nandi et al. (2000) Curr. Biol. 10: 215-218
Needleman and Wunsch (1970) J. Mol. Biol. 48: 443-453
Nesi et al. (2002). Plant Cell 14: 2463-2479
Nicholass et al. (1995) Plant Mol. Biol. 28: 423-435
Nover et al. (1996) Cell Stress Chaperones 1:215-223
Odell et al. (1985) Nature 313: 810-812
Odell et al. (1994) Plant Physiol. 106: 447-458
Ohl et al. (1990) Plant Cell 2: 837-848
Oeller et al. (1991) Science 254: 437-439
Okamuro et al. (1997) Proc. Natl. Acad. Sci. USA 94: 7076-7081
O'Neil et al. (1990) Science 250: 646-651
Ooka et al. (2003). DNA Res. 10: 239-247
Ori et al. (2000) Development 127: 5523-5532
Paddison et al. (2002) Genes & Dev. 16:948-958
Pearson and Lipman (1988) Proc. Natl. Acad. Sci. 85: 2444-2448
Peng et al. (1997) Genes Development 11: 3194-3205
Peng et al. (1999) Nature 400: 256-261
Peng et al. (1999) Nature 400: 256-261
Piazza et al. (2002) Plant Physiol. 128: 1077-1086
Preiss et al. (1985) Nature 313: 27-32
Putterill et al. (1997) Plant Physiol. 114: 396
Ratcliffe et al. (2001) Plant Physiol. 126: 122-132
Remm et al. (2001) J. Mol. Biol. 314: 1041-1052
Riechmann et al. (2000) Science 290: 2105-2110
Rieger et al. (1976) Glossary of Genetics and Cytogenetics: Classical and Molecular, 4th ed., Springer Verlag, Berlin
Ringli and Keller (1998) Plant Mol. Biol. 37: 977-988
Robson et al. (2001) Plant J. 28: 619-631
Ronen et al. (1999) Plant J. 17: 341-351
Rose and Bennett (1999) Trends Plant Sci. 4: 176-183
Rosenberg et al. (1985) Nature 313: 703-706
Rubio et al. (2001) Genes Devel. 15: 2122-2133
Sabatini et al (2003) Genes Dev. 17: 354-358
Sadowski et al. (1988) Nature 335: 563-564
Sambrook et al. (1989) Molecular Cloning: A Laboratory Manual, 2nd Ed., Vol. 1-3, Cold Spring Harbor Laboratory, Cold Spring Harbor, N.Y. (“Sambrook”)
Sakuma et al. (2002) Biochem. Biophys. Res. Commun. 290: 998-1009
Schaffner and Sheen (1991) Plant Cell 3: 997-1012
Schellmann et al. (2002) EMBO J. 21: 5036-5046
Sharp (1999) Genes and Development 13: 139-141
Shewmaker et al. (1999) Plant J. 20: 401-412
Shi et al. (1998) Plant Mol. Biol. 38: 1053-1060
Shimamoto et al. (1989) Nature 338: 274-276
Shpaer (1997) Methods Mol. Biol. 70: 173-187
Siebertz et al. (1989) Plant Cell 1: 961-968
Sjodahl et al. (1995) Planta 197: 264-271
Smith and Waterman (1981) Adv. Appl. Math. 2: 482-489
Smith et al. (1988) Nature, 334: 724-726
Smith et al. (1990) Plant Mol. Biol. 14: 369-379
Smith et al. (1992) Protein Engineering 5: 35-51
Sonnhammer et al. (1997) Proteins 28: 405-420
Stemmer (1994a) Nature 370: 389-391
Stemmer (1994b) Proc. Natl. Acad. Sci. 91: 10747-10751
Stracke et al. (2001) Curr. Opin. Plant Biol. 4: 447-456
Suzuki et al. (2001) Plant J. 28: 409-418
Tague and Goodman (1995) Plant Mol. Biol. 28: 267-279
Taylor and Scheuring (1994) Mol. Gen. Genet. 243: 148-157
Thoma et al. (1994) Plant Physiol. 105: 35-45
Thompson et al. (1994) Nucleic Acids Res. 22: 46734680
Timmons and Fire (1998) Nature 395: 854
Toledo-Ortiz et al. (2003) Plant Cell 15: 1749-1770
Tudge (2000) in The Variety of Life, Oxford University Press, New York, N.Y., pp. 547-606
Vasil et al. (1990) Bio/Technol. 8: 429-434
Vasil et al. (1992) Bio/Technol. 10:667-674
Vasil (1993a) Bio/Technology 10: 667674
Vasil et al. (1993b) Bio/Technol. 11:1553-1558
Vasil (1994) Plant Mol. Biol. 25: 925-937
Vrebalov et al. (2002) Science 296: 343-346
Wada et al. (1997) Science 277: 1113-1116
Wahl and Berger (1987) Methods Enzymol. 152: 399-407
Wan and Lemeaux (1994) Plant Physiol. 104: 37-48
Wanner and Gruissem (1991) Plant Cell 3: 1289-1303
Weeks et al. (1993) Plant Physiol. 102: 1077-1084
Weigel and Nilsson (1995) Nature 377: 482-500
Weissbach and Weissbach (1989) Methods for Plant Molecular Biology, Academic Press
Wilkinson et al. (1995) et al. Science 270: 1807-1809
Wilkinson et al. (1997) Nat. Biotechnol. 15: 444-447
Willmott et al. (1998) Plant Molec. Biol. 38: 817-825
Winans (1992) Microbiol. Rev. 56: 12-31
Wu, ed. (993) Methods Enzymol. (vol. 217, Academic Press, San Diego)
Wysocka-Diller et al (2000) Development 127: 595-603
Xu et al. (2001) Proc. Natl. Acad. Sci., USA, 98: 15089-15094
Zamore (2001) Nature Struct. Biol., 8: 746-750
Zhang et al. (1999) Proc. Natl. Acad. Sci. USA 96: 6523-6528
Zhang et al. (2000) J. Biol. Chem. 275: 33850-33860

What is claimed is:

1. A transgenic plant having an altered trait compared to a wild-type plant of the same species, wherein the transgenic plant comprises:
a recombinant polynucleotide having a nucleotide sequence encoding a polypeptide having a conserved domain with at least 80% sequence identity to a conserved domain of SEQ ID NO: 2N, where N=1 to 201 or 413 to 419; and
wherein the altered trait is selected from the group consisting of increased yield, increased fungal disease tolerance, increased fruit weight, increased fruit number, and increased plant size, increased fungal disease tolerance, increased lycopene levels, reduced fruit softening, and increased soluble solids, increased levels of leaf chlorophylls, increased levels of leaf carotenoids, increased volume, and increased biomass.

2. The transgenic plant of claim 1, wherein the transgenic plant has greater vegetative yield than the wild-type plant.

3. The transgenic plant of claim 1, wherein the polypeptide has a conserved domain with at least 85% sequence identity to the conserved domain of SEQ ID NO: 2N, where N=1 to 201 or 413 to 419.

4. The transgenic plant of claim 1, wherein the polypeptide has a conserved domain with at least 88% sequence identity to the conserved domain of SEQ ID NO: 2N, where N=1 to 201 or 413 to 419.

5. The transgenic plant of claim 1, further comprising a constitutive, inducible, or tissue-specific promoter operably linked to said nucleotide sequence.

6. The transgenic plant of claim 5, wherein the constitutive, inducible, or tissue-specific promoter is a LIPID TRANSFER PROTEIN 1 promoter or a POLYGALACTURONASE promoter.

7. The transgenic plant of claim 1, wherein the transgenic plant is a tomato plant.

8. Seed produced from the transgenic plant according to claim 1, wherein the seed comprises the recombinant polynucleotide of claim 1.

9. A method for producing a transgenic plant, wherein
(a) a plant cell is genetically modified by integrating into the nuclear genome of said plant cell a recombinant polynucleotide encoding a polypeptide having a conserved domain with at least 80% sequence identity to a conserved domain of SEQ ID NO: 2N, where N=1 to 201 or 413 to 419; and
(b) a transgenic plant is generated from the plant cell produced according to step (a);
wherein expression of said polypeptide results in increased yield, increased fungal disease tolerance, increased fruit weight, increased fruit number, and increased plant size, increased fungal disease tolerance, increased lycopene levels, reduced fruit softening, and increased soluble solids, increased levels of leaf chlorophylls, increased levels of leaf carotenoids, increased volume, and increased biomass of the transgenic plant in comparison to a wild-type plant of the same species.

10. The method of claim 9, wherein the transgenic plant has greater vegetative yield than the wild-type plant.

11. The method of claim 9, wherein the polypeptide has a conserved domain with at least 85% sequence identity to the conserved domain of SEQ ID NO: 2N, where N=1 to 201 or 413 to 419.

12. The method of claim 9, wherein the polypeptide has a conserved domain with at least 88% sequence identity to the conserved domain of SEQ ID NO: 2N, where N=1 to 201 or 413 to 419.

13. The method of claim 9, further comprising a constitutive, inducible, or tissue-specific promoter operably linked to said nucleotide sequence.

14. The method of claim 13, wherein the constitutive, inducible, or tissue-specific promoter is a LIPID TRANSFER PROTEIN 1 promoter or a POLYGALACTURONASE promoter.

15. The method of claim 9, wherein the transgenic plant is a tomato plant.

16. The method of claim 9, the method steps further comprising:
(c) selfing or crossing the transgenic plant with itself or another plant, respectively, to produce seed; and
(d) growing a progeny plant from the seed.

17. Seed produced from a transgenic plant produced according to the method of claim 9, wherein the seed comprises the recombinant polynucleotide of claim 9.
